(19) United States
(12) Patent Application Publication
(10) Pub. No.: US 2024/0307438 A1
(43) Pub. Date: Sep. 19, 2024

(54) Regulatory T Cell (Treg) Extracellular Vesicle Compositions and Methods

(71) Applicant: The Methodist Hospital, Houston, TX (US)

(72) Inventors: Stanley Hersh Appel, Houston, TX (US); Aaron Drew Thome, Richmond, TX (US); Jason Robert Thonhoff, Houston, TX (US)

(21) Appl. No.: 18/278,736

(22) PCT Filed: Feb. 25, 2022
(86) PCT No.: PCT/US2022/017990

§ 371 Date: Aug. 24, 2023

Related U.S. Application Data
(60) Provisional application No. 63/208,395, filed on Jun. 8, 2021.
(60) Provisional application No. 63/154,449, filed on Feb. 26, 2021.

(51) Int. Cl. A61K 35/17; A61P 29/00; C12N 5/0783

(52) U.S. Cl. CPC A61K 35/17; A61P 29/00; C12N 5/0637

(57) ABSTRACT

The present disclosure provides anti-inflammatory extracellular vesicles (EVs) derived from ex vivo-expanded human suppressive immune cells. e.g., regulatory T cells (Tregs). Such EVs are useful in the treatment of diseases such as amyotrophic lateral sclerosis (AES). Alzheimer's disease, and other neurological diseases, as well as inflammatory and autoimmune diseases or dysfunctions.

### CROSS-REFERENCE TO RELATED APPLICATIONS

This application claims the benefit of U.S. Provisional Application No. 63/208,395, filed Jun. 8, 2021, and U.S. Provisional Application No. 63/154,449, filed Feb. 26, 2021, each of which is incorporated by reference herein in its entirety.

### 1. FIELD

The present disclosure provides anti-inflammatory and restorative extracellular vesicles (EVs) that are derived from ex vivo-expanded human suppressive immune cells, e.g., regulatory T cells (Tregs) and that are useful in the treatment of diseases such as amyotrophic lateral sclerosis (ALS), Alzheimer's disease, and other neurological diseases, as well as inflammatory, metabolic, and autoimmune diseases or dysfunctions.

### 2. BACKGROUND

Inflammatory and neuroinflammatory mechanisms contribute to a wide variety of devastating diseases, including such neurodegenerative diseases as amyotrophic lateral sclerosis (ALS), Parkinson's disease and multiple sclerosis. Neurodegenerative diseases such as this direct a tremendous health and economic burden that will only exacerbate further over time.

Currently, no disease-modifying treatments for such diseases are available. Anti-inflammatory treatments have been utilized for decades in attempting to ameliorate a multitude of neurodegenerative diseases. Little progress, however, has been made with single drug/target approaches.

Increasingly, studies point to immune system involvement in the etiology of diseases such as this, and point to dysfunction of immune cells as a chief mediator of disease pathogenesis. The complex signaling mechanisms and built-in redundancies of the immune system and its constituents may help explain the ineffectiveness of such single drug/single target anti-inflammatory approaches.

Recently great promise has been demonstrated with regulatory T cell (Treg) cell therapy, which may represent a more global approach to suppressing immune system dysfunction contributing to disease. For example, clinical trials involving administration of expanded autologous Tregs to ALS patients report that the Treg therapy slowed progression rates during early and later stages of the disease, and that Treg suppressive function correlated with the slowing of disease progression (Thonhoff, J. R. et al., 2018, Neurology-Neuroimmunology Neuroinflammation 5(4)).

Nonetheless, there still exists a need for development of additional treatments that can suppress inflammatory and/or promote anti-inflammatory immune system components, and can do so in the pro-inflammatory, toxic microenvironment of the disease state.

### 3. SUMMARY

Presented herein are extracellular vesicles (EVs) that exhibit impressive anti-inflammatory activity, both in vitro and in vivo. The EVs presented herein are derived from ex vivo-expanded human suppressive immune cells, for example regulatory T cells (Tregs). As demonstrated herein, the EVs of the present disclosure retain the immune suppressive activities of the cells from which they are derived. Moreover, as EVs are not themselves cells, they avoid potential cell-based issues such as immune rejection and the possibility of polarization to a pro-inflammatory cell type. As such, the anti-inflammatory EVs presented herein are particularly useful for treatment of a variety of diseases such as, for example, neurodegenerative disorders such as amyotrophic lateral sclerosis (ALS).

Results presented herein demonstrate that the EVs of the present disclosure are able to potently suppress T responder cell proliferation and pro-inflammatory myeloid, e.g., macrophage, activity in vitro, and also exert potent anti-inflammatory effects in vivo, via either intravenous or intranasal administration. For example, in vivo results presented herein using anti-inflammatory Treg EV compositions of the disclosure demonstrate an anti-inflammatory effect in a model of inflammation and a motor neuron degenerative disease modeling ALS. For example, results presented herein demonstrate that the EVs are able to suppress brain and peripheral inflammation in an in vivo model of neuroinflammation, and are also able to suppress inflammation and extend survival in an in vivo model of amyotrophic lateral sclerosis (ALS). The results presented herein also demonstrate that the Treg EVs have a greater suppressive effect on pro-inflammatory immune cells than EVs derived from mesenchymal stem cells (MSCs).

Moreover, the anti-inflammatory EVs presented herein exhibit remarkable batch-to-batch consistency in size, stability and activity and exhibit a unique structural signature as, for example, characterized by Treg EV surface marker and RNA profiles. Still further, as demonstrated herein, the methods presented herein yield potent anti-inflammatory EVs exhibiting similar structural and suppressive activity characteristics whether the original Treg starting material is obtained from healthy subjects or ALS patients.

In one aspect, presented herein is an isolated, cell-free population of anti-inflammatory extracellular vesicles (EVs), wherein the anti-inflammatory EVs are derived from ex vivo-expanded human suppressive immune cells, wherein: i) the population exhibits a size diameter distribution of about 50 nm to about 150 nm; ii) the population comprises EV surface CD2, CD25 and HLA-DRDPDQ; iii) the population comprises hsa-miR-1290, hsa-miR-146a-5p, and hsa-miR-155-5p micro-RNAs (miRNAs); and iv) the population exhibits an ability to suppress myeloid cells, for example, macrophages, as measured by an ability to reduce pro-inflammatory cytokine production by the myeloid cells (e.g., exhibit an ability to decrease the expression of IL-6, IL-8, IL1β or Interferon-γ in the myeloid cells) and an ability to increase the expression of one or more anti-inflammatory markers in the myeloid cells (e.g., an ability to increase the expression of IL-10, Arg1 and/or CD206 in the myeloid cells), or as measured by an ability to suppress proliferation of responder T cells; and wherein the human suppressive immune cells are regulatory T cells (Tregs). In certain embodiments, the human Tregs are from a healthy human subject. In certain embodiments, the human Tregs are from a human subject diagnosed with or suspected of having Amyotrophic Lateral Sclerosis (ALS).

In certain embodiments, the population of anti-inflammatory EVs further comprises EV surface CD44, CD29, CD4 and CD45. In certain embodiments, the population of anti-inflammatory EVs further comprises EV surface CD44, CD29, CD4 and CD45. In certain embodiments, the population of anti-inflammatory EVs further comprises EV surface CD9, CD63 and CD81. In certain embodiments, the population of anti-inflammatory EVs substantially lacks EV surface CD3, CD19, CD8, CD56, CD105, CD1c, CD49e, ROR1, CD209, SSEA-4, CD40, CD62P, CD11c, CD40, MSCP, CD146, CD86, CD326, CD133, CD142, CD31 and CD14. In certain embodiments, the population of anti-inflammatory EVs further comprises EV surface CD44, CD29, CD4 and CD45. In certain embodiments, the population of anti-inflammatory EVs further comprises EV surface CD44, CD29, CD4 and CD45, CD9, CD63 and CD81, and ii) substantially lacks EV surface CD3, CD19, CD8, CD56, CD105, CD1c, CD49e, ROR1, CD209, SSEA-4, CD40, CD62P, CD11c, CD40, MSCP, CD146, CD86, CD326, CD133, CD142, CD31 and CD14.

In certain embodiments, the ratio of hsa-miR-146a-5p to hsa-miR-155-5p present in the population of anti-inflammatory EVs is about 2 to about 3. In certain embodiments, the abundance of hsa-miR-1290 in the population of anti-inflammatory EVs is at least 2-fold that of hsa-mir-155-5p. In specific embodiments, the ratio of hsa-miR-146a-5p to hsa-miR-155-5p present in the population of anti-inflammatory EVs is about 2 to about 3 and the abundance of hsa-miR-1290 in the population of anti-inflammatory EVs is at least 2-fold that of hsa-mir-155-5p.

In particular embodiments, at least about 90% of the EVs of the population of anti-inflammatory exhibit a size diameter of about 50 nm to about 150 nm. In certain embodiments, the population of anti-inflammatory EVs exhibits a mean size diameter of about 80 nm to about 110 nm. In certain embodiments, the population of anti-inflammatory EVs exhibits a median size diameter of about 70 nm to about 110 nm. In certain embodiments, the population of anti-inflammatory EVs exhibits a mode size diameter of about 65 nm to about 95 nm. In specific embodiments, at least about 90% of the EVs in the population of anti-inflammatory EVs exhibit a size diameter of about 50 to about 150 nm, and the population exhibits a mean size diameter of about 80 nm to about 110 nm, a median size diameter of about 70 nm to about 110 nm, and a mode size diameter of about 65 nm to about 95 nm.

Presented herein are isolated, cell-free populations of anti-inflammatory EVs, wherein the anti-inflammatory EVs are derived from ex vivo-expanded human suppressive immune cells, for example regulatory T cells (Tregs). Also presented herein are pharmaceutical compositions and cryopreserved compositions comprising an isolated, cell-free population of anti-inflammatory EVs described herein, methods of producing the EV populations and methods of using the EVs for treatment of diseases, such as neurodegenerative diseases, e.g., ALS.

In one aspect, provided herein is an isolated, cell-free population of anti-inflammatory extracellular vesicles (EVs), wherein the anti-inflammatory EVs are derived from ex vivo-expanded human suppressive immune cells. In some embodiments, the human suppressive immune cells are regulatory T cells (Tregs). In some embodiments, the Tregs are from a healthy human subject.

In some embodiments, the Tregs are from a human subject diagnosed with or suspected of having a neurodegenerative disorder. In some embodiments, the neurodegenerative disorder is Alzheimer's disease. In some embodiments, the neurodegenerative disorder is Amyotrophic Lateral Sclerosis (ALS). In some embodiments, the neurodegenerative disease is multiple sclerosis (MS). In some embodiments, the neurodegenerative disease is Parkinson's Disease.

In some embodiments, the Tregs are from a human subject who is diagnosed as having, or suspected of having had, a stroke.

In some embodiments, the Tregs are from a geriatric human subject.

In some embodiments, the Tregs are from multiple human subjects. In some embodiments, the Tregs are from multiple unrelated human subjects.

In some embodiments, the anti-inflammatory EVs exhibit an ability to increase the expression of one or more anti-inflammatory markers in inflammatory cells. In some embodiments, the inflammatory cells are myeloid cells. In some embodiments, the anti-inflammatory EVs exhibit an ability to increase the expression of IL-10, Arg1 and/or CD206 in inflammatory cells.

In some embodiments, the anti-inflammatory EVs exhibits an ability to suppress inflammatory cells, as measured by pro-inflammatory cytokine production by the inflammatory cells. In some embodiments, the inflammatory cells are myeloid cells. In some embodiments, the myeloid cells are monocytes, macrophages, or microglia. In some embodiments, the macrophages are M1 macrophages. In some embodiments, the M1 macrophages are induced pluripotent stem cell (iPSC)-derived M1 macrophages.

In some embodiments, the ability to suppress inflammatory cells is measured by IL-6, IL-8, TNFα, IL1β and/or Interferon-γ production by the inflammatory cells.

In some embodiments, the anti-inflammatory EVs exhibit an ability to increase the expression of IL-1-, Arg1 and/or CD206 and an ability to suppress IL-6, IL-8, TNFα, IL1β and/or Interferon-γ production in inflammatory cells, e.g., myeloid cells, for example, macrophages.

In some embodiments, the anti-inflammatory EVs exhibit a suppressive function, as determined by suppression of proliferation of responder T cells. In some embodiments, the proliferation of responder T cells is determined by flow cytometry or thymidine incorporation.

In some embodiments, the population is a saline-containing population of anti-inflammatory EVs. In some embodiments, the population is a physiological saline-containing population of anti-inflammatory EVs. In some embodiments, the population is a phosphate-buffered saline-containing population of anti-inflammatory EVs.

In some embodiments, the population of anti-inflammatory EVs comprises exosomes and microvesicles. In some embodiments, the majority of the EVs are exosomes. In some embodiments, at least about 80%, about 90%, or about 95% of the EVs are exosomes. In some embodiments, the majority of the EVs are microvesicles. In some embodiments, at least about 80%, about 90%, or about 95% of the EVs are microvesicles.

In some embodiments, the population of anti-inflammatory EVs comprises at least about 50% exosomes. In some embodiments, at least about 60% of the EVs are exosomes. In some embodiments, at least about 70% of the EVs are exosomes.

In some embodiments, the population of anti-inflammatory EVs comprises at least about 50% microvesicles. In some embodiments, at least about 60% of the EVs are microvesicles. In some embodiments, at least about 70% of the EVs are microvesicles.

In some embodiments, the majority of the EVs in a population of anti-inflammatory EVs provided herein have diameters from about 30 nm to about 1000 nm. In some embodiments, the majority of the EVs have diameters from about 30 nm to about 100 nm, about 30 nm to about 150 nm, about 30 to about 200 nm, about 40 to about 100 nm, about 80 to about 100 nm, about 80 to about 110 nm, about 80 to about 125 nm, or about 100 to about 120 nm. In some embodiments, the majority of the EVs have diameters from about 60 nm to about 1000 nm, about 70 nm to about 1000 nm, about 80 nm to about 1000 nm, 100 to about 1000 nm, about 200 to about 1000 nm, or about 300 to about 1000 nm. In some embodiments, the majority of the EVs have diameters from about 20 nm to about 300 nm, about 20 nm to about 275 nm, about 20 to about 250 nm, about 20 to about 200 nm, or about 20 nm to about 175 nm.

In another aspect, provided herein is a pharmaceutical composition comprising an isolated, cell-free population of anti-inflammatory EVs provided herein. In certain embodiments, the pharmaceutical composition comprising an isolated, cell-free population of anti-inflammatory EVs provided herein in saline. In some embodiments, the population of anti-inflammatory EVs comprises about 1×10⁶ to about 1×10¹⁴ EVs, about 1×10⁸ to about 1×10¹⁴ EVs, about 1×10⁸ to about 1×10¹² EVs, about 1×10⁸ to about 1×10¹⁰ EVs, about 1×10¹⁰ to about 1×10¹⁴ EVs, or about 1×10¹⁰ to about 1×10¹² EVs. In some embodiments, the population of anti-inflammatory EVs comprises about 1×10⁹ EVs, about 5×10⁹ EVs, about 1×10¹⁰ EVs, about 5×10¹⁰ EVs, about 1×10¹¹ EVs, about 5×10¹¹ EVs, or about 1×10¹² EVs. In some embodiments, the population of anti-inflammatory EVs comprises about 1×10⁶ to about 1×10¹⁴ EVs/ml, about 1×10⁸ to about 1×10¹⁴ EVs/ml, about 1×10⁸ to about 1×10¹² EVs/ml, about 1×10⁸ to about 1×10¹⁰ EVs/ml, about 1×10¹⁰ to about 1×10¹⁴ EVs/ml, or about 1×10¹⁰ to about 1×10¹² EVs/ml. In some embodiments, the population of anti-inflammatory EVs comprises about 5×10⁸ EVs/ml, about 1×10⁹ EVs/ml, about 2.5×10⁹ EVs/ml, about 5×10⁹ EVs/ml, about 1×10¹⁰ EVs/ml, about 2.5×10¹⁰ EVs/ml, about 5×10¹⁰ EVs/ml, about 1×10¹¹ EVs/ml, about 2.5×10¹¹ EVs/ml, about 5×10¹¹ EVs/ml, or about 1×10¹² EVs/ml.

In some embodiments, the population of anti-inflammatory EVs comprises in a pharmaceutical composition provided herein comprises about 1 μg to about 200 mg EVs. In some embodiments, the population of anti-inflammatory EVs comprises about 1 μg to about 15 mg EVs. In some embodiments, the population of anti-inflammatory EVs comprises about 1 μg to about 15 mg EV/ml.

In some embodiments, the pharmaceutical composition is a cryopreserved pharmaceutical composition. In some embodiments, the pharmaceutical composition had previously been cryopreserved.

In another aspect, provided herein is a cryopreserved composition comprising an isolated, cell-free population of anti-inflammatory EVs provided herein.

In another aspect, provided herein is a method of producing an isolated, cell-free population of anti-inflammatory extracellular vesicles (EVs), said method comprising the steps of: (a) ex-vivo expanding a human suppressive immune cell population in culture media to produce a culture comprising the cells, the culture media and anti-inflammatory EVs; and (b) isolating the anti-inflammatory EVs from the culture. In some embodiments, the human suppressive immune cell population is a population of regulatory T cells (Tregs).

In some embodiments, step (b) comprises removing cells from the culture, followed by polyethylene glycol precipitation of the culture. In some embodiments, step (b) comprises: (i) removing the cells from the culture to produce a cell-free, anti-inflammatory EV-containing solution; and (ii) isolating the anti-inflammatory EVs from the cell-free, anti-inflammatory EV-containing solution of (i).

In some embodiments, step (i) comprises passing the culture through a filter such that the cells are retained by the filter, and thereby removed from the culture. In some embodiments, step (i) comprises microfiltration.

In some embodiments, step (ii) comprises step (ii-a): passing the cell-free, anti-inflammatory EV-containing solution through a filter such that the anti-inflammatory EVs are retained by the filter. In some embodiments, the filter has a molecular weight cut-off (MWCO) of about 200 kilodaltons (kDa) to about 600 kDa. In some embodiments, the filter has an MWCO of about 500 kDa.

In some embodiments, step (ii) comprises ultrafiltration. In some embodiments, step (ii) further comprises step (ii-b): performing buffer exchange such that the isolated, cell-free population of anti-inflammatory EVs produced is a buffer-containing isolated, cell-free population of anti-inflammatory EVs. In some embodiments, the buffer is a saline-containing buffer. In some embodiments, the saline-containing buffer is physiological saline. In some embodiments, the saline-containing buffer is PBS.

In some embodiments, step (ii-b) comprises diafiltration.

In some embodiments, steps (ii-a) and (ii-b) are performed simultaneously.

In some embodiments, step (b) comprises tangential flow filtration.

In some embodiments, the culture media in step (a) is serum-free. In some embodiments, the culture media in step (a) comprises serum. In some embodiments, the serum is human AB serum. In some embodiments, the serum is depleted for serum-derived EVs.

In some embodiments, a method of producing an isolated, cell-free population of anti-inflammatory EVs further comprises, prior to step (a), the step of enriching Tregs from a cell sample suspected of containing Tregs, to produce a baseline Treg cell population that is the population of Tregs that is then expanded in step (a). In some embodiments, the cell sample is a leukapheresis cell sample. In some embodiments, the method further comprises obtaining the cell sample from a donor by leukapheresis. In some embodiments, the cell sample is not stored overnight or frozen before carrying out the enriching step. In some embodiments, the cell sample is obtained within 30 minutes before initiation of enriching step. In some embodiments, the enriching step comprises depleting CD8+/CD19+ cells then enriching for CD25+ cells. In some embodiments, step (a) is carried out within 30 minutes of the enriching step.

In some embodiments, step (a) of a method of producing an isolated, cell-free population of anti-inflammatory EVs comprises culturing the Tregs in a culture media that comprises beads coated with anti-CD3 antibodies and anti-CD28 antibodies. In some embodiments, the beads are first added to the culture media within about 24 hours of the initiation of the culturing. In some embodiments, beads coated with anti-CD3 antibodies and anti-CD28 antibodies are added to the culture media about 14 days after beads coated with anti-CD3 antibodies and anti-CD28 antibodies were first added to the culture medium.

In some embodiments, step (a) further comprises adding IL-2 to the culture medium within about 6 days of the initiation of culturing. In some embodiments, step (a) further comprises replenishing the culture medium with IL-2 about every 2-3 days after IL-2 is first added to the culture medium.

In some embodiments, step (a) further comprises adding rapamycin to the culture medium within about 24 hours of the initiation of the culturing. In some embodiments, step a) further comprises replenishing the culture medium with rapamycin every 2-3 days after the rapamycin is first added to the culture medium.

In some embodiments, step (a) is automated. In some embodiments, step a) takes place in a bioreactor.

In some embodiments, step (b) of a method of producing an isolated, cell-free population of anti-inflammatory EVs may commence at any point during step a).

In some embodiments, the Tregs enriched in step (a) are from a healthy human subject. In some embodiments, the Tregs are from a human subject diagnosed with or suspected of having a neurodegenerative disorder. In some embodiments, the neurodegenerative disorder is Alzheimer's disease, Amyotrophic Lateral Sclerosis (ALS), multiple sclerosis (MS), or Parkinson's Disease. In some embodiments, the Tregs are from a human subject who is diagnosed as having, or suspected of having had, a stroke. In some embodiments, the Tregs are from a geriatric human subject. In some embodiments, the Tregs are from multiple human subjects.

In some embodiments, the human suppressive immune cell population expanded in step (a) is a genetically engineered human suppressive immune cell population.

In some embodiments, the population of Tregs expanded in step (a) is a genetically engineered population of Tregs.

In another aspect, provided herein is a pharmaceutical composition comprising an isolated, cell-free population of anti-inflammatory EVs, wherein the population is made by any one of the methods described herein.

In some embodiments, a method of producing an isolated, cell-free population of anti-inflammatory EVs further comprises (c) cryopreserving the isolated, cell-free population of anti-inflammatory EVs, thereby producing a cryopreserved, isolated, cell-free population of anti-inflammatory EVs. Also presented herein are cryopreserved compositions comprising an isolated, cell-free population of anti-inflammatory EVs, wherein the cryopreserved compositions are made using such methods.

In some embodiments, the method further comprises thawing the cryopreserved, isolated cell-free population of anti-inflammatory EVs after cryopreservation for about 1 week, 1 month, about 3 months, about 6 months, about 9 months, about 12 months, about 18 months or about 24 months. Also provided herein are compositions, for example, pharmaceutical compositions, comprising an isolated, cell-free population of anti-inflammatory EVs, wherein the compositions, for example, pharmaceutical compositions, are made using such methods.

In another aspect, provided herein is an isolated, cell-free population of anti-inflammatory EVs, wherein the anti-inflammatory EVs are derived from an ex vivo-expanded Treg cell population that exhibits an ability to suppress inflammatory cells, as measured by pro-inflammatory cytokine production by the inflammatory cells, wherein the inflammatory cells are macrophages or monocytes from human donors or generated from induced pluripotent stem cells, wherein the ex vivo-expanded Treg cell population has been expanded from baseline Tregs, and wherein, in the ex vivo-expanded Treg cell population: (a) expression of one or more dysfunctional baseline signature gene products listed in Table 3 and/or Table 4 is decreased relative to the expression of the one or more gene products in baseline Tregs; (b) expression of one or more dysfunctional baseline signature gene products listed in Table 5 is decreased relative to the expression of the one or more gene products in baseline Tregs; (c) expression of one or more Treg-associated signature gene products listed in Table 6 is increased relative to the expression of the one or more gene products in baseline Tregs; (d) expression of one or more mitochondria signature gene products listed in Table 7 is increased relative to the expression of the one or more gene products in baseline Tregs; (e) expression of one or more cell proliferation signature gene products listed in Table 8 is increased relative to the expression of the one or more gene products in baseline Tregs; or (f) expression of one or more highest protein expression signature gene products listed in Table 9 is increased relative to the expression of the one or more gene products in baseline Tregs. In some embodiments, provided herein is a pharmaceutical composition comprising the isolated, cell-free population of anti-inflammatory EVs.

In another aspect, provided herein is a method of treating a disorder associated with Treg dysfunction, the method comprising administering to a subject in need of said treatment a pharmaceutical composition provided herein.

In another aspect, provided herein is a method of treating a disorder associated with Treg deficiency, the method comprising administering to a subject in need of said treatment a pharmaceutical composition provided herein.

In another aspect, provided herein is a method of treating a disorder associated with overactivation of the immune system, the method comprising administering to a subject in need of said treatment a pharmaceutical composition provided herein.

In another aspect, provided herein is a method of treating an inflammatory condition driven by a T cell response, the method comprising administering to a subject in need of said treatment a pharmaceutical composition provided herein.

In another aspect, provided herein is a method of treating an inflammatory condition driven by a myeloid cell response, the method comprising administering to a subject in need of said treatment a pharmaceutical composition provided herein. In some embodiments, the myeloid cell is a monocyte, macrophage or microglia.

In another aspect, provided herein is a method of treating a neurodegenerative disorder in a subject in need thereof, the method comprising administering to a subject in need of said treatment a pharmaceutical composition provided herein. In some embodiments, the neurodegenerative disease is ALS, Alzheimer's disease, Parkinson's disease, frontotemporal dementia or Huntington's disease.

In some embodiments, the neurodegenerative disease is ALS, Alzheimer's disease, Parkinson's disease, frontotemporal dementia, multiple sclerosis or Huntington's disease.

In another aspect, provided herein is a method of treating an autoimmune disorder in a subject in need thereof, the method comprising administering to a subject in need of said treatment a pharmaceutical composition provided herein. In some embodiments, the autoimmune disorder is polymyositis, ulcerative colitis, inflammatory bowel disease, Crohn's disease, celiac disease, systemic sclerosis (scleroderma), multiple sclerosis (MS), rheumatoid arthritis (RA), Type I diabetes, psoriasis, dermatomyosititis, lupus, e.g., systemic lupus erythematosus, or cutaneous lupus, myasthenia gravis, autoimmune nephropathy, autoimmune hemolytic anemia, autoimmune cytopenia, autoimmune encephalitis, autoimmune hepatitis, autoimmune uveitis, alopecia, thyroiditis or pemphigus.

In another aspect, provided herein is a method of treating graft-versus-host disease in a subject in need thereof, the method comprising administering to a subject in need of said treatment a pharmaceutical composition provided herein. In some embodiments, the subject has received a bone marrow transplant, kidney transplant or liver transplant.

In another aspect, provided herein is a method of improving islet graft survival in a subject in need thereof, the method comprising administering to a subject in need of said treatment a pharmaceutical composition provided herein.

In another aspect, provided herein is a method of treating cardio-inflammation in a subject in need thereof, the method comprising administering to a subject in need of said treatment a pharmaceutical composition provided herein. In some embodiments, the cardio-inflammation is associated with atherosclerosis, myocardial infarction, ischemic cardiomyopathy or heart failure.

In another aspect, provided herein is a method of treating neuroinflammation in a subject in need thereof, the method comprising administering to a subject in need of said treatment a pharmaceutical composition provided herein. In some embodiments, the neuroinflammation is associated with stroke, acute disseminated encephalomyelitis, acute optic neuritis, acute inflammatory demyelinating polyradiculoneuropathy, chronic inflammatory demyelinating polyradiculoneuropathy, Guillain-Barre syndrome, transverse myelitis, neuromyelitis optica, epilepsy, traumatic brain injury, spinal cord injury, encephalitis, central nervous system vasculitis, neurosarcoidosis, autoimmune or post-infectious encephalitis or chronic meningitis.

In another aspect, provided herein is a method of treating a Tregopathy in a subject in need thereof, comprising administering to a subject in need of said treatment a pharmaceutical composition provided herein. In some embodiments, the Tregopathy is caused by a FOXP3, CD25, cytotoxic T lymphocyte-associated antigen 4 (CTLA4), LPS-responsive and beige-like anchor protein (LRBA), or BTB domain and CNC homolog 2 (BACH2) gene loss-of-function mutation, or a signal transducer and activator of transcription 3 (STAT3) gain-of-function mutation.

In some embodiments, the anti-inflammatory EVs administered to a subject are derived from Tregs that are autologous to the subject. In some embodiments, the anti-inflammatory EVs are derived from Tregs that are allogeneic to the subject.

In some embodiments, the pharmaceutical composition is administered via intranasal administration. In some embodiments, the intranasal administration is via aerosol inhalation or nasal drip. In some embodiments, the pharmaceutical composition is administered intravenously. In some embodiments, the pharmaceutical composition is administered by local injection.

In some embodiments, a method of treatment provided herein further comprises administering to the subject a pharmaceutical composition comprising a therapeutic population of Tregs, wherein the Tregs had been ex vivo expanded and cryopreserved, and wherein the Tregs are not further expanded prior to the administering. In some embodiments, the therapeutic population of Tregs is autologous to the subject. In some embodiments, the therapeutic population of Tregs is allogeneic to the subject. In some embodiments, the pharmaceutical composition comprising the therapeutic population of Tregs is administered intravenously. In some embodiments, the pharmaceutical composition comprising the anti-inflammatory EVs and the pharmaceutical composition comprising the therapeutic population of Tregs are administered to the patient on the same day.

In certain embodiments, the methods of treatment presented herein comprise administering to a subject in need of treatment a pharmaceutical composition comprising an isolated, cell-free population of anti-inflammatory EVs, wherein the EVs had been cryopreserved and thawed prior to being administered to the subject. In certain embodiments, the methods of treatment presented herein comprise administering to a subject in need of treatment a pharmaceutical composition comprising an isolated, cell-free population of anti-inflammatory EVs, wherein the EVs are stored at 4° C., for example, are stored overnight at 4° C., prior to being administered to the subject. In particular embodiments, the methods of treatment presented herein comprise administering to a subject in need of treatment a pharmaceutical composition comprising an isolated, cell-free population of anti-inflammatory EVs wherein the EVs had been cryopreserved, thawed and stored at 4° C., for example, stored overnight at 4° C., prior to being administered to the subject.

In certain embodiments, the methods of treatment presented herein comprise administering to a subject in need of treatment a pharmaceutical composition comprising an isolated, cell-free population of anti-inflammatory EVs wherein the EVs had undergone at least two freeze/thaw cycles prior to being administered to the subject, e.g., had undergone about 2 to about 20 freeze/thaw cycles prior to being administered to the subject.

Further illustrative embodiments are as follows:

1. An isolated, cell-free population of anti-inflammatory extracellular vesicles (EVs),
wherein the anti-inflammatory EVs are derived from ex vivo-expanded human suppressive immune cells,
wherein:
i) the population exhibits a size diameter distribution of about 50 nm to about 150 nm;
ii) the population comprises EV surface CD2, CD25 and HLA-DRDPDQ;
iii) the population comprises hsa-miR-1290, hsa-miR-146a-5p, and hsa-miR-155-5p micro-RNAs (miRNAs);
iv) the population exhibits an ability to suppress myeloid cells, as measured by an ability to reduce pro-inflammatory cytokine production by the myeloid cells and an ability to increase the expression of one or more anti-inflammatory markers in the myeloid cells, or as measured by an ability to suppress proliferation of responder T cells; and
wherein the human suppressive immune cells are regulatory T cells (Tregs).

2. The population of anti-inflammatory EVs of embodiment 1, wherein at least about 90% of the EVs in the population exhibit a size diameter of about 50 nm to about 150 nm.

3. The population of anti-inflammatory EVs of embodiment 1 or 2, wherein the population exhibits a mean size diameter of about 80 nm to about 110 nm.

4. The population of anti-inflammatory EVs of any one of embodiments 1-3, wherein the population exhibits a median size diameter of about 70 nm to about 110 nm.

5. The population of anti-inflammatory EVs of any one of embodiments 1-4, wherein the population exhibits a mode size diameter of about 65 nm to about 95 nm.

6. The population of anti-inflammatory EVs of embodiment 1, wherein at least about 90% of the EVs in the population exhibit a size diameter of about 50 to about 150 nm, and the population exhibits a mean size diameter of about 80 nm to about 110 nm, a median size diameter of about 70 nm to about 110 nm, and a mode size diameter of about 65 nm to about 95 nm.

7. The population of anti-inflammatory EVs of any one of embodiments 1-6, wherein the population further comprises EV surface CD44, CD29, CD4 and CD45.

8. The population of anti-inflammatory EVs of any one of embodiments 1-7, wherein the population further comprises EV surface CD9, CD63 and CD81.

9. The population of anti-inflammatory EVs of any one of embodiments 1-8, wherein the population substantially lacks EV surface CD3, CD19, CD8, CD56, CD105, CD1c, CD49e, ROR1, CD209, SSEA-4, CD40, CD62P, CD11c, CD40, MSCP, CD146, CD86, CD326, CD133, CD142, CD31 and CD14.

10. The population of anti-inflammatory EVs of embodiment 1 or 6, wherein the population further comprises EV surface CD44, CD29, CD4, CD45, CD9, CD63 and CD81, and wherein the population substantially lacks EV surface CD3, CD19, CD8, CD56, CD105, CD1c, CD49e, ROR1, CD209, SSEA-4, CD40, CD62P, CD11c, CD40, MSCP, CD146, CD86, CD326, CD133, CD142, CD31 and CD14.

11. The population of anti-inflammatory EVs of any one of embodiments 1-10, wherein the ratio of hsa-miR-146a-5p to hsa-miR-155-5p in the population is about 2 to about 3.

12. The population of anti-inflammatory EVs of any one of embodiments 1-11, the abundance of hsa-miR-1290 is at least 2-fold that of hsa-mir-155-5p.

13. The population of anti-inflammatory EVs of any one of embodiments 1-12, wherein the Tregs are from a healthy human subject.

14. The population of anti-inflammatory EVs of any one of embodiments 1-12, wherein the Tregs are from a human subject diagnosed with or suspected of having a neurodegenerative disorder.

15. The population of anti-inflammatory EVs of any one of embodiments 1-14, wherein the anti-inflammatory EVs exhibit an ability to increase the expression of IL-10, Arg1 and/or CD206 in the myeloid cells.

16. The population of anti-inflammatory EVs of any one of embodiments 1-15, wherein the anti-inflammatory EVs exhibit an ability to decrease the expression of IL-6, IL-8, IL1β or Interferon-γ in the myeloid cells.

17. The population of anti-inflammatory EVs of embodiment 1, wherein the proliferation of responder T cells is determined by flow cytometry or thymidine incorporation.

18. The population of anti-inflammatory EVs of any one of embodiments 1-17, wherein the population is a saline-containing population of anti-inflammatory EVs.

19. An isolated, cell-free population of anti-inflammatory extracellular vesicles (EVs), wherein the anti-inflammatory EVs are derived from ex vivo-expanded human suppressive immune cells.

20. The population of anti-inflammatory EVs of embodiment 19, wherein the human suppressive immune cells are regulatory T cells (Tregs).

21. The population of anti-inflammatory EVs of embodiment 20, wherein the Tregs are from a healthy human subject.

22. The population of anti-inflammatory EVs of embodiment 21, wherein the Tregs are from a human subject diagnosed with or suspected of having a neurodegenerative disorder.

23. The population of anti-inflammatory EVs of embodiment 22, wherein the neurodegenerative disorder is Alzheimer's disease.

24. The population of anti-inflammatory EVs of embodiment 22, wherein the neurodegenerative disorder is Amyotrophic Lateral Sclerosis (ALS).

25. The population of anti-inflammatory EVs of embodiment 22, wherein the neurodegenerative disease is multiple sclerosis (MS).

26. The population of anti-inflammatory EVs of embodiment 22, wherein the neurodegenerative disease is Parkinson's Disease.

27. The population of anti-inflammatory EVs of embodiment 20, wherein the Tregs are from a human subject who is diagnosed as having, or suspected of having had, a stroke.

28. The population of anti-inflammatory EVs of embodiment 20 wherein the Tregs are from a geriatric human subject.

29. The population of anti-inflammatory EVs of any one of embodiments 20-28, wherein the Tregs are from multiple human subjects.

30. The population of anti-inflammatory EVs of embodiment 29, wherein the Tregs are from multiple unrelated human subjects.

31. The population of anti-inflammatory EVs of any one of embodiments 19-30, wherein the anti-inflammatory EVs exhibit an ability to increase the expression of one or more anti-inflammatory markers in inflammatory cells.

32. The population of anti-inflammatory EVs of embodiment 31, wherein the inflammatory cells are myeloid cells.

33. The population of anti-inflammatory EVs of embodiment 31 or 32, wherein the anti-inflammatory EVs exhibit an ability to increase the expression of IL-10, Arg1 and/or CD206 in inflammatory cells.

34. The population of anti-inflammatory EVs of any one of embodiments 19-33, wherein the anti-inflammatory EVs exhibits an ability to suppress inflammatory cells, as measured by pro-inflammatory cytokine production by the inflammatory cells.

35. The method of embodiment 34, wherein the inflammatory cells are myeloid cells.

36 The population of anti-inflammatory EVs of embodiment 35, wherein the myeloid cells are monocytes, macrophages, or microglia.

37. The population of anti-inflammatory EVs of embodiment 36, wherein the macrophages are M1 macrophages.

38. The population of anti-inflammatory EVs of embodiment 37, wherein the M1 macrophages are induced pluripotent stem cell (iPSC)-derived M1 macrophages.

39. The population of anti-inflammatory EVs of any one of embodiments 31-38, wherein the ability to suppress inflammatory cells is measured by IL-6, IL-8, TNFα, IL1β and/or Interferon-γ production by the inflammatory cells.

40. The population of anti-inflammatory EVs of any one of embodiments 19-39 wherein the anti-inflammatory EVs exhibit a suppressive function, as determined by suppression of proliferation of responder T cells.

41. The population of anti-inflammatory EVs of embodiment 40, wherein the proliferation of responder T cells is determined by flow cytometry or thymidine incorporation.

42. The population of anti-inflammatory EVs of any one of embodiments 19-41, wherein the population is a saline-containing population of anti-inflammatory EVs.

43. The population of anti-inflammatory EVs of any one of embodiments 19-41, wherein the population is a physiological saline-containing population of anti-inflammatory EVs.

44. The population of anti-inflammatory EVs of any one of embodiments 19-41, wherein the population is a phosphate-buffered saline-containing population of anti-inflammatory EVs.

45. The population of anti-inflammatory EVs of any one of any one of embodiments 19-44, wherein the population of anti-inflammatory EVs comprises exosomes and microvesicles.

46 The population of anti-inflammatory EVs of embodiment 45, wherein the majority of the EVs are exosomes.

47. The population of anti-inflammatory EVs of embodiment 46, wherein at least about 80%, about 90%, or about 95% of the EVs are exosomes.

48. The population of anti-inflammatory EVs of embodiment 47 wherein the majority of the EVs are microvesicles.

49. The population of anti-inflammatory EVs of embodiment 48, wherein at least about 80%, about 90%, or about 95% of the EVs are microvesicles.

50. The population of anti-inflammatory EVs of embodiment 45, wherein the majority of the EVs have diameters from about 30 nm to about 1000 nm.

51. The population of anti-inflammatory EVs of embodiment 45, wherein the majority of the EVs have diameters from about 30 nm to about 100 nm, about 30 nm to about 150 nm, about 30 to about 200 nm, about 40 to about 100 nm, about 80 to about 100 nm, about 80 to about 110 nm, about 80 to about 125 nm, or about 100 to about 120 nm.

52. The population of anti-inflammatory EVs of embodiment 25 wherein the majority of the EVs have diameters from about 60 nm to about 1000 nm, about 70 nm to about 1000 nm, about 80 nm to about 1000 nm, 100 to about 1000 nm, about 200 to about 1000 nm, or about 300 to about 1000 nm.

53. A pharmaceutical composition comprising an isolated, cell-free population of anti-inflammatory EVs of any one of embodiments 1-52.

54. The pharmaceutical composition of embodiment 53, wherein the population of anti-inflammatory EVs comprises about 1×10⁶ to about 1×10¹⁴ EVs, about 1×10⁸ to about 1×10¹⁴ EVs, about 1×10⁸ to about 1×10¹² EVs, about 1×10⁸ to about 1×10¹⁰ EVs, about 1×10¹⁰ to about 1×10¹⁴ EVs, or about 1×10¹⁰ to about 1×10¹² EVs.

55. The pharmaceutical composition of embodiment 53, wherein the population of anti-inflammatory EVs comprises about 1×10⁶ to about 1×10¹⁴ EVs/ml, about 1×10⁸ to about 1×10¹⁴ EVs/ml, about 1×10⁸ to about 1×10¹² EVs/ml, about 1×10⁸ to about 1×10¹⁰ EVs/ml, about 1×10¹⁰ to about 1×10¹⁴ EVs/ml, or about 1×10¹⁰ to about 1×10¹² EVs/ml.

56. The pharmaceutical composition of embodiment 53, wherein the population of anti-inflammatory EVs comprises about 1 μg to about 200 mg EVs.

57. The pharmaceutical composition of embodiment 53, wherein the population of anti-inflammatory EVs comprises about 1 μg to about 15 mg EVs.

58. The pharmaceutical composition of embodiment 53, wherein the population of anti-inflammatory EVs comprises about 1 μg to about 15 mg EV/ml.

59. The pharmaceutical composition of any one of embodiments 53-58, wherein the pharmaceutical composition is a cryopreserved pharmaceutical composition.

60 The pharmaceutical composition of any one of embodiments 53-58, wherein the pharmaceutical composition had previously been cryopreserved.

61. A cryopreserved composition comprising an isolated, cell-free population of anti-inflammatory EVs of any one of embodiments 1-53.

62. A method of producing an isolated, cell-free population of anti-inflammatory extracellular vesicles (EVs), said method comprising the steps of:
a. ex-vivo expanding a human suppressive immune cell population in culture media to produce a culture comprising the cells, the culture media and anti-inflammatory EVs; and
b. isolating the anti-inflammatory EVs from the culture.

63. The method of embodiment 62, wherein the human suppressive immune cell population is a population of regulatory T cells (Tregs).

64. The method of embodiment 62 or 63 wherein step b) comprises removing cells from the culture, followed by polyethylene glycol precipitation of the culture.

65. The method of embodiment 62 or 63, wherein step b) comprises:
i) removing the cells from the culture to produce a cell-free, anti-inflammatory EV-containing solution; and
ii) isolating the anti-inflammatory EVs from the cell-free, anti-inflammatory EV-containing solution of i).

66. The method of embodiment 65, wherein step i) comprises passing the culture through a filter such that the cells are retained by the filter, and thereby removed from the culture.

67. The method of embodiment 65 or 66, wherein step i) comprises microfiltration.

68. The method of any one of embodiments 65-67, wherein step ii) comprises step ii-a): passing the cell-free, anti-inflammatory EV-containing solution through a filter such that the anti-inflammatory EVs are retained by the filter.

69. The method of embodiment 68, wherein the filter has a molecular weight cut-off (MWCO) of about 200 kilodaltons (kDa) to about 600 kDa.

70. The method of embodiment 69, wherein the filter has an MWCO of about 500 kDa.

71. The method of any one of embodiments 65-70, wherein step ii) comprises ultrafiltration.

72. The method of any one of embodiments 68-71, wherein step ii) further comprises step ii-b): performing buffer exchange such that the isolated, cell-free population of anti-inflammatory EVs produced is a buffer-containing isolated, cell-free population of anti-inflammatory EVs.

73. The method of embodiment 72, wherein the buffer is a saline-containing buffer.

74. The method of embodiment 73, wherein the saline-containing buffer is physiological saline.

75. The method of embodiment 74, wherein the saline-containing buffer is PBS.

76. The method of any one of embodiments 73-75, wherein step ii-b) comprises diafiltration.

77. The method of any one of embodiment 73-76 wherein steps ii-a) and ii-b) are performed simultaneously.

78. The method of any one of embodiments 62-77, wherein step b) comprises tangential flow filtration.

79. The method of any one of embodiments 62-78, wherein the culture media in step a) is serum-free.

80. The method of any one of embodiments 62-79, wherein the culture media in step a) comprises serum.

81. The method of embodiment 80, wherein the serum is human AB serum.

82. The method of embodiment 80 or 81, wherein the serum is depleted for serum-derived EVs.

83. The method of any one of embodiments 62-82 further comprising, prior to step a), the step of enriching Tregs from a cell sample suspected of containing Tregs, to produce a baseline Treg cell population that is the population of Tregs that is then expanded in a).

84. The method of embodiment 83, wherein the cell sample is a leukapheresis cell sample.

85. The method of embodiment 83 or 84, wherein the method further comprises obtaining the cell sample from a donor by leukapheresis.

86. The method of any one of embodiments 83-85, wherein the cell sample is not stored overnight or frozen before carrying out the enriching step.

87. The method of any one of embodiments 83-86, wherein the cell sample is obtained within 30 minutes before initiation of enriching step.

88. The method of any one of embodiments 82-87, wherein the enriching step comprises depleting CD8+/CD19+ cells then enriching for CD25+ cells.

89 The method of any one of embodiments 62-88, wherein step a) is carried out within 30 minutes of the enriching step.

90. The method of any one of embodiments 62-89, wherein step a) comprises culturing the Tregs in a culture media that comprises beads coated with anti-CD3 antibodies and anti-CD28 antibodies.

91. The method of embodiment 90, wherein the beads are first added to the culture media within about 24 hours of the initiation of the culturing.

92. The method of embodiment 90 or 91, wherein beads coated with anti-CD3 antibodies and anti-CD28 antibodies are added to the culture media about 14 days after beads coated with anti-CD3 antibodies and anti-CD28 antibodies were first added to the culture medium.

93 The method of any one of embodiments 90-92, wherein step a) further comprises adding IL-2 to the culture medium within about 6 days of the initiation of culturing.

94. The method of embodiment 93, wherein step a) further comprises replenishing the culture medium with IL-2 about every 2-3 days after IL-2 is first added to the culture medium.

95. The method of any one of embodiments 90-94, wherein step a) further comprises adding rapamycin to the culture medium within about 24 hours of the initiation of the culturing.

96. The method of embodiment 95, wherein step a) further comprises replenishing the culture medium with rapamycin every 2-3 days after the rapamycin is first added to the culture medium.

97. The method of any one of embodiments 62-96, wherein step a) is automated.

98. The method of any one of embodiments 62-97, wherein step a) takes place in a bioreactor.

99. The method of any one of embodiments 62-98, wherein step b) may commence at any point during step a).

100. The method of any one of embodiments 63-99, wherein the Tregs are from a healthy human subject.

101. The method of any one of embodiments 63-99, wherein the Tregs are from a human subject diagnosed with or suspected of having a neurodegenerative disorder.

102. The method of embodiment 101, wherein the neurodegenerative disorder is Alzheimer's disease, Amyotrophic Lateral Sclerosis (ALS), multiple sclerosis (MS), or Parkinson's Disease.

103. The method of any one of embodiments 63-102, wherein the Tregs are from a human subject who is diagnosed as having, or suspected of having had, a stroke.

104. The method of any one of embodiments 63-102, wherein the Tregs are from a geriatric human subject.

105. The method of any one of embodiments 63-104, wherein the Tregs are from multiple human subjects.

106. The method of embodiment 62, wherein the human suppressive immune cell population is a genetically engineered human suppressive immune cell population.

107. The method of any one of embodiments 63-106, wherein the population of Tregs is a genetically engineered population of Tregs.

108. A pharmaceutical composition comprising an isolated, cell-free population of anti-inflammatory EVs, wherein the population is made by any one of the methods of embodiment 62-107.

109. The method of any one of embodiments 62-107, further comprising: c) cryopreserving the isolated, cell-free population of anti-inflammatory EVs, thereby producing a cryopreserved, isolated, cell-free population of anti-inflammatory EVs.

110. The method of embodiment 109, further comprises thawing the cryopreserved, isolated cell-free population of anti-inflammatory EVs after cryopreservation for about 1 week, 1 month, about 3 months, about 6 months, about 9 months, about 12 months, about 18 months or about 24 months.

111. A pharmaceutical composition comprising the isolated, cell-free population of anti-inflammatory EVs of embodiment 110.

112. An isolated, cell-free population of anti-inflammatory EVs, wherein the anti-inflammatory EVs are derived from an ex vivo-expanded Treg cell population that exhibits an ability to suppress inflammatory cells, as measured by pro-inflammatory cytokine production by the inflammatory cells, wherein the inflammatory cells are macrophages or monocytes from human donors or generated from induced pluripotent stem cells, wherein the ex vivo-expanded Treg cell population has been expanded from baseline Tregs, and wherein, in the ex vivo-expanded Treg cell population:
a) expression of one or more dysfunctional baseline signature gene products listed in Table 3 and/or Table 4 is decreased relative to the expression of the one or more gene products in baseline Tregs;
b) expression of one or more dysfunctional baseline signature gene products listed in Table 5 is decreased relative to the expression of the one or more gene products in baseline Tregs;
c) expression of one or more Treg-associated signature gene products listed in Table 6 is increased relative to the expression of the one or more gene products in baseline Tregs;
d) expression of one or more mitochondria signature gene products listed in Table 7 is increased relative to the expression of the one or more gene products in baseline Tregs;
e) expression of one or more cell proliferation signature gene products listed in Table 8 is increased relative to the expression of the one or more gene products in baseline Tregs; or
f) expression of one or more highest protein expression signature gene products listed in Table 9 is increased relative to the expression of the one or more gene products in baseline Tregs.

113. A pharmaceutical composition comprising the isolated, cell-free population of anti-inflammatory EVs of embodiment 112.

114. A method of treating a disorder associated with Treg dysfunction, the method comprising administering to a subject in need of said treatment the composition of any one of embodiments 53-60, 108, 111, or 113.

115. A method of treating a disorder associated with Treg deficiency, the method comprising administering to a subject in need of said treatment the pharmaceutical composition of any one of embodiments 53-60, 108, 111, or 113.

116. A method of treating a disorder associated with overactivation of the immune system, the method comprising administering to a subject in need of said treatment the pharmaceutical composition of any one of embodiments 53-60, 108, 111, or 113.

117. A method of treating an inflammatory condition driven by a T cell response, the method comprising administering to a subject in need of said treatment the pharmaceutical composition of any one of embodiments 53-60, 108, 111, or 113.

118. A method of treating an inflammatory condition driven by a myeloid cell response, the method comprising administering to a subject in need of said treatment the pharmaceutical composition of any one of embodiments 53-60, 108, 111, or 113.

119. The method of embodiment 118, wherein the myeloid cell is a monocyte, macrophage or microglia.

120. A method of treating a neurodegenerative disorder in a subject in need thereof, the method comprising administering to a subject in need of said treatment the pharmaceutical composition of any one of embodiments 53-60, 108, 111, or 113.

121. The method of embodiment 120, wherein the neurodegenerative disease is ALS, Alzheimer's disease, Parkinson's disease, frontotemporal dementia or Huntington's disease.

122. A method of treating an autoimmune disorder in a subject in need thereof, the method comprising administering to a subject in need of said treatment the pharmaceutical composition of any one of embodiments 53-60, 108, 111, or 113.

123. The method of embodiment 122, wherein the autoimmune disorder is polymyositis, ulcerative colitis, inflammatory bowel disease, Crohn's disease, celiac disease, systemic sclerosis (scleroderma), multiple sclerosis (MS), rheumatoid arthritis (RA), Type I diabetes, psoriasis, dermatomyosititis, systemic lupus erythematosus, cutaneous lupus, myasthenia gravis, autoimmune nephropathy, autoimmune hemolytic anemia, autoimmune cytopenia, autoimmune encephalitis, autoimmune hepatitis, autoimmune uveitis, alopecia, thyroiditis or pemphigus.

124. A method of treating graft-versus-host disease in a subject in need thereof, the method comprising administering to a subject in need of said treatment the pharmaceutical composition of any one of embodiments 53-60, 108, 111, or 113. The method of embodiment 106, wherein the subject has received a bone marrow transplant, kidney transplant or liver transplant.

125. A method of improving islet graft survival in a subject in need thereof, the method comprising administering to a subject in need of said treatment the pharmaceutical composition of any one of embodiments 53-60, 108, 111, or 113.

126. A method of treating cardio-inflammation in a subject in need thereof, the method comprising administering to a subject in need of said treatment the pharmaceutical composition of any one of embodiments 53-60, 108, 111, or 113.

127. The method of embodiment 126, wherein the cardio-inflammation is associated with atherosclerosis, myocardial infarction, ischemic cardiomyopathy or heart failure.

128. A method of treating neuroinflammation in a subject in need thereof, the method comprising administering to a subject in need of said treatment the pharmaceutical composition of any one of embodiments 53-60, 108, 111, or 113.

129. The method of embodiment 128, wherein the neuroinflammation is associated with stroke, acute disseminated encephalomyelitis, acute optic neuritis, acute inflammatory demyelinating polyradiculoneuropathy, chronic inflammatory demyelinating polyradiculoneuropathy, Guillain-Barre syndrome, transverse myelitis, neuromyelitis optica, epilepsy, traumatic brain injury, spinal cord injury, encephalitis, central nervous system vasculitis, neurosarcoidosis, autoimmune or post-infectious encephalitis or chronic meningitis.

130. A method of treating a Tregopathy in a subject in need thereof, comprising administering to a subject in need of said treatment the pharmaceutical composition of any one of embodiments 53-60, 108, 111, or 113.

131. The method of embodiment 130, wherein the Tregopathy is caused by a FOXP3, CD25, cytotoxic T lymphocyte-associated antigen 4 (CTLA4), LPS-responsive and beige-like anchor protein (LRBA), or BTB domain and CNC homolog 2 (BACH2) gene loss-of-function mutation, or a signal transducer and activator of transcription 3 (STAT3) gain-of-function mutation.

132. The method of any one of embodiments 114-131, wherein the anti-inflammatory EVs are derived from Tregs that are autologous to the subject.

133. The method of any one of embodiments 114-131 wherein the anti-inflammatory EVs are derived from Tregs that are allogeneic to the subject.

134. The method of any one of embodiment 114-133, wherein the pharmaceutical composition is administered via intranasal administration.

135. The method of embodiment 134 wherein the intranasal administration is via aerosol inhalation or nasal drip.

136. The method of any one of embodiment 114-135, wherein the pharmaceutical composition is administered intravenously.

137. The method of any one of embodiment 114-135, wherein the pharmaceutical composition is administered by local injection.

138. The method of any one of embodiments 114-137, wherein the method further comprises administering to the subject a pharmaceutical composition comprising a therapeutic population of Tregs, wherein the Tregs had been ex vivo expanded and cryopreserved, and wherein the Tregs are not further expanded prior to the administering.

139. The method of embodiment 138, wherein the therapeutic population of Tregs is autologous to the subject.

140. The method of embodiment 138, wherein the therapeutic population of Tregs is allogeneic to the subject.

141. The method of any one of embodiments 138-140, wherein the pharmaceutical composition comprising the therapeutic population of Tregs is administered intravenously.

142. The method of any one of embodiments 138-141, wherein the pharmaceutical composition comprising the anti-inflammatory EVs and the pharmaceutical composition comprising the therapeutic population of Tregs are administered to the patient on the same day.

143. The method of any one of embodiments 114-140, wherein the isolated, cell-free population of anti-inflammatory EVs had been cryopreserved and thawed prior to being administered to the subject.

144. The method of any one of embodiments 114-140, wherein the isolated, cell-free population of anti-inflammatory EVs are stored overnight at 4° C. prior to being administered to the subject.

145. The method of embodiment 144, wherein the isolated, cell-free population of anti-inflammatory EVs had been cryopreserved then thawed and stored at 4° C. overnight prior to being administered to the subject.

146. The method of any one of embodiments 114-140, wherein the isolated, cell-free population of anti-inflammatory EVs had undergone at least two freeze/thaw cycles prior to being administered to the subject.

147. The method of embodiment 146, wherein the isolated, cell-free population of anti-inflammatory EVs had undergone about 2 to about 20 freeze/thaw cycles prior to being administered to the subject.

### 4. BRIEF DESCRIPTION OF THE FIGURES

FIG. 1. Process flow diagram for an exemplary process of Treg isolation, enrichment and ex-vivo expansion.

FIGS. 2A-2K. FIG. 2A: graphic depicting the two EV populations (mixed Treg-derived EVs and enriched or pure Treg EVs) which were generated, and references to which populations are utilized in experiments depicted in FIGS. 2B-2K. The mixed EV population obtained from Treg cultures were produced using the improved Treg ex-vivo expansion protocol described in Example 1. The Tregs were obtained from ALS patients and the culture medium utilized during this expansion process contains 5% Human AB serum. Thus, the anti-inflammatory EVs isolated from this culture are present together with EVs the media serum. It is estimated that the Treg-derived anti-inflammatory EV population is approximately 20-30% of the total EV population. The second Treg population was collected from healthy patient samples and expanded using the improved Treg ex-vivo expansion protocol described in Example 1, but using culture medium containing exosome-depleted fetal bovine serum (FBS) instead of human AB serum. Thus, the anti-inflammatory EV population produced from this culture constitutes a pure batch of EVs derived from ex-vivo expanded human Tregs. FIGS. 2B-2F and 21-2K: The experiments utilized EV populations isolated using PEG. FIGS. 2G-2H: The experiments utilized EV populations isolated using tangential flow filtration (TFF). FIG. 2B: Treg mixed EVs reduce iPSC-derived M1 IL-6 protein by ˜70% following co-culture of 1×10⁸ Treg EVs per 50,000 M1 cells stimulated with LPS/IFNγ overnight. FIG. 2C: The mixed Treg EVs are able to suppress Tresp proliferation at escalated dosing. FIG. 2D: The pure Treg EV batches demonstrate the ability to suppress IL-6 transcript. FIG. 2E: Pure Treg EVs suppress M1 Il-6 protein following overnight stimulation. FIG. 2F: Pure Treg EVs suppress Tresp proliferation at escalated dosing. FIG. 2G: Mixed Treg EVs were shown to be able to suppress M1 IL-6 protein production regardless of whether isolation was performed via PEG precipitation or TFF (n=3; the PEG and TFF isolation protocols were performed on expanded Tregs from the same three patients from the clinical trial) FIG. 2H: Mixed Treg EVs were shown to be able to suppress Tresp proliferation regardless of whether isolation was performed via PEG precipitation or TFF (n=3; the PEG and TFF isolation protocols were performed on expanded Tregs from the same three patients). FIG. 2I: Exemplary size profile of Treg EV produced and as described in this example, which demonstrates a single peak distribution within a 20-200 nm. FIG. 2J: Graph depicting Miltenyi MACSPlex Exosome Kit (Miltenyi Biotec) analysis of Treg (mixed) EVs and media EVs. FIG. 2K: Graph depicting Miltenyi MACSPlex Exosome KIT (Miltenyi Biotec) analysis of Treg (mixed) EVs and media EVs. ALS Treg EVs n=7; media EVs n=3. Numbers shown as averages+/−SEM with analysis via one-way ANOVA with Tukey's post hoc testing. ** indicates a p-value of less than 0.01; *** indicates a p-value of less than 0.001.

FIGS. 3A-3D. The anti-inflammatory effects of Treg EVs were evaluated in an LPS-induced neuroinflammation model. Briefly, 2 mg/kg LPS were injected intraperitoneally. Two hours after the injection, pure Treg EVs were administered intranasally. Brain regions and spleen CD11b+myeloid cells were isolated following 12 hours post-intranasal administration. Pro-inflammatory transcripts were analyzed to assess anti-inflammatory effects. FIG. 3A: Graphic describing the LPS-induced neuroinflammation model and Treg EV treatment paradigm. FIG. 3B: Intranasal Treg EVs reduce IL-6 and IL-1β transcripts in the hippocampus. FIG. 3C: Reduced IL-6 transcripts following intranasal Treg EV treatment in the cortex of mice. FIG. 3D: Reduction in peripheral myeloid cell activation following intranasal treatment of Treg EVs; demonstrates reduced IL-6 and TNF transcripts in spleen-derived, CD11b+ myeloid cells. P-values are *p<0.05 and ** p<0.01.

FIGS. 4A-4F. Treg EVs were given every two weeks in SOD1 mice intranasally starting at day 90 (approximately 20 days after symptoms start to manifest in this model) to assess the mouse clinical benefit of multiple rounds of intranasal Treg EVs. After the sacrifice of the mouse, inflammatory markers in the inflamed lumbar section of the spinal cord were assessed through RNA analysis. FIG. 4A: Graphic depicting the intranasal Treg EV treatment paradigm for the SOD1 mouse model of ALS. FIG. 4B: Intranasal Treg EV treatment increases the probability of survival compared to intranasal PBS treatments. FIG. 4C: The Treg EV treatments slowed the progression of disease as defined by a modified scoring system used to assess mouse ALS progression; the effects were more prominent when the mice were going through the rapid phase of their disease progression. FIG. 4D: The treatment significantly prolonged disease duration. FIG. 4E: The average lifespan is increased in animals who received the Treg EV treatment. FIG. 4F: Lumbar spinal cords were dissected after animals reached their ethical endpoints. RNA analysis was done using the lumbar spinal cord tissue to examine inflammatory markers. Decreased levels of inflammatory markers were observed in the spinal cord, while increased signals of Tregs (FOXP3) and anti-inflammatory M2 macrophages (CD206) were observed in the treated animals. Numbers shown as averages+/−SEM and with one-way ANOVA analysis with hTukey's post hoc test (PBS n=3, LPS+PBS n=4, LPS+Treg EV n=4, Trev EV only n=3. * indicates a p-value of less than 0.05; *** indicates a p-value of less than 0.001.

FIGS. 5A-5C. FIG. 5A: Treg EVs were able to suppress M1 pro-inflammatory IL-6 protein by 46% at a dose of 1×10⁸ EVs and 30.6% at a dose of 1×10⁷ EV compared to MSC EVs that suppressed 13.7% and 3.3%, respectively. FIG. 5B: Treg EVs suppressed M1-derived pro-inflammatory IL-8 protein by 60% at a dose of 1×10⁸ and 50% at a dose of 1×10⁷ dose compared to MSC EV that showed a 20% suppression at the dose of 1×10⁸ dose. “Control exo” in FIGS. 5A and 5B: EVs derived from non-exosome depleted media without cell culture. FIG. 5C: Treg EVs suppressed T cell proliferation more than MSC EVs in a comparison study.

FIGS. 6A-6C. Treg EV stability and function were evaluated after 1 to 20 freeze/thaw cycles and after storage at −20° C. for 3 months, 6 months, or 12 months. FIG. 6A: No loss in Treg EV particle number was observed following multiple (up to 20) freeze/thaw cycles. FIG. 6B: No significant deviation in Treg EV particle size was observed during the same freeze/thaw cycles. FIG. 6C: Treg EV suppression of T cell proliferation did not decrease over time in frozen −20° C. storage.

FIGS. 7A-7B. FIG. 7A: Treg EV concentrations (EV particles/ml media) after EV isolation from expansion media. EV concentration from media alone (“Media”) is also shown. FIG. 7B: Fold-increase in EVs from ex vivo-expanded Treg cell populations compared to EVs from media alone.

FIG. 8. Exemplary size profile of Treg EVs isolated using a TFF protocol. The profile shows an EV mean of 92.1 nm±4.2 nm and a mode of 73.3 nm±6.1 nm.

FIGS. 9A-9B. FIG. 9A: Treg EVs derived from ex vivo-expanded ALS patient Tregs induce M1 cells to increase Arg1 mRNA expression in an EV concentration-dependent manner. FIG. 9B: Treg EVs derived from ex vivo-expanded ALS patient Tregs induce M1 cells to increase CD206 mRNA expression in an EV concentration-dependent manner.

FIGS. 10A-10C. FIG. 10A: Treg EVs were shown to be significantly more potent than MSC EVs in suppressing M1 pro-inflammatory IL-6 protein production (n=3 for each group; *** indicates a p-value of less than 0.001, compared to the corresponding MSC EVs). FIG. 10B: Treg EVs were shown to be significantly more potent than MSC EVs in suppressing T cell proliferation (n=3 for each group; *** indicates a p-value of less than 0.001, compared to the corresponding MSC EVs). FIG. 10C: Treg EVs were shown to be significantly more potent than MSC EVs in suppressing M1 pro-inflammatory IL-8 protein production (n=3 for each group; *** indicates a p-value of less than 0.001, compared to the corresponding MSC EVs). The Treg EVs utilized for these experiments were pure Treg EVs and the MSC EVs utilized for these experiments were pure MSC EVs.

FIGS. 11A-11B. FIG. 11A: The mean of particle size of EVs isolated via TFF. FIG. 11B: The mode of particle size of EVs isolated via TFF.

FIG. 12. Recovery of EVs isolated via TFF (n=6, results reported as mean±SD).

FIG. 13. A flow chart of a process of producing a population of Tregs in a bioreactor.

FIGS. 14A-14F. FIG. 14A: Graphic describing the LPS-induced model of acute inflammation where WT mice are administered LPS via intraperitoneal injection and subsequently treated with single tail vein (IV) injections of different doses of Treg EVs. Following overnight treatment, mice were sacrificed and peripheral immune cells were isolated from the mice spleens for subsequence inflammatory transcript analysis. FIG. 14B: Graphic describing the LPS-induced model of acute inflammation where WT mice are administered LPS via intraperitoneal injection and subsequently treated with single tail vein (IV) injections of different doses of Treg EVs. Following overnight treatment, mice were sacrificed, brain tissue (hippocampus and cortex) was isolated and neuroinflammatory marker transcript analysis was performed. FIG. 14C: Graphs depicting pro-inflammatory transcript fold changes for IL6 and iNOS in CD11b+ myeloid cells from the spleen following IV treatment of Treg EVs. FIG. 14D: Graphs depicting pro-inflammatory transcript fold changes for IL1b and IFNγ in CD11b+ myeloid cells from the spleen following IV treatment of Treg EVs. FIG. 14E: Graphs depicting fold changes of anti-inflammatory associated transcripts of CD206 (MRC1) and CD163 in CD11b+ myeloid cells following treatment with Treg EVs. FIG. 14F: Graphs depict fold changes in FOXP3 and IL2RA (CD25) in fresh spleen isolated CD4+CD25+Tregs following Treg EV treatment. Data shown as averages±SEM and statistical analysis done with one-way ANOVA analysis with Tukey's post hoc test (PBS n=5, LPS n=5, LPS+Treg EV 1×10⁹ n=5 (peripheral tissue), 4-5 (brain tissue), LPS+Treg EV 1×10¹⁰ n=5 (peripheral tissue), 4-5 (brain tissue), LPS+Treg EV 1×10¹¹ n=5 (peripheral tissue), 4-5 (brain tissue)). p-values are *p<0.05, ** p<0.01 and *** p<0.001.

FIGS. 15A-15B. FIG. 15A: Graphs depict fold changes in IL-6, IL1b, and TNF RNA in the hippocampus following IV Treg EV treatment. FIG. 15B: Graphs depict fold changes in IL-6, IL1b, and TNF in the cortex following Treg EV treatment. Data shown as averages±SEM and with one-way ANOVA analysis with Tukey's post hoc testing (PBS n=5, LPS n=5, LPS+Treg EV 1×10⁹ n=4-5, LPS+Treg EV 1×10¹⁰ n=4-5, LPS+Treg EV 1×10¹¹ n=5).

FIGS. 16A-16B. FIG. 16A: Size distribution of TFF isolated Treg EVs generated via nanoparticle tracking analysis. FIG. 16B: Particle size data from TFF isolated Treg EVs generated via nanoparticle tracking analysis.

FIG. 17. Treg EVs suppression of T cell proliferation in vitro (n=6).

FIG. 18. Quantification of Treg functional proteins in Treg EVs using ELISA.

FIGS. 19A-19B. Quantification of residual IL2 and albumin concentrations as a percent of original total amount, in the concentrated batch following TFF, and in final exemplary dose formulation.

FIGS. 20A-20E. FIG. 20A: Stability of Treg EVs at room temperature and 4° C. Presented are the aggregate of results using Treg EVs from bioreactor runs BioR4-6 through 8 hour timepoints and BioR5 and BioR6 for all timepoints. FIG. 20B: Stability of Treg EVs at room temperature and 4° C., showing the breakout of the individual sample results that were aggregated into the result shown in FIG. 20A. FIG. 20C: Stability of particle size distribution of Treg EVs at room temperature and 4° C. As with FIG. 20A, presented are the aggregate of results using Treg EVs from bioreactor runs BioR4-6 through 8 hour timepoints and BioR5 and BioR6 for all timepoints. FIG. 20D: Stability of Treg EVs after prolonged storage at −20° C. and −80° C. For each of the timepoints, n=3 (Treg EVs from BioR4-6). FIG. 20E: Stability of particle size distribution of Treg EVs after prolonged storage at −20° C. and −80° C. For each of the timepoints, n=3 (Treg EVs from BioR4-6).

FIG. 21. Ability of Treg EVs to suppress activated, pro-inflammatory M1 cells.** indicates a p-value of less than 0.01; *** indicates a p-value of less than 0.001.

FIG. 22. Signature of EV surface proteins. Top panel: Bioreactor TFF Treg EVs. Bottom panel: Media EVs.

FIG. 23. Signature of ALS patient Treg EV surface proteins.

FIGS. 24A-24B. Graphs depicting levels of exosome markers associated with ALS expanded Treg EVs and Control expanded Treg EVs (n=3 for ALS expanded Treg EVs and n=6 for Control Expanded Treg EVs).

### 5. DETAILED DESCRIPTION

Described herein are anti-inflammatory EV populations derived from ex vivo-expanded human suppressive immune cells, for example regulatory T cells (Tregs). The EVs presented herein exhibit impressive anti-inflammatory activity, both in vitro and in vivo. For example, results presented herein demonstrate that the EVs of the present disclosure are able to potently suppress T responder cell proliferation and pro-inflammatory myeloid, e.g., macrophage, activity in vitro, and also exert potent anti-inflammatory effects in vivo. Briefly, results presented herein demonstrate that the EVs are able to suppress brain and peripheral inflammation in an in vivo model of neuroinflammation, and are also able to suppress inflammation, extend survival and slow later stage disease progression in vivo model of amyotrophic lateral sclerosis (ALS). The anti-inflammatory EVs exhibit suppressive effects on pro-inflammatory responses via either intravenous or intranasal administration. The results presented herein demonstrate that the Treg EVs have a greater suppressive effect on pro-inflammatory immune cells than EVs derived from mesenchymal stem cells (MSCs).

Moreover, the anti-inflammatory EVs presented herein exhibit remarkable batch-to-batch consistency in size, stability and activity and exhibit a unique structural signature as, for example, characterized by Treg EV surface marker and RNA profiles. Still further, as demonstrated herein, the methods presented herein yield potent anti-inflammatory EVs exhibiting similar structural and suppressive activity characteristics whether the original Treg starting material is obtained from healthy subjects or ALS patients.

Without wishing to be bound by theory or mechanism, it appears that EVs of the present disclosure retain the immune suppressive activities of the cells from which they are derived. Moreover, as EVs are not themselves cells, they avoid potential cell-based issues such as immune rejection and the possibility of polarization to a pro-inflammatory cell type. As such, the anti-inflammatory EVs presented herein are particularly useful for treatment of a variety of diseases such as, for example, neurodegenerative disorders such as amyotrophic lateral sclerosis (ALS).

Presented herein are isolated, cell-free populations of anti-inflammatory EVs, wherein the anti-inflammatory EVs are derived from ex vivo-expanded human suppressive immune cells, for example regulatory T cells (Tregs). Also presented herein are pharmaceutical compositions and cryopreserved compositions comprising an isolated, cell-free population of anti-inflammatory EVs described herein, methods of producing the EV populations and methods of using the EVs for treatment of diseases, such as neurodegenerative diseases, e.g., ALS.

Recitation of ranges of values herein are merely intended to serve as a shorthand method of referring individually to each separate value falling within the range, unless otherwise indicated herein, and each separate value is incorporated into the specification as if it were individually recited herein.

Unless specifically stated or apparent from context, as used herein, the terms “a”, “an”, and “the” are understood to be singular or plural, and denote “one or more.”

The terms “include,” “such as,” and the like are intended to convey inclusion without limitation, unless otherwise specifically indicated.

The terms “or” and “and” can be used interchangeably and can be understood to mean “and/or.”

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

The terms “about” and “approximately” as used herein, are interchangeable, and should generally be understood to refer to a range of numbers around a given number, as well as to all numbers in a recited range of numbers (e.g., “about 5 to 15” means “about 5 to about 15” unless otherwise stated). Moreover, all numerical ranges herein should be understood to include each whole integer within the range. In particular, unless otherwise noted the terms mean within plus or minus 10% of a given value or range. In instances where an integer is required, the terms mean within plus or minus 10% of a given value or range, rounded either up or down to the nearest integer.

### 5.1 Anti-Inflammatory Extracellular Vesicles (EVs)

Presented herein are isolated, cell-free population of anti-inflammatory extracellular vesicles (EVs), wherein the anti-inflammatory EVs are derived from ex vivo-expanded human suppressive immune cells. In certain embodiments, the anti-inflammatory EVs are derived from human regulatory T cells (Tregs).

In certain embodiments, an isolated, cell-free population of anti-inflammatory EVs is produced by a method described herein, for example, as described in Section 5.2, below.

For ease of reference, unless otherwise noted, the terms “extracellular vesicles,” “EVs”, “extracellular vesicle particles,” and “EV particles” are used interchangeable herein. Moreover, as should be self-evident, it is to be understood that, as used herein, reference to “anti-inflammatory extracellular vesicles,” “anti-inflammatory EVs,” “anti-inflammatory exosomes,” and the like, includes the Treg EVs and Treg exosomes described in detail herein. While, for ease of description, not every embodiment presented herein is reproduced to recite, e.g., both “anti-inflammatory EVs” and “Treg EVs,” it is to be understood that each embodiment reciting such an “anti-inflammatory” embodiment includes and may be substituted for a corresponding “Treg EV” and “Treg exosome” as these are described herein.

EVs are membrane-bound particles released by cells. Generally, EVs comprise one or more constituents from the cells from which they are released, e.g., one or more DNA, RNA (e.g., coding and/or non-coding RNA, for example, mRNA microRNA, and/or long non-coding RNA), protein (e.g., signaling proteins, receptors, other surface proteins, glycoproteins and/or enzymes) or non-protein, e.g., lipid, constituents. EVs generally range in size from about 30 nm to about 1000 nm in diameter. Larger EVs are sometimes referred to as “microvesicles.” Roughly speaking, microvesicles have size diameters larger than about 200 nm. Smaller EVs are sometimes referred to as “exosomes.” Roughly speaking, exosomes have size diameters that range from about 30-40 nm to about 150-200 nm. Methods for determining EV particle size and concentration are well known to those of skill in the art.

Methods for determining EV particle size, concentration and purity are well known, including determinations that use dynamic light scattering or single particle tracking analysis, or utilize techniques such as flow cytometry, ELISA, or electron microscopy. See, e.g., Balaj et al. (2015) Sci Rep 5, 10266, Nakai et al. (2016) Sci Rep 6, 33935 and Carnino et al. (2019) Respiratory Research 20:240. In a particular embodiment, routine determination may be performed using nanoparticle analyzers, e.g., NanoSight (Malvern Panalytical) nanoparticle analyzers.

In some embodiments, EVs may be analyzed for the presence of exosome markers and/or Treg markers (e.g., CD25) by protein analysis using Western blot, ELISA, and other protein-associated assays, or commercially available arrays such as the Exo-Check™ Exosome Antibody Array (System Biosciences) and/or MACSPlex Exosome Kit (Miltenyi Biotec). In certain embodiments, a population of EVs may be analyzed for the presence of proteins associated with serum. In particular embodiments, an EV population described herein is substantially free of proteins associated with serum.

In certain embodiments, the anti-inflammatory EVs described herein exhibit an ability to increase the expression of one or more anti-inflammatory markers in inflammatory cells. For example, in particular embodiments, the anti-inflammatory EVs described herein exhibit an ability to increase the transcription of and/or level of mRNA expression of one or more genes encoding anti-inflammatory protein in inflammatory cells. In another example, in particular embodiments, the anti-inflammatory EVs described herein exhibit an ability to increase translation, processing, secretion and/or activation of one or more anti-inflammatory protein produced by inflammatory cells.

In specific embodiments, the anti-inflammatory marker is IL-10, Arg1, and/or CD206. In specific examples, the inflammatory cells are myeloid cells, for example, monocytes, macrophages or microglia, e.g., human inflammatory cells, for example, human monocytes, macrophages or microglia.

In certain embodiments, the anti-inflammatory EVs described herein exhibit an ability to suppress inflammatory cells. For example, in certain embodiments, the anti-inflammatory EVs described herein exhibit an ability to suppress inflammatory cells as measured by pro-inflammatory cytokine production by the inflammatory cells.

In some embodiments, the ability to suppress inflammatory cells is measured by IL-6, TNFα, IL1β, IL8, and/or Interferon-γ production by the inflammatory cells. In some embodiments, the ability to suppress inflammatory cells is measured by IL-6 production by the inflammatory cells.

In particular embodiments, the inflammatory cells are myeloid cells, for example, monocytes, macrophages or microglia e.g., human inflammatory cells, for example, human myeloid cells, such as human monocytes, macrophages or microglia. In specific examples, the myeloid cells, e.g., monocytes, macrophages or microglia, are from human donors or generated from induced pluripotent stem cells. In certain embodiments, the macrophages are M1 macrophages, such as induced pluripotent stem cell (iPSC)-derived M1 macrophages.

In certain embodiments, the anti-inflammatory EVs described herein exhibit an ability to suppress pro-inflammatory M1 cells. In some embodiments, the ability to suppress pro-inflammatory M1 cells is measured by IL-6 production by the pro-inflammatory M1 cells.

In certain embodiments, the anti-inflammatory EVs described herein exhibit an ability to suppress pro-inflammatory M1 cell IL-6 protein production by about 20% to about 70%, about 20% to about 50%, about 25% to about 50%, about 30% to about 50%, or about 25% to about 45%.

In certain embodiments, the anti-inflammatory EVs described herein exhibit an ability to suppress inflammatory cells as determined by suppression of proliferation of responder T cells. In particular embodiments, the proliferation of responder T cells is determined by flow cytometry or thymidine incorporation, e.g., tritiated thymidine incorporation.

In certain embodiments, the anti-inflammatory EVs described herein exhibit an ability to suppress inflammatory cells (e.g., as measured by pro-inflammatory cytokine production and/or responder T cell proliferation) and an ability to increase expression of one or more inflammatory markers in inflammatory cells.

In certain aspects, a population of anti-inflammatory EVs as described herein comprises exosomes. In other aspects, a population of anti-inflammatory EVs described herein comprises microvesicles. In yet other aspects, a population of anti-inflammatory EVs as described herein comprises exosomes and microvesicles.

In certain embodiments, the majority of EVs of a population of anti-inflammatory EVs as described herein are exosomes. For example, in certain embodiments at least about 50%, at least about 55%, at least about 60%, at least about 65%, at least about 70%, at least about 75%, at least about 80%, at least about 85%, at least about 90%, at least about 95%, at least about 98%, at least about 99% or more of the EVs of a population of anti-inflammatory exosomes described herein are exosomes.

In certain embodiments, the majority of EVs of a population of anti-inflammatory EVs as described herein are microvesicles. For example, in certain embodiments at least about 50%, at least about 55%, at least about 60%, at least about 65%, at least about 70%, at least about 75%, at least about 80%, at least about 85%, at least about 90%, at least about 95%, at least about 98%, at least about 99% or more of the EVs of a population of anti-inflammatory exosomes described herein are microvesicles.

In certain embodiments, the EVs of a population of anti-inflammatory EVs as described herein have size diameters of about 5 nm to about 1000 nm. In certain embodiments, the EVs of a population of anti-inflammatory EVs as described herein have size diameters of about 10 nm to about 1000 nm. In certain embodiments, the EVs of a population of anti-inflammatory EVs as described herein have size diameters of about 15 nm to about 1000 nm. In certain embodiments, the EVs of a population of anti-inflammatory EVs as described herein have size diameters of about 20 nm to about 1000 nm. In certain embodiments, the EVs of a population of anti-inflammatory EVs as described herein have size diameters of about 30 nm to about 1000 nm. In certain embodiments, the EVs of a population of anti-inflammatory EVs as described herein have size diameters of about 20 nm to about 300 nm. In certain embodiments, the EVs of a population of anti-inflammatory EVs as described herein have size diameters of about 20 nm to about 275 nm. In certain embodiments, the EVs of a population of anti-inflammatory EVs as described herein have size diameters of about 20 nm to about 250 nm. In certain embodiments, the EVs of a population of anti-inflammatory EVs as described herein have size diameters of about 20 nm to about 200 nm. In certain embodiments, the EVs of a population of anti-inflammatory EVs as described herein have size diameters of about 20 nm to about 175 nm. In certain embodiments, the EVs of a population of anti-inflammatory EVs as described herein have size diameters of about 50 nm to about 200 nm. In certain embodiments, the EVs of a population of anti-inflammatory EVs as described herein have size diameters of about 50 nm to about 175 nm. In certain embodiments, the EVs of a population of anti-inflammatory EVs as described herein have size diameters of about 50 nm to about 150 nm.

In certain embodiments, the majority (e.g., at least about 50%, at least about 55%, at least about 60%, at least about 65%, at least about 70%, at least about 75%, at least about 80%, at least about 85%, at least about 90%, at least about 95%, at least about 98%, at least about 99% or more) of the EVs of a population of anti-inflammatory EVs as described herein have size diameters of about 5 nm to about 1000 nm, about 10 nm to about 1000 nm, about 15 nm to about 1000 nm, about 20 nm to about 1000 nm, or about 30 nm to about 1000 nm.

In certain embodiments, the majority (e.g., at least about 50%, at least about 55%, at least about 60%, at least about 65%, at least about 70%, at least about 75%, at least about 80%, at least about 85%, at least about 90%, at least about 95%, at least about 98%, at least about 99% or more) of the EVs of a population of anti-inflammatory EVs as described herein have size diameters of about 20 nm to about 300 nm, about 20 nm to about 275 nm, about 20 nm to about 250 nm, about 20 nm to about 200 nm, or about 20 nm to about 175 nm.

In certain embodiments, the majority (e.g., at least about 50%, at least about 55%, at least about 60%, at least about 65%, at least about 70%, at least about 75%, at least about 80%, at least about 85%, at least about 90%, at least about 95%, at least about 98%, at least about 99% or more) of the EVs of a population of anti-inflammatory EVs as described herein have size diameters of about 20 nm to about 200 nm.

In certain embodiments, the majority (e.g., at least about 50%, at least about 55%, at least about 60%, at least about 65%, at least about 70%, at least about 75%, at least about 80%, at least about 85%, at least about 90%, at least about 95%, at least about 98%, at least about 99% or more) of the EVs of a population of anti-inflammatory EVs as described herein have size diameters of about 50 nm to about 200 nm, about 50 nm to about 175 nm, or about 50 nm to about 150 nm.

In certain embodiments, the majority (e.g., at least about 50%, at least about 55%, at least about 60%, at least about 65%, at least about 70%, at least about 75%, at least about 80%, at least about 85%, at least about 90%, at least about 95%, at least about 98%, at least about 99% or more) of the EVs of a population of anti-inflammatory EVs as described herein have size diameters of about 50 nm to about 150 nm.

In certain embodiments, the majority of EVs of a population of anti-inflammatory EVs as described herein have size diameters less than about 300 nm, less than about 200 nm, less than about 150 nm or less than about 100 nm. For example, in certain embodiments at least about 50%, at least about 55%, at least about 60%, at least about 65%, at least about 70%, at least about 75%, at least about 80%, at least about 85%, at least about 90%, at least about 95%, at least about 98%, at least about 99% or more of the EVs of a population of anti-inflammatory exosomes described herein have size diameters less than about 300 nm, less than about 200 nm, less than about 150 nm, or less than about 100 nm.

In certain embodiments, the EVs of a population of anti-inflammatory EVs as described herein have size diameters of about 30 nm to about 300 nm, about 30 nm to about 250 nm, about 30 nm to about 200 nm, about 30 nm to about 160 nm, about 30 nm to about 150 nm, about 30 nm to about 100 nm, about 40 nm to about 300 nm, about 40 nm to about 200 nm, about 40 nm to about 160 nm, about 40 nm to about 150 nm, about 40 nm to about 100 nm, about 60 nm to about 300 nm, about 60 nm to about 200 nm, about 60 nm to about 160 nm, about 60 nm to about 150 nm, about 60 nm to about 125 nm, about 60 nm to about 110 nm, about 60 nm to about 100 nm, about 60 nm to about 80 nm, about 70 nm to about 300 nm, about 70 nm to about 200 nm, about 70 nm to about 160 nm, about 70 nm to about 150 nm, about 70 nm to about 125 nm, about 70 nm to about 110 nm, about 70 nm to about 100 nm, about 80 nm to about 300 nm, about 80 nm to about 200 nm, about 80 nm to about 160 nm, about 80 nm to about 150 nm, about 80 nm to about 125 nm, about 80 nm to about 110 nm, about 80 nm to about 100 nm, or about 110 nm to about 120 nm.

In certain embodiments, the majority (e.g., at least about 50%, at least about 55%, at least about 60%, at least about 65%, at least about 70%, at least about 75%, at least about 80%, at least about 85%, at least about 90%, at least about 95%, at least about 98%, at least about 99% or more) of the EVs of a population of anti-inflammatory EVs as described herein have size diameters of about 30 nm to about 300 nm, about 30 nm to about 250 nm, about 30 nm to about 200 nm, about 30 nm to about 160 nm, about 30 nm to about 150 nm, about 30 nm to about 100 nm, about 40 nm to about 300 nm, about 40 nm to about 200 nm, about 40 nm to about 160 nm, about 40 nm to about 150 nm, about 40 nm to about 100 nm, about 60 nm to about 300 nm, about 60 nm to about 200 nm, about 60 nm to about 160 nm, about 60 nm to about 150 nm, about 60 nm to about 125 nm, about 60 nm to about 110 nm, about 60 nm to about 100 nm, about 60 nm to about 80 nm, about 70 nm to about 300 nm, about 70 nm to about 200 nm, about 70 nm to about 160 nm, about 70 nm to about 150 nm, about 70 nm to about 125 nm, about 70 nm to about 110 nm, about 70 nm to about 100 nm, about 80 nm to about 300 nm, about 80 nm to about 200 nm, about 80 nm to about 160 nm, about 80 nm to about 150 nm, about 80 nm to about 125 nm, about 80 nm to about 110 nm, about 80 nm to about 100 nm, or about 110 nm to about 120 nm.

In certain embodiments, the majority (e.g., at least about 50%, at least about 55%, at least about 60%, at least about 65%, at least about 70%, at least about 75%, at least about 80%, at least about 85%, at least about 90%, at least about 95%, at least about 98%, at least about 99% or more) of the EVs of a population of anti-inflammatory EVs as described herein have a size diameter of about 30 nm, about 40 nm, about 50 nm, about 60 nm, about 65 nm, about 70 nm, about 75 nm, 80 nm, about 85 nm, about 90 nm, about 95 nm, about 100 nm, about 110 nm to about 120 nm, about 150 nm, about 175 nm, about 200 nm, about 250 nm or about 300 nm.

In certain embodiments, the majority (e.g., at least about 50%, at least about 55%, at least about 60%, at least about 65%, at least about 70%, at least about 75%, at least about 80%, at least about 85%, at least about 90%, at least about 95%, at least about 98%, at least about 99% or more) of the EVs of a population of anti-inflammatory EVs as described herein have size diameters greater than about 300 nm, greater than about 400 nm, greater than about 500 nm, greater than about 500 nm, greater than about 700 nm, or greater than about 800 nm.

In certain embodiments, the EVs of a population of anti-inflammatory EVs as described herein have size diameters of about 200 nm to about 1000 nm, about 300 nm to about 1000 nm, about 400 nm to about 1000 nm, about 500 nm to about 1000 nm, about 600 nm to about 1000 nm, about 700 nm to about 1000 nm, about 800 nm to about 1000 nm, about 200 nm to about 800 nm, about 300 nm to about 800 nm, about 400 nm to about 800 nm, about 500 nm to about 800 nm, about 600 nm to about 800 nm, about 200 nm to about 600 nm, about 300 nm to about 600 nm, about 400 nm to about 600 nm, about 200 nm to about 500 nm, or about 300 nm to about 500 nm.

In certain embodiments, the majority (e.g., at least about 50%, at least about 55%, at least about 60%, at least about 65%, at least about 70%, at least about 75%, at least about 80%, at least about 85%, at least about 90%, at least about 95%, at least about 98%, at least about 99% or more) of EVs of a population of anti-inflammatory EVs as described herein have size diameters of about 200 nm to about 1000 nm, about 300 nm to about 1000 nm, about 400 nm to about 1000 nm, about 500 nm to about 1000 nm, about 600 nm to about 1000 nm, about 700 nm to about 1000 nm, about 800 nm to about 1000 nm, about 200 nm to about 800 nm, about 300 nm to about 800 nm, about 400 nm to about 800 nm, about 500 nm to about 800 nm, about 600 nm to about 800 nm, about 200 nm to about 600 nm, about 300 nm to about 600 nm, about 400 nm to about 600 nm, about 200 nm to about 500 nm, or about 300 nm to about 500 nm.

In certain embodiments, the majority (e.g., at least about 50%, at least about 55%, at least about 60%, at least about 65%, at least about 70%, at least about 75%, at least about 80%, at least about 85%, at least about 90%, at least about 95%, at least about 98%, at least about 99% or more) of the EVs of a population of anti-inflammatory EVs as described herein have a size diameter of about 400 nm, about 450 nm, about 500 nm, about 600 nm, about 650 nm, about 700 nm, about 750 nm, 800 nm, about 850 nm, about 900 nm, about 950 nm, or about 1000 nm.

In certain embodiments, the EVs of a population of anti-inflammatory EVs as described herein have a mean size diameter of about 30 nm to about 1000 nm. In certain embodiments, the EVs of a population of anti-inflammatory EVs as described herein have a mean size diameter of less than about 300 nm, less than about 200 nm, less than about 150 nm or less than about 100 nm. In certain embodiments, the EVs of a population of anti-inflammatory EVs as described herein have a mean size diameter of about 30 nm to about 300 nm, about 30 nm to about 250 nm, about 30 nm to about 200 nm, about 30 nm to about 160 nm, about 30 nm to about 150 nm, about 30 nm to about 100 nm, about 40 nm to about 300 nm, about 40 nm to about 200 nm, about 40 nm to about 160 nm, about 40 nm to about 150 nm, about 40 nm to about 100 nm, about 60 nm to about 300 nm, about 60 nm to about 200 nm, about 60 nm to about 160 nm, about 60 nm to about 150 nm, about 60 nm to about 125 nm, about 60 nm to about 110 nm, about 60 nm to about 100 nm, about 60 nm to about 80 nm, about 70 nm to about 300 nm, about 70 nm to about 200 nm, about 70 nm to about 160 nm, about 70 nm to about 150 nm, about 70 nm to about 125 nm, about 70 nm to about 110 nm, about 70 nm to about 100 nm, about 80 nm to about 300 nm, about 80 nm to about 200 nm, about 80 nm to about 160 nm, about 80 nm to about 150 nm, about 80 nm to about 125 nm, about 80 nm to about 110 nm, about 80 nm to about 100 nm, or about 110 nm to about 120 nm.

In certain embodiments, the EVs of a population of anti-inflammatory EVs as described herein have a mean size diameter of about 30 nm, about 40 nm, about 50 nm, about 60 nm, about 65 nm, about 70 nm, about 75 nm, 80 nm, about 85 nm, about 90 nm, about 95 nm, about 100 nm, about 110 nm to about 120 nm, about 150 nm, about 175 nm, about 200 nm, about 250 nm or about 300 nm.

In certain embodiments, the EVs of a population of anti-inflammatory EVs as described herein have a mean size diameter of about 80 nm to about 110 nm, about 80 nm to about 100 nm, about 80 to about 95 nm, about 80-90 nm, about 85 nm to about 110 nm, about 85 nm to about 100 nm, about 85 to about 95 nm, about 90 nm to about 110 nm, about 90 nm to about 100 nm, or about 90 to about 95 nm.

In certain embodiments, the EVs of a population of anti-inflammatory EVs as described herein have a mean size diameter greater than about 300 nm, greater than about 400 nm, greater than about 500 nm, greater than about 500 nm, greater than about 700 nm, or greater than about 800 nm. In certain embodiments, the EVs of a population of anti-inflammatory EVs as described herein have a mean size diameter of about 200 nm to about 1000 nm, about 300 nm to about 1000 nm, about 400 nm to about 1000 nm, about 500 nm to about 1000 nm, about 600 nm to about 1000 nm, about 700 nm to about 1000 nm, about 800 nm to about 1000 nm, about 200 nm to about 800 nm, about 300 nm to about 800 nm, about 400 nm to about 800 nm, about 500 nm to about 800 nm, about 600 nm to about 800 nm, about 200 nm to about 600 nm, about 300 nm to about 600 nm, about 400 nm to about 600 nm, about 200 nm to about 500 nm, or about 300 nm to about 500 nm.

In certain embodiments, the EVs of a population of anti-inflammatory EVs as described herein have a mean size diameter of about 400 nm, about 450 nm, about 500 nm, about 600 nm, about 650 nm, about 700 nm, about 750 nm, 800 nm, about 850 nm, about 900 nm, about 950 nm, or about 1000 nm. In certain embodiments, the EVs of a population of anti-inflammatory EVs as described herein have a mean size diameter of about 80 nm to about 110 nm. In certain embodiments, the EVs of a population of anti-inflammatory EVs as described herein have a mean size diameter of about 85 nm to about 100 nm. In certain embodiments, the EVs of a population of anti-inflammatory EVs as described herein have a mean size diameter of about 80 nm to about 100 nm. In certain embodiments, the EVs of a population of anti-inflammatory EVs as described herein have a mean size diameter of about 85 nm to about 95 nm.

In certain embodiments, the EVs of a population of anti-inflammatory EVs as described herein have a median size diameter of about 30 nm to about 1000 nm. In certain embodiments, the EVs of a population of anti-inflammatory EVs as described herein have a median size diameter of less than about 300 nm, less than about 200 nm, less than about 150 nm or less than about 100 nm. In certain embodiments, the EVs of a population of anti-inflammatory EVs as described herein have a median size diameter of about 30 nm to about 300 nm, about 30 nm to about 250 nm, about 30 nm to about 200 nm, about 30 nm to about 160 nm, about 30 nm to about 150 nm, about 30 nm to about 100 nm, about 40 nm to about 300 nm, about 40 nm to about 200 nm, about 40 nm to about 160 nm, about 40 nm to about 150 nm, about 40 nm to about 100 nm, about 60 nm to about 300 nm, about 60 nm to about 200 nm, about 60 nm to about 160 nm, about 60 nm to about 150 nm, about 60 nm to about 125 nm, about 60 nm to about 110 nm, about 60 nm to about 100 nm, about 60 nm to about 80 nm, about 70 nm to about 300 nm, about 70 nm to about 200 nm, about 70 nm to about 160 nm, about 70 nm to about 150 nm, about 70 nm to about 125 nm, about 70 nm to about 110 nm, about 70 nm to about 100 nm, about 75 nm to about 100 nm, about 75 nm to about 195 nm, about 75 nm to about 90 nm, about 75 nm to about 85 nm, about 80 nm to about 300 nm, about 80 nm to about 200 nm, about 80 nm to about 160 nm, about 80 nm to about 150 nm, about 80 nm to about 125 nm, about 80 nm to about 110 nm, about 80 nm to about 100 nm, about 80 nm to about 95 nm, about 85 nm to about 95 nm, or about 110 nm to about 120 nm. In certain embodiments, the EVs of a population of anti-inflammatory EVs as described herein have a median size diameter of about 30 nm to about 1000 nm.

In certain embodiments, the EVs of a population of anti-inflammatory EVs as described herein have a median size diameter of about 70 nm to about 100 nm. In certain embodiments, the EVs of a population of anti-inflammatory EVs as described herein have a median size diameter of about 75 nm to about 100 nm. In certain embodiments, the EVs of a population of anti-inflammatory EVs as described herein have a median size diameter of about 75 nm to about 95 nm.

In certain embodiments, the EVs of a population of anti-inflammatory EVs as described herein have a median size diameter of about 30 nm, about 40 nm, about 50 nm, about 60 nm, about 65 nm, about 70 nm, about 75 nm, 80 nm, about 85 nm, about 90 nm, about 95 nm, about 100 nm, about 110 nm to about 120 nm, about 150 nm, about 175 nm, about 200 nm, about 250 nm or about 300 nm.

In certain embodiments, the EVs of a population of anti-inflammatory EVs as described herein have a median size diameter greater than about 300 nm, greater than about 400 nm, greater than about 500 nm, greater than about 500 nm, greater than about 700 nm, or greater than about 800 nm. In certain embodiments, the EVs of a population of anti-inflammatory EVs as described herein have a median size diameter of about 200 nm to about 1000 nm, about 300 nm to about 1000 nm, about 400 nm to about 1000 nm, about 500 nm to about 1000 nm, about 600 nm to about 1000 nm, about 700 nm to about 1000 nm, about 800 nm to about 1000 nm, about 200 nm to about 800 nm, about 300 nm to about 800 nm, about 400 nm to about 800 nm, about 500 nm to about 800 nm, about 600 nm to about 800 nm, about 200 nm to about 600 nm, about 300 nm to about 600 nm, about 400 nm to about 600 nm, about 200 nm to about 500 nm, or about 300 nm to about 500 nm.

In certain embodiments, the EVs of a population of anti-inflammatory EVs as described herein have a median size diameter of about 400 nm, about 450 nm, about 500 nm, about 600 nm, about 650 nm, about 700 nm, about 750 nm, 800 nm, about 850 nm, about 900 nm, about 950 nm, or about 1000 nm.

In certain embodiments, the EVs of a population of anti-inflammatory EVs as described herein have a mode size diameter of about 30 nm to about 1000 nm. In certain embodiments, the EVs of a population of anti-inflammatory EVs as described herein have a mode size diameter of less than about 300 nm, less than about 200 nm, less than about 150 nm or less than about 100 nm. In certain embodiments, the EVs of a population of anti-inflammatory EVs as described herein have a mode size diameter of about 30 nm to about 300 nm, about 30 nm to about 250 nm, about 30 nm to about 200 nm, about 30 nm to about 160 nm, about 30 nm to about 150 nm, about 30 nm to about 100 nm, about 40 nm to about 300 nm, about 40 nm to about 200 nm, about 40 nm to about 160 nm, about 40 nm to about 150 nm, about 40 nm to about 100 nm, about 60 nm to about 300 nm, about 60 nm to about 200 nm, about 60 nm to about 160 nm, about 60 nm to about 150 nm, about 60 nm to about 125 nm, about 60 nm to about 110 nm, about 60 nm to about 100 nm, about 60 nm to about 80 nm, about 70 nm to about 300 nm, about 70 nm to about 200 nm, about 70 nm to about 160 nm, about 70 nm to about 150 nm, about 70 nm to about 125 nm, about 70 nm to about 110 nm, about 70 nm to about 100 nm, about 80 nm to about 300 nm, about 80 nm to about 200 nm, about 80 nm to about 160 nm, about 80 nm to about 150 nm, about 80 nm to about 125 nm, about 80 nm to about 110 nm, about 80 nm to about 100 nm, or about 110 nm to about 120 nm. In certain embodiments, the EVs of a population of anti-inflammatory EVs as described herein have a mode size diameter of about 65 nm to about 85 nm, about 65 nm to about 80 nm, about 65 nm to about 75 nm, about 70 nm to about 85 nm, about 70 nm to about 80 nm, or about 70 nm to about 75 nm.

In certain embodiments, the EVs of a population of anti-inflammatory EVs as described herein have a mode size diameter of about 65 nm to about 95 nm. In certain embodiments, the EVs of a population of anti-inflammatory EVs as described herein have a mode size diameter of about 75 nm to about 85 nm. In certain embodiments, the EVs of a population of anti-inflammatory EVs as described herein have a mode size diameter of about 75 nm to about 85 nm.

In certain embodiments, the EVs of a population of anti-inflammatory EVs as described herein have a mode size diameter of about 30 nm, about 40 nm, about 50 nm, about 60 nm, about 65 nm, about 70 nm, about 75 nm, 80 nm, about 85 nm, about 90 nm, about 95 nm, about 100 nm, about 110 nm to about 120 nm, about 150 nm, about 175 nm, about 200 nm, about 250 nm or about 300 nm.

In certain embodiments, the EVs of a population of anti-inflammatory EVs as described herein have a mode size diameter greater than about 300 nm, greater than about 400 nm, greater than about 500 nm, greater than about 500 nm, greater than about 700 nm, or greater than about 800 nm. In certain embodiments, the EVs of a population of anti-inflammatory EVs as described herein have a mode size diameter of about 200 nm to about 1000 nm, about 300 nm to about 1000 nm, about 400 nm to about 1000 nm, about 500 nm to about 1000 nm, about 600 nm to about 1000 nm, about 700 nm to about 1000 nm, about 800 nm to about 1000 nm, about 200 nm to about 800 nm, about 300 nm to about 800 nm, about 400 nm to about 800 nm, about 500 nm to about 800 nm, about 600 nm to about 800 nm, about 200 nm to about 600 nm, about 300 nm to about 600 nm, about 400 nm to about 600 nm, about 200 nm to about 500 nm, or about 300 nm to about 500 nm.

In certain embodiments, the EVs of a population of anti-inflammatory EVs as described herein have a mode size diameter of about 400 nm, about 450 nm, about 500 nm, about 600 nm, about 650 nm, about 700 nm, about 750 nm, 800 nm, about 850 nm, about 900 nm, about 950 nm, or about 1000 nm.

In certain aspects a population of anti-inflammatory EVs as described herein is a buffer-containing population of anti-inflammatory EVs. The anti-inflammatory EVs described herein are derived from ex vivo-expanded human suppressive immune cells, e.g., Tregs. As explained in detailed below, a population of such anti-inflammatory EVs may be isolated from a culture comprising ex vivo expanding human suppressive immune cells, e.g., Tregs and culture media. In certain instances, as part of the process of isolating EVs from the culture, the culture media may be replaced with a buffer, for example a sterile buffer, e.g., a buffer suitable for administration to a human, such as suitable for administration to a human for therapeutic use. In such instances, the resulting isolated, cell-free population of anti-inflammatory EVs may be referred to as a buffer-containing population of anti-inflammatory EVs.

Similarly, in certain embodiments, a population of anti-inflammatory EVs as described herein is a saline-containing population of anti-inflammatory EVs. In particular embodiments, a population of anti-inflammatory EVs as described herein is a normal saline-containing population of anti-inflammatory EVs. In particular embodiments, a population of anti-inflammatory EVs as described herein is a 0.9% saline-containing population of anti-inflammatory EVs. In particular embodiments, a population of anti-inflammatory EVs as described herein is a phosphate-buffer saline-containing population of anti-inflammatory EVs.

The isolated, cell-free populations of anti-inflammatory EVs described herein are substantially free of cellular material, microparticles or other contaminants (e.g., organelles, lipids, cholesterol) from the cell or tissue source from which the EVs are derived, e.g., from the human suppressive immune cells, for example Tregs, from which the EVs are derived. For example, the isolated, cell-free populations of anti-inflammatory EVs described herein generally contain less than about 5 weight percent, less than about 1 weight percent, less than about 0.5 weight percent, less than about 0.1 weight percent, or less than about 0.01 weight percent of free of cellular material, microparticles or other contaminants (e.g., organelles, lipids, cholesterol) from the cell or tissue source from which the EVs are derived, e.g., from the human suppressive immune cells, for example Tregs, from which the EVs are derived.

In certain embodiments, the isolated, cell-free populations of anti-inflammatory EVs described herein are present in a composition that is substantially free of other EVs. For example, in certain embodiments, the isolated, cell-free populations of anti-inflammatory EVs described herein are present in a composition that contains less than about 20%, less than about 10%, less than about 5%, or less than about 1% other EVs.

In certain embodiments, an isolated, cell-free population of anti-inflammatory EVs described herein is present in a composition that comprises other EVs, wherein the isolated, cell-free population of anti-inflammatory EVs makes up about 10%, about 20%, about 25%, about 30%, about 35%, about 40%, about 50%, about 60%, about 70%, about 80%, about 90%, about 95%, or greater than about 95% of the EVs in the composition. In specific embodiments, the other EVs are serum EVs, for example, bovine serum EVs or human serum EVs.

In certain embodiments, the cell-free population of anti-inflammatory EVs are made up of anti-inflammatory EVs that comprise one or more cargos, e.g., drugs, detectable labels, proteins, nucleic acids, e.g., RNAs such as mRNAs and or miRNAs heterologous to the EVs. The cargo or cargos of the resulting anti-inflammatory EVs may be present within the Evs and/or present on the EV surface.

In certain embodiments, the cell-free population of anti-inflammatory EVs have been produced from Treg cells that have been genetically engineered, e.g., genetically engineered to express a cargo that is loaded into the EVs as they are produced. Alternatively, one or more cargos may, for example, be introduced into the culture media during EV production process and loaded into the EVs as the EVs are produced. In yet other alternatives one or more cargos may be introduced into the EVs via such techniques as electroporation or sonication. The cargo or cargos of the resulting anti-inflammatory EVs may be present within the Evs and/or present on the EV surface.

### 5.1.1. Gene Product Expression Profile

The populations of anti-inflammatory EVs provided herein may be characterized by their gene product expression profiles. For example, if the population of anti-inflammatory EVs is derived from a population of Tregs cultured in a serum-containing medium that contains serum EVs (also referred to as “media EVs”), the gene product expression profile of the population of anti-inflammatory EVs may be compared to the media EVs. In this context of EV gene product expression as discussed herein, two values can be considered to be substantially the same when the difference between them is not statistically significant (e.g., p≥0.1, ≥0.05, ≥0.01, or ≥ 0.001) and/or if the fold-difference between the two values is less than about 1.5-fold or less than about 2-fold (increase or decrease).

Changes in gene product expression may be expressed as “-fold increase” or “-fold decrease” or as the binary logarithm (or “log 2”) of the -fold change. In some embodiments, gene product expression is increased at least about 4-fold. In some embodiments, gene product expression is increased about 5-10-fold, about 10-15-fold, about 15-20-fold, about 20-25-fold, about 25-30-fold, about 30-35-fold, about 35-40-fold, about 40-45-fold, about 45-50-fold, about 50-60-fold, about 60-70 fold, about 70-80-fold, about 80-90-fold, about 90-100-fold, or at least about 100-fold. In some embodiments, gene product expression is decreased at least about 4-fold. In some embodiments, gene product expression is increased about 5-10-fold, about 10-15-fold, about 15-20-fold, about 20-25-fold, about 25-30-fold, about 30-35-fold, about 35-40-fold, about 40-45-fold, about 45-50-fold, about 50-60-fold, about 60-70 fold, about 70-80-fold, about 80-90-fold, about 90-100-fold, or at least about 100-fold.

In some embodiments, a population of anti-inflammatory EVs provided herein is characterized by its gene product expression profile as depicted in Table 10. In some embodiments, a population of anti-inflammatory EVs provided herein is characterized by the detectable expression of at least 5, at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, or all of the 191 gene products listed in Table 10. In a particular embodiment, such gene products include at least one of MIF, LGALS3 and S100A4. In other particular embodiments, such gene products include at least MIF, LGALS3 and S100A4.

In some embodiments, a population of anti-inflammatory EVs provided herein is characterized by an increased expression (e.g., an at least 1.5-fold, at least 2-fold, at least 3-fold, at least 4-fold, at least 5-fold, at least 10-fold, at least 20-fold, at least 50-fold, at least 100-fold, at least 200-fold, at least 500-fold, or at least 1000-fold expression, an expression at a log 2 fold of at least 0.5, at least 1, at least 2, at least 3, at least 5, at least 7, at least 8, at least 9, or at least 10, and/or a statistically significant increased expression (e.g., p<0.1, <0.05, <0.01, or <0.001)) of at least 5, at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, or all of the 191 gene products listed in Table 10, compared to a reference EV, for example, compared to the corresponding media EVs when the anti-inflammatory EVs are cultured in a serum-containing media. In a particular embodiment, such gene products include at least one of MIF, LGALS3 and S100A4. In other particular embodiments, such gene products include at least MIF, LGALS3 and S100A4.

Gene product expression may be determined by any method known in the art, for example, quantitative real-time PCR, Fluidigm™ Chip assays, RNA sequencing, or proteomics analysis (e.g., single-shot proteomics). In a specific embodiment, gene product expression is determined by proteomics analysis (e.g., single-shot proteomics). In a specific embodiment, the proteomics analysis is performed by mass spectrometry.

Similarly, the populations of Tregs from which the anti-inflammatory EVs provided herein are derived herein may be characterized by their gene product expression profiles. For example, the gene product expression profile of a population of Tregs provided herein may be compared to the Tregs at baseline. In this context, the term “baseline,” or “baseline Treg cell population denotes a population of Tregs that has been enriched from a patient sample but has not yet been expanded. In this context of Treg gene product expression as discussed herein, two values are considered to be substantially the same when the difference between them is not statistically significant (i.e., p>0.05) and/or if the binary log of the fold-difference between the two values is less than about 2-fold (increase or decrease).

Changes in gene product expression may be expressed as “-fold increase” or “-fold decrease” or as the binary logarithm (or “log 2”) of the fold change. In some embodiments, gene product expression is increased at least about 4-fold. In some embodiments, gene product expression is increased about 5-10-fold, about 10-15-fold, about 15-20-fold, about 20-25-fold, about 25-30-fold, about 30-35-fold, about 35-40-fold, about 40-45-fold, about 45-50-fold, about 50-60-fold, about 60-70 fold, about 70-80-fold, about 80-90-fold, about 90-100-fold, or at least about 100-fold. In some embodiments, gene product expression is decreased at least about 4-fold. In some embodiments, gene product expression is increased about 5-10-fold, about 10-15-fold, about 15-20-fold, about 20-25-fold, about 25-30-fold, about 30-35-fold, about 35-40-fold, about 40-45-fold, about 45-50-fold, about 50-60-fold, about 60-70 fold, about 70-80-fold, about 80-90-fold, about 90-100-fold, or at least about 100-fold.

The level of gene product expression may be above or below the limit of detection of the method being utilized to measure gene product expression. In some embodiments, the expression level of a gene product listed in any of Table 3-Table 7 may be undetectable (i.e., below the level of detection for the method utilized such as single-shot proteomics) in a population of Tregs from which the anti-inflammatory EVs provided herein are derived. In some embodiments, the expression level of a gene product listed in any of Table 3-Table 7 may be detectable (i.e., above the level of detection for the method utilized such as single-shot proteomics) in a population of Tregs from which the anti-inflammatory EVs provided herein are derived. In some embodiments, the expression level of a gene product listed in any of Table 3-Table 7 may become detectable or undetectable in a population of Tregs from which the anti-inflammatory EVs provided herein are derived upon enrichment or expansion of the population of Tregs. Gene product expression may be determined by any method known in the art, for example, quantitative real-time PCR, Fluidigm™ Chip assays, RNA sequencing, or proteomics analysis (e.g., single-shot proteomics). In a specific embodiment, gene product expression is determined by proteomics analysis (e.g., single-shot proteomics). In a specific embodiment, the proteomics analysis is performed by mass spectrometry.

In some embodiments, expression of one or more of the gene products listed in Table 3 and/or Table 4 is decreased in the Tregs from which the anti-inflammatory EVs provided herein are derived post-expansion compared to the Tregs at baseline. In some embodiments, expression of one or more of the gene products listed in Table 3 is decreased in the Tregs from which the anti-inflammatory EVs provided herein are derived post-expansion compared to the Tregs at baseline. In some embodiments, expression of one or more of the gene products listed in Table 4 is decreased in the Tregs from which the anti-inflammatory EVs provided herein are derived post-expansion compared to the Tregs at baseline.

In some embodiments, expression of one or more of the gene products listed in Table 5-Table 9 is increased in the Tregs from which the anti-inflammatory EVs provided herein are derived post-expansion compared to the Tregs at baseline. In some embodiments, the expression of one or more gene products associated with a dysfunctional Treg phenotype (e.g., one or more of the gene products listed in Table 3) is decreased in the Tregs from which the anti-inflammatory EVs provided herein are derived post-expansion compared to the Tregs at baseline. A dysfunctional Treg phenotype includes, for example, dysregulated calcium dynamics, loss of MECP2 binding ability to 5-Hydroxymethylcytosine (5 hmC)-DNA, dysregulation of MECP2 expression or activity, and loss of MECP2 regulation, phosphorylation or binding abilities.

### 5.1.2. Treg EV Surface Marker Profile

The populations of anti-inflammatory EVs provided herein may be characterized by their Treg EV surface marker profiles. If the population of anti-inflammatory EVs is derived from a population of Tregs cultured in a serum-containing medium that contains serum EVs (also referred to as “media EVs”), a Treg EV surface marker profile of the population of anti-inflammatory EVs may be compared to the surface marker profile of the media EVs. For example such a comparison may be used to identify and, optionally remove or subtract from the profile aspects contributed by the media EVs.

In particular embodiments, an EV surface marker profile may be produced by using antibody-based methods of detecting epitopes on the surface of the EVs. For example, well known techniques utilizing cocktails of various fluorescently labeled bead populations, each coated with a specific antibody binding to a surface epitope of interest in conjunction with flow cytometry methods may be used to produce an EV surface marker profile. For example, the MACSPlex Exosome Kit (Miltenyi Biotec) utilizes such a technique. In a particular embodiment, a population of anti-inflammatory EVs provided herein is characterized by a Treg surface marker profile as generated using a MACSPlex Exosome Kit (Miltenyi Biotec).

An EV surface marker profile may, for example, be presented using relative intensity units, for example, median intensity units, e.g., median fluorescence intensity (MFI) units, such that the EV surface marker profile conveys the detectable presence or absence of markers being assayed and additionally conveys the relative amounts of such markers.

In a specific example, a population of anti-inflammatory EVs as described herein exhibits a Treg EV surface marker profile as shown in Example 21 (“Treg EV surface marker signature”), below. In a specific example, a population of anti-inflammatory EVs as described herein exhibits a Treg EV surface marker profile as shown in Example 21 (“Treg EV surface marker signature”), below, generated using the techniques described in Example 21, e.g., generated using a MACSPlex Exosome Kit (Miltenyi Biotec).

Each of the surface markers (e.g., CD2, CD25 etc.) are well-known, as are antibodies specific for epitopes present on such markers. It is noted that “HLA-DRDPDQ” refers to HLA-Class II molecules HLA-DR, HLA-DP and HLA-DQ. As the Treg EVs described herein are generally obtained from human Treg cells that have been ex vivo expanded, in particular embodiments, such surface markers are human (e.g., human CD2, human HLA-DRDPDQ, CD25 etc.).

In certain embodiments, a population of anti-inflammatory EVs provided herein is characterized by a Treg EV surface marker profile comprising at least one, two or all three of CD2, HLA-DRDPDQ, and/or CD25. In particular embodiments, a population of anti-inflammatory EVs provided herein is characterized by a Treg EV surface marker profile comprising CD2, HLA-DRDPDQ, and CD25. In particular embodiments, a population of anti-inflammatory EVs provided herein is characterized by a Treg EV surface marker profile comprising CD2, HLA-DRDPDQ, and CD25, wherein CD2 is present at an amount at least 1-fold, 2-fold or 5-fold higher than HLA-DRDPDQ and/or CD25, as measured using the assay used to measure or identify the Treg EV surface marker profile, e.g, a MACSPlex Exosome Kit assay.

As used herein, when a population of anti-inflammatory EVs provided herein is described as being characterized by a Treg surface marker profile comprising one or marker recited markers, this refers to the population of anti-inflammatory EVs comprising a detectable amount of the marker using the assay used to measure or identify the Treg EV surface marker profile. Such assays are well known in the art and, for example, an antibody-based detection assay, e.g., an antibody-based detection technique comprising detection of directly or indirectly labeled, e.g., fluorescently labeled, antibodies specific for a surface marker epitope. Such an assay may, for example comprise utilizing differentially labeled beads coated with antibodies specific for a surface marker epitope in conjunction with flow cytometry. In specific embodiments, such an assay may utilize MACSPlex Exosome Kit assay such as, for example, described in Example 21 (“Treg EV surface marker signature”), below.

In certain embodiments, a population of anti-inflammatory EVs provided herein is characterized by a Treg EV surface marker profile comprising at least one, two or all three of CD2, HLA-DRDPDQ, and/or CD25, and substantially lacking at least one, two, three, four, five, six, seven, eight, nine, ten, eleven, twelve, thirteen, fourteen, fifteen, sixteen, seventeen, eighteen, nineteen, twenty, twenty-one or all of CD3, CD19, CD8, CD56, CD105, CD1c, CD49e, ROR1, CD209, SSEA-4, CD40, CD62P, CD11c, CD40, MSCP (mesenchymal stem cell-like protein), CD146, CD86, CD326, CD133, CD142, CD31, and/or CD14.

In certain embodiments, a population of anti-inflammatory EVs provided herein is characterized by a Treg EV surface marker profile comprising CD2, HLA-DRDPDQ, and CD25, and substantially lacking at least one, two, three, four, five, six, seven, eight, nine, ten, eleven, twelve, thirteen, fourteen, fifteen, sixteen, seventeen, eighteen, nineteen, twenty, twenty-one or all of CD3, CD19, CD8, CD56, CD105, CD1c, CD49e, ROR1, CD209, SSEA-4, CD40, CD62P, CD11c, CD40, MSCP (mesenchymal stem cell-like protein), CD146, CD86, CD326, CD133, CD142, CD31, and/or CD14.

In certain embodiments, a population of anti-inflammatory EVs provided herein is characterized by a Treg EV surface marker profile comprising CD2, HLA-DRDPDQ, and CD25, and substantially lacking CD3, CD19, CD8, CD56, CD105, CD1c, CD49e, ROR1, CD209, SSEA-4, CD40, CD62P, CD11c, CD40, MSCP (mesenchymal stem cell-like protein), CD146, CD86, CD326, CD133, CD142, CD31, and CD14.

In particular embodiments, “substantially lacking” as used herein may refer to a level that is below the level of detection using the assay used to measure or identify the Treg EV surface marker profile, e.g., a MACSPlex Exosome Kit assay. In particular embodiments, “substantially lacking,” as used herein, may refer to a level that is at least 5-fold, 10-fold, 15-fold, or 20-fold less than the level of CD2, HLA-DRDPDQ and/or CD25, if such markers are part of the Treg EV surface marker profile, as measured using the assay used to measure or identify the Treg EV surface marker profile, e.g, a MACSPlex Exosome Kit assay. In particular embodiments CD2 is part of the Treg EV surface marker profile and “substantially lacking,” as used herein, refers to a level that is at least 5-fold, 10-fold, 15-fold, or 20-fold less than the level of CD2, as measured using the assay used to measure or identify the Treg EV surface marker profile, e.g, a MACSPlex Exosome Kit assay.

In certain embodiments, a population of anti-inflammatory EVs provided herein is characterized by a Treg EV surface marker profile comprising at least one, two or all three of CD2, HLA-DRDPDQ, and/or CD25 and further comprising at least one of CD44, CD29, CD4 and/or CD45. In certain embodiments, a population of anti-inflammatory EVs provided herein is characterized by a Treg EV surface marker profile comprising CD2, HLA-DRDPDQ, and CD25 and further comprising at least one of CD44, CD29, CD4 and/or CD45. In certain embodiments, a population of anti-inflammatory EVs provided herein is characterized by a Treg EV surface marker profile comprising CD2, HLA-DRDPDQ, and CD25 and further comprising CD44, CD29, CD4 and CD45. In certain embodiments, a population of anti-inflammatory EVs provided herein is characterized by a Treg EV surface marker profile comprising CD2, HLA-DRDPDQ, and CD25 and further comprising CD44, CD29, CD4 and CD45, wherein CD2 is present at an amount at least 1-fold, 2-fold or 5-fold higher than HLA-DRDPDQ and/or CD25, and CD44, CD29, CD4 and CD45, as measured using the assay used to measure or identify the Treg EV surface marker profile, e.g, a MACSPlex Exosome Kit assay.

In particular embodiments of any such population of anti-inflammatory EVs described directly hereinabove, such a population is further characterized by a Treg EV surface marker profile substantially lacking at least one, two, three, four, five, six, seven, eight, nine, ten, eleven, twelve, thirteen, fourteen, fifteen, sixteen, seventeen, eighteen, nineteen, twenty, twenty-one or all of CD3, CD19, CD8, CD56, CD105, CD1c, CD49e, ROR1, CD209, SSEA-4, CD40, CD62P, CD11c, CD40, MSCP (mesenchymal stem cell-like protein), CD146, CD86, CD326, CD133, CD142, CD31, and/or CD14. In particular embodiments of any such population of anti-inflammatory EVs described directly hereinabove, such a population is further characterized by a Treg EV surface marker profile substantially lacking CD3, CD19, CD8, CD56, CD105, CD1c, CD49e, ROR1, CD209, SSEA-4, CD40, CD62P, CD11c, CD40, MSCP (mesenchymal stem cell-like protein), CD146, CD86, CD326, CD133, CD142, CD31, and CD14.

In certain embodiments, a population of anti-inflammatory EVs provided herein is characterized by a Treg EV surface marker profile comprising: i) at least one, two or all three of CD2, HLA-DRDPDQ, and/or CD25, ii) further comprising at least one of CD44, CD29, CD4 and/or CD45, and iii) further comprising at least one of HLA-ABC, CD24, CD69, CD41b, and/or CD42a. In certain embodiments, a population of anti-inflammatory EVs provided herein is characterized by a Treg EV surface marker profile comprising: i) CD2, HLA-DRDPDQ, and CD25, ii) further comprising at least one of CD44, CD29, CD4 and/or CD45, and iii) further comprising at least one of HLA-ABC, CD24, CD69, CD41b, and/or CD42a, for example, at least one of HLA-ABC, CD24 and/or CD69. In certain embodiments, a population of anti-inflammatory EVs provided herein is characterized by a Treg EV surface marker profile comprising: i) CD2, HLA-DRDPDQ, and CD25, ii) further comprising CD44, CD29, CD4 and CD45, and iii) further comprising at least one of HLA-ABC, CD24, CD69, CD41b, and/or CD42a, for example, at least one of HLA-ABC, CD24 and/or CD69. In certain embodiments, a population of anti-inflammatory EVs provided herein is characterized by a Treg EV surface marker profile comprising: i) CD2, HLA-DRDPDQ, and CD25, ii) further comprising CD44, CD29, CD4 and CD45, and iii) further comprising HLA-ABC, CD24, and CD69, and, optionally, CD41b and CD42a. In certain embodiments, a population of anti-inflammatory EVs provided herein is characterized by a Treg EV surface marker profile comprising: i) CD2, HLA-DRDPDQ, and CD25, ii) further comprising CD44, CD29, CD4 and CD45, and iii) further comprising HLA-ABC, CD24, and CD69, and, optionally, CD41b and CD42a. wherein CD2 is present at an amount at least 1-fold, 2-fold or 5-fold higher than HLA-DRDPDQ and/or CD25, and CD44, CD29, CD4, CD45, HLA-ABC, CD24, CD69, and (if present) CD41b and CD42a, as measured using the assay used to measure or identify the Treg EV surface marker profile, e.g, a MACSPlex Exosome Kit assay.

In particular embodiments of any such population of anti-inflammatory EVs described directly hereinabove, such a population is further characterized by a Treg EV surface marker profile substantially lacking at least one, two, three, four, five, six, seven, eight, nine, ten, eleven, twelve, thirteen, fourteen, fifteen, sixteen, seventeen, eighteen, nineteen, twenty, twenty-one or all of CD3, CD19, CD8, CD56, CD105, CDIc, CD49e, ROR1, CD209, SSEA-4, CD40, CD62P, CD11c, CD40, MSCP (mesenchymal stem cell-like protein), CD146, CD86, CD326, CD133, CD142, CD31, and/or CD14. In particular embodiments of any such population of anti-inflammatory EVs described directly hereinabove, such a population is further characterized by a Treg EV surface marker profile substantially lacking CD3, CD19, CD8, CD56, CD105, CD1c, CD49e, ROR1, CD209, SSEA-4, CD40, CD62P, CD11c, CD40, MSCP (mesenchymal stem cell-like protein), CD146, CD86, CD326, CD133, CD142, CD31, and CD14.

In certain embodiments, a population of anti-inflammatory EVs provided herein is characterized by a Treg EV surface marker profile comprising: i) at least one, two or all three of CD2, HLA-DRDPDQ, and/or CD25, ii) further comprising at least one of CD44, CD29, CD4 and/or CD45, iii) further comprising at least one of HLA-ABC, CD24, CD69, CD41b, and/or CD42a, for example, at least one of HLA-ABC, CD24 and/or CD69; and iv) further comprising at least one exosome or EV marker, for example at least one, two or all three of CD81, CD63 and/or CD9. In certain embodiments, a population of anti-inflammatory EVs provided herein is characterized by a Treg EV surface marker profile comprising: i) CD2, HLA-DRDPDQ, and CD25, ii) further comprising at least one of CD44, CD29, CD4 and/or CD45, iii) further comprising at least one of HLA-ABC, CD24, CD69, CD41b, and/or CD42a, for example, at least one of HLA-ABC, CD24 and/or CD69; and iv) further comprising at least one exosome or EV marker, for example at least one, two or all three of CD81, CD63 and/or CD9. In certain embodiments, a population of anti-inflammatory EVs provided herein is characterized by a Treg EV surface marker profile comprising: i) CD2, HLA-DRDPDQ, and CD25, ii) further comprising CD44, CD29, CD4 and CD45, iii) further comprising at least one of HLA-ABC, CD24, CD69, CD41b, and/or CD42a, for example, at least one of HLA-ABC, CD24 and/or CD69; and iv) further comprising at least one exosome or EV marker, for example at least one, two or all three of CD81, CD63 and/or CD9. In certain embodiments, a population of anti-inflammatory EVs provided herein is characterized by a Treg EV surface marker profile comprising: i) CD2, HLA-DRDPDQ, and CD25, ii) further comprising CD44, CD29, CD4 and CD45, iii) further comprising at least one of HLA-ABC, CD24, CD69, and, optionally, CD41b, and CD42a; and iv) further comprising at least one exosome or EV marker, for example at least one, two or all three of CD81, CD63 and/or CD9. In certain embodiments, a population of anti-inflammatory EVs provided herein is characterized by a Treg EV surface marker profile comprising: i) CD2, HLA-DRDPDQ, and CD25, ii) further comprising CD44, CD29, CD4 and CD45, iii) further comprising at least one of HLA-ABC, CD24, CD69, and, optionally, CD41b, and CD42a; and iv) further comprising at least one, two or all three of CD81, CD63 and/or CD9. In certain embodiments, a population of anti-inflammatory EVs provided herein is characterized by a Treg EV surface marker profile comprising: i) CD2, HLA-DRDPDQ, and CD25, ii) further comprising CD44, CD29, CD4 and CD45, iii) further comprising HLA-ABC, CD24, CD69, and, optionally, CD41b, and CD42a; and iv) further comprising at least one, two or all three of CD81, CD63 and CD9. In certain embodiments, a population of anti-inflammatory EVs provided herein is characterized by a Treg EV surface marker profile comprising: i) CD2, HLA-DRDPDQ, and CD25, ii) further comprising CD44, CD29, CD4 and CD45, iii) further comprising HLA-ABC, CD24, CD69, and, optionally, CD41b, and CD42a; and iv) further comprising CD81, CD63 and CD9. In certain embodiments, a population of anti-inflammatory EVs provided herein is characterized by a Treg EV surface marker profile comprising: i) CD2, HLA-DRDPDQ, and CD25, ii) further comprising CD44, CD29, CD4 and CD45, iii) further comprising HLA-ABC, CD24, CD69, CD41b, and CD42a; and iv) further comprising CD81, CD63 and CD9. In certain embodiments, a population of anti-inflammatory EVs provided herein is characterized by a Treg EV surface marker profile comprising: i) CD2, HLA-DRDPDQ, and CD25, ii) further comprising CD44, CD29, CD4 and CD45, iii) further comprising HLA-ABC, CD24, CD69, CD41b, and CD42a; and iv) further comprising CD81, CD63 and CD9, wherein CD2 is present at an amount at least 1-fold, 2-fold or 5-fold higher than HLA-DRDPDQ and/or CD25, and CD44, CD29, CD4, CD45, HLA-ABC, CD24, CD69, CD42a, CD81, CD63 and CD9, as measured using the assay used to measure or identify the Treg EV surface marker profile, e.g, a MACSPlex Exosome Kit assay.

In particular embodiments of any such population of anti-inflammatory EVs described directly hereinabove, such a population is further characterized by a Treg EV surface marker profile substantially lacking at least one, two, three, four, five, six, seven, eight, nine, ten, eleven, twelve, thirteen, fourteen, fifteen, sixteen, seventeen, eighteen, nineteen, twenty, twenty-one or all of CD3, CD19, CD8, CD56, CD105, CDIc, CD49e, ROR1, CD209, SSEA-4, CD40, CD62P, CD11c, CD40, MSCP (mesenchymal stem cell-like protein), CD146, CD86, CD326, CD133, CD142, CD31, and/or CD14. In particular embodiments of any such population of anti-inflammatory EVs described directly hereinabove, such a population is further characterized by a Treg EV surface marker profile substantially lacking CD3, CD19, CD8, CD56, CD105, CDIc, CD49e, ROR1, CD209, SSEA-4, CD40, CD62P, CD11c, CD40, MSCP (mesenchymal stem cell-like protein), CD146, CD86, CD326, CD133, CD142, CD31, and CD14.

In certain embodiments, a population of anti-inflammatory EVs provided herein is characterized by a Treg EV surface marker profile comprising: i) CD2, HLA-DRDPDQ, and CD25, ii) further comprising CD44, CD29, CD4 and CD45, iii) further comprising HLA-ABC, CD24, CD69, CD41b, and CD42a; iv) further comprising CD81, CD63 and CD9; and wherein the Treg EV surface marker profile is characterized by substantially lacking CD3, CD19, CD8, CD56, CD105, CD1c, CD49e, ROR1, CD209, SSEA-4, CD40, CD62P, CD11c, CD40, MSCP (mesenchymal stem cell-like protein), CD146, CD86, CD326, CD133, CD142, CD31, and CD14.

In certain embodiments, a population of anti-inflammatory EVs provided herein is characterized by a Treg EV surface marker profile comprising: i) CD2, HLA-DRDPDQ, and CD25, ii) further comprising CD44, CD29, CD4 and CD45, iii) further comprising HLA-ABC, CD24, CD69, CD41b, and CD42a; iv) further comprising CD81, CD63 and CD9, wherein CD2 is present at an amount at least 1-fold, 2-fold or 5-fold higher than HLA-DRDPDQ and/or CD25, and CD44, CD29, CD4, CD45, HLA-ABC, CD24, CD69, CD42a, CD81, CD63 and CD9, as measured using the assay used to measure or identify the Treg EV surface marker profile, e.g, a MACSPlex Exosome Kit assay; and wherein the Treg EV surface marker profile is characterized by substantially lacking CD3, CD19, CD8, CD56, CD105, CD1c, CD49e, ROR1, CD209, SSEA-4, CD40, CD62P, CD11c, CD40, MSCP (mesenchymal stem cell-like protein), CD146, CD86, CD326, CD133, CD142, CD31, and CD14.

In certain embodiments, a population of anti-inflammatory EVs provided herein is characterized by a Treg EV surface marker profile comprising: i) at least one, two or all three of CD2, HLA-DRDPDQ, and/or CD25, ii) further comprising at least one of CD44, CD29, CD4 and/or CD45, and iii) further comprising at least one of HLA-ABC, CD24 and/or CD69. In certain embodiments, a population of anti-inflammatory EVs provided herein is characterized by a Treg EV surface marker profile comprising: i) CD2, HLA-DRDPDQ, and CD25, ii) further comprising at least one of CD44, CD29, CD4 and/or CD45, and iii) further comprising at least one of HLA-ABC, CD24 and/or CD69. In certain embodiments, a population of anti-inflammatory EVs provided herein is characterized by a Treg EV surface marker profile comprising: i) CD2, HLA-DRDPDQ, and CD25, ii) further comprising CD44, CD29, CD4 and CD45, and iii) further comprising at least one of HLA-ABC, CD24 and/or CD69. In certain embodiments, a population of anti-inflammatory EVs provided herein is characterized by a Treg EV surface marker profile comprising: i) CD2, HLA-DRDPDQ, and CD25, ii) further comprising CD44, CD29, CD4 and CD45, and iii) further comprising HLA-ABC, CD24 and CD69.

In particular embodiments of any such population of anti-inflammatory EVs described directly hereinabove, such a population is further characterized by a Treg EV surface marker profile substantially lacking at least one, two, three, four, five, six, seven, eight, nine, ten, eleven, twelve, thirteen, fourteen, fifteen, sixteen, seventeen, eighteen, nineteen, twenty, twenty-one or all of CD3, CD19, CD8, CD56, CD105, CDIc, CD49e, ROR1, CD209, SSEA-4, CD40, CD62P, CD11c, CD40, MSCP (mesenchymal stem cell-like protein), CD146, CD86, CD326, CD133, CD142, CD31, and/or CD14. In particular embodiments of any such population of anti-inflammatory EVs described directly hereinabove, such a population is further characterized by a Treg EV surface marker profile substantially lacking CD3, CD19, CD8, CD56, CD105, CD1c, CD49e, ROR1, CD209, SSEA-4, CD40, CD62P, CD11c, CD40, MSCP (mesenchymal stem cell-like protein), CD146, CD86, CD326, CD133, CD142, CD31, and CD14.

In certain embodiments, a population of anti-inflammatory EVs provided herein is characterized by a Treg EV surface marker profile comprising: i) at least one, two or all three of CD2, HLA-DRDPDQ, and/or CD25, ii) further comprising at least one of CD44, CD29, CD4 and/or CD45, iii) further comprising at least one of HLA-ABC, CD24 and/or CD69; and iv) further comprising at least one exosome or EV marker, for example at least one, two or all three of CD81, CD63 and/or CD9. In certain embodiments, a population of anti-inflammatory EVs provided herein is characterized by a Treg EV surface marker profile comprising: i) CD2, HLA-DRDPDQ, and CD25, ii) further comprising at least one of CD44, CD29, CD4 and/or CD45, iii) further comprising at least one of HLA-ABC, CD24 and/or CD69; and iv) further comprising at least one exosome or EV marker, for example at least one, two or all three of CD81, CD63 and/or CD9. In certain embodiments, a population of anti-inflammatory EVs provided herein is characterized by a Treg EV surface marker profile comprising: i) CD2, HLA-DRDPDQ, and CD25, ii) further comprising CD44, CD29, CD4 and CD45, iii) further comprising at least one of HLA-ABC, CD24 and/or CD69; and iv) further comprising at least one exosome or EV marker, for example at least one, two or all three of CD81, CD63 and/or CD9. In certain embodiments, a population of anti-inflammatory EVs provided herein is characterized by a Treg EV surface marker profile comprising: i) CD2, HLA-DRDPDQ, and CD25, ii) further comprising CD44, CD29, CD4 and CD45, iii) further comprising at least one of HLA-ABC, CD24 and CD69; and iv) further comprising at least one exosome or EV marker, for example at least one, two or all three of CD81, CD63 and/or CD9. In certain embodiments, a population of anti-inflammatory EVs provided herein is characterized by a Treg EV surface marker profile comprising: i) CD2, HLA-DRDPDQ, and CD25, ii) further comprising CD44, CD29, CD4 and CD45, iii) further comprising at least one of HLA-ABC, CD24 and CD69; and iv) further comprising at least one, two or all three of CD81, CD63 and/or CD9. In certain embodiments, a population of anti-inflammatory EVs provided herein is characterized by a Treg EV surface marker profile comprising: i) CD2, HLA-DRDPDQ, and CD25, ii) further comprising CD44, CD29, CD4 and CD45, iii) further comprising at least one of HLA-ABC, CD24 and CD69; and iv) further comprising CD81, CD63 and CD9.

In particular embodiments of any such population of anti-inflammatory EVs described directly hereinabove, such a population is further characterized by a Treg EV surface marker profile substantially lacking at least one, two, three, four, five, six, seven, eight, nine, ten, eleven, twelve, thirteen, fourteen, fifteen, sixteen, seventeen, eighteen, nineteen, twenty, twenty-one or all of CD3, CD19, CD8, CD56, CD105, CD1c, CD49e, ROR1, CD209, SSEA-4, CD40, CD62P, CD11c, CD40, MSCP (mesenchymal stem cell-like protein), CD146, CD86, CD326, CD133, CD142, CD31, and/or CD14. In particular embodiments of any such population of anti-inflammatory EVs described directly hereinabove, such a population is further characterized by a Treg EV surface marker profile substantially lacking CD3, CD19, CD8, CD56, CD105, CD1c, CD49e, ROR1, CD209, SSEA-4, CD40, CD62P, CD11c, CD40, MSCP (mesenchymal stem cell-like protein), CD146, CD86, CD326, CD133, CD142, CD31, and CD14.

### 5.1.3. Treg EV RNA Profile

The populations of anti-inflammatory EVs provided herein may be characterized by their Treg EV RNA profile, which characterizes the micro-RNA (miRNA) profile of an anti-inflammatory EV population as described herein.

A Treg EV RNA profile may be generated using well-known techniques, for example, may be generated using techniques such as those described in Example 22 (“Treg EV RNA Profile”), below. For example, RNA may be isolated from an EV population, may be enriched for small RNAs, e.g., RNAs of about 15-200, e.g., 17-200, nucleotides in length, and sequenced, wherein sequences are counted when they are identified as corresponding to a sequence associated with known small miRNA. Such “read counts” may be used to assess abundance of a given miRNA within an EV population.

In one embodiment, a population of anti-inflammatory EVs provided herein is described as being characterized by a Treg RNA profile that comprises three, four, or all five of hsa-miR-1290, hsa-miR-146a-5p, hsa-miR-155-5p, hsa-let-7a-5p, and/or hsa-miR-21-5p. In certain embodiments, a population of anti-inflammatory EVs provided herein is described as being characterized by a Treg RNA profile that comprises each of hsa-miR-191-5p, hsa-miR-1246, hsa-let-7b-5p, hsa-miR-29a-3p, and hsa-miR-320a-3p.

In certain embodiments of any such population of anti-inflammatory EVs described directly hereinabove, such a population comprises hsa-miR-146a-5p and hsa-miR-155-5p in a hsa-miR-146a-5p/hsa-miR-155-5p ratio of about 1-10, for example, 1.5-6, e.g., about 2, about 3, about 2 to about 3, about 2 to about 4, about 4, or about 5. In certain embodiments of any such population of anti-inflammatory EVs described directly hereinabove, such a population comprises hsa-miR-1290 and hsa-miR-155-5p and such a population comprises hsa-miR-1290 at an abundance of at least 2-fold, 3-fold, 4-fold, 5-fold, 6-fold, 7-fold, 8-fold, 9-fold, or 10-fold higher than hsa-mir-155-5p.

In one embodiment, a population of anti-inflammatory EVs provided herein is described as being characterized by a Treg RNA profile that comprises: i) three, four, or all five of hsa-miR-1290, hsa-miR-146a-5p, hsa-miR-155-5p, hsa-let-7a-5p, and/or hsa-miR-21-5p; and ii) any 1-5 of hsa-miR-191-5p, hsa-miR-1246, hsa-let-7b-5p, hsa-miR-29a-3p, and/or hsa-miR-320a-3p. In certain embodiments, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p and ii) each of hsa-miR-191-5p, hsa-miR-1246, hsa-let-7b-5p, hsa-miR-29a-3p, and hsa-miR-320a-3p.

In certain embodiments of any such population of anti-inflammatory EVs described directly hereinabove, such a population comprises hsa-miR-146a-5p and hsa-miR-155-5p in a hsa-miR-146a-5p/hsa-miR-15-5p ratio of about 1-10, for example, 1.5-6, e.g., about 2, about 3, about 2 to about 3, about 2 to about 4, about 4, or about 5. In certain embodiments of any such population of anti-inflammatory EVs described directly hereinabove, such a population comprises hsa-miR-1290 and hsa-miR-155-5p and such a population comprises hsa-miR-1290 at an abundance of at least 2-fold, 3-fold, 4-fold, 5-fold, 6-fold, 7-fold, 8-fold, 9-fold, or 10-fold higher than hsa-mir-155-5p. In other embodiments of any such population of anti-inflammatory EVs described directly hereinabove, such a population comprises at least one of hsa-miR-1290 or hsa-miR-146a-5p at an abundance at least 2-fold, 3-fold, 4-fold, 5-fold, 6-fold, 7-fold, 8-fold, 9-fold, or 10-fold higher than that of any of the miRNAs of those listed at ii). In certain embodiments of any such population of anti-inflammatory EVs described directly hereinabove, such a population comprises hsa-miR-146a-5p and hsa-miR-155-5p in a hsa-miR-146a-5p/hsa-miR-155-5p ratio of about 1-10, for example, 1.5-6, e.g., about 2, about 3, about 2 to about 3, about 2 to about 4, about 4, or about 5; and such a population comprises hsa-miR-1290 and hsa-miR-146a-5p at an abundance at least 2-fold, 3-fold, 4-fold, 5-fold, 6-fold, 7-fold, 8-fold, 9-fold, or 10-fold higher than that of any of the miRNAs of those listed at ii).

In one embodiment, a population of anti-inflammatory EVs provided herein is described as being characterized by a Treg RNA profile that comprises: i) three, four, or all five of hsa-miR-1290, hsa-miR-146a-5p, hsa-miR-155-5p, hsa-let-7a-5p, and/or hsa-miR-21-5p; ii) any 1-5 of hsa-miR-191-5p, hsa-miR-1246, hsa-let-7b-5p, hsa-miR-29a-3p, and/or hsa-miR-320a-3p; and iii) any 1-5 of hsa-miR-423-5p, hsa-let-7f-5p, hsa-let-7i-5p, hsa-miR-16-5p, and/or hsa-miR-26a-5p. In certain embodiments, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) any 1-5 five of hsa-miR-191-5p, hsa-miR-1246, hsa-let-7b-5p, hsa-miR-29a-3p, and/or hsa-miR-320a-3p; and iii) any 1-5 of hsa-miR-423-5p, hsa-let-7f-5p, hsa-let-7i-5p, hsa-miR-16-5p, and/or hsa-miR-26a-5p. In certain embodiments, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p;ii) each of hsa-miR-191-5p, hsa-miR-1246, hsa-let-7b-5p, hsa-miR-29a-3p, and hsa-miR-320a-3p; and iii) each of hsa-miR-423-5p, hsa-let-7f-5p, hsa-let-7i-5p, hsa-miR-16-5p, and hsa-miR-26a-5p.

In certain embodiments of any such population of anti-inflammatory EVs described directly hereinabove, such a population comprises hsa-miR-146a-5p and hsa-miR-155-5p in a hsa-miR-146a-5p/hsa-miR-15-5p ratio of about 1-10, for example, 1.5-6, e.g., about 2, about 3, about 2 to about 3, about 2 to about 4, about 4, or about 5. In certain embodiments of any such population of anti-inflammatory EVs described directly hereinabove, such a population comprises hsa-miR-1290 and hsa-miR-155-5p and such a population comprises hsa-miR-1290 at an abundance of at least 2-fold, 3-fold, 4-fold, 5-fold, 6-fold, 7-fold, 8-fold, 9-fold, or 10-fold higher than hsa-mir-155-5p. In other embodiments of any such population of anti-inflammatory EVs described directly hereinabove, such a population comprises at least one of hsa-miR-1290 or hsa-miR-146a-5p at an abundance at least 2-fold, 3-fold, 4-fold, 5-fold, 6-fold, 7-fold, 8-fold, 9-fold, or 10-fold higher than that of any of the miRNAs of those listed at ii) and iii). In certain embodiments of any such population of anti-inflammatory EVs described directly hereinabove, such a population comprises hsa-miR-146a-5p and hsa-miR-155-5p in a hsa-miR-146a-5p/hsa-miR-15-5p ratio of about 1-10, for example, 1.5-6, e.g., about 2, about 3, about 2 to about 3, about 2 to about 4, about 4, or about 5; and such a population comprises hsa-miR-1290 and hsa-miR-146a-5p at an abundance at least 2-fold, 3-fold, 4-fold, 5-fold, 6-fold, 7-fold, 8-fold, 9-fold, or 10-fold higher than that of any of the miRNAs of those listed at ii) and iii).

In one embodiment, a population of anti-inflammatory EVs provided herein is described as being characterized by a Treg RNA profile that comprises: i) three, four, or all five of hsa-miR-1290, hsa-miR-146a-5p, hsa-miR-155-5p, hsa-let-7a-5p, and/or hsa-miR-21-5p; ii) any 1-5 of hsa-miR-191-5p, hsa-miR-1246, hsa-let-7b-5p, hsa-miR-29a-3p, and/or hsa-miR-320a-3p; iii) any 1-5 of hsa-miR-423-5p, hsa-let-7f-5p, hsa-let-7i-5p, hsa-miR-16-5p, and/or hsa-miR-26a-5p; and iv) any 1-5 of hsa-let-7g-5p, hsa-miR-221-3p, hsa-miR-222-3p, hsa-miR-26b-5p, and/or hsa-miR-342-3p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) any 1-5 of hsa-miR-191-5p, hsa-miR-1246, hsa-let-7b-5p, hsa-miR-29a-3p, and/or hsa-miR-320a-3p; iii) any 1-5 of hsa-miR-423-5p, hsa-let-7f-5p, hsa-let-7i-5p, hsa-miR-16-5p, and/or hsa-miR-26a-5p; and any 1-5 of hsa-let-7g-5p, hsa-miR-221-3p, hsa-miR-222-3p, hsa-miR-26b-5p, and/or hsa-miR-342-3p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) any 1-5 of hsa-miR-423-5p, hsa-let-7f-5p, hsa-let-7i-5p, hsa-miR-16-5p, and/or hsa-miR-26a-5p; and any 1-5 of hsa-let-7g-5p, hsa-miR-221-3p, hsa-miR-222-3p, hsa-miR-26b-5p, and/or hsa-miR-342-3p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) each of hsa-miR-423-5p, hsa-let-7f-5p, hsa-let-7i-5p, hsa-miR-16-5p, and hsa-miR-26a-5p; and any 1-5 of hsa-let-7g-5p, hsa-miR-221-3p, hsa-miR-222-3p, hsa-miR-26b-5p, and/or hsa-miR-342-3p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) each of hsa-miR-423-5p, hsa-let-7f-5p, hsa-let-7i-5p, hsa-miR-16-5p, and hsa-miR-26a-5p; and each of hsa-let-7g-5p, hsa-miR-221-3p, hsa-miR-222-3p, hsa-miR-26b-5p, and hsa-miR-342-3p.

In certain embodiments of any such population of anti-inflammatory EVs described directly hereinabove, such a population comprises hsa-miR-146a-5p and hsa-miR-155-5p in a hsa-miR-146a-5p/hsa-miR-15-5p ratio of about 1-10, for example, 1.5-6, e.g., about 2, about 3, about 2 to about 3, about 2 to about 4, about 4, or about 5. In certain embodiments of any such population of anti-inflammatory EVs described directly hereinabove, such a population comprises hsa-miR-1290 and hsa-miR-155-5p and such a population comprises hsa-miR-1290 at an abundance of at least 2-fold, 3-fold, 4-fold, 5-fold, 6-fold, 7-fold, 8-fold, 9-fold, or 10-fold higher than hsa-mir-155-5p. In other embodiments of any such population of anti-inflammatory EVs described directly hereinabove, such a population comprises at least one of hsa-miR-1290 or hsa-miR-146a-5p at an abundance at least 2-fold, 3-fold, 4-fold, 5-fold, 6-fold, 7-fold, 8-fold, 9-fold, or 10-fold higher than that of any of the miRNAs of those listed at ii) and iii). In certain embodiments of any such population of anti-inflammatory EVs described directly hereinabove, such a population comprises hsa-miR-146a-5p and hsa-miR-155-5p in a hsa-miR-146a-5p/hsa-miR-15-5p ratio of about 1-10, for example, 1.5-6, e.g., about 2, about 3, about 2 to about 3, about 2 to about 4, about 4, or about 5; and such a population comprises hsa-miR-1290 and hsa-miR-146a-5p at an abundance at least 2-fold, 3-fold, 4-fold, 5-fold, 6-fold, 7-fold, 8-fold, 9-fold, or 10-fold higher than that of any of the miRNAs of those listed at ii) through iv).

In one embodiment, a population of anti-inflammatory EVs provided herein is described as being characterized by a Treg RNA profile that comprises: i) three, four, or all five of hsa-miR-1290, hsa-miR-146a-5p, hsa-miR-155-5p, hsa-let-7a-5p, and/or hsa-miR-21-5p; ii) any 1-5 of hsa-miR-191-5p, hsa-miR-1246, hsa-let-7b-5p, hsa-miR-29a-3p, and/or hsa-miR-320a-3p; iii) any 1-5 of hsa-miR-423-5p, hsa-let-7f-5p, hsa-let-7i-5p, hsa-miR-16-5p, and/or hsa-miR-26a-5p; iv) any 1-5 of hsa-let-7g-5p, hsa-miR-221-3p, hsa-miR-222-3p, hsa-miR-26b-5p, and/or hsa-miR-342-3p; and v) any 1-5 of hsa-miR-146b-5p, hsa-miR-92a-3p, hsa-miR-93-5p, hsa-miR-23a-3p, and/or hsa-miR-181a-5p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) any 1-5 of hsa-miR-191-5p, hsa-miR-1246, hsa-let-7b-5p, hsa-miR-29a-3p, and/or hsa-miR-320a-3p; iii) any 1-5 of hsa-miR-423-5p, hsa-let-7f-5p, hsa-let-7i-5p, hsa-miR-16-5p, and/or hsa-miR-26a-5p; iv) any 1-5 of hsa-let-7g-5p, hsa-miR-221-3p, hsa-miR-222-3p, hsa-miR-26b-5p, and/or hsa-miR-342-3p; and v) any 1-5 of hsa-miR-146b-5p, hsa-miR-92a-3p, hsa-miR-93-5p, hsa-miR-23a-3p, and/or hsa-miR-181a-5p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) any 1-5 of hsa-miR-423-5p, hsa-let-7f-5p, hsa-let-7i-5p, hsa-miR-16-5p, and/or hsa-miR-26a-5p; iv) any 1-5 of hsa-let-7g-5p, hsa-miR-221-3p, hsa-miR-222-3p, hsa-miR-26b-5p, and/or hsa-miR-342-3p; and v) any 1-5 of hsa-miR-146b-5p, hsa-miR-92a-3p, hsa-miR-93-5p, hsa-miR-23a-3p, and/or hsa-miR-181a-5p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) each of hsa-miR-423-5p, hsa-let-7f-5p, hsa-let-7i-5p, hsa-miR-16-5p, and hsa-miR-26a-5p; iv) any 1-5 of hsa-let-7g-5p, hsa-miR-221-3p, hsa-miR-222-3p, hsa-miR-26b-5p, and/or hsa-miR-342-3p; and v) any 1-5 of hsa-miR-146b-5p, hsa-miR-92a-3p, hsa-miR-93-5p, hsa-miR-23a-3p, and/or hsa-miR-181a-5p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) each of hsa-miR-423-5p, hsa-let-7f-5p, hsa-let-7i-5p, hsa-miR-16-5p, and hsa-miR-26a-5p; iv) each of hsa-let-7g-5p, hsa-miR-221-3p, hsa-miR-222-3p, hsa-miR-26b-5p, and hsa-miR-342-3p; and v) any 1-5 of hsa-miR-146b-5p, hsa-miR-92a-3p, hsa-miR-93-5p, hsa-miR-23a-3p, and/or hsa-miR-181a-5p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) each of hsa-miR-423-5p, hsa-let-7f-5p, hsa-let-7i-5p, hsa-miR-16-5p, and hsa-miR-26a-5p; iv) each of hsa-let-7g-5p, hsa-miR-221-3p, hsa-miR-222-3p, hsa-miR-26b-5p, and hsa-miR-342-3p; and v) each of hsa-miR-146b-5p, hsa-miR-92a-3p, hsa-miR-93-5p, hsa-miR-23a-3p, and hsa-miR-181a-5p.

In certain embodiments of any such population of anti-inflammatory EVs described directly hereinabove, such a population comprises hsa-miR-146a-5p and hsa-miR-155-5p in a hsa-miR-146a-5p/hsa-miR-15-5p ratio of about 1-10, for example, 1.5-6, e.g., about 2, about 3, about 2 to about 3, about 2 to about 4, about 4, or about 5. In certain embodiments of any such population of anti-inflammatory EVs described directly hereinabove, such a population comprises hsa-miR-1290 and hsa-miR-155-5p and such a population comprises hsa-miR-1290 at an abundance of at least 2-fold, 3-fold, 4-fold, 5-fold, 6-fold, 7-fold, 8-fold, 9-fold, or 10-fold higher than hsa-mir-155-5p. In other embodiments of any such population of anti-inflammatory EVs described directly hereinabove, such a population comprises at least one of hsa-miR-1290 or hsa-miR-146a-5p at an abundance at least 2-fold, 3-fold, 4-fold, 5-fold, 6-fold, 7-fold, 8-fold, 9-fold, or 10-fold higher than that of any of the miRNAs of those listed at ii) and iii). In certain embodiments of any such population of anti-inflammatory EVs described directly hereinabove, such a population comprises hsa-miR-146a-5p and hsa-miR-155-5p in a hsa-miR-146a-5p/hsa-miR-15-5p ratio of about 1-10, for example, 1.5-6, e.g., about 2, about 3, about 2 to about 3, about 2 to about 4, about 4, or about 5; and such a population comprises hsa-miR-1290 and hsa-miR-146a-5p at an abundance at least 2-fold, 3-fold, 4-fold, 5-fold, 6-fold, 7-fold, 8-fold, 9-fold, or 10-fold higher than that of any of the miRNAs of those listed at ii) through v).

In one embodiment, a population of anti-inflammatory EVs provided herein is described as being characterized by a Treg RNA profile that comprises: i) three, four, or all five of hsa-miR-1290, hsa-miR-146a-5p, hsa-miR-155-5p, hsa-let-7a-5p, and/or hsa-miR-21-5p; ii) any 1-5 of hsa-miR-191-5p, hsa-miR-1246, hsa-let-7b-5p, hsa-miR-29a-3p, and/or hsa-miR-320a-3p; iii) any 1-5 of hsa-miR-423-5p, hsa-let-7f-5p, hsa-let-7i-5p, hsa-miR-16-5p, and/or hsa-miR-26a-5p; iv) any 1-5 of hsa-let-7g-5p, hsa-miR-221-3p, hsa-miR-222-3p, hsa-miR-26b-5p, and/or hsa-miR-342-3p; v) any 1-5 of hsa-miR-146b-5p, hsa-miR-92a-3p, hsa-miR-93-5p, hsa-miR-23a-3p, and/or hsa-miR-181a-5p; and vi) any 1-5 of hsa-miR-150-5p, hsa-miR-20a-5p, hsa-miR-27a-3p, hsa-let-7d-5p, and/or hsa-miR-17-5p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) any 1-5 of hsa-miR-191-5p, hsa-miR-1246, hsa-let-7b-5p, hsa-miR-29a-3p, and/or hsa-miR-320a-3p; iii) any 1-5 of hsa-miR-423-5p, hsa-let-7f-5p, hsa-let-7i-5p, hsa-miR-16-5p, and/or hsa-miR-26a-5p; iv) any 1-5 of hsa-let-7g-5p, hsa-miR-221-3p, hsa-miR-222-3p, hsa-miR-26b-5p, and/or hsa-miR-342-3p; v) any 1-5 of hsa-miR-146b-5p, hsa-miR-92a-3p, hsa-miR-93-5p, hsa-miR-23a-3p, and/or hsa-miR-181a-5p; and vi) any 1-5 of hsa-miR-150-5p, hsa-miR-20a-5p, hsa-miR-27a-3p, hsa-let-7d-5p, and/or hsa-miR-17-5p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) any 1-5 of hsa-miR-423-5p, hsa-let-7f-5p, hsa-let-7i-5p, hsa-miR-16-5p, and hsa-miR-26a-5p; iv) any 1-5 of hsa-let-7g-5p, hsa-miR-221-3p, hsa-miR-222-3p, hsa-miR-26b-5p, and/or hsa-miR-342-3p; v) any 1-5 of hsa-miR-146b-5p, hsa-miR-92a-3p, hsa-miR-93-5p, hsa-miR-23a-3p, and/or hsa-miR-181a-5p; and vi) any 1-5 of hsa-miR-150-5p, hsa-miR-20a-5p, hsa-miR-27a-3p, hsa-let-7d-5p, and/or hsa-miR-17-5p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) each of hsa-miR-423-5p, hsa-let-7f-5p, hsa-let-7i-5p, hsa-miR-16-5p, and hsa-miR-26a-5p; iv) any 1-5 of hsa-let-7g-5p, hsa-miR-221-3p, hsa-miR-222-3p, hsa-miR-26b-5p, and/or hsa-miR-342-3p; v) any 1-5 of hsa-miR-146b-5p, hsa-miR-92a-3p, hsa-miR-93-5p, hsa-miR-23a-3p, and/or hsa-miR-181a-5p; and vi) any 1-5 of hsa-miR-150-5p, hsa-miR-20a-5p, hsa-miR-27a-3p, hsa-let-7d-5p, and/or hsa-miR-17-5p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) each of hsa-miR-423-5p, hsa-let-7f-5p, hsa-let-7i-5p, hsa-miR-16-5p, and hsa-miR-26a-5p; iv) each of hsa-let-7g-5p, hsa-miR-221-3p, hsa-miR-222-3p, hsa-miR-26b-5p, and hsa-miR-342-3p; v) any 1-5 of hsa-miR-146b-5p, hsa-miR-92a-3p, hsa-miR-93-5p, hsa-miR-23a-3p, and/or hsa-miR-181a-5p; and vi) any 1-5 of hsa-miR-150-5p, hsa-miR-20a-5p, hsa-miR-27a-3p, hsa-let-7d-5p, and/or hsa-miR-17-5p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) each of hsa-miR-423-5p, hsa-let-7f-5p, hsa-let-7i-5p, hsa-miR-16-5p, and hsa-miR-26a-5p; iv) each of hsa-let-7g-5p, hsa-miR-221-3p, hsa-miR-222-3p, hsa-miR-26b-5p, and hsa-miR-342-3p; v) each of hsa-miR-146b-5p, hsa-miR-92a-3p, hsa-miR-93-5p, hsa-miR-23a-3p, and hsa-miR-181a-5p; and vi) any 1-5 of hsa-miR-150-5p, hsa-miR-20a-5p, hsa-miR-27a-3p, hsa-let-7d-5p, and/or hsa-miR-17-5p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) each of hsa-miR-423-5p, hsa-let-7f-5p, hsa-let-7i-5p, hsa-miR-16-5p, and hsa-miR-26a-5p; iv) each of hsa-let-7g-5p, hsa-miR-221-3p, hsa-miR-222-3p, hsa-miR-26b-5p, and hsa-miR-342-3p; v) each of hsa-miR-146b-5p, hsa-miR-92a-3p, hsa-miR-93-5p, hsa-miR-23a-3p, and hsa-miR-181a-5p; and vi) each of hsa-miR-150-5p, hsa-miR-20a-5p, hsa-miR-27a-3p, hsa-let-7d-5p, and hsa-miR-17-5p.

In certain embodiments of any such population of anti-inflammatory EVs described directly hereinabove, such a population comprises hsa-miR-146a-5p and hsa-miR-155-5p in a hsa-miR-146a-5p/hsa-miR-15-5p ratio of about 1-10, for example, 1.5-6, e.g., about 2, about 3, about 2 to about 3, about 2 to about 4, about 4, or about 5. In certain embodiments of any such population of anti-inflammatory EVs described directly hereinabove, such a population comprises hsa-miR-1290 and hsa-miR-155-5p and such a population comprises hsa-miR-1290 at an abundance of at least 2-fold, 3-fold, 4-fold, 5-fold, 6-fold, 7-fold, 8-fold, 9-fold, or 10-fold higher than hsa-mir-155-5p. In other embodiments of any such population of anti-inflammatory EVs described directly hereinabove, such a population comprises at least one of hsa-miR-1290 or hsa-miR-146a-5p at an abundance at least 2-fold, 3-fold, 4-fold, 5-fold, 6-fold, 7-fold, 8-fold, 9-fold, or 10-fold higher than that of any of the miRNAs of those listed at ii) and iii). In certain embodiments of any such population of anti-inflammatory EVs described directly hereinabove, such a population comprises hsa-miR-146a-5p and hsa-miR-155-5p in a hsa-miR-146a-5p/hsa-miR-15-5p ratio of about 1-10, for example, 1.5-6, e.g., about 2, about 3, about 2 to about 3, about 2 to about 4, about 4, or about 5; and such a population comprises hsa-miR-1290 and hsa-miR-146a-5p at an abundance at least 2-fold, 3-fold, 4-fold, 5-fold, 6-fold, 7-fold, 8-fold, 9-fold, or 10-fold higher than that of any of the miRNAs of those listed at ii) through vi).

In one embodiment, a population of anti-inflammatory EVs provided herein is described as being characterized by a Treg RNA profile that comprises: i) three, four, or all five of hsa-miR-1290, hsa-miR-146a-5p, hsa-miR-155-5p, hsa-let-7a-5p, and/or hsa-miR-21-5p; ii) any 1-5 of hsa-miR-191-5p, hsa-miR-1246, hsa-let-7b-5p, hsa-miR-29a-3p, and/or hsa-miR-320a-3p; iii) any 1-5 of hsa-miR-423-5p, hsa-let-7f-5p, hsa-let-7i-5p, hsa-miR-16-5p, and/or hsa-miR-26a-5p; iv) any 1-5 of hsa-let-7g-5p, hsa-miR-221-3p, hsa-miR-222-3p, hsa-miR-26b-5p, and/or hsa-miR-342-3p; v) any 1-5 of hsa-miR-146b-5p, hsa-miR-92a-3p, hsa-miR-93-5p, hsa-miR-23a-3p, and/or hsa-miR-181a-5p; vi) any 1-5 of hsa-miR-150-5p, hsa-miR-20a-5p, hsa-miR-27a-3p, hsa-let-7d-5p, and/or hsa-miR-17-5p; and vii) any 1-5 of hsa-miR-142-3p, hsa-miR-130b-3p, hsa-miR-25-3p, hsa-miR-142-5p, and/or hsa-miR-103a-3p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) any 1-5 of hsa-miR-191-5p, hsa-miR-1246, hsa-let-7b-5p, hsa-miR-29a-3p, and/or hsa-miR-320a-3p; iii) any 1-5 of hsa-miR-423-5p, hsa-let-7f-5p, hsa-let-7i-5p, hsa-miR-16-5p, and/or hsa-miR-26a-5p; iv) any 1-5 of hsa-let-7g-5p, hsa-miR-221-3p, hsa-miR-222-3p, hsa-miR-26b-5p, and/or hsa-miR-342-3p; v) any 1-5 of hsa-miR-146b-5p, hsa-miR-92a-3p, hsa-miR-93-5p, hsa-miR-23a-3p, and/or hsa-miR-181a-5p; vi) any 1-5 of hsa-miR-150-5p, hsa-miR-20a-5p, hsa-miR-27a-3p, hsa-let-7d-5p, and/or hsa-miR-17-5p; and vii) any 1-5 of hsa-miR-142-3p, hsa-miR-130b-3p, hsa-miR-25-3p, hsa-miR-142-5p, and/or hsa-miR-103a-3p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) any 1-5 of hsa-miR-423-5p, hsa-let-7f-5p, hsa-let-7i-5p, hsa-miR-16-5p, and/or hsa-miR-26a-5p; iv) any 1-5 of hsa-let-7g-5p, hsa-miR-221-3p, hsa-miR-222-3p, hsa-miR-26b-5p, and/or hsa-miR-342-3p; v) any 1-5 of hsa-miR-146b-5p, hsa-miR-92a-3p, hsa-miR-93-5p, hsa-miR-23a-3p, and/or hsa-miR-181a-5p; vi) any 1-5 of hsa-miR-150-5p, hsa-miR-20a-5p, hsa-miR-27a-3p, hsa-let-7d-5p, and/or hsa-miR-17-5p; and vii) any 1-5 of hsa-miR-142-3p, hsa-miR-130b-3p, hsa-miR-25-3p, hsa-miR-142-5p, and/or hsa-miR-103a-3p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) each of hsa-miR-423-5p, hsa-let-7f-5p, hsa-let-7i-5p, hsa-miR-16-5p, and hsa-miR-26a-5p; iv) any 1-5 of hsa-let-7g-5p, hsa-miR-221-3p, hsa-miR-222-3p, hsa-miR-26b-5p, and/or hsa-miR-342-3p; v) any 1-5 of hsa-miR-146b-5p, hsa-miR-92a-3p, hsa-miR-93-5p, hsa-miR-23a-3p, and/or hsa-miR-181a-5p; vi) any 1-5 of hsa-miR-150-5p, hsa-miR-20a-5p, hsa-miR-27a-3p, hsa-let-7d-5p, and/or hsa-miR-17-5p; and vii) any 1-5 of hsa-miR-142-3p, hsa-miR-130b-3p, hsa-miR-25-3p, hsa-miR-142-5p, and/or hsa-miR-103a-3p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) each of hsa-miR-423-5p, hsa-let-7f-5p, hsa-let-7i-5p, hsa-miR-16-5p, and hsa-miR-26a-5p; iv) each of hsa-let-7g-5p, hsa-miR-221-3p, hsa-miR-222-3p, hsa-miR-26b-5p, and hsa-miR-342-3p; v) any 1-5 of hsa-miR-146b-5p, hsa-miR-92a-3p, hsa-miR-93-5p, hsa-miR-23a-3p, and/or hsa-miR-181a-5p; vi) any 1-5 of hsa-miR-150-5p, hsa-miR-20a-5p, hsa-miR-27a-3p, hsa-let-7d-5p, and/or hsa-miR-17-5p; and vii) any 1-5 of hsa-miR-142-3p, hsa-miR-130b-3p, hsa-miR-25-3p, hsa-miR-142-5p, and/or hsa-miR-103a-3p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) each of hsa-miR-423-5p, hsa-let-7f-5p, hsa-let-7i-5p, hsa-miR-16-5p, and hsa-miR-26a-5p; iv) each of hsa-let-7g-5p, hsa-miR-221-3p, hsa-miR-222-3p, hsa-miR-26b-5p, and hsa-miR-342-3p; v) each of hsa-miR-146b-5p, hsa-miR-92a-3p, hsa-miR-93-5p, hsa-miR-23a-3p, and hsa-miR-181a-5p; vi) any 1-5 of hsa-miR-150-5p, hsa-miR-20a-5p, hsa-miR-27a-3p, hsa-let-7d-5p, and/or hsa-miR-17-5p; and vii) any 1-5 of hsa-miR-142-3p, hsa-miR-130b-3p, hsa-miR-25-3p, hsa-miR-142-5p, and/or hsa-miR-103a-3p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) each of hsa-miR-423-5p, hsa-let-7f-5p, hsa-let-7i-5p, hsa-miR-16-5p, and hsa-miR-26a-5p; iv) each of hsa-let-7g-5p, hsa-miR-221-3p, hsa-miR-222-3p, hsa-miR-26b-5p, and hsa-miR-342-3p; v) each of hsa-miR-146b-5p, hsa-miR-92a-3p, hsa-miR-93-5p, hsa-miR-23a-3p, and hsa-miR-181a-5p; vi) each of hsa-miR-150-5p, hsa-miR-20a-5p, hsa-miR-27a-3p, hsa-let-7d-5p, and hsa-miR-17-5p; and vii) any 1-5 of hsa-miR-142-3p, hsa-miR-130b-3p, hsa-miR-25-3p, hsa-miR-142-5p, and/or hsa-miR-103a-3p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) each of hsa-miR-423-5p, hsa-let-7f-5p, hsa-let-7i-5p, hsa-miR-16-5p, and hsa-miR-26a-5p; iv) each of hsa-let-7g-5p, hsa-miR-221-3p, hsa-miR-222-3p, hsa-miR-26b-5p, and hsa-miR-342-3p; v) each of hsa-miR-146b-5p, hsa-miR-92a-3p, hsa-miR-93-5p, hsa-miR-23a-3p, and hsa-miR-181a-5p; vi) each of hsa-miR-150-5p, hsa-miR-20a-5p, hsa-miR-27a-3p, hsa-let-7d-5p, and hsa-miR-17-5p; and vii) each of hsa-miR-142-3p, hsa-miR-130b-3p, hsa-miR-25-3p, hsa-miR-142-5p, and hsa-miR-103a-3p.

In certain embodiments of any such population of anti-inflammatory EVs described directly hereinabove, such a population comprises hsa-miR-146a-5p and hsa-miR-155-5p in a hsa-miR-146a-5p/hsa-miR-15-5p ratio of about 1-10, for example, 1.5-6, e.g., about 2, about 3, about 2 to about 3, about 2 to about 4, about 4, or about 5. In certain embodiments of any such population of anti-inflammatory EVs described directly hereinabove, such a population comprises hsa-miR-1290 and hsa-miR-155-5p and such a population comprises hsa-miR-1290 at an abundance of at least 2-fold, 3-fold, 4-fold, 5-fold, 6-fold, 7-fold, 8-fold, 9-fold, or 10-fold higher than hsa-mir-155-5p. In other embodiments of any such population of anti-inflammatory EVs described directly hereinabove, such a population comprises at least one of hsa-miR-1290 or hsa-miR-146a-5p at an abundance at least 2-fold, 3-fold, 4-fold, 5-fold, 6-fold, 7-fold, 8-fold, 9-fold, or 10-fold higher than that of any of the miRNAs of those listed at ii) and iii). In certain embodiments of any such population of anti-inflammatory EVs described directly hereinabove, such a population comprises hsa-miR-146a-5p and hsa-miR-155-5p in a hsa-miR-146a-5p/hsa-miR-15-5p ratio of about 1-10, for example, 1.5-6, e.g., about 2, about 3, about 2 to about 3, about 2 to about 4, about 4, or about 5; and such a population comprises hsa-miR-1290 and hsa-miR-146a-5p at an abundance at least 2-fold, 3-fold, 4-fold, 5-fold, 6-fold, 7-fold, 8-fold, 9-fold, or 10-fold higher than that of any of the miRNAs of those listed at ii) through vii).

In one embodiment, a population of anti-inflammatory EVs provided herein is described as being characterized by a Treg RNA profile that comprises: i) three, four, or all five of hsa-miR-1290, hsa-miR-146a-5p, hsa-miR-155-5p, hsa-let-7a-5p, and/or hsa-miR-21-5p; ii) any 1-5 of hsa-miR-191-5p, hsa-miR-1246, hsa-let-7b-5p, hsa-miR-29a-3p, and/or hsa-miR-320a-3p; iii) any 1-5 of hsa-miR-423-5p, hsa-let-7f-5p, hsa-let-7i-5p, hsa-miR-16-5p, and/or hsa-miR-26a-5p; iv) any 1-5 of hsa-let-7g-5p, hsa-miR-221-3p, hsa-miR-222-3p, hsa-miR-26b-5p, and/or hsa-miR-342-3p; v) any 1-5 of hsa-miR-146b-5p, hsa-miR-92a-3p, hsa-miR-93-5p, hsa-miR-23a-3p, and/or hsa-miR-181a-5p; vi) any 1-5 of hsa-miR-150-5p, hsa-miR-20a-5p, hsa-miR-27a-3p, hsa-let-7d-5p, and/or hsa-miR-17-5p; vii) any 1-5 of hsa-miR-142-3p, hsa-miR-130b-3p, hsa-miR-25-3p, hsa-miR-142-5p, and/or hsa-miR-103a-3p; and viii) any 1-5 of hsa-miR-28-3p, hsa-let-7e-5p, hsa-miR-425-5p, hsa-miR-186-5p, and/or hsa-miR-625-5p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) any 1-5 of hsa-miR-191-5p, hsa-miR-1246, hsa-let-7b-5p, hsa-miR-29a-3p, and/or hsa-miR-320a-3p; iii) any 1-5 of hsa-miR-423-5p, hsa-let-7f-5p, hsa-let-7i-5p, hsa-miR-16-5p, and/or hsa-miR-26a-5p; iv) any 1-5 of hsa-let-7g-5p, hsa-miR-221-3p, hsa-miR-222-3p, hsa-miR-26b-5p, and/or hsa-miR-342-3p; v) any 1-5 of hsa-miR-146b-5p, hsa-miR-92a-3p, hsa-miR-93-5p, hsa-miR-23a-3p, and/or hsa-miR-181a-5p; vi) any 1-5 of hsa-miR-150-5p, hsa-miR-20a-5p, hsa-miR-27a-3p, hsa-let-7d-5p, and/or hsa-miR-17-5p; vii) any 1-5 of hsa-miR-142-3p, hsa-miR-130b-3p, hsa-miR-25-3p, hsa-miR-142-5p, and/or hsa-miR-103a-3p; and viii) any 1-5 of hsa-miR-28-3p, hsa-let-7e-5p, hsa-miR-425-5p, hsa-miR-186-5p, and/or hsa-miR-625-5p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) any 1-5 of hsa-miR-423-5p, hsa-let-7f-5p, hsa-let-7i-5p, hsa-miR-16-5p, and/or hsa-miR-26a-5p; iv) any 1-5 of hsa-let-7g-5p, hsa-miR-221-3p, hsa-miR-222-3p, hsa-miR-26b-5p, and/or hsa-miR-342-3p; v) any 1-5 of hsa-miR-146b-5p, hsa-miR-92a-3p, hsa-miR-93-5p, hsa-miR-23a-3p, and/or hsa-miR-181a-5p; vi) any 1-5 of hsa-miR-150-5p, hsa-miR-20a-5p, hsa-miR-27a-3p, hsa-let-7d-5p, and/or hsa-miR-17-5p; vii) any 1-5 of hsa-miR-142-3p, hsa-miR-130b-3p, hsa-miR-25-3p, hsa-miR-142-5p, and/or hsa-miR-103a-3p; and viii) any 1-5 of hsa-miR-28-3p, hsa-let-7e-5p, hsa-miR-425-5p, hsa-miR-186-5p, and/or hsa-miR-625-5p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) each of hsa-miR-423-5p, hsa-let-7f-5p, hsa-let-7i-5p, hsa-miR-16-5p, and hsa-miR-26a-5p; iv) any 1-5 of hsa-let-7g-5p, hsa-miR-221-3p, hsa-miR-222-3p, hsa-miR-26b-5p, and/or hsa-miR-342-3p; v) any 1-5 of hsa-miR-146b-5p, hsa-miR-92a-3p, hsa-miR-93-5p, hsa-miR-23a-3p, and/or hsa-miR-181a-5p; vi) any 1-5 of hsa-miR-150-5p, hsa-miR-20a-5p, hsa-miR-27a-3p, hsa-let-7d-5p, and/or hsa-miR-17-5p; vii) any 1-5 of hsa-miR-142-3p, hsa-miR-130b-3p, hsa-miR-25-3p, hsa-miR-142-5p, and/or hsa-miR-103a-3p; and viii) any 1-5 of hsa-miR-28-3p, hsa-let-7e-5p, hsa-miR-425-5p, hsa-miR-186-5p, and/or hsa-miR-625-5p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) each of hsa-miR-423-5p, hsa-let-7f-5p, hsa-let-7i-5p, hsa-miR-16-5p, and hsa-miR-26a-5p; iv) each of hsa-let-7g-5p, hsa-miR-221-3p, hsa-miR-222-3p, hsa-miR-26b-5p, and hsa-miR-342-3p; v) any 1-5 of hsa-miR-146b-5p, hsa-miR-92a-3p, hsa-miR-93-5p, hsa-miR-23a-3p, and/or hsa-miR-181a-5p; vi) any 1-5 of hsa-miR-150-5p, hsa-miR-20a-5p, hsa-miR-27a-3p, hsa-let-7d-5p, and/or hsa-miR-17-5p; vii) any 1-5 of hsa-miR-142-3p, hsa-miR-130b-3p, hsa-miR-25-3p, hsa-miR-142-5p, and/or hsa-miR-103a-3p; and viii) any 1-5 of hsa-miR-28-3p, hsa-let-7e-5p, hsa-miR-425-5p, hsa-miR-186-5p, and/or hsa-miR-625-5p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) each of hsa-miR-423-5p, hsa-let-7f-5p, hsa-let-7i-5p, hsa-miR-16-5p, and hsa-miR-26a-5p; iv) each of hsa-let-7g-5p, hsa-miR-221-3p, hsa-miR-222-3p, hsa-miR-26b-5p, and hsa-miR-342-3p; v) each of hsa-miR-146b-5p, hsa-miR-92a-3p, hsa-miR-93-5p, hsa-miR-23a-3p, and hsa-miR-181a-5p; vi) any 1-5 of hsa-miR-150-5p, hsa-miR-20a-5p, hsa-miR-27a-3p, hsa-let-7d-5p, and/or hsa-miR-17-5p; vii) any 1-5 of hsa-miR-142-3p, hsa-miR-130b-3p, hsa-miR-25-3p, hsa-miR-142-5p, and/or hsa-miR-103a-3p; and viii) any 1-5 of hsa-miR-28-3p, hsa-let-7e-5p, hsa-miR-425-5p, hsa-miR-186-5p, and/or hsa-miR-625-5p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) each of hsa-miR-423-5p, hsa-let-7f-5p, hsa-let-7i-5p, hsa-miR-16-5p, and hsa-miR-26a-5p; iv) each of hsa-let-7g-5p, hsa-miR-221-3p, hsa-miR-222-3p, hsa-miR-26b-5p, and hsa-miR-342-3p; v) each of hsa-miR-146b-5p, hsa-miR-92a-3p, hsa-miR-93-5p, hsa-miR-23a-3p, and hsa-miR-181a-5p; vi) each of hsa-miR-150-5p, hsa-miR-20a-5p, hsa-miR-27a-3p, hsa-let-7d-5p, and hsa-miR-17-5p; vii) any 1-5 of hsa-miR-142-3p, hsa-miR-130b-3p, hsa-miR-25-3p, hsa-miR-142-5p, and/or hsa-miR-103a-3p; and viii) any 1-5 of hsa-miR-28-3p, hsa-let-7e-5p, hsa-miR-425-5p, hsa-miR-186-5p, and/or hsa-miR-625-5p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) each of hsa-miR-423-5p, hsa-let-7f-5p, hsa-let-7i-5p, hsa-miR-16-5p, and hsa-miR-26a-5p; iv) each of hsa-let-7g-5p, hsa-miR-221-3p, hsa-miR-222-3p, hsa-miR-26b-5p, and hsa-miR-342-3p; v) each of hsa-miR-146b-5p, hsa-miR-92a-3p, hsa-miR-93-5p, hsa-miR-23a-3p, and hsa-miR-181a-5p; vi) each of hsa-miR-150-5p, hsa-miR-20a-5p, hsa-miR-27a-3p, hsa-let-7d-5p, and hsa-miR-17-5p; vii) each of hsa-miR-142-3p, hsa-miR-130b-3p, hsa-miR-25-3p, hsa-miR-142-5p, and hsa-miR-103a-3p; and viii) any 1-5 of hsa-miR-28-3p, hsa-let-7e-5p, hsa-miR-425-5p, hsa-miR-186-5p, and/or hsa-miR-625-5p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) each of hsa-miR-423-5p, hsa-let-7f-5p, hsa-let-7i-5p, hsa-miR-16-5p, and hsa-miR-26a-5p; iv) each of hsa-let-7g-5p, hsa-miR-221-3p, hsa-miR-222-3p, hsa-miR-26b-5p, and hsa-miR-342-3p; v) each of hsa-miR-146b-5p, hsa-miR-92a-3p, hsa-miR-93-5p, hsa-miR-23a-3p, and hsa-miR-181a-5p; vi) each of hsa-miR-150-5p, hsa-miR-20a-5p, hsa-miR-27a-3p, hsa-let-7d-5p, and hsa-miR-17-5p; vii) each of hsa-miR-142-3p, hsa-miR-130b-3p, hsa-miR-25-3p, hsa-miR-142-5p, and hsa-miR-103a-3p; and viii) each of hsa-miR-28-3p, hsa-let-7e-5p, hsa-miR-425-5p, hsa-miR-186-5p, and hsa-miR-625-5p.

In certain embodiments of any such population of anti-inflammatory EVs described directly hereinabove, such a population comprises hsa-miR-146a-5p and hsa-miR-155-5p in a hsa-miR-146a-5p/hsa-miR-15-5p ratio of about 1-10, for example, 1.5-6, e.g., about 2, about 3, about 2 to about 3, about 2 to about 4, about 4, or about 5. In certain embodiments of any such population of anti-inflammatory EVs described directly hereinabove, such a population comprises hsa-miR-1290 and hsa-miR-155-5p and such a population comprises hsa-miR-1290 at an abundance of at least 2-fold, 3-fold, 4-fold, 5-fold, 6-fold, 7-fold, 8-fold, 9-fold, or 10-fold higher than hsa-mir-155-5p. In other embodiments of any such population of anti-inflammatory EVs described directly hereinabove, such a population comprises at least one of hsa-miR-1290 or hsa-miR-146a-5p at an abundance at least 2-fold, 3-fold, 4-fold, 5-fold, 6-fold, 7-fold, 8-fold, 9-fold, or 10-fold higher than that of any of the miRNAs of those listed at ii) and iii). In certain embodiments of any such population of anti-inflammatory EVs described directly hereinabove, such a population comprises hsa-miR-146a-5p and hsa-miR-155-5p in a hsa-miR-146a-5p/hsa-miR-15-5p ratio of about 1-10, for example, 1.5-6, e.g., about 2, about 3, about 2 to about 3, about 2 to about 4, about 4, or about 5; and such a population comprises hsa-miR-1290 and hsa-miR-146a-5p at an abundance at least 2-fold, 3-fold, 4-fold, 5-fold, 6-fold, 7-fold, 8-fold, 9-fold, or 10-fold higher than that of any of the miRNAs of those listed at ii) through viii).

In one embodiment, a population of anti-inflammatory EVs provided herein is described as being characterized by a Treg RNA profile that comprises: i) three, four, or all five of hsa-miR-1290, hsa-miR-146a-5p, hsa-miR-155-5p, hsa-let-7a-5p, and/or hsa-miR-21-5p; ii) any 1-5 of hsa-miR-191-5p, hsa-miR-1246, hsa-let-7b-5p, hsa-miR-29a-3p, and/or hsa-miR-320a-3p; iii) any 1-5 of hsa-miR-423-5p, hsa-let-7f-5p, hsa-let-7i-5p, hsa-miR-16-5p, and/or hsa-miR-26a-5p; iv) any 1-5 of hsa-let-7g-5p, hsa-miR-221-3p, hsa-miR-222-3p, hsa-miR-26b-5p, and/or hsa-miR-342-3p; v) any 1-5 of hsa-miR-146b-5p, hsa-miR-92a-3p, hsa-miR-93-5p, hsa-miR-23a-3p, and/or hsa-miR-181a-5p; vi) any 1-5 of hsa-miR-150-5p, hsa-miR-20a-5p, hsa-miR-27a-3p, hsa-let-7d-5p, and/or hsa-miR-17-5p; vii) any 1-5 of hsa-miR-142-3p, hsa-miR-130b-3p, hsa-miR-25-3p, hsa-miR-142-5p, and/or hsa-miR-103a-3p; viii) any 1-5 of hsa-miR-28-3p, hsa-let-7e-5p, hsa-miR-425-5p, hsa-miR-186-5p, and/or hsa-miR-625-5p; and ix) any 1-5 of hsa-miR-4516, hsa-miR-22-3p, hsa-miR-24-3p, hsa-miR-486-5p, and/or hsa-miR-98-5p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) any 1-5 of hsa-miR-191-5p, hsa-miR-1246, hsa-let-7b-5p, hsa-miR-29a-3p, and/or hsa-miR-320a-3p; iii) any 1-5 of hsa-miR-423-5p, hsa-let-7f-5p, hsa-let-7i-5p, hsa-miR-16-5p, and/or hsa-miR-26a-5p; iv) any 1-5 of hsa-let-7g-5p, hsa-miR-221-3p, hsa-miR-222-3p, hsa-miR-26b-5p, and/or hsa-miR-342-3p; v) any 1-5 of hsa-miR-146b-5p, hsa-miR-92a-3p, hsa-miR-93-5p, hsa-miR-23a-3p, and/or hsa-miR-181a-5p; vi) any 1-5 of hsa-miR-150-5p, hsa-miR-20a-5p, hsa-miR-27a-3p, hsa-let-7d-5p, and/or hsa-miR-17-5p; vii) any 1-5 of hsa-miR-142-3p, hsa-miR-130b-3p, hsa-miR-25-3p, hsa-miR-142-5p, and/or hsa-miR-103a-3p; viii) any 1-5 of hsa-miR-28-3p, hsa-let-7e-5p, hsa-miR-425-5p, hsa-miR-186-5p, and/or hsa-miR-625-5p; and ix) any 1-5 of hsa-miR-4516, hsa-miR-22-3p, hsa-miR-24-3p, hsa-miR-486-5p, and/or hsa-miR-98-5p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) any 1-5 of hsa-miR-423-5p, hsa-let-7f-5p, hsa-let-7i-5p, hsa-miR-16-5p, and/or hsa-miR-26a-5p; iv) any 1-5 of hsa-let-7g-5p, hsa-miR-221-3p, hsa-miR-222-3p, hsa-miR-26b-5p, and/or hsa-miR-342-3p; v) any 1-5 of hsa-miR-146b-5p, hsa-miR-92a-3p, hsa-miR-93-5p, hsa-miR-23a-3p, and/or hsa-miR-181a-5p; vi) any 1-5 of hsa-miR-150-5p, hsa-miR-20a-5p, hsa-miR-27a-3p, hsa-let-7d-5p, and/or hsa-miR-17-5p; vii) any 1-5 of hsa-miR-142-3p, hsa-miR-130b-3p, hsa-miR-25-3p, hsa-miR-142-5p, and/or hsa-miR-103a-3p; viii) any 1-5 of hsa-miR-28-3p, hsa-let-7e-5p, hsa-miR-425-5p, hsa-miR-186-5p, and/or hsa-miR-625-5p; and ix) any 1-5 of hsa-miR-4516, hsa-miR-22-3p, hsa-miR-24-3p, hsa-miR-486-5p, and/or hsa-miR-98-5p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) each of hsa-miR-423-5p, hsa-let-7f-5p, hsa-let-7i-5p, hsa-miR-16-5p, and hsa-miR-26a-5p; iv) any 1-5 of hsa-let-7g-5p, hsa-miR-221-3p, hsa-miR-222-3p, hsa-miR-26b-5p, and/or hsa-miR-342-3p; v) any 1-5 of hsa-miR-146b-5p, hsa-miR-92a-3p, hsa-miR-93-5p, hsa-miR-23a-3p, and/or hsa-miR-181a-5p; vi) any 1-5 of hsa-miR-150-5p, hsa-miR-20a-5p, hsa-miR-27a-3p, hsa-let-7d-5p, and/or hsa-miR-17-5p; vii) any 1-5 of hsa-miR-142-3p, hsa-miR-130b-3p, hsa-miR-25-3p, hsa-miR-142-5p, and/or hsa-miR-103a-3p; viii) any 1-5 of hsa-miR-28-3p, hsa-let-7e-5p, hsa-miR-425-5p, hsa-miR-186-5p, and/or hsa-miR-625-5p; and ix) any 1-5 of hsa-miR-4516, hsa-miR-22-3p, hsa-miR-24-3p, hsa-miR-486-5p, and/or hsa-miR-98-5p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) each of hsa-miR-423-5p, hsa-let-7f-5p, hsa-let-7i-5p, hsa-miR-16-5p, and hsa-miR-26a-5p; iv) each of hsa-let-7g-5p, hsa-miR-221-3p, hsa-miR-222-3p, hsa-miR-26b-5p, and hsa-miR-342-3p; v) any 1-5 of hsa-miR-146b-5p, hsa-miR-92a-3p, hsa-miR-93-5p, hsa-miR-23a-3p, and/or hsa-miR-181a-5p; vi) any 1-5 of hsa-miR-150-5p, hsa-miR-20a-5p, hsa-miR-27a-3p, hsa-let-7d-5p, and/or hsa-miR-17-5p; vii) any 1-5 of hsa-miR-142-3p, hsa-miR-130b-3p, hsa-miR-25-3p, hsa-miR-142-5p, and/or hsa-miR-103a-3p; viii) any 1-5 of hsa-miR-28-3p, hsa-let-7e-5p, hsa-miR-425-5p, hsa-miR-186-5p, and/or hsa-miR-625-5p; and ix) any 1-5 of hsa-miR-4516, hsa-miR-22-3p, hsa-miR-24-3p, hsa-miR-486-5p, and/or hsa-miR-98-5p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) each of hsa-miR-423-5p, hsa-let-7f-5p, hsa-let-7i-5p, hsa-miR-16-5p, and hsa-miR-26a-5p; iv) each of hsa-let-7g-5p, hsa-miR-221-3p, hsa-miR-222-3p, hsa-miR-26b-5p, and hsa-miR-342-3p; v) each of hsa-miR-146b-5p, hsa-miR-92a-3p, hsa-miR-93-5p, hsa-miR-23a-3p, and hsa-miR-181a-5p; vi) any 1-5 of hsa-miR-150-5p, hsa-miR-20a-5p, hsa-miR-27a-3p, hsa-let-7d-5p, and/or hsa-miR-17-5p; vii) any 1-5 of hsa-miR-142-3p, hsa-miR-130b-3p, hsa-miR-25-3p, hsa-miR-142-5p, and/or hsa-miR-103a-3p; viii) any 1-5 of hsa-miR-28-3p, hsa-let-7e-5p, hsa-miR-425-5p, hsa-miR-186-5p, and/or hsa-miR-625-5p; and ix) any 1-5 of hsa-miR-4516, hsa-miR-22-3p, hsa-miR-24-3p, hsa-miR-486-5p, and/or hsa-miR-98-5p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) each of hsa-miR-423-5p, hsa-let-7f-5p, hsa-let-7i-5p, hsa-miR-16-5p, and hsa-miR-26a-5p; iv) each of hsa-let-7g-5p, hsa-miR-221-3p, hsa-miR-222-3p, hsa-miR-26b-5p, and hsa-miR-342-3p; v) each of hsa-miR-146b-5p, hsa-miR-92a-3p, hsa-miR-93-5p, hsa-miR-23a-3p, and hsa-miR-181a-5p; vi) each of hsa-miR-150-5p, hsa-miR-20a-5p, hsa-miR-27a-3p, hsa-let-7d-5p, and hsa-miR-17-5p; vii) any 1-5 of hsa-miR-142-3p, hsa-miR-130b-3p, hsa-miR-25-3p, hsa-miR-142-5p, and/or hsa-miR-103a-3p; viii) any 1-5 of hsa-miR-28-3p, hsa-let-7e-5p, hsa-miR-425-5p, hsa-miR-186-5p, and/or hsa-miR-625-5p; and ix) any 1-5 of hsa-miR-4516, hsa-miR-22-3p, hsa-miR-24-3p, hsa-miR-486-5p, and/or hsa-miR-98-5p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) each of hsa-miR-423-5p, hsa-let-7f-5p, hsa-let-7i-5p, hsa-miR-16-5p, and hsa-miR-26a-5p; iv) each of hsa-let-7g-5p, hsa-miR-221-3p, hsa-miR-222-3p, hsa-miR-26b-5p, and hsa-miR-342-3p; v) each of hsa-miR-146b-5p, hsa-miR-92a-3p, hsa-miR-93-5p, hsa-miR-23a-3p, and hsa-miR-181a-5p; vi) each of 1-5 of hsa-miR-150-5p, hsa-miR-20a-5p, hsa-miR-27a-3p, hsa-let-7d-5p, and hsa-miR-17-5p; vii) each of hsa-miR-142-3p, hsa-miR-130b-3p, hsa-miR-25-3p, hsa-miR-142-5p, and hsa-miR-103a-3p; viii) any 1-5 of hsa-miR-28-3p, hsa-let-7e-5p, hsa-miR-425-5p, hsa-miR-186-5p, and/or hsa-miR-625-5p; and ix) any 1-5 of hsa-miR-4516, hsa-miR-22-3p, hsa-miR-24-3p, hsa-miR-486-5p, and/or hsa-miR-98-5p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) each of hsa-miR-423-5p, hsa-let-7f-5p, hsa-let-7i-5p, hsa-miR-16-5p, and hsa-miR-26a-5p; iv) each of hsa-let-7g-5p, hsa-miR-221-3p, hsa-miR-222-3p, hsa-miR-26b-5p, and hsa-miR-342-3p; v) each of hsa-miR-146b-5p, hsa-miR-92a-3p, hsa-miR-93-5p, hsa-miR-23a-3p, and hsa-miR-181a-5p; vi) each of hsa-miR-150-5p, hsa-miR-20a-5p, hsa-miR-27a-3p, hsa-let-7d-5p, and hsa-miR-17-5p; vii) any 1-5 of hsa-miR-142-3p, hsa-miR-130b-3p, hsa-miR-25-3p, hsa-miR-142-5p, and/or hsa-miR-103a-3p; viii) any 1-5 of hsa-miR-28-3p, hsa-let-7e-5p, hsa-miR-425-5p, hsa-miR-186-5p, and/or hsa-miR-625-5p; and ix) any 1-5 of hsa-miR-4516, hsa-miR-22-3p, hsa-miR-24-3p, hsa-miR-486-5p, and/or hsa-miR-98-5p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) each of hsa-miR-423-5p, hsa-let-7f-5p, hsa-let-7i-5p, hsa-miR-16-5p, and hsa-miR-26a-5p; iv) each of hsa-let-7g-5p, hsa-miR-221-3p, hsa-miR-222-3p, hsa-miR-26b-5p, and hsa-miR-342-3p; v) each of hsa-miR-146b-5p, hsa-miR-92a-3p, hsa-miR-93-5p, hsa-miR-23a-3p, and hsa-miR-181a-5p; vi) each of 1-5 of hsa-miR-150-5p, hsa-miR-20a-5p, hsa-miR-27a-3p, hsa-let-7d-5p, and hsa-miR-17-5p; vii) each of hsa-miR-142-3p, hsa-miR-130b-3p, hsa-miR-25-3p, hsa-miR-142-5p, and hsa-miR-103a-3p; viii) each of hsa-miR-28-3p, hsa-let-7e-5p, hsa-miR-425-5p, hsa-miR-186-5p, and hsa-miR-625-5p; and ix) any 1-5 of hsa-miR-4516, hsa-miR-22-3p, hsa-miR-24-3p, hsa-miR-486-5p, and/or hsa-miR-98-5p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) each of hsa-miR-423-5p, hsa-let-7f-5p, hsa-let-7i-5p, hsa-miR-16-5p, and hsa-miR-26a-5p; iv) each of hsa-let-7g-5p, hsa-miR-221-3p, hsa-miR-222-3p, hsa-miR-26b-5p, and hsa-miR-342-3p; v) each of hsa-miR-146b-5p, hsa-miR-92a-3p, hsa-miR-93-5p, hsa-miR-23a-3p, and hsa-miR-181a-5p; vi) each of 1-5 of hsa-miR-150-5p, hsa-miR-20a-5p, hsa-miR-27a-3p, hsa-let-7d-5p, and hsa-miR-17-5p; vii) each of hsa-miR-142-3p, hsa-miR-130b-3p, hsa-miR-25-3p, hsa-miR-142-5p, and hsa-miR-103a-3p; viii) each of hsa-miR-28-3p, hsa-let-7e-5p, hsa-miR-425-5p, hsa-miR-186-5p, and hsa-miR-625-5p; and ix) each of hsa-miR-4516, hsa-miR-22-3p, hsa-miR-24-3p, hsa-miR-486-5p, and hsa-miR-98-5p.

In certain embodiments of any such population of anti-inflammatory EVs described directly hereinabove, such a population comprises hsa-miR-146a-5p and hsa-miR-155-5p in a hsa-miR-146a-5p/hsa-miR-15-5p ratio of about 1-10, for example, 1.5-6, e.g., about 2, about 3, about 2 to about 3, about 2 to about 4, about 4, or about 5. In certain embodiments of any such population of anti-inflammatory EVs described directly hereinabove, such a population comprises hsa-miR-1290 and hsa-miR-155-5p and such a population comprises hsa-miR-1290 at an abundance of at least 2-fold, 3-fold, 4-fold, 5-fold, 6-fold, 7-fold, 8-fold, 9-fold, or 10-fold higher than hsa-mir-155-5p. In other embodiments of any such population of anti-inflammatory EVs described directly hereinabove, such a population comprises at least one of hsa-miR-1290 or hsa-miR-146a-5p at an abundance at least 2-fold, 3-fold, 4-fold, 5-fold, 6-fold, 7-fold, 8-fold, 9-fold, or 10-fold higher than that of any of the miRNAs of those listed at ii) and iii). In certain embodiments of any such population of anti-inflammatory EVs described directly hereinabove, such a population comprises hsa-miR-146a-5p and hsa-miR-155-5p in a hsa-miR-146a-5p/hsa-miR-15-5p ratio of about 1-10, for example, 1.5-6, e.g., about 2, about 3, about 2 to about 3, about 2 to about 4, about 4, or about 5; and such a population comprises hsa-miR-1290 and hsa-miR-146a-5p at an abundance at least 2-fold, 3-fold, 4-fold, 5-fold, 6-fold, 7-fold, 8-fold, 9-fold, or 10-fold higher than that of any of the miRNAs of those listed at ii) through ix).

In one embodiment, a population of anti-inflammatory EVs provided herein is described as being characterized by a Treg RNA profile that comprises: i) three, four, or all five of hsa-miR-1290, hsa-miR-146a-5p, hsa-miR-155-5p, hsa-let-7a-5p, and/or hsa-miR-21-5p; ii) any 1-5 of hsa-miR-191-5p, hsa-miR-1246, hsa-let-7b-5p, hsa-miR-29a-3p, and/or hsa-miR-320a-3p; iii) any 1-5 of hsa-miR-423-5p, hsa-let-7f-5p, hsa-let-7i-5p, hsa-miR-16-5p, and/or hsa-miR-26a-5p; iv) any 1-5 of hsa-let-7g-5p, hsa-miR-221-3p, hsa-miR-222-3p, hsa-miR-26b-5p, and/or hsa-miR-342-3p; v) any 1-5 of hsa-miR-146b-5p, hsa-miR-92a-3p, hsa-miR-93-5p, hsa-miR-23a-3p, and/or hsa-miR-181a-5p; vi) any 1-5 of hsa-miR-150-5p, hsa-miR-20a-5p, hsa-miR-27a-3p, hsa-let-7d-5p, and/or hsa-miR-17-5p; vii) any 1-5 of hsa-miR-142-3p, hsa-miR-130b-3p, hsa-miR-25-3p, hsa-miR-142-5p, and/or hsa-miR-103a-3p; viii) any 1-5 of hsa-miR-28-3p, hsa-let-7e-5p, hsa-miR-425-5p, hsa-miR-186-5p, and/or hsa-miR-625-5p; ix) any 1-5 of hsa-miR-4516, hsa-miR-22-3p, hsa-miR-24-3p, hsa-miR-486-5p, and/or hsa-miR-98-5p; and x) any 1-5 of hsa-miR-181b-5p, hsa-miR-378a-3p, hsa-miR-30d-5p, hsa-miR-454-3p, and/or hsa-miR-342-5p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) any 1-5 of hsa-miR-191-5p, hsa-miR-1246, hsa-let-7b-5p, hsa-miR-29a-3p, and/or hsa-miR-320a-3p; iii) any 1-5 of hsa-miR-423-5p, hsa-let-7f-5p, hsa-let-7i-5p, hsa-miR-16-5p, and/or hsa-miR-26a-5p; iv) any 1-5 of hsa-let-7g-5p, hsa-miR-221-3p, hsa-miR-222-3p, hsa-miR-26b-5p, and/or hsa-miR-342-3p; v) any 1-5 of hsa-miR-146b-5p, hsa-miR-92a-3p, hsa-miR-93-5p, hsa-miR-23a-3p, and/or hsa-miR-181a-5p; vi) any 1-5 of hsa-miR-150-5p, hsa-miR-20a-5p, hsa-miR-27a-3p, hsa-let-7d-5p, and/or hsa-miR-17-5p; vii) any 1-5 of hsa-miR-142-3p, hsa-miR-130b-3p, hsa-miR-25-3p, hsa-miR-142-5p, and/or hsa-miR-103a-3p; viii) any 1-5 of hsa-miR-28-3p, hsa-let-7e-5p, hsa-miR-425-5p, hsa-miR-186-5p, and/or hsa-miR-625-5p; ix) any 1-5 of hsa-miR-4516, hsa-miR-22-3p, hsa-miR-24-3p, hsa-miR-486-5p, and/or hsa-miR-98-5p; and x) any 1-5 of hsa-miR-181b-5p, hsa-miR-378a-3p, hsa-miR-30d-5p, hsa-miR-454-3p, and/or hsa-miR-342-5p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) any 1-5 of hsa-miR-423-5p, hsa-let-7f-5p, hsa-let-7i-5p, hsa-miR-16-5p, and/or hsa-miR-26a-5p; iv) any 1-5 of hsa-let-7g-5p, hsa-miR-221-3p, hsa-miR-222-3p, hsa-miR-26b-5p, and/or hsa-miR-342-3p; v) any 1-5 of hsa-miR-146b-5p, hsa-miR-92a-3p, hsa-miR-93-5p, hsa-miR-23a-3p, and/or hsa-miR-181a-5p; vi) any 1-5 of hsa-miR-150-5p, hsa-miR-20a-5p, hsa-miR-27a-3p, hsa-let-7d-5p, and/or hsa-miR-17-5p; vii) any 1-5 of hsa-miR-142-3p, hsa-miR-130b-3p, hsa-miR-25-3p, hsa-miR-142-5p, and/or hsa-miR-103a-3p; viii) any 1-5 of hsa-miR-28-3p, hsa-let-7e-5p, hsa-miR-425-5p, hsa-miR-186-5p, and/or hsa-miR-625-5p; ix) any 1-5 of hsa-miR-4516, hsa-miR-22-3p, hsa-miR-24-3p, hsa-miR-486-5p, and/or hsa-miR-98-5p; and x) any 1-5 of hsa-miR-181b-5p, hsa-miR-378a-3p, hsa-miR-30d-5p, hsa-miR-454-3p, and/or hsa-miR-342-5p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) each of hsa-miR-423-5p, hsa-let-7f-5p, hsa-let-7i-5p, hsa-miR-16-5p, and hsa-miR-26a-5p; iv) any 1-5 of hsa-let-7g-5p, hsa-miR-221-3p, hsa-miR-222-3p, hsa-miR-26b-5p, and/or hsa-miR-342-3p; v) any 1-5 of hsa-miR-146b-5p, hsa-miR-92a-3p, hsa-miR-93-5p, hsa-miR-23a-3p, and/or hsa-miR-181a-5p; vi) any 1-5 of hsa-miR-150-5p, hsa-miR-20a-5p, hsa-miR-27a-3p, hsa-let-7d-5p, and/or hsa-miR-17-5p; vii) any 1-5 of hsa-miR-142-3p, hsa-miR-130b-3p, hsa-miR-25-3p, hsa-miR-142-5p, and/or hsa-miR-103a-3p; viii) any 1-5 of hsa-miR-28-3p, hsa-let-7e-5p, hsa-miR-425-5p, hsa-miR-186-5p, and/or hsa-miR-625-5p; ix) any 1-5 of hsa-miR-4516, hsa-miR-22-3p, hsa-miR-24-3p, hsa-miR-486-5p, and/or hsa-miR-98-5p; and x) any 1-5 of hsa-miR-181b-5p, hsa-miR-378a-3p, hsa-miR-30d-5p, hsa-miR-454-3p, and/or hsa-miR-342-5p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) each of hsa-miR-423-5p, hsa-let-7f-5p, hsa-let-7i-5p, hsa-miR-16-5p, and hsa-miR-26a-5p; iv) each of hsa-let-7g-5p, hsa-miR-221-3p, hsa-miR-222-3p, hsa-miR-26b-5p, and hsa-miR-342-3p; v) any 1-5 of hsa-miR-146b-5p, hsa-miR-92a-3p, hsa-miR-93-5p, hsa-miR-23a-3p, and/or hsa-miR-181a-5p; vi) any 1-5 of hsa-miR-150-5p, hsa-miR-20a-5p, hsa-miR-27a-3p, hsa-let-7d-5p, and/or hsa-miR-17-5p; vii) any 1-5 of hsa-miR-142-3p, hsa-miR-130b-3p, hsa-miR-25-3p, hsa-miR-142-5p, and/or hsa-miR-103a-3p; viii) any 1-5 of hsa-miR-28-3p, hsa-let-7e-5p, hsa-miR-425-5p, hsa-miR-186-5p, and/or hsa-miR-625-5p; ix) any 1-5 of hsa-miR-4516, hsa-miR-22-3p, hsa-miR-24-3p, hsa-miR-486-5p, and/or hsa-miR-98-5p; and x) any 1-5 of hsa-miR-181b-5p, hsa-miR-378a-3p, hsa-miR-30d-5p, hsa-miR-454-3p, and/or hsa-miR-342-5p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) each of hsa-miR-423-5p, hsa-let-7f-5p, hsa-let-7i-5p, hsa-miR-16-5p, and hsa-miR-26a-5p; iv) each of hsa-let-7g-5p, hsa-miR-221-3p, hsa-miR-222-3p, hsa-miR-26b-5p, and hsa-miR-342-3p; v) each of hsa-miR-146b-5p, hsa-miR-92a-3p, hsa-miR-93-5p, hsa-miR-23a-3p, and hsa-miR-181a-5p; vi) any 1-5 of hsa-miR-150-5p, hsa-miR-20a-5p, hsa-miR-27a-3p, hsa-let-7d-5p, and/or hsa-miR-17-5p; vii) any 1-5 of hsa-miR-142-3p, hsa-miR-130b-3p, hsa-miR-25-3p, hsa-miR-142-5p, and/or hsa-miR-103a-3p; viii) any 1-5 of hsa-miR-28-3p, hsa-let-7e-5p, hsa-miR-425-5p, hsa-miR-186-5p, and hsa-miR-625-5p; ix) any 1-5 of hsa-miR-4516, hsa-miR-22-3p, hsa-miR-24-3p, hsa-miR-486-5p, and hsa-miR-98-5p; and x) any 1-5 of hsa-miR-181b-5p, hsa-miR-378a-3p, hsa-miR-30d-5p, hsa-miR-454-3p, and hsa-miR-342-5p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) each of hsa-miR-423-5p, hsa-let-7f-5p, hsa-let-7i-5p, hsa-miR-16-5p, and hsa-miR-26a-5p; iv) each of hsa-let-7g-5p, hsa-miR-221-3p, hsa-miR-222-3p, hsa-miR-26b-5p, and hsa-miR-342-3p; v) each of hsa-miR-146b-5p, hsa-miR-92a-3p, hsa-miR-93-5p, hsa-miR-23a-3p, and hsa-miR-181a-5p; vi) each of hsa-miR-150-5p, hsa-miR-20a-5p, hsa-miR-27a-3p, hsa-let-7d-5p, and hsa-miR-17-5p; vii) any 1-5 of hsa-miR-142-3p, hsa-miR-130b-3p, hsa-miR-25-3p, hsa-miR-142-5p, and/or hsa-miR-103a-3p; viii) any 1-5 of hsa-miR-28-3p, hsa-let-7e-5p, hsa-miR-425-5p, hsa-miR-186-5p, and/or hsa-miR-625-5p; ix) any 1-5 of hsa-miR-4516, hsa-miR-22-3p, hsa-miR-24-3p, hsa-miR-486-5p, and/or hsa-miR-98-5p; and x) any 1-5 of hsa-miR-181b-5p, hsa-miR-378a-3p, hsa-miR-30d-5p, hsa-miR-454-3p, and/or hsa-miR-342-5p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) each of hsa-miR-423-5p, hsa-let-7f-5p, hsa-let-7i-5p, hsa-miR-16-5p, and hsa-miR-26a-5p; iv) each of hsa-let-7g-5p, hsa-miR-221-3p, hsa-miR-222-3p, hsa-miR-26b-5p, and hsa-miR-342-3p; v) each of hsa-miR-146b-5p, hsa-miR-92a-3p, hsa-miR-93-5p, hsa-miR-23a-3p, and hsa-miR-181a-5p; vi) each of hsa-miR-150-5p, hsa-miR-20a-5p, hsa-miR-27a-3p, hsa-let-7d-5p, and hsa-miR-17-5p; vii) each of hsa-miR-142-3p, hsa-miR-130b-3p, hsa-miR-25-3p, hsa-miR-142-5p, and hsa-miR-103a-3p; viii) any 1-5 of hsa-miR-28-3p, hsa-let-7e-5p, hsa-miR-425-5p, hsa-miR-186-5p, and/or hsa-miR-625-5p; ix) any 1-5 of hsa-miR-4516, hsa-miR-22-3p, hsa-miR-24-3p, hsa-miR-486-5p, and/or hsa-miR-98-5p; and x) any 1-5 of hsa-miR-181b-5p, hsa-miR-378a-3p, hsa-miR-30d-5p, hsa-miR-454-3p, and/or hsa-miR-342-5p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) each of hsa-miR-423-5p, hsa-let-7f-5p, hsa-let-7i-5p, hsa-miR-16-5p, and hsa-miR-26a-5p; iv) each of hsa-let-7g-5p, hsa-miR-221-3p, hsa-miR-222-3p, hsa-miR-26b-5p, and hsa-miR-342-3p; v) each of hsa-miR-146b-5p, hsa-miR-92a-3p, hsa-miR-93-5p, hsa-miR-23a-3p, and hsa-miR-181a-5p; vi) each of hsa-miR-150-5p, hsa-miR-20a-5p, hsa-miR-27a-3p, hsa-let-7d-5p, and hsa-miR-17-5p; vii) each of hsa-miR-142-3p, hsa-miR-130b-3p, hsa-miR-25-3p, hsa-miR-142-5p, and hsa-miR-103a-3p; viii) each of hsa-miR-28-3p, hsa-let-7e-5p, hsa-miR-425-5p, hsa-miR-186-5p, and hsa-miR-625-5p; ix) any 1-5 of hsa-miR-4516, hsa-miR-22-3p, hsa-miR-24-3p, hsa-miR-486-5p, and/or hsa-miR-98-5p; and x) any 1-5 of hsa-miR-181b-5p, hsa-miR-378a-3p, hsa-miR-30d-5p, hsa-miR-454-3p, and/or hsa-miR-342-5p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) each of hsa-miR-423-5p, hsa-let-7f-5p, hsa-let-7i-5p, hsa-miR-16-5p, and hsa-miR-26a-5p; iv) each of hsa-let-7g-5p, hsa-miR-221-3p, hsa-miR-222-3p, hsa-miR-26b-5p, and hsa-miR-342-3p; v) each of hsa-miR-146b-5p, hsa-miR-92a-3p, hsa-miR-93-5p, hsa-miR-23a-3p, and hsa-miR-181a-5p; vi) each of hsa-miR-150-5p, hsa-miR-20a-5p, hsa-miR-27a-3p, hsa-let-7d-5p, and hsa-miR-17-5p; vii) each of hsa-miR-142-3p, hsa-miR-130b-3p, hsa-miR-25-3p, hsa-miR-142-5p, and hsa-miR-103a-3p; viii) each of hsa-miR-28-3p, hsa-let-7e-5p, hsa-miR-425-5p, hsa-miR-186-5p, and hsa-miR-625-5p; ix) each of hsa-miR-4516, hsa-miR-22-3p, hsa-miR-24-3p, hsa-miR-486-5p, and hsa-miR-98-5p; and x) any 1-5 of hsa-miR-181b-5p, hsa-miR-378a-3p, hsa-miR-30d-5p, hsa-miR-454-3p, and/or hsa-miR-342-5p. In one embodiment, a population of anti-inflammatory EVs provided herein is described as being characterized by a Treg RNA profile that comprises: i) each of hsa-miR-1290, hsa-miR-146a-5p, hsa-miR-155-5p, hsa-let-7a-5p, and hsa-miR-21-5p; ii) each of hsa-miR-191-5p, hsa-miR-1246, hsa-let-7b-5p, hsa-miR-29a-3p, and hsa-miR-320a-3p; iii) each of hsa-miR-423-5p, hsa-let-7f-5p, hsa-let-7i-5p, hsa-miR-16-5p, and hsa-miR-26a-5p; iv) each of hsa-let-7g-5p, hsa-miR-221-3p, hsa-miR-222-3p, hsa-miR-26b-5p, and hsa-miR-342-3p; v) each of hsa-miR-146b-5p, hsa-miR-92a-3p, hsa-miR-93-5p, hsa-miR-23a-3p, and hsa-miR-181a-5p; vi) each of hsa-miR-150-5p, hsa-miR-20a-5p, hsa-miR-27a-3p, hsa-let-7d-5p, and hsa-miR-17-5p; vii) each of hsa-miR-142-3p, hsa-miR-130b-3p, hsa-miR-25-3p, hsa-miR-142-5p, and hsa-miR-103a-3p; viii) each of hsa-miR-28-3p, hsa-let-7e-5p, hsa-miR-425-5p, hsa-miR-186-5p, and hsa-miR-625-5p; ix) each of hsa-miR-4516, hsa-miR-22-3p, hsa-miR-24-3p, hsa-miR-486-5p, and hsa-miR-98-5p; and x) each of hsa-miR-181b-5p, hsa-miR-378a-3p, hsa-miR-30d-5p, hsa-miR-454-3p, and hsa-miR-342-5p.

In certain embodiments of any such population of anti-inflammatory EVs described directly hereinabove, such a population comprises hsa-miR-146a-5p and hsa-miR-155-5p in a hsa-miR-146a-5p/hsa-miR-15-5p ratio of about 1-10, for example, 1.5-6, e.g., about 2, about 3, about 2 to about 3, about 2 to about 4, about 4, or about 5. In certain embodiments of any such population of anti-inflammatory EVs described directly hereinabove, such a population comprises hsa-miR-1290 and hsa-miR-155-5p and such a population comprises hsa-miR-1290 at an abundance of at least 2-fold, 3-fold, 4-fold, 5-fold, 6-fold, 7-fold, 8-fold, 9-fold, or 10-fold higher than hsa-mir-155-5p. In other embodiments of any such population of anti-inflammatory EVs described directly hereinabove, such a population comprises at least one of hsa-miR-1290 or hsa-miR-146a-5p at an abundance at least 2-fold, 3-fold, 4-fold, 5-fold, 6-fold, 7-fold, 8-fold, 9-fold, or 10-fold higher than that of any of the miRNAs of those listed at ii) and iii). In certain embodiments of any such population of anti-inflammatory EVs described directly hereinabove, such a population comprises hsa-miR-146a-5p and hsa-miR-155-5p in a hsa-miR-146a-5p/hsa-miR-15-5p ratio of about 1-10, for example, 1.5-6, e.g., about 2, about 3, about 2 to about 3, about 2 to about 4, about 4, or about 5; and such a population comprises hsa-miR-1290 and hsa-miR-146a-5p at an abundance at least 2-fold, 3-fold, 4-fold, 5-fold, 6-fold, 7-fold, 8-fold, 9-fold, or 10-fold higher than that of any of the miRNAs of those listed at ii) through x).

### 5.2 Methods of Producing Anti-Inflammatory EVs

In certain aspects, presented herein are methods for producing an isolated, cell-free population of anti-inflammatory EVs.

In certain embodiments, presented herein are methods for producing an isolated, cell-free population of anti-inflammatory EVs, wherein the method comprises: a) ex-vivo expanding a human suppressive immune cell population in culture media to produce a culture comprising the human suppressive immune cell population, the culture media and anti-inflammatory EVs (without wishing to be bound by theory or mechanism, it is presumed that the EVs are released by the human suppressive immune cells into the culture during the ex vivo expanding), and b) isolating the anti-inflammatory EVs from the culture.

In certain embodiments, presented herein are methods for producing an isolated, cell-free population of anti-inflammatory EVs, wherein the method comprises: a) ex-vivo expanding a human suppressive immune cell population, wherein the human suppressive immune cell population is a population of regulatory T cells (Tregs), in culture media to produce a culture comprising the Treg population, the culture media and anti-inflammatory EVs, and b) isolating the anti-inflammatory EVs from the culture.

Methods for obtaining, optionally enriching, and ex vivo expanding human suppressive immune cells, e.g., Tregs, are well known. Exemplary methods are presented below.

For ease of description, methods for producing an isolated, cell-free population of anti-inflammatory EVs, may be presented herein as comprising: a) ex-vivo expanding a human suppressive immune cell population in culture media to produce a culture comprising the human suppressive immune cell population, the culture media and anti-inflammatory EVs, and b) isolating the anti-inflammatory EVs from the culture. It is to be understood, however, that isolating the anti-inflammatory EVs from the culture may be performed at any point once the ex-vivo expanding begins, or may be performed repeatedly during the ex-vivo expanding period.

For example, the ex-vivo expanding may comprise multiple rounds of expansion. In one non-limiting example, culture media comprising EVs may be collected at the end of one or more of the rounds of expansion, from which the EVs may be isolated. In another non-limiting example, culture media comprising EVs may be collected during expansion at points when the culture media of the culture is replenished or changed. In another non-limiting example, EVs may be isolated at the end of the ex-vivo expansion process. In yet another non-limiting example, EVs may be isolated at multiple points during the ex-vivo expansion process, e.g., at one or more of the points noted above.

In certain embodiments, the ex vivo expansion period lasts about 24 h, 48h, or 72h. In certain embodiments, the ex vivo expansion period lasts 1 day, 2 days, 3 days, 4 days, 5 days, 6 days, 7 days, 1 week, 10 days, 14 days, 2 weeks, 3 weeks or more.

In some embodiments, EVs may be isolated after about 24 h, 48h, or 72h of expanding. In some embodiments, EVs may be isolated about 24h, about 48h, or about 72h after the culture medium is replenished or changed. In some embodiments, EVs are isolated every 2, 3, 4, or 5 days.

In certain embodiments, culture media comprising EVs may be collected at one or more points during the expansion process and the isolating of the EVs begins when the culture media is collected, e.g., within 30 minutes, 1 hour, 2 hours, 3 hours, 4 hours, 8 hours or overnight after the culture media is collected. In particular embodiments, culture media comprising EVs may be collected at one or more points during the expansion process and stored at 4° C. prior to the isolating of the EVs. In specific embodiments, for example, culture media comprising EVs may be collected at one or more points during the expansion process and may be stored at 4° C. for about 30 minutes, 1 hour, 2 hours, 3 hours, 4 hours, 8 hours or overnight prior to isolating of the EVs from the culture media.

In certain embodiments, culture media comprising EVs may be collected at one or more points during the expansion process and stored, for example, frozen prior to isolating of the EVs.

In particular embodiments, EVs may be isolated from cell culture by centrifugation, for example differential centrifugation. In certain embodiments, differential centrifugation may be used to isolate a desired subpopulation of EVs. For example, differential centrifugation may be employed to isolate a subpopulation of EVs enriched for a smaller particle diameter size (e.g., exosomes; EVs with a particle size less than about 300 nm, less than about 200 nm, less than about 160 nm, less than about 150 nm, less than about 130 nm, less than about 100 nm, or less than about 80 nm). In a particular, non-limiting example, centrifugation steps at 2,000g (3,000 rpm) for 20 min may be employed to remove cell debris and dead cells and at 16,500g (9,800 rpm) for 45 min, or at 100,000g (26,450 rpm) for 2 h, to specifically isolate exosomes.

EVs may also be purified using gradient density centrifugation, which separates EVs from the culture based on their based on their buoyant density in solutions of either sucrose, iohexol, or iodixanol.

Additional examples of methods used to isolate EVs include precipitation with organic solvents (e.g., polyethylene glycol, sodium acetate or protamine), immunoprecipitation, separation using antibody-coated magnetic beads, microfluidic devices, and ultrafiltration, which are described, for example, in Carnino et al. Respiratory Research (2019) 20:240 and Momen-Heravi et al. Biol. Chem. 2013; 394(10): 1253-1262. Further exemplary methods are isolation using heparin-conjugated agarose beads (see, e.g., Balaj et al. (2015) Sci Rep 5, 10266) and purification using Tim4-affinity purification (see, e.g., Nakai et al. (2016) Sci Rep 6, 33935).

Commercial kits for the isolation of EVs are also available. Non-limiting examples include the exoEasy Kit (Qiagen), ExoQuick® kits (Systems Bioscience), and the EasySep™ Human Pan-Extracellular Vesicle Positive Selection Kit (Stem Cell Technologies).

In some embodiments, a method of producing an isolated, cell-free population of anti-inflammatory EVs provided herein comprises the steps of (a) ex-vivo expanding a human suppressive immune cell population (e.g., a Treg cell population) in culture media to produce a culture comprising the cells, the culture media and anti-inflammatory EVs; and (b) isolating the anti-inflammatory EVs from the culture.

In certain embodiments, a method of producing an isolated, cell-free population of anti-inflammatory EVs, isolating the anti-inflammatory EVs from the culture comprises polyethylene glycol (PEG) precipitation. In a specific embodiment, PEG is added to the culture such that the EVs are precipitated out of the culture. Following removal of the EV-containing precipitate from the culture, the EVs are washed to produce an isolated, cell-free population of anti-inflammatory EVs.

An exemplary, non-limiting protocol for the isolation of EVs from cells (e.g., Tregs) using PEG precipitation may comprise the steps of (i) centrifuging media from cell culture (e.g., ex vivo-expanded human suppressive immune cell, for example Treg, cell culture) at 3000×g for 15 minutes to remove cells and debris; (ii) adding PEG reagent to the supernatant, for example at a 1:5 ratio of PEG:supernatant; (iii) mixing thoroughly; (iv) refrigerating overnight at 4° C.; (v) centrifuging at 1500×g for 30 minutes (vi) aspirating the supernatant (vii) centrifuging again at 1500×g for 10 minutes; (viii) removing the supernatant, e.g., removing the supernatant via aspiration; and (ix) resuspending the resulting EV pellet in sterile buffer, e.g., sterile PBS.

EVs may also be isolated from cell culture using filtration, for example, tangential flow filtration (TFF). TFF, for example, may be utilized to efficiently isolate and concentrate EV populations in a scalable and reproducible manner even when beginning with a large culture volume.

In particular embodiments, for example, the isolation step (b) comprises removing the cells from the culture to produce a cell-free, population of anti-inflammatory EVs.

In particular embodiments, for example, the isolation step (b) comprises the steps of (i) removing the cells from the culture to produce a cell-free, anti-inflammatory EV-containing solution; and (ii) isolating the anti-inflammatory EVs from the cell-free, anti-inflammatory EV-containing solution of (i). Steps (i) and (ii) may be performed separately, e.g., sequentially, as separate steps, or may be accomplished as a single step.

In some embodiments, step (b) comprises filtration, for example, one or more filtration steps. In particular embodiments the filtration comprises TFF. For example, some or all of the filtration may utilize TFF. Filtration, for example, may be utilized to remove cell and debris from the culture. Filtration may also be used to isolate and concentrate EVs, for example, to isolate and concentrate EVs of a particular size or size range. In certain embodiments, removal of cell and debris and isolation of EVs, for example, a particular size or size range of EVs, may be accomplished using a single filtration step. In other embodiments, for example, a series (two or more) of filtration steps may be utilized to remove cell and debris and isolate EV, isolate a particular size range of EVs. For example, one or more filtration steps may be utilized to first remove cell and debris to produce an EV-containing solution, followed by one or more filtration steps that isolate and concentrate the EV population from the solution, e.g., isolate and concentrate a particular size or size range of EVs from the solution.

In some embodiments, step (b), for example, step (i), comprises filtration, e.g., microfiltration (for example, microfiltration by TFF). For example, the culture may be passed through a filter, e.g., a 0.05 μm, 0.1 μm, 0.2 μm, 0.45 μm, 0.65 μm or 0.8 μm filter, to remove the cells and any debris from the culture to produce a cell-free anti-inflammatory EV-containing solution comprising the anti-inflammatory population. In a specific embodiment, the culture may be passed through a 0.65 μm filter to remove the cells and any debris from the culture to produce a cell-free anti-inflammatory EV-containing solution comprising the anti-inflammatory population. In particular embodiments, the culture may be circulated through a filter, e.g., a 0.05 μm, 0.1 μm, 0.2 μm, 0.45 μm, 0.65 μm or 0.8 μm filter, using TFF to remove the cell and any debris from the culture to produce a cell-free anti-inflammatory EV-containing solution comprising the anti-inflammatory EV population. In a particular embodiment, the culture may be circulated through a 0.65 μm filter using TFF to remove the cell and any debris from the culture to produce a cell-free anti-inflammatory EV-containing solution comprising the anti-inflammatory EV population. In specific embodiments, the filter used in step (i) has a membrane area of 85 cm². In specific embodiments, the filter used in step (i) is a hollow fiber filter. In a specific embodiment, the filter used in step (i) is a hollow fiber filter with a fiber diameter of 0.75 mm. One or more rounds of filtration may be utilized. One or more sizes of filter may be utilized. In addition to removal of cells and debris, it is to be understood that such filtration may also serve to isolate a particular size or size range of EVs.

In specific embodiments, the microfiltration in step (i) (for example, microfiltration by TFF) is performed at a flow rate of 20-1000 mL/min. In specific embodiments, the microfiltration in step (i) (for example, microfiltration by TFF) is performed at a flow rate of 50-500 mL/min. In specific embodiments, the microfiltration in step (i) (for example, microfiltration by TFF) is performed at a flow rate of 100-200 mL/min. In a specific embodiment, the microfiltration in step (i) (for example, microfiltration by TFF) is performed at a flow rate of about 100 mL/min. In a specific embodiment, the microfiltration in step (i) (for example, microfiltration by TFF) is performed at a flow rate of about 150 mL/min. In a specific embodiment, the microfiltration in step (i) (for example, microfiltration by TFF) is performed at a flow rate of about 200 mL/min.

In specific embodiments, the microfiltration in step (i) (for example, microfiltration by TFF) is performed using a hollow fiber filter with a shear rate of about 2,000-5,000 s⁻¹. In specific embodiments, the microfiltration in step (i) (for example, microfiltration by TFF) is performed using a hollow fiber filter with a shear rate of about 2,000-3,000 s⁻¹. In specific embodiments, the microfiltration in step (i) (for example, microfiltration by TFF) is performed using a hollow fiber filter with a shear rate of about 3,000-4,000 s⁻¹. In specific embodiments, the microfiltration in step (i) (for example, microfiltration by TFF) is performed using a hollow fiber filter with a shear rate of about 4,000-5,000 s⁻¹. In a specific embodiment, the microfiltration in step (i) (for example, microfiltration by TFF) is performed using a hollow fiber filter with a shear rate of about 2,000 s⁻¹. In a specific embodiment, the microfiltration in step (i) (for example, microfiltration by TFF) is performed using a hollow fiber filter with a shear rate of about 3,000 s⁻¹. In a specific embodiment, the microfiltration in step (i) (for example, microfiltration by TFF) is performed using a hollow fiber filter with a shear rate of about 4,000 s″ 1. In a specific embodiment, the microfiltration in step (i) (for example, microfiltration by TFF) is performed using a hollow fiber filter with a shear rate of about 5,000 s⁻¹. Shear rate is a term used for hollow fiber membranes and is affected by flow rate and radius of the fiber. While the typical range of shear rate is 2000-12000 s⁻¹, preferably the shear rate maintained in step (i) is about 2,000-5,000 s-′ (and not higher) so as to avoid shredding of EVs and to result in a high efficiency of EV recovery (e.g., recovery of more than 90% or more than 95% EVs). In specific embodiments, the shear rate maintained in step (i) is about 2,000-5,000 s⁻¹, with a flow rate of 100-200 mL/min and using a hollow fiber filter that has a fiber diameter of 0.75 mm.

In certain embodiments, the retentate pressure of step (i) is maintained at about 5 psi. In a specific embodiment, the shear rate maintained in step (i) is about 2,000-5,000 s⁻¹, with a flow rate of 100-200 mL/min and using a hollow fiber filter that has a fiber diameter of 0.75 mm, resulting in a retentate pressure of about 5 psi.

In some embodiments, step (b) comprises step (ii), and step (ii) may comprise filtration, for example, ultrafiltration (for example, ultrafiltration by TFF). In particular embodiments, step (ii) comprises a step of passing the cell-free, anti-inflammatory EV-containing solution through a filter such that the anti-inflammatory EVs, for example, a particular size or size range of anti-inflammatory EVs, are retained by the filter. In particular embodiments, step (ii) comprises a step of circulating the cell-free, anti-inflammatory EV-containing solution through a filter using TFF such that the anti-inflammatory EVs are retained by the filter. One or more rounds of filtration may be utilized. One or more sizes of filter may be utilized. Step (ii) may also serve to concentrate the EVs. In specific embodiments, the final volume of the EV-containing solution after concentration is about 5-200 mLs. In specific embodiments, the final volume of the EV-containing solution after concentration is about 10-100 mLs. In specific embodiments, the final volume of the EV-containing solution after concentration is about 10-50 mLs. In a specific embodiment, the final volume of the EV-containing solution after concentration is about 10 mL. In a specific embodiment, the final volume of the EV-containing solution after concentration is about 15 mL. In a specific embodiment, the final volume of the EV-containing solution after concentration is about 20 mL. In a specific embodiment, the final volume of the EV-containing solution after concentration is about 25 mL. In a specific embodiment, the final volume of the EV-containing solution after concentration is about 30 mL.

In some embodiments, at least one filter used in step (ii) has a molecular weight cut-off (MWCO) of about 50 kilodaltons (kDa) to about 750 kDa, about 100 kDa to about 750 kDa, about 300 kDa to about 750 kDa, or about 300 kDa to about 500 kDa. In some embodiments, the filter has an MWCO of about 50 kDa, about 60 kDA, about 70 kDa, about 80 kDa, about 90 kDa, about 100 kDa, about 110 kDa, about 120 kDa, about 150 kDa, about 200 kDa, about 300 kDa, about 400 kDa, about 500 kDa about 600 kDa, about 700 kDa or about 750 kDa. In one embodiment, the filter has an MWCO of about 500 kDa. In some embodiments, a filter used in step (ii) has a pore size of about 0.3 μm, about 0.22 μm, about 0.2 μm or about 0.1 μm. In specific embodiments, the filter used in step (ii) has a membrane area of 115 cm². In specific embodiments, the filter used in step (ii) is a hollow fiber filter. In a specific embodiment, the filter used in step (ii) is a hollow fiber filter with a fiber diameter of 0.5 mm.

In certain embodiments, step (ii) is designed to retain EVs of a particle size or size range, e.g., to retain EVs greater than about 50 nm to about 60 nm, about 60 nm to about 80 nm, about 60 nm to about 70 nm, about 70 nm to about 80 nm, about 80 nm, about 100 nm, about 150 nm, or about 200 nm.

In certain embodiments, step (ii) is designed to retain EVs greater than about 50 nm to about 60 nm and comprises use of a filter with an MWCO of about 300 kDa. In certain embodiments, step (ii) is designed to retain EVs greater than about 50 nm and comprises use of a filter with an MWCO of about 300 kDa. In certain embodiments, step (ii) is designed to retain EVs greater than about 70 nm to about 80 nm and comprises use of a filter with an MWCO of about 500 kDa. In certain embodiments, step (ii) is designed to retain EVs greater than about 70 nm and comprises use of a filter with an MWCO of about 500 kDa. In certain embodiments, step (ii) is designed to retain EVs greater than about 80 nm and comprises use of a filter with an MWCO of about 500 kDa. In certain embodiments, step (ii) is designed to retain EVs greater than about 60 nm and comprises use of a filter with an MWCO of about 500 kDa.

In some embodiments, step (b), for example, step (b)(ii), comprises performing buffer exchange such that the isolated, cell-free population of anti-inflammatory EVs produced is a buffer-containing isolated, cell-free population of anti-inflammatory EVs. In particular embodiments, buffer exchange comprises diafiltration. In specific embodiments, buffer exchange comprises TFF and diafiltration. In specific embodiments, the diafiltration is performed at 2×-100×. In specific embodiments, the diafiltration is performed at 5×-50×. In specific embodiments, the diafiltration is performed at 5×-20×. In a specific embodiment, the diafiltration is performed at 5×. In a specific embodiment, the diafiltration is performed at 10×. In a specific embodiment, the diafiltration is performed at 15×. In a specific embodiment, the diafiltration is performed at 20×.

For example, in some embodiments, step (b) comprises step (ii), and step (ii) may comprise a step of circulating the cell-free, anti-inflammatory EV-containing solution through a filter using TFF such that the anti-inflammatory EVs are retained by the filter, wherein the circulating comprises incorporation of a suitable buffer into the solution, so that over the course of the process the buffer replaces the solution, thereby results in a buffer-containing isolated, cell-free population of anti-inflammatory EVs.

In certain embodiments, the buffer is a sterile buffer. In certain embodiments, the buffer is a sterile buffer suitable for administration to a human, e.g., is suitable for administration to a human for therapeutic use. In a specific embodiment, the buffer is a saline-containing buffer. In one embodiment, the buffer is saline. In one embodiment, the buffer is physiological saline. In one embodiment, the buffer is normal saline. In one embodiment, the buffer is 0.9% saline. In one embodiment, the buffer is phosphate-buffered saline (PBS).

In specific embodiments, the ultrafiltration (and optionally diafiltration) in step (ii) (for example, ultrafiltration, and optionally diafiltration, by TFF) is performed at a flow rate of 20-1000 mL/min. In specific embodiments, the ultrafiltration (and optionally diafiltration) in step (ii) (for example, ultrafiltration, and optionally diafiltration, by TFF) is performed at a flow rate of 50-500 mL/min. In specific embodiments, the ultrafiltration (and optionally diafiltration) in step (ii) (for example, ultrafiltration, and optionally diafiltration, by TFF) is performed at a flow rate of 80-200 mL/min. In specific embodiments, the ultrafiltration (and optionally diafiltration) in step (ii) (for example, ultrafiltration, and optionally diafiltration, by TFF) is performed at a flow rate of 80-175 mL/min. In a specific embodiment, the ultrafiltration (and optionally diafiltration) in step (ii) (for example, ultrafiltration, and optionally diafiltration, by TFF) is performed at a flow rate of about 80 mL/min. In a specific embodiment, the ultrafiltration (and optionally diafiltration) in step (ii) (for example, ultrafiltration, and optionally diafiltration, by TFF) is performed at a flow rate of about 100 mL/min. In a specific embodiment, the ultrafiltration (and optionally diafiltration) in step (ii) (for example, ultrafiltration, and optionally diafiltration, by TFF) is performed at a flow rate of about 125 mL/min. In a specific embodiment, the ultrafiltration (and optionally diafiltration) in step (ii) (for example, ultrafiltration, and optionally diafiltration, by TFF) is performed at a flow rate of about 150 mL/min. In a specific embodiment, the ultrafiltration (and optionally diafiltration) in step (ii) (for example, ultrafiltration, and optionally diafiltration, by TFF) is performed at a flow rate of about 175 mL/min. In a specific embodiment, the ultrafiltration (and optionally diafiltration) in step (ii) (for example, ultrafiltration, and optionally diafiltration, by TFF) is performed at a flow rate of about 200 mL/min.

In specific embodiments, the ultrafiltration (and optionally diafiltration) in step (ii) (for example, ultrafiltration, and optionally diafiltration, by TFF) is performed using a hollow fiber filter with a shear rate of about 2,000-8,000 s⁻¹. In specific embodiments, the ultrafiltration (and optionally diafiltration) in step (ii) (for example, ultrafiltration, and optionally diafiltration, by TFF) is performed using a hollow fiber filter with a shear rate of about 2,000-7,500 s⁻¹. In specific embodiments, the ultrafiltration (and optionally diafiltration) in step (ii) (for example, ultrafiltration, and optionally diafiltration, by TFF) is performed using a hollow fiber filter with a shear rate of about 2,000-7,000 s⁻¹. In specific embodiments, the ultrafiltration (and optionally diafiltration) in step (ii) (for example, ultrafiltration, and optionally diafiltration, by TFF) is performed using a hollow fiber filter with a shear rate of about 2,000-3,000 s⁻¹. In specific embodiments, the ultrafiltration (and optionally diafiltration) in step (ii) (for example, ultrafiltration, and optionally diafiltration, by TFF) is performed using a hollow fiber filter with a shear rate of about 3,000-4,000 s⁻¹. In specific embodiments, the ultrafiltration (and optionally diafiltration) in step (ii) (for example, ultrafiltration, and optionally diafiltration, by TFF) is performed using a hollow fiber filter with a shear rate of about 4,000-5,000 s⁻¹. In specific embodiments, the ultrafiltration (and optionally diafiltration) in step (ii) (for example, ultrafiltration, and optionally diafiltration, by TFF) is performed using a hollow fiber filter with a shear rate of about 5,000-6,000 s⁻¹. In specific embodiments, the ultrafiltration (and optionally diafiltration) in step (ii) (for example, ultrafiltration, and optionally diafiltration, by TFF) is performed using a hollow fiber filter with a shear rate of about 6,000-7,000 s⁻¹. In specific embodiments, the ultrafiltration (and optionally diafiltration) in step (ii) (for example, ultrafiltration, and optionally diafiltration, by TFF) is performed using a hollow fiber filter with a shear rate of about 7,000-8,000 s⁻¹. In a specific embodiment, the ultrafiltration (and optionally diafiltration) in step (ii) (for example, ultrafiltration, and optionally diafiltration, by TFF) is performed using a hollow fiber filter with a shear rate of about 2,000 s⁻¹. In a specific embodiment, the ultrafiltration (and optionally diafiltration) in step (ii) (for example, ultrafiltration, and optionally diafiltration, by TFF) is performed using a hollow fiber filter with a shear rate of about 3,000 s⁻¹. In a specific embodiment, the ultrafiltration (and optionally diafiltration) in step (ii) (for example, ultrafiltration, and optionally diafiltration, by TFF) is performed using a hollow fiber filter with a shear rate of about 4,000 s⁻¹. In a specific embodiment, the ultrafiltration (and optionally diafiltration) in step (ii) (for example, ultrafiltration, and optionally diafiltration, by TFF) is performed using a hollow fiber filter with a shear rate of about 5,000 s⁻¹. In a specific embodiment, the ultrafiltration (and optionally diafiltration) in step (ii) (for example, ultrafiltration, and optionally diafiltration, by TFF) is performed using a hollow fiber filter with a shear rate of about 6,000 s⁻¹. In a specific embodiment, the ultrafiltration (and optionally diafiltration) in step (ii) (for example, ultrafiltration, and optionally diafiltration, by TFF) is performed using a hollow fiber filter with a shear rate of about 7,000 s⁻¹. In a specific embodiment, the ultrafiltration (and optionally diafiltration) in step (ii) (for example, ultrafiltration, and optionally diafiltration, by TFF) is performed using a hollow fiber filter with a shear rate of about 7,500 s⁻¹. In a specific embodiment, the ultrafiltration (and optionally diafiltration) in step (ii) (for example, ultrafiltration, and optionally diafiltration, by TFF) is performed using a hollow fiber filter with a shear rate of about 8,000 s⁻¹. Shear rate is a term used for hollow fiber membranes and is affected by flow rate and radius of the fiber. While the typical range of shear rate is 2000-12000 s⁻¹, preferably the shear rate maintained in step (ii) is about 2,000-8,000 s⁻¹, about 2,000-7,500 s⁻¹, or about 2,000-7,000 s⁻¹ (and not higher) so as to avoid shredding of EVs and to result in a high efficiency of EV recovery (e.g., recovery of more than 90% or more than 95% EVs). In specific embodiments, the shear rate maintained in step (ii) is about 2,000-7,500 s⁻¹, with a flow rate of 80-200 mL/min and using a hollow fiber filter that has a fiber diameter of 0.5 mm.

In certain embodiments, the transmembrane pressure of step (ii) is maintained at about 10 psi. In a specific embodiment, the shear rate maintained in step (ii) is about 2,000-7,500 s⁻¹, with a flow rate of 80-200 mL/min and using a hollow fiber filter that has a fiber diameter of 0.5 mm, resulting in a transmembrane pressure of about 10 psi.

In certain embodiments, the isolation step (b) comprises: a step (i) that comprises microfiltration as described above, and a step (ii) that comprises ultrafiltration as described above and optionally diafiltration as described above.

In certain embodiments, the step (i) described above is performed using one or more pumps (e.g., one or more automated pumps), such as a main pump and an auxiliary pump. In certain embodiments, the step (ii) described above is performed using one or more pumps (e.g., one or more automated pumps), such as a main pump and an auxiliary pump. In certain embodiments, step (i) and step (ii) described above are each performed using one or more pumps (e.g., one or more automated pumps), such as a main pump and an auxiliary pump.

In a particular, non-limiting example, a Repligen KR2i TFF system can be used to isolate, concentrate, and diafiltrate the EVs from cell culture into an appropriate buffer for therapeutic use. For example, EV isolation using TFF may comprise the steps of (i) circulating the culture media using TFF and a Midi 20 cm 0.65 μm Spectrum mPES Hollow Fiber filter (D02-E65U-07-N) with a membrane area of 85 cm² and fiber diameter of 0.75 mm to filter out cells and debris (e.g., utilizing a flow rate of 100-200 mL/min that results in a shear rate of about 2,000-5,000 s⁻¹ while maintaining a variable transmembrane pressure (TMP) driven by a retentate pressure of 5 psi) and (ii) using the permeate of the process to concentrate and diafiltrate the EV product. For example, the process may utilize a TFF system and a Midi 20 cm 500 kD Spectrum mPES Hollow Fiber filter (D02-E500-05-N) with a membrane area of 115 cm² filter and fiber diameter of 0.5 mm to retain/concentrate particles greater than about 60-80 nm into the retentate with continuous circulation (e.g., utilizing a flow rate of 80-200 mL/min that results in a shear rate of 2,000-7,500 s⁻¹ while maintaining and driving the filtration at 10 psi TMP). Incorporation of a suitable buffer into the circulation (for example, sterile saline or sterile PBS) may be performed to diafiltrate and replace the existing solution so that the EVs end up in a sterile solution that is acceptable for therapeutic use.

In certain embodiments, the isolated EVs may be stored at −20° C. In particular embodiments, the isolated EVs may be stored at −20° C. while limiting freeze/thaw cycles.

In certain embodiments, the isolated EVs may be stored at about 2° C. to about 8° C. (e.g., at about 4° C.), e.g., may be stored for up to about one week at about 2° C. to about 8° C. (e.g., at about 4° C.), for example, may be stored about overnight, for up to about 1 day, up to about 2 days, up to about 3 days, up to about 4 days, up to about 5 days, up to about 6 days, or up to about 7 days at about 2° C. to about 8° C. (e.g., at about 4° C.). In certain embodiments, the isolated EVs are stored at 4° C. for less than about 2 weeks, e.g., are stored for less than about 14 days, less than about 13 days, less than about 12 days, less than about 11 days, less than about 10 days, less than about 9 days, less than about 8 days, at about 2° C. to about 8° C. (e.g., at about 4° C.).

In certain embodiments, the methods presented herein for producing an isolated, cell-free population of anti-inflammatory EVs results in a yield of about 1×10⁸ to about 1×10¹⁰ EVs/ml of culture media. In certain embodiments, the methods presented herein for producing an isolated, cell-free population of anti-inflammatory EVs results in a yield of about 5×10⁸ to about 1×10¹⁰ EVs/ml of culture media. In certain embodiments, the methods presented herein for producing an isolated, cell-free population of anti-inflammatory EVs results in a yield of about 1×10⁹ to about 1×10¹⁰ EVs/ml of culture media. In certain embodiments, the methods presented herein for producing an isolated, cell-free population of anti-inflammatory EVs results in a yield of about 5×10⁹ to about 1×10¹⁰ EVs/ml of culture media. In certain embodiments, the methods presented herein for producing an isolated, cell-free population of anti-inflammatory EVs results in a yield of about 1×10⁹ EVs/ml, about 2×10⁹ EVs/ml, about 3×10⁹ EVs/ml, about 4×10⁹ EVs/ml, about 5×10⁹ EVs/ml, about 6×10⁹ EVs/ml, about 7×10⁹ EVs/ml, about 8×10⁹ EVs/ml, about 9×10⁹ EVs/ml, or about 1×10¹⁰ EVs/ml of culture media.

The anti-inflammatory EVs presented herein may be derived from ex vivo-expanded human suppressive immune cells, e.g., Tregs. Exemplary methods for expanding Tregs are presented herein.

In some embodiments, the anti-inflammatory EVs presented herein are derived from ex vivo-expanded human suppressive immune cells, e.g., Tregs. Exemplary methods for expanding Tregs are presented herein.

### 5.2.1. Culture, Enrichment, and Expansion of Human Suppressive Immune Cells

The isolated, cell-free populations of anti-inflammatory EVs presented herein are derived from ex vivo-expanded human suppressive immune cells. In certain aspects, the isolated, cell-free populations of anti-inflammatory EVs presented herein are derived from ex vivo-expanded human Tregs.

In some embodiments, the human suppressive immune cells, e.g., Tregs, are from a healthy human subject. In some embodiments, the human suppressive immune cells, e.g., Tregs, are from greater than one healthy human subject. In particular embodiments, for example, the human suppressive immune cells, e.g., Tregs, are from 2, 3, 4, 5, 6, 7, 8, 9, 10, 11, 12, 13, 14, 15, 20, 25, 30, 35, 40, 45, 50 or more healthy human subjects. In other particular embodiments, for example, the human suppressive immune cells, e.g., Tregs, are from 2-50, 2-5, 2-10, 5-10, 5-50, 5-25, 10-15, 10-50, 10-25, 15-25, 25-30, 30-35, 35-40, 40-45, or 45-50 subjects. In some embodiments, the subjects are related. In some embodiments, the subjects are not unrelated.

In particular embodiments where the human suppressive immune cells, e.g., Tregs, are from more than one human subject, a method of producing an isolated, cell-free population of anti-inflammatory EVs may comprise pooling the cells from the more than one human subject together prior to ex vivo-expanding the cells. In other particular embodiments where the human suppressive immune cells, e.g., Tregs, are from more than one human subject, a method of producing an isolated, cell-free population of anti-inflammatory EVs may comprise ex vivo-expanding the cells from one or more of the human subjects separately and pooling the anti-inflammatory EVs resulting from each culture.

In some embodiments, the human suppressive immune cells, e.g., Tregs, are from a donor subject diagnosed with or is suspected of having a disorder associated with Treg dysfunction. In some embodiments, the donor subject is diagnosed with or is suspected of having a disorder associated with Treg deficiency. In some embodiments, the donor subject is diagnosed with or is suspected of having a condition driven by a T cell response.

In some embodiments, the human suppressive immune cells, e.g., Tregs, are from a donor subject diagnosed with or is suspected of having a neurodegenerative disease. In some embodiments, the donor subject is diagnosed with or is suspected of having Alzheimer's disease, Amyotrophic Lateral Sclerosis, multiple sclerosis (MS), Parkinson's Disease, Huntington's disease or frontotemporal dementia.

In some embodiments, the human suppressive immune cells, e.g., Tregs, are from a donor subject diagnosed with or is suspected of having a disorder that would benefit from downregulation of the immune system.

In some embodiments, the human suppressive immune cells, e.g., Tregs, are from a donor subject diagnosed with or suspected of having an autoimmune disease. The autoimmune disease may be, for example, systemic sclerosis (scleroderma), polymyositis, ulcerative colitis, inflammatory bowel disease, Crohn's disease, celiac disease, multiple sclerosis (MS), rheumatoid arthritis (RA), Type I diabetes, psoriasis, dermatomyositis, lupus, e.g., systemic lupus erythematosus, or cutaneous lupus, myasthenia gravis, autoimmune nephropathy, autoimmune hemolytic anemia, autoimmune cytopenia autoimmune hepatitis, autoimmune uveitis, alopecia, thyroiditis or pemhigus.

In some embodiments, the human suppressive immune cells, e.g., Tregs, are from a donor subject diagnosed with or suspected of having heart failure or ischemic cardiomyopathy. In some embodiments, the donor subject is diagnosed with or suspected of having graft-versus-host disease, e.g., after undergoing organ transplantation (such as a kidney transplantation or a liver transplantation), or after undergoing stem cell transplantation (such as hematopoietic stem cell transplantation).

In some embodiments, the human suppressive immune cells, e.g., Tregs, are from a donor subject diagnosed with or suspected of having neuroinflammation. Neuroinflammation may be associated, for example, with stroke, acute disseminated encephalitis, acute optic neuritis, transverse myelitis, neuromyelitis optica, epilepsy, traumatic brain injury, spinal cord injury, encephalitis central nervous system (CNS) vasculitis, neurosarcoidosis, autoimmune or post-infectious encephalitis or chronic meningitis.

In some embodiments, the human suppressive immune cells, e.g., Tregs, are from a donor subject diagnosed with or suspected of having chronic inflammatory demyelinating polyradiculoneuropathy (CIDP). In some embodiments, the donor subject is diagnosed with or suspected of having acute inflammatory demyelinating polyneuropathy (AIDP). In some embodiments, the donor subject is diagnosed with or suspected of having Guillain-Barré syndrome (GBS).

In some embodiments, the human suppressive immune cells, e.g., Tregs, are from a donor subject diagnosed with or suspected of having cardo-inflammation, e.g., cardio-inflammation associated with myocardial infarction, ischemic cardiomyopathy, with heart failure.

In some embodiments, the human suppressive immune cells, e.g., Tregs, are from a donor subject who has had a stroke.

In some embodiments, the human suppressive immune cells, e.g., Tregs, are from a donor subject diagnosed with or suspected of having cancer, e.g., a blood cancer.

In some embodiments, the human suppressive immune cells, e.g., Tregs, are from a donor subject diagnosed with or suspected of having asthma.

In some embodiments, the human suppressive immune cells, e.g., Tregs, are from a donor subject diagnosed with or suspected of having eczema.

In some embodiments, the human suppressive immune cells, e.g., Tregs, are from a donor subject diagnosed with or suspected of having a disorder associated with overactivation of the immune system.

In some embodiments, the human suppressive immune cells, e.g., Tregs, are from a donor subject diagnosed with or suspected of having Tregopathy. The Tregopathy may, for example, be caused by a FOXP3, CD25, cytotoxic T lymphocyte-associated antigen 4 (CTLA4), LPS-responsive and beige-like anchor protein (LRBA), or BTB domain and CNC homolog 2 (BACH2) gene loss-of-function mutation, or a signal transducer and activator of transcription 3 (STAT3) gain-of-function mutation.

In some embodiments, the human suppressive immune cells, e.g., Tregs, are from one or more adult subjects, for example, one or more healthy adult subjects. In certain embodiments, the one or more subjects are of at least 18, 20, 25, 30, 35, 40, 45, 50 or 55 years of age. In particular embodiments, for example, the human suppressive immune cells, e.g., Tregs, are from one or more adult subjects, wherein the one or more healthy adult subjects are about 18-55, about 18-50, about 18-45, about 18-40, about 18-35, about 18-30, about 18-25, about 20-55, about 25-55, about 30-55, about 35-55, about 40-55, about 25-50, about 30-50, about 35-45, about 25-45 about 40-50 years of age.

In some embodiments, the human suppressive immune cells, e.g., Tregs, are from a geriatric subject, for example, a healthy geriatric subject, e.g., a subject of at least 65, at least 70, at least 75, at least 80, at least 85 or at least 90 years of age.

In some embodiments, the anti-inflammatory EVs provided herein are derived from a genetically engineered population of human suppressive immune cells, e.g., Tregs.

5.2.1.1 An improved method of ex-vivo expanding Tregs

In some embodiments, an isolated, cell-free population of anti-inflammatory EVs provided herein is derived from an ex-vivo expanded population of human Tregs. Methods of expanding Tregs are well known. Described in this section is an improved method of ex-vivo expanding Tregs.

In certain embodiments, a biological donor sample, e.g., a peripheral blood sample or thymic tissue, containing or suspected of containing Tregs may be obtained, from which Tregs may be obtained and enriched for prior to ex-vivo expanding. Exemplary methods of producing obtaining, enriching for and ex-vivo expanding a population of Tregs are described in International Patent Application No. PCT/US2020/63378, which is incorporated by reference herein in its entirety. In some embodiments, the Tregs from which EVs are obtained are expanded according to the disclosure set forth in section 5.2.2, below. In some embodiments, the Tregs from which EVs are obtained are expanded according to the disclosure set forth in section 6.12, below.

In certain embodiments, the enrichment step is automated. In certain embodiments, the enrichment step takes place in a closed system. In certain embodiments, the enrichment step is automated and takes place in a closed system. In specific embodiments, the enrichment step takes place in a CliniMACS Prodigy® system. In specific embodiments, the enrichment step takes place in a CliniMACS® Plus system. In certain embodiments, the expansion step is automated. In certain embodiments, the expansion step takes place in a closed system. In certain embodiments, the expansion step is automated and takes place in a closed system. In specific embodiments, the expansion step takes place in a bioreactor (e.g., a Terumo BCT QuantumR Cell Expansion System). In some embodiments, the enrichment step and the expansion step take place in different systems (for example, the enrichment step takes place in a CliniMACS Prodigy® system and the expansion step takes place in a Terumo BCT Quantum R Cell Expansion System, or the enrichment step takes place in a CliniMACS® Plus system and the expansion step takes place in a Terumo BCT Quantum® Cell Expansion System). In specific embodiments, the enriched cell population produced by the enrichment step are transferred to the system where the expansion step takes place in a closed step. In other embodiments, the enrichment step and the expansion step take place in the same system. In specific embodiments, the same system is a closed system.

In some embodiments, the population of Tregs is obtained from a serum sample suspected of containing Tregs. In some embodiments, the population of Tregs is obtained from a cell sample suspected of containing Tregs, obtained from a donor via leukapheresis or obtained from a donor via blood sample. In some embodiments, the population of Tregs is obtained from a biological sample suspected of containing Tregs.

In some embodiments, the population of Tregs is enriched from a biological sample from a human donor subject. In some embodiments, the donor of the biological sample is the patient subject to be treated by the population of anti-inflammatory EVs derived from the population of Tregs. In other embodiments, the donor of the biological sample is different from the patient subject to be treated by the population of anti-inflammatory EVs derived from the population of Tregs. The biological sample can be any sample suspected of containing Tregs, likely to contain Tregs or known to contain Tregs. Such biological samples may be taken directly from the subject, or may be samples resulting from one or more processing steps, such as separation, e.g. selection or enrichment, centrifugation, washing, and/or incubation. Biological samples include, but are not limited to, body fluids, such as blood, plasma, serum, cerebrospinal fluid, synovial fluid, tissue and organ samples, including processed samples derived therefrom.

In some aspects, the sample is blood or a blood-derived sample, or is or is derived from an apheresis or leukapheresis product. Exemplary samples include whole blood, peripheral blood mononuclear cells (PBMCs), leukocytes, bone marrow, and thymus.

In some embodiments, the biological sample is a blood-derived sample, e.g., a samples derived from whole blood, serum, or plasma. In some embodiments, the biological sample is or includes peripheral blood mononuclear cells. In some embodiments, the biological sample is a peripheral blood or serum sample. In some embodiments, the biological sample is a lymph node sample.

Methods of obtaining a population of cells suspected to contain, likely to contain or known to contain Tregs from such biological donor samples are known in the art. For example, lymphocytes may be obtained from a peripheral blood sample by leukapheresis. In some embodiments, Tregs are enriched from a population of lymphocytes. In some embodiments, repeated peripheral blood samples are obtained from a donor for producing Tregs. In some embodiments, two or more peripheral blood samples are obtained from a donor. In some embodiments, the donor sample undergoes volume reduction during the enrichment process. In some embodiments, biological samples (e.g., leukapheresis samples or blood samples) from more than one donor are pooled prior to the enrichment process to generate an allogeneic population of Tregs. In some embodiments, biological samples (e.g., leukapheresis samples or blood samples) from more than one unrelated donor are pooled prior to the enrichment process to generate an allogeneic population of Tregs. In some embodiments, biological samples (e.g., leukapheresis or blood samples from 2, 3, 4, 5, 10, 20, 50 or more donors) are pooled.

Tregs may be enriched from a biological sample by any method known in the art. In some embodiments, Tregs are enriched from a sample using magnetic bead separation (e.g., CliniMACS Tubing Set LS (162-01), CliniMACSR Plus Instrument or CliniMACS Prodigy® Instrument), fluorescent cell sorting, and/or disposable closed cartridge based cell sorters.

Enrichment involves enriching for cells expressing one or more markers, and may refer to increasing the number or percentage of such cells in the population of cells, but does not necessarily result in a complete absence of cells not expressing the marker. Depletion of cells expressing one or more markers refers to decreasing the number or percentage of such cells in the population of cells, but does not necessarily result in a complete removal of all cells expressing such marker or markers.

In some embodiments, the enrichment comprises a step of affinity- or

immunoaffinity-based separation of cells expressing one or more markers (e.g., Treg cell surface markers). Such separation steps can be based on positive selection, in which the cells expressing one or more markers are retained, and/or on negative selection (depletion), in which the cells not expressing one or more markers are retained.

The separation may be based on the expression (e.g., positive or negative expression) or expression level (e.g., high or low expression) of one or more markers (e.g., Treg cell surface markers). In this context, “high expression” and “low expression” are generally relative to the whole population of cells. In some embodiments, separation of cells may be based on CD8 expression. In some embodiments, separation of cells may be based on CD19 expression. In some embodiments, separation of cells may be based on high CD25 expression.

In some embodiments, separation of cells may be based on high CD9 expression. In some embodiments, separation of cells may be based on high CD63 expression. In some embodiments, separation of cells may be based on high CD81 expression. In some embodiments, separation of cells may be based on high CD44 expression. In some embodiments, separation of cells may be based on high CD29 expression. In some embodiments, separation of cells may be based on high CD45 expression.

Thus, in some embodiments, enrichment of Tregs may comprise incubation with an antibody or binding partner that specifically binds to a marker (e.g., a Treg cell surface marker), followed generally by washing steps and separation of cells having bound the antibody or binding partner from those cells having not bound to the antibody or binding partner.

In some embodiments, the antibody or binding partner is bound to a solid support or matrix, such as a sphere or bead, for example a nanoparticle, microbeads, nanobeads, including agarose, magnetic bead or paramagnetic beads. In some embodiments, the spheres or beads can be packed into a column to effect immunoaffinity chromatography. In some embodiments, the antibody or binding partner is detectably labeled. In some embodiments, the antibody or binding partner is attached to small, magnetically responsive particles or microparticles, such as nanoparticles or paramagnetic beads. Such beads are known and are commercially available (e.g., Dynabeads® (Life Technologies, Carlsbad, CA), MACS® beads (Miltenyi Biotec, San Diego, CA) or Streptamer® bead reagents (IBA, Germany)). Such particles or microparticles may be incubated with the population of cells to be enriched and then placed in a magnetic field. This results in those cells that are attached to the particles or microparticles via the antibody or binding partner being attracted to the magnet and separated from the unbound cells. This method allows for retention of the cells attached to the magnet (positive selection) or removal of the cells attracted to the magnet (negative selection).

In some embodiments, a method of producing a population of Tregs provided herein comprises both positive and negative selection during the enrichment step.

In some embodiments, the biological sample is obtained within about 25-35 min, about 35-45 min, about 45-60 min, about 60-75 min, about 75-90 min, about 90-120 min, about 120-150 min, about 150-180 min, about 2-3h, about 3-4h, about 4-5h or about 5-6h of the beginning of the enriching step. In some embodiments, the sample is obtained within about 30 min of the beginning of the enriching step. In some embodiments, the biological sample is not stored (e.g., stored at 4° C.) overnight.

In some embodiments, enrichment of Tregs from a human sample comprises depleting the sample of CD8+ cells. In some embodiments, enrichment of Tregs from a human sample comprises depleting a sample of CD19+ cells. In some embodiments, enrichment of Tregs from a biological sample comprises depleting the sample of CD8+ cells and CD19+ cells. In some embodiments, enrichment of Tregs from a biological sample comprises enriching the cell population for CD25^{high} cells. In some embodiments, enrichment of Tregs from a biological sample comprises enriching the cell population for CD25+ cells. In some embodiments, enrichment of Tregs from a biological sample comprises depletion of CD8+ cells and CD19+ cells from the sample and enriching the cell population for CD25^{high} cells. In some embodiments, enrichment of Tregs from a biological sample comprises depleting CD8+/CD19+ cells and enriching for CD25+ cells.

In some embodiments, the population of cells enriched for Tregs comprises an increased proportion of CD4+CD25^{high} Tregs relative to the proportion of CD4+CD25^{high} Tregs in the Tregs prior to enrichment as determined by flow cytometry. In specific embodiments, the proportion of CD4+CD25^{high} Tregs is increased by about 2-fold to about 4-fold, about 4-fold to about 6-fold, about 6-fold to about 8-fold, about 8-fold to about 10-fold, about 10-fold to about 15-fold, about 15-fold to about 20-fold, about 20-fold to about 25-fold, about 25-fold to about 30-fold, about 30-fold to about 35-fold, about 35-fold to about 40-fold, about 40-fold to about 45-fold, about 45-fold to about 50-fold.

In some embodiments, the population of cells enriched for Tregs comprises an increased proportion of CD4+CD25^{high}CD 127^{low} Tregs relative to the proportion of CD4+CD25^{high}CD127^{low} Tregs in the Tregs prior to enrichment as determined by flow cytometry. In specific embodiments, the proportion of CD4+CD25^{high}CD127^{low} Tregs is increased by about 2-fold to about 4-fold, about 4-fold to about 6-fold, about 6-fold to about 8-fold, about 8-fold to about 10-fold, about 10-fold to about 15-fold, about 15-fold to about 20-fold, about 20-fold to about 25-fold, about 25-fold to about 30-fold, about 30-fold to about 35-fold, about 35-fold to about 40-fold, about 40-fold to about 45-fold, about 45-fold to about 50-fold.

In some embodiments, the population of cells enriched for Tregs comprises CD25+Tregs wherein the expression of CD25 in the Tregs is increased relative to the expression of CD25 in the Tregs prior to enrichment, as determined by flow cytometry. In specific embodiments, the expression of CD25 is increased by at least about 5-fold, at least about 10-fold, at least about 15-fold, at least about 20-fold, at least about 25-fold, at least about 30-fold, at least about 35-fold, at least about 40-fold, at least about 45-fold, or at least about 50-fold.

In some embodiments, the population of cells enriched for Tregs comprises CD127+Tregs wherein the expression of CD127 in the Tregs is increased relative to the expression of CD127 in the Tregs prior to enrichment, as determined by flow cytometry. In specific embodiments, the expression of CD127 is increased by at least about 1.5-fold, at least about 2-fold, at least about 2.5-fold, or at least about 3-fold.

In some embodiments, the granularity of the Tregs in the enriched population of Tregs is increased relative to the granularity of the Tregs prior to enrichment, as determined by flow cytometry. In specific embodiments, the granularity of the Tregs increased by at least about 1.5-fold, at least about 2-fold, at least about 2.5-fold, or at least about 3-fold.

In some embodiments, the size of the Tregs in the enriched population of Tregs is increased relative to the size of the Tregs prior to enrichment, as determined by flow cytometry. In specific embodiments, the size of the Tregs increased by at least about 1.2-fold, at least about 1.5-fold, or at least about 2-fold.

In another aspect, the population of Tregs used to isolate the anti-inflammatory EVs provided herein is enriched from a biological sample and is further expanded.

In some embodiments, the expansion step is carried out within about 4-5 days after the completion of the enrichment step. In some embodiments, the expansion step is carried out within about 3-4 days after the completion of the enrichment step. In some embodiments, the expansion step is carried out within about 2-3 days after the completion of the enrichment step. In some embodiments, the expansion step is carried out within about 1-2 days after the completion of the enrichment step. In some embodiments, the expansion step is carried out within about 24 hours after the completion of the enrichment step. In some embodiments, the expansion step is carried out within about 12 hours after the completion of the enrichment step. In some embodiments, the expansion step is carried out within about 6 hours after the completion of the enrichment step. In some embodiments, the expansion step is carried out within about 3 hours after the completion of the enrichment step. In some embodiments, the expansion step is carried out within about 2 hours after the completion of the enrichment step. In some embodiments, the expansion step is carried out within about 1 hour after the completion of the enrichment step. In some embodiments, the expansion step is carried out within about 30 minutes after the completion of the enrichment step.

This expansion of the population of Tregs may comprise culturing the cells that have been enriched from a biological samples in media, for example, in serum-free media (e.g., TexMACS Medium), in serum-depleted media, or in serum-containing media.

In certain embodiments, the expansion step comprises culturing the Tregs in a culture medium that comprises human serum (e.g., TexMACS GMP Medium supplemented with human serum). In specific embodiments, the culture medium comprises 5% or less human serum. In specific embodiments, the culture medium comprises 4% or less human serum. In specific embodiments, the culture medium comprises 3% or less human serum. In specific embodiments, the culture medium comprises 2% or less human serum. In specific embodiments, the culture medium comprises 1% or less human serum. In specific embodiments, the culture medium comprises 0.5% or less human serum. In specific embodiments, the culture medium comprises less than 5% human serum. In specific embodiments, the culture medium comprises less than 4% human serum. In specific embodiments, the culture medium comprises less than 3% human serum. In specific embodiments, the culture medium comprises less than 2% human serum. In specific embodiments, the culture medium comprises less than 1% human serum. In specific embodiments, the culture medium comprises less than 0.5% human serum. In specific embodiments, the culture medium comprises 0-0.5% human serum. In specific embodiments, the culture medium comprises 0.5-1% human serum. In specific embodiments, the culture medium comprises 1-2% human serum. In specific embodiments, the culture medium comprises 2-3% human serum. In specific embodiments, the culture medium comprises 3-4% human serum. In specific embodiments, the culture medium comprises 4-5% human serum. In a specific embodiment, the culture medium comprises about 0.5% human serum. In another specific embodiment, the culture medium comprises about 1% human serum. In another specific embodiment, the culture medium comprises about 2% human serum. In another specific embodiment, the culture medium comprises about 3% human serum. In another specific embodiment, the culture medium comprises about 4% human serum. In another specific embodiment, the culture medium comprises about 5% human serum.

In certain embodiments, the expansion step comprises culturing the Tregs in a culture medium that comprises human AB serum (e.g., TexMACS GMP Medium supplemented with human AB serum). In specific embodiments, the culture medium comprises 5% or less human AB serum. In specific embodiments, the culture medium comprises 4% or less human AB serum. In specific embodiments, the culture medium comprises 3% or less human AB serum. In specific embodiments, the culture medium comprises 2% or less human AB serum. In specific embodiments, the culture medium comprises 1% or less human AB serum. In specific embodiments, the culture medium comprises 0.5% or less human AB serum. In specific embodiments, the culture medium comprises less than 5% human AB serum. In specific embodiments, the culture medium comprises less than 4% human AB serum. In specific embodiments, the culture medium comprises less than 3% human AB serum. In specific embodiments, the culture medium comprises less than 2% human AB serum. In specific embodiments, the culture medium comprises less than 1% human AB serum. In specific embodiments, the culture medium comprises less than 0.5% human AB serum. In specific embodiments, the culture medium comprises 0-0.5% human AB serum. In specific embodiments, the culture medium comprises 0.5-1% human AB serum. In specific embodiments, the culture medium comprises 1-2% human AB serum. In specific embodiments, the culture medium comprises 2-3% human AB serum. In specific embodiments, the culture medium comprises 3-4% human AB serum. In specific embodiments, the culture medium comprises 4-5% human AB serum. In a specific embodiment, the culture medium comprises about 0.5% human AB serum. In another specific embodiment, the culture medium comprises about 1% human AB serum. In another specific embodiment, the culture medium comprises about 2% human AB serum. In another specific embodiment, the culture medium comprises about 3% human AB serum. In another specific embodiment, the culture medium comprises about 4% human AB serum. In another specific embodiment, the culture medium comprises about 5% human AB serum.

In some embodiments, the cells enriched from a biological sample are cultured about 37° C. and about 5% CO₂. In some embodiments, the cells enriched from a biological sample are cultured out under good manufacturing practice (GMP) conditions. In some embodiments, the cells enriched from a biological sample are cultured in a closed system.

In some embodiments, the cells enriched from a biological sample are cultured in an automated system. In some embodiments, the cells enriched from a biological sample are cultured in a closed and automated system. In some embodiments, the cells enriched from a biological sample are cultured in a Terumo BCT QuantumR Cell Expansion System.

In some embodiments, the expansion of the population of Tregs begins within 25-35 min, within 20-40 min, within 15-45 min or within 10-50 min of the enrichment from a biological sample. In some embodiments, the expansion of the population of Tregs begins within about 30 min of the enrichment from a biological sample.

As noted above, once expansion begins, isolation of EVs may be performed at any point during or upon completion of the expansion process.

Tregs may be expanded ex vivo by culturing the cells in the presence of one or more expansion agents. In some embodiments, the expansion agent is IL-2. The appropriate concentration of IL-2 in the culture media can be determined by a person of skill in the art. In some embodiments, the concentration of IL-2 in the cell culture media is about 5-10 IU/mL, about 10-20 IU/mL, about 20-30 IU/mL, about 30-40 IU/mL, about 40-50 IU/mL, about 50-100 IU/mL, about 100-200 IU/mL, about 200-300 IU/mL, about 300-400 IU/mL, about 400-500 IU/mL, about 500-600 IU/mL, about 600-700 IU/mL, about 700-800 IU/mL, about 800-900 IU/mL, about 900-1000 IU/mL, about 1000-1500 IU/mL, about 1500-2000 IU/mL, about 2000-2500 IU/mL, about 2500-3000 IU/mL, about 3000-3500 IU/mL, about 3500-4000 IU/mL, about 4000-4500 IU/mL, about 4500-5000 IU/mL, about 5000-6000 IU/mL, about 6000-7000 IU/mL, about 7000-8000 IU/mL, about 8000-9000 IU/mL, or about 9000-10,000 IU/mL. In specific embodiments, the concentration of IL-2 in the cell culture media is about 100 IU/mL. In specific embodiments, the concentration of IL-2 in the cell culture media is about 150 IU/mL. In specific embodiments, the concentration of IL-2 in the cell culture media is about 200 IU/mL. In specific embodiments, the concentration of IL-2 in the cell culture media is about 250 IU/mL. In specific embodiments, the concentration of IL-2 in the cell culture media is about 300 IU/mL. In specific embodiments, the concentration of IL-2 in the cell culture media is about 400 IU/mL. In specific embodiments, the concentration of IL-2 in the cell culture media is about 500 IU/mL. In specific embodiments, the concentration of IL-2 in the cell culture media is about 600 IU/mL. In specific embodiments, the concentration of IL-2 in the cell culture media is about 700 IU/mL. In specific embodiments, the concentration of IL-2 in the cell culture media is about 800 IU/mL. In certain embodiments, the expansion step comprises adjusting IL-2 concentration depending on cell number. The cell number means the number of all cells in culture, including the enriched Treg cells, which represent a majority of the cells in culture and in specific embodiments represent more than 70%, more than 80%, more than 90%, more than 95%, more than 99%, or 100% of the cells in culture. In a specific embodiment, the expansion step comprises culturing the Tregs in a culture medium containing about 200 IU/mL IL-2 until the cell number reaches 600×10⁶, and then culturing the Tregs in a culture medium containing about 250 IU/mL IL-2.

In some embodiments, IL-2 is first added to the culture within about 4-5 days of initiating culture. In some embodiments, IL-2 is first added to the culture within about 3-4 days of initiating culture. In some embodiments, IL-2 is first added to the culture within about 2-3 days of initiating culture. In some embodiments, IL-2 is first added to the culture within about 1-2 days of initiating culture. In some embodiments, IL-2 is first added to the culture within about 24 hours of initiating culture. In some embodiments, IL-2 is first added to the culture within about 12 hours of initiating culture. In some embodiments, IL-2 is first added to the culture within about 6 hours of initiating culture. In some embodiments, IL-2 is first added to the culture within about 3 hours of initiating culture. In some embodiments, IL-2 is first added to the culture within about 2 hours of initiating culture. In some embodiments, IL-2 is first added to the culture within about 1 hour of initiating culture. In some embodiments, IL-2 is first added to the culture within about 30 minutes of initiating culture. In some embodiments, IL-2 is first added to the culture within about 4-5 days after the completion of the enrichment step. In some embodiments, IL-2 is first added to the culture within about 3-4 days after the completion of the enrichment step. In some embodiments, IL-2 is first added to the culture within about 2-3 days after the completion of the enrichment step. In some embodiments, IL-2 is first added to the culture within about 1-2 days after the completion of the enrichment step. In some embodiments, IL-2 is first added to the culture within about 24 hours after the completion of the enrichment step. In some embodiments, IL-2 is first added to the culture within about 12 hours after the completion of the enrichment step. In some embodiments, IL-2 is first added to the culture within about 6 hours after the completion of the enrichment step. In some embodiments, IL-2 is first added to the culture within about 3 hours after the completion of the enrichment step. In some embodiments, IL-2 is first added to the culture within about 2 hours after the completion of the enrichment step. In some embodiments, IL-2 is first added to the culture within about 1 hour after the completion of the enrichment step. In some embodiments, IL-2 is first added to the culture within about 30 minutes after the completion of the enrichment step. In some embodiments, IL-2 is replenished about every 1, 2, 3, 4, or 5 days. In some embodiments, IL-2 is replenished about every 1-2 days. In some embodiments, IL-2 is replenished about every 2-3 days. In some embodiments, IL-2 is replenished about every 3-4 days. In some embodiments, IL-2 is replenished about every 4-5 days.

In some embodiments, the expansion agent activates CD3, e.g., the expansion agent is an anti-CD3 antibody. In some embodiments, the expansion agent activates CD28, e.g., the expansion agent is an anti-CD28 antibody.

In some embodiments, the expansion agent is a soluble anti-CD3 antibody. In particular embodiments, the anti-CD3 antibody is OKT3. In some embodiments, the concentration of soluble anti-CD3 antibody in the culture media is about 0.1-0.2 ng/ml, about 0.2-0.3 ng/mL, about 0.3-0.4 ng/ml, about 0.4-0.5 ng/ml about 0.5-1 ng/ml, about 1-5 ng/ml, about 5-10 ng/ml, about 10-15 ng/ml, about 15-20 ng/ml, about 20-25 ng/mL, about 25-30 ng/mL, about 30-35 ng/mL, about 35-40 ng/mL, about 40-45 ng/mL, about 45-50 ng/mL, about 50-60 ng/ml, about 60-70 ng/ml, about 70-80 ng/ml, about 80-90 ng/ml, or about 90-100 ng/mL.

In some embodiments, the expansion agent is a soluble anti-CD28 antibody. Non-limiting examples of anti-CD28 antibodies include NA/LE (e.g. BD Pharmingen), IM1376 (e.g. Beckman Coulter), or 15×10⁸ (e.g. Miltenyi Biotec). In some embodiments, the concentration of soluble anti-CD28 antibody in the culture media is about 1-2 ng/ml, about 2-3 ng/mL, about 3-4 ng/mL, about 4-5 ng/mL, about 5-10 ng/mL, about 10-15 ng/ml, about 15-20 ng/ml, about 20-25 ng/mL, about 25-30 ng/ml, about 30-35 ng/ml, about 35-40 ng/mL, about 40-45 ng/ml, about 45-50 ng/ml, about 50-60 ng/mL, about 60-70 ng/mL, about 70-80 ng/mL, about 80-90 ng/mL, about 90-100 ng/mL, about 100-200 ng/mL, about 200-300 ng/ml, about 300-400 ng/ml, about 400-500 ng/mL, 500-600 ng/ml, 600-700 ng/ml, about 700-800 ng/ml, about 800-900 ng/mL, or about 900-1000 ng/mL.

In some embodiments, both an anti-CD3 antibody and an anti-CD28 antibody are present in the cell culture media. In some embodiments, the anti-CD3 antibody and the anti-CD28 antibody are attached to a solid surface. In some embodiments, the anti-CD3 antibody and the anti-CD28 antibody are attached to beads. In some embodiments, beads (e.g., 3.5 μm particles) loaded with CD28 antibodies, anti-biotin antibodies and CD3-Biotin are present in the cell culture medium. Such beads are commercially available (e.g., MACS GMP ExpAct Treg Kit, DYNABEADSR M-450 CD3/CD28 T Cell Expander). In specific embodiments, the ratio of anti-CD3 antibody to anti-CD28 antibody on the beads is about 100:1, 90:1, 80:1, 70:1, 60:1, 50:1, 40:1, 30:1, 20:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:20, 1:30, 1:40, 1:50, 1:60, 1:70, 1:80, 1:90 or 1:100. In some embodiments, the population of Tregs is cultured in the presence of both IL-2 and beads loaded with CD28 antibodies, anti-biotin antibodies and CD3-Biotin. In some embodiments, the beads coated with anti-CD3 and anti-CD28 antibody are first added to the culture within about 4-5 days of initiating culture. In some embodiments, the beads coated with anti-CD3 and anti-CD28 antibody are first added to the culture within about 3-4 days of initiating culture. In some embodiments, the beads coated with anti-CD3 and anti-CD28 antibody are first added to the culture within about 2-3 days of initiating culture. In some embodiments, the beads coated with anti-CD3 and anti-CD28 antibody are first added to the culture within about 1-2 days of initiating culture. In some embodiments, the beads coated with anti-CD3 and anti-CD28 antibody are first added to the culture within about 24 hours of initiating culture. In some embodiments, the beads coated with anti-CD3 and anti-CD28 antibody are first added to the culture within about 12 hours of initiating culture. In some embodiments, the beads coated with anti-CD3 and anti-CD28 antibody are first added to the culture within about 6 hours of initiating culture. In some embodiments, the beads coated with anti-CD3 and anti-CD28 antibody are first added to the culture within about 3 hours of initiating culture. In some embodiments, the beads coated with anti-CD3 and anti-CD28 antibody are first added to the culture within about 2 hours of initiating culture. In some embodiments, the beads coated with anti-CD3 and anti-CD28 antibody are first added to the culture within about 1 hour of initiating culture. In some embodiments, the beads coated with anti-CD3 and anti-CD28 antibody are first added to the culture within about 30 minutes of initiating culture. In some embodiments, the beads coated with anti-CD3 and anti-CD28 antibody are first added to the culture within about 4-5 days after the completion of the enrichment step. In some embodiments, the beads coated with anti-CD3 and anti-CD28 antibody are first added to the culture within about 3-4 days after the completion of the enrichment step. In some embodiments, the beads coated with anti-CD3 and anti-CD28 antibody are first added to the culture within about 2-3 days after the completion of the enrichment step. In some embodiments, the beads coated with anti-CD3 and anti-CD28 antibody are first added to the culture within about 1-2 days after the completion of the enrichment step. In some embodiments, the beads coated with anti-CD3 and anti-CD28 antibody are first added to the culture within about 24 hours after the completion of the enrichment step. In some embodiments, the beads coated with anti-CD3 and anti-CD28 antibody are first added to the culture within about 12 hours after the completion of the enrichment step. In some embodiments, the beads coated with anti-CD3 and anti-CD28 antibody are first added to the culture within about 6 hours after the completion of the enrichment step. In some embodiments, the beads coated with anti-CD3 and anti-CD28 antibody are first added to the culture within about 3 hours after the completion of the enrichment step. In some embodiments, the beads coated with anti-CD3 and anti-CD28 antibody are first added to the culture within about 2 hours after the completion of the enrichment step. In some embodiments, the beads coated with anti-CD3 and anti-CD28 antibody are first added to the culture within about 1 hour after the completion of the enrichment step. In some embodiments, the beads coated with anti-CD3 and anti-CD28 antibody are first added to the culture within about 30 minutes after the completion of the enrichment step. In some embodiments, the beads coated with anti-CD3 and anti-CD28 antibody are again added to the culture medium about 14 days after the beads coated with anti-CD3 and anti-CD28 antibody were first added to the culture medium (e.g., if the cell number by then has not reached a target cell number). In some embodiments, the beads coated with anti-CD3 and anti-CD28 antibody are again added to the culture medium about 13 days after the beads coated with anti-CD3 and anti-CD28 antibody were first added to the culture medium (e.g., if the cell number by then has not reached a target cell number). In some embodiments, the beads coated with anti-CD3 and anti-CD28 antibody are again added to the culture medium about 12 days after the beads coated with anti-CD3 and anti-CD28 antibody were first added to the culture medium (e.g., if the cell number by then has not reached a target cell number). In some embodiments, the beads coated with anti-CD3 and anti-CD28 antibody are again added to the culture medium about 11 days after the beads coated with anti-CD3 and anti-CD28 antibody were first added to the culture medium (e.g., if the cell number by then has not reached a target cell number). In some embodiments, the beads coated with anti-CD3 and anti-CD28 antibody are again added to the culture medium about 10 days after the beads coated with anti-CD3 and anti-CD28 antibody were first added to the culture medium (e.g., if the cell number by then has not reached a target cell number). In some embodiments, the beads coated with anti-CD3 and anti-CD28 antibody are again added to the culture medium about 9 days after the beads coated with anti-CD3 and anti-CD28 antibody were first added to the culture medium (e.g., if the cell number by then has not reached a target cell number). In some embodiments, the beads coated with anti-CD3 and anti-CD28 antibody are again added to the culture medium about 8 days after the beads coated with anti-CD3 and anti-CD28 antibody were first added to the culture medium (e.g., if the cell number by then has not reached a target cell number). The cell number means the number of all cells in culture, including the enriched Treg cells, which represent a majority of the cells in culture and in specific embodiments represent more than 70%, more than 80%, more than 90%, more than 95%, more than 99%, or 100% of the cells in culture. In certain embodiments, the target cell number is 1×10⁸ to 1×10¹⁰ cells. In certain embodiments, the target cell number is 1×10⁹ to 5×10⁹ cells. In certain embodiments, the target cell number is 2×10⁹ to 5×10⁹ cells. In certain embodiments, the target cell number is 2×10⁹ to 2.5×10⁹ cells. In a specific embodiment, the target cell number is 1×10⁹ cells. In another specific embodiment, the target cell number is 1.5×10⁹ cells. In another specific embodiment, the target cell number is 2×10⁹ cells. In another specific embodiment, the target cell number is 2.5×10⁹ cells. In another specific embodiment, the target cell number is 3×10⁹ cells. In another specific embodiment, the target cell number is 3.5×10⁹ cells. In another specific embodiment, the target cell number is 4×10⁹ cells. In another specific embodiment, the target cell number is 4.5×10⁹ cells. In another specific embodiment, the target cell number is 5×10⁹ cells. In specific embodiments, the ratio of beads to cells in the culture is 10:1, 9:1, 8:1, 7:1, 6:1, 5:1, 4:1, 3:1, 2:1 or 1:1.

The expansion agent or agents may be added to the culture medium every 1, 2, 3, 4, or 5 days. In specific embodiments, the expansion agent is added to the culture medium every 1-2 days. In specific embodiments, the expansion agent is added to the culture medium every 2-3 days. In specific embodiments, the expansion agent is added to the culture medium every 3-4 days. In specific embodiments, the expansion agent is added to the culture medium every 4-5 days. In other specific embodiments, the expansion agent is added to the culture medium on day 6, 8, and 11, wherein day 0 is the day on which the biological sample is obtained from the subject. In some specific embodiments, the expansion agent is not added to the culture medium on day 13, wherein day 0 is the day on which the biological sample is obtained from the subject.

In some embodiments, the one or more expansion agents are first added to the culture within about 30 minutes⁻¹ hour, within 1-2 hours, within 2-4 hours, within 4-6 hours, within 6-8 hours, within 8-10 hours, within 10-12 hours, within 12-14 hours, within 14-16 hours, within 16-18 hours, within 18-24 hours, within 24-36 hours, within 36-48 hours, within about 30 minutes, within about 1 hour, within about 2 hours, within about 3 hours, within about 6 hours, within about 12 hours, within about 24 hours, within about 2 days, within about 3 days, within about 4 days, within about 5 days, within about 6 days, or within about 7 days of initiating culture. In some embodiments, the one or more expansion agents are first added to the culture within about 4-5 days of initiating culture. In some embodiments, the one or more expansion agents are first added to the culture within about 3-4 days of initiating culture. In some embodiments, the one or more expansion agents are first added to the culture within about 2-3 days of initiating culture. In some embodiments, the one or more expansion agents are first added to the culture within about 1-2 days of initiating culture. In some embodiments, the one or more expansion agents are first added to the culture within about 24 hours of initiating culture. In some embodiments, the one or more expansion agents are first added to the culture within about 12 hours of initiating culture. In some embodiments, the one or more expansion agents are first added to the culture within about 6 hours of initiating culture. In some embodiments, the one or more expansion agents are first added to the culture within about 3 hours of initiating culture. In some embodiments, the one or more expansion agents are first added to the culture within about 2 hours of initiating culture. In some embodiments, the one or more expansion agents are first added to the culture within about 1 hour of initiating culture. In some embodiments, the one or more expansion agents are first added to the culture within about 30 minutes of initiating culture. In some embodiments, the one or more expansion agents are first added to the culture within about 30 minutes⁻¹ hour, within 1-2 hours, within 2-4 hours, within 4-6 hours, within 6-8 hours, within 8-10 hours, within 10-12 hours, within 12-14 hours, within 14-16 hours, within 16-18 hours, within 18-24 hours, within 24-36 hours, within 36-48 hours, within about 30 minutes, within about 1 hour, within about 2 hours, within about 3 hours, within about 6 hours, within about 12 hours, within about 24 hours, within about 2 days, within about 3 days, within about 4 days, within about 5 days, within about 6 days, or within about 7 days after the completion of the enrichment step. In some embodiments, the one or more expansion agents are first added to the culture within about 4-5 days after the completion of the enrichment step. In some embodiments, the one or more expansion agents are first added to the culture within about 3-4 days after the completion of the enrichment step. In some embodiments, the one or more expansion agents are first added to the culture within about 2-3 days after the completion of the enrichment step. In some embodiments, the one or more expansion agents are first added to the culture within about 1-2 days after the completion of the enrichment step. In some embodiments, the one or more expansion agents are first added to the culture within about 24 hours after the completion of the enrichment step. In some embodiments, the one or more expansion agents are first added to the culture within about 12 hours after the completion of the enrichment step. In some embodiments, the one or more expansion agents are first added to the culture within about 6 hours after the completion of the enrichment step. In some embodiments, the one or more expansion agents are first added to the culture within about 3 hours after the completion of the enrichment step. In some embodiments, the one or more expansion agents are first added to the culture within about 2 hours after the completion of the enrichment step. In some embodiments, the one or more expansion agents are first added to the culture within about 1 hour after the completion of the enrichment step. In some embodiments, the one or more expansion agents are first added to the culture within about 30 minutes after the completion of the enrichment step. In some embodiments, the one or more expansion agents are again added to the culture medium about 14 days after the expansion agent(s) were first added to the culture medium. In some embodiments, the one or more expansion agents are again added to the culture medium about 13 days after the expansion agent(s) were first added to the culture medium. In some embodiments, the one or more expansion agents are again added to the culture medium about 12 days after the expansion agent(s) were first added to the culture medium. In some embodiments, the one or more expansion agents are again added to the culture medium about 11 days after the expansion agent(s) were first added to the culture medium. In some embodiments, the one or more expansion agents are again added to the culture medium about 10 days after the expansion agent(s) were first added to the culture medium. In some embodiments, the one or more expansion agents are again added to the culture medium about 9 days after the expansion agent(s) were first added to the culture medium. In some embodiments, the one or more expansion agents are again added to the culture medium about 8 days after the expansion agent(s) were first added to the culture medium.

If no expansion agent is added to the culture on a given day, that day is considered a “rest day.” In some embodiments, no expansion agent is administered during the day preceding the day on which the population of Tregs is harvested. In some embodiments, no expansion agent is administered during the 2 days, 3 days, 4 days, 5 days or 6 days preceding the day on which the population of Tregs is harvested.

In some embodiments, the population of Tregs may be expanded ex vivo by culturing the cells in the presence of one or more agents that inhibit mammalian target of rapamycin (mTor). In some embodiments, the mTor inhibitor is rapamycin. In some embodiments, the m Tor inhibitor is an analog of rapamycin (a “rapalog,” e.g., Temsirolimus, Everolimus, or Ridaforolimus). In some embodiments, the mTor inhibitor is ICSN3250, OSU-53, or AZD8055. In some embodiments, the concentration of rapamycin in the cell culture medium is about 1-20 nmol/L, about 20-30 nmol/L, about 30-40 nmol/L, about 40-50 nmol/L, about 50-60 nmol/L, about 60-70 nmol/L, about 70-80 nmol/L, about 80-90 nmol/L, about 90-100 nmol/L, about 100-150 nmol/L, about 150-200 nmol/L, about 200-250 nmol/L, about 250-300 nmol/L, about 300-350 nmol/L, about 350-400 nmol/L, about 400-450 nmol/L, about 450-500 nmol/L, about 500-600 nmol/L, about 600-700 nmol/L, about 700-800 nmol/L, about 800-900 nmo/L or about 900-1000 nmol/L. In some embodiments, the concentration of rapamycin in the cell culture media is about 100 nmol/L.

In some embodiments, the mTor inhibitor is first added to the culture within about 30 minutes⁻¹ hour, within 1-2 hours, within 2-4 hours, within 4-6 hours, within 6-8 hours, within 8-10 hours, within 10-12 hours, within 12-14 hours, within 14-16 hours, within 16-18 hours, within 18-24 hours, within 24-36 hours, within 36-48 hours, within about 30 minutes, within about 1 hour, within about 2 hours, within about 3 hours, within about 6 hours, within about 12 hours, within about 1 day, within about 2 days, about 3 days, about 4 days, about 5 days, about 6 days, or about 7 days of initiating culture. In some embodiments, the mTor inhibitor is added to the culture medium about every 1, 2, 3, 4 or 5 days. In some embodiments, the mTor inhibitor is added to the culture medium about every 4-5 days. In some embodiments, the mTor inhibitor is added to the culture medium about every 3-4 days. In some embodiments, the mTor inhibitor is added to the culture medium about every 2-3 days. In some embodiments, the mTor inhibitor is added to the culture medium about every 1-2 days.

In certain embodiments, the expansion step takes place in a bioreactor comprising an extracapillary space. In some embodiments, flow rate of an extracapillary (EC) medium of the bioreactor can be maintained at about 0-1 mL/min, about 0-0.8 mL/min, about 0-0.6 mL/min, about 0-0.4 mL/min, about 0-0.2 mL/min, about 0.2-1 mL/min, about 0.2-0.8 mL/min, about 0.2-0.6 mL/min, about 0.2-0.4 mL/min, about 0.4-1 mL/min, about 0.4-0.8 mL/min, about 0.4-0.6 mL/min, about 0.6-1 mL/min, about 0.6-0.8 mL/min, or about 0.8-1 mL/min. In some embodiments, flow rate of an EC medium of the bioreactor can be maintained at about 0 mL/min, about 0.1 mL/min, about 0.2 mL/min, about 0.3 mL/min, about 0.4 mL/min, about 0.5 mL/min, about 0.6 mL/min, about 0.7 mL/min, about 0.8 mL/min, about 0.9 mL/min, or about 1 mL/min. In some embodiments, the expansion step comprises adjusting flow rate of an EC medium of the bioreactor depending on cell number. The cell number means the number of all cells in culture, including the enriched Treg cells, which represent a majority of the cells in culture and in specific embodiments represent more than 70%, more than 80%, more than 90%, more than 95%, more than 99%, or 100% of the cells in culture. In a specific embodiment, the expansion step comprises maintaining the flow rate of the EC medium at 0 until the cell number reaches 500×10⁶, then increasing the flow rate of the EC medium to about 0.2 mL/min and maintaining the flow rate of the EC medium at about 0.2 mL/min until the cell number reaches 750×10⁶, then increasing the flow rate of the EC medium to about 0.4 mL/min and maintaining the flow rate of the EC medium at about 0.4 mL/min until the cell number reaches about 1,000×10⁶, then increasing the flow rate of the EC medium to about 0.6 mL/min and maintaining the flow rate of the EC medium at about 0.6 mL/min until the cell number reaches about 1,500×10⁶, and then increasing the flow rate of the EC medium to about 0.8 mL/min and maintaining the flow rate of the EC medium at about 0.8 mL/min. In certain embodiments, the extracapillary medium comprises rapamycin.

The population of Tregs may be expanded by culturing them for an appropriate duration of time to produce a sufficiently expanded population of Tregs. For example, the population of Tregs may be expanded for a time sufficient to obtain a desired number or amount of anti-inflammatory EVs. In another example, the population of Tregs may be expanded for a time sufficient to attain one or more characteristics. In certain embodiments, anti-inflammatory EVs may be isolated upon once the Treg population attains one or more characteristics.

For example, the proportion of CD4+CD25+ cells present within an expanding Treg culture may be monitored, e.g., monitored using flow cytometry. For example, in some embodiments, a sufficiently expanded population of Tregs is a population of cells that contains more than 70% CD4+CD25+ cells as determined by flow cytometry.

The number of CD4+CD25+ cells may be determined every day, or every 2, 3, 4, or 5 days. In certain embodiments, if the culture does not contain a sufficiently expanded population of Tregs on Day 15 (wherein day 0 is the day on which the biological sample is obtained from the subject), the cells may be re-activated with one or more expansion agents. In certain embodiments, if the culture does not contain a sufficiently expanded therapeutic population of Tregs on Day 14 (wherein day 0 is the day on which the biological sample is obtained from the subject), the cells may be re-activated with one or more expansion agents. In certain embodiments, if the culture does not contain a sufficiently expanded therapeutic population of Tregs on Day 13 (wherein day 0 is the day on which the biological sample is obtained from the subject), the cells may be re-activated with one or more expansion agents. In certain embodiments, if the culture does not contain a sufficiently expanded therapeutic population of Tregs on Day 12 (wherein day 0 is the day on which the biological sample is obtained from the subject), the cells may be re-activated with one or more expansion agents. In certain embodiments, if the culture does not contain a sufficiently expanded therapeutic population of Tregs on Day 11 (wherein day 0 is the day on which the biological sample is obtained from the subject), the cells may be re-activated with one or more expansion agents. In certain embodiments, if the culture does not contain a sufficiently expanded therapeutic population of Tregs on Day 10 (wherein day 0 is the day on which the biological sample is obtained from the subject), the cells may be re-activated with one or more expansion agents. In certain embodiments, if the culture does not contain a sufficiently expanded therapeutic population of Tregs on Day 9 (wherein day 0 is the day on which the biological sample is obtained from the subject), the cells may be re-activated with one or more expansion agents. In certain embodiments, if the culture does not contain a sufficiently expanded therapeutic population of Tregs on Day 8 (wherein day 0 is the day on which the biological sample is obtained from the subject), the cells may be re-activated with one or more expansion agents.

In some embodiments, a population of Tregs is expanded by culturing for about 6-30 days, about 10-30 days, about 15-25 days, or about 18-22 days. In some embodiments, a population of Tregs is expanded by culturing for about 15, 16, 18, 18, 19, 20, 21, 22, 23, 24, or 25 days. In certain embodiments, for example, embodiments comprising automation, partial automation or at least one automated step, a population of Tregs is expanded by culturing for about 6-15 days, about 8-15, about 8-12 days, or about 6, 7, 8, 9, 10, 11, 12, 13, 14 or 15 days.

Viability of the cells being expanded in culture may be determined using any method known in the art. For example, the viability of cells being expanded in culture may be determined using trypan blue exclusion. Trypan blue is a dye which is excluded by cells with an intact membrane (viable cells) but taken up by cells with compromised membrane integrity (non-viable cells). Thus, viable cells appear clear under a light microscope, whereas non-viable cells appear blue. Equal amounts of trypan blue and cell suspension are mixed and counted. Viability is expressed as a percentage of trypan blue excluding cells. In some embodiments, a population of Tregs comprises about 60%, 65% or 70% viable cells as determined by trypan blue exclusion. In some embodiments, a population of Tregs comprises more than about 70% viable cells as determined by trypan blue exclusion. For example, in certain embodiments a population of Tregs comprises about 75%, 80%, 85%, 90%, 95% or greater that 95% viable cells as determined by trypan blue exclusion. In some embodiments, viability of the cells being expanded in culture is determined every 2-3 days. In some embodiments, viability of the cells being expanded in culture is determined every day or every 2, 3, 4, or 5 days.

In some embodiments, the cells are washed one or more times during the culturing to remove agents present during the incubation or culturing and/or to replenish the culture medium with one or more additional agents. In some embodiments, the cells are washed during the incubation or culturing to reduce or remove the expansion agent(s). The culture medium may be replaced about every 2, 3, 4, 5, 6 or 7 days, for example, every 2-3 days or every 3-4 days. In some embodiments, only part of the culture medium (e.g., about 50% of the culture medium) is replaced. In other embodiments, the entire culture medium is replaced. In some embodiments, the cell culture is not centrifuged during a change of culture medium. In certain embodiments, anti-inflammatory EVs may be isolated culture media removed during any or each of such points during the expansion process.

To avoid EVs from serum, the cultures from which the EVs are isolated may comprise cells that are cultured in medium containing EV-depleted, for example, EV-free serum. For example, the cells may be cultured in medium containing EV-depleted or EV-free fetal bovine serum (FBS). In another example, the cells may be cultured in medium containing EV-depleted or EV-free human serum, e.g., human AB serum. In particular examples, the cells may be cultured in medium containing exosome-depleted, for example, exosome-free serum, e.g., exosome-depleted or exosome-free FBS, or exosome-depleted or exosome-free human serum, for example, human AB serum.

In particular, non-limiting examples, the cultures from which the EVs are isolated may comprise cells that are cultured in medium containing EV-depleted, for example, EV-free serum, for a period of 16 hrs, 24 hrs or 48 hrs preceding the isolation. For example, the cells may be cultured in medium containing EV-depleted or EV-free fetal bovine serum (FBS) for a period of 16 hrs, 24 hrs or 48 hrs preceding the isolation. In another example, the cells may be cultured in medium containing EV-depleted or EV-free human serum, e.g., human AB serum, for a period of 16 hrs, 24 hrs or 48 hrs preceding the isolation. In particular examples, the cells may be cultured in medium containing exosome-depleted, for example, exosome-free serum, e.g., exosome-depleted or exosome-free FBS, or exosome-depleted or exosome-free human serum, for example, human AB serum, for a period of 16 hrs, 24 hrs, or 48 hrs preceding the isolation.

5.2.2. Exemplary Protocol for Isolation and Expansion of Regulatory T Cells from a leukapheresis or blood sample product

In some embodiments, EVs may be isolated from Tregs expanded using the following protocol. This protocol may be applied to isolation and expansion of leukapheresis products or blood sample products from, e.g., ALS patients, Alzheimer's Disease patients, or patients exhibiting a different disorder, for example a different neurodegenerative disorder, or from healthy subjects.

### 5.2.2.1 Step 1: Patient Leukapheresis/Blood Sample Product Processing

Leukapheresis or blood sample products should be processed within 24 hours.

With respect to leukapheresis, the total volume of the leukapheresis product should be between 100 mL and 840 mL. If the leukapheresis product is less than 100 mL, an equal volume of CliniMACS Buffer with 1% human serum albumin (HAS) should be added. Volume reduction of the leukapheresis product may be carried out using the GE Healthcare/Biosafe Sepax 2 RM with the PeriCell Protocol and CS490.1 kit (PeriCell).

Leukapheresis or blood products may be purified using the GE Healthcare-Biosafe Sepax 2 RM NeatCell Protocol and CS900.2 kit.

5.2.2.2 Step 2: Treg enrichment

CD8+ and CD19+Cells (may be depleted using CliniMACS kit according to manufacturer's instructions. This comprises labeling cells with CD8+ and CD 19+ micro beads for depletion and then using automatic cell separation using the CliniMACS® Plus Instrument in combination with CliniMACS PBS/EDTA Buffer in 1% HSA, the CliniMACS Tubing Set LS and software sequence DEPLETION 2.1.

Subsequently, the population may be enriched for CD25+ Tregs by positive selection using CliniMACS. This comprises labeling cells with CD25 for Enrichment CD25 Micro-Beads and then using automatic cell separation using the CliniMACS® Plus Instrument in combination with CliniMACS PBS/EDTA Buffer in 1% HSA, the CliniMACS Tubing Set LS and software sequence ENRICHMENT 3.2.

### 5.2.2.3 Step 3: Treg Expansion

Treg expansion is initiated on Day 0 from CD25+ enriched leukapheresis/blood sample product.

The CD25+ enriched leukapheresis product is centrifuged, the pellet washed in TexMACS Medium with 5% Human AB Serum, centrifuged again and the resulting pellet is resuspended in TexMACS media with 5% Human AB Serum at a density of 0.8-1.0×10⁶ cells/mL. The cells are transferred in to flasks and incubated for 16-18 hours at 37° C. in a humidified mixture of 95% air and 5% CO₂.

The cell concentration should be maintained between 0.5×10⁶ cells/mL and 1.2×10⁶ cells/mL after each medium change. EVs may be isolated from the medium which is removed from the Treg culture at or more of the media changes. The medium may be frozen before EVs are isolated.

For cell cultures medium removal, flasks are stood upright for at least 20 minutes without disturbing them, and then 50% of the total medium volume is removed.

Viability is assessed by trypan blue. If cell viability is over 90%, cells are expanded by changing the cell culture media to obtain 0.5×10⁶ cells/mL-1.2×10⁶ cells/mL.

On Day 1, the cells are stimulated with CD3/CD28 beads using the MACS GMP ExpAct Treg Kit. This kit contain 3.5 μm particles, which are preloaded with CD28 antibodies, anti-biotin antibodies and CD3-Biotin. Each vial contains 1×10⁹ ExpAct Treg Beads (2×10⁵/μL). MACS GMP ExpAct Treg Beads and Treg cells should be at a bead-to-cell ratio of 4:1 for initial stimulation. For activation, the cell concentration should be about 0.5-0.7×10⁶ cells/mL for MACS GMP ExpAct Treg Kit (CD3/CD28 Beads). Activation is carried out on Day 1 and again on Day 15.

The cells are expanded in TexMACS Medium with 5% Human AB Serum supplemented with 100 nmol/L rapamycin and 500 IU/ml IL-2.

The culture medium is changed and rapamycin is replenished on Day 4, Day 6, Day 8, Day 11, Day 13, Day 15, Day 18, Day 20, and Day 22. The IL-2 is replenished on Day 6, Day 8, Day 11, Day 15, Day 18, and Day 20. EVs may be isolated from the medium which is removed from the Treg culture at or more of the media changes. The medium may be frozen before EVs are isolated.

### 5.2.2.4 Step 4: Treg Harvesting

In certain embodiments, the Tregs may be harvested. For example, in particular embodiments, on Day 25, Tregs may be harvested. The MACS GMP Activation Beads may be removed, for example, using CliniMACS Depletion Tubing Set LS (168-01) and software DEPLETION 2.1. according to manufacturer's instructions or standard operating procedure. In certain embodiments, the expanded Treg cell product may satisfy the criteria shown in Table 1. In certain embodiments, the final harvested Treg cell product may satisfy the criteria shown in Table 1.

EVs may be isolated from the medium on the day the Tregs would be harvested (whether or not Treg harvesting is performed). For example, EVs may be isolated from media removed from a Treg culture at the time of harvesting or at the time the Treg would be harvested. The medium may be frozen before EVs are isolated.

TABLE 1
Expanded Treg/Treg Criteria

| Test | Specification |
|---|---|
| Visual Inspection | No evidence of contamination |
| Viability | ≥70% |
| Endotoxin (LAL) | <5 EU/kg |
| Gram Stain | Negative |
| Flow Analysis: CD8+ | <20% |
| Flow Analysis: CD4+CD25+ | ≥70% |
| Sterility - 14 days | Aerobic: No growth |
| (Aerobic and Anaerobic cultures) | Anaerobic: No growth |

### 5.3 Compositions

In certain aspects, compositions are provided comprising an isolated, cell-free population of anti-inflammatory EVs as described herein. For example, provided herein are compositions comprising an isolated, cell-free population of anti-inflammatory EVs suitable for administration to a subject, for example, a human subject.

In certain aspects, provided are pharmaceutical compositions comprising an isolated, cell-free population of anti-inflammatory EVs described herein. In certain embodiments, provided herein is a pharmaceutical composition comprising an isolated, cell-free population of anti-inflammatory EVs and a buffer, for example, a sterile buffer, e.g., a saline-containing buffer. In particular embodiments, the pharmaceutical composition comprises an isolated, cell-free population of anti-inflammatory EVs and physiological saline. In particular embodiments, the pharmaceutical composition comprises an isolated, cell-free population of anti-inflammatory EVs and normal saline. In particular embodiments, the pharmaceutical composition comprises an isolated, cell-free population of anti-inflammatory EVs and 0.9% saline. In particular embodiments, the pharmaceutical composition comprises an isolated, cell-free population of anti-inflammatory EVs and phosphate-buffered saline.

In some embodiments, a composition provided herein is a pharmaceutical composition comprising a population of anti-inflammatory EVs provided herein and a pharmaceutically acceptable carrier, excipient, or diluent. In some embodiments, a composition provided herein is a pharmaceutical composition comprising an effective amount of a population of anti-inflammatory EVs provided herein and a carrier, excipient, or diluent, that is, an amount of a population of anti-inflammatory EVs provided herein which is sufficient to result in a desired outcome.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

The carrier, excipient, or diluent may be any pharmaceutically acceptable carrier, excipient or diluent, known in the art. Examples of pharmaceutically acceptable carriers include non-toxic solids, semisolids, or liquid fillers, diluents, encapsulating materials, formulation auxiliaries or carriers. A pharmaceutically acceptable carrier can include all solvents, dispersion media, coatings, antibacterial and antifungal agents, isotonic and absorption delaying agents, and the like, compatible with pharmaceutical administration. Examples of such carriers or diluents include, but are not limited to, water, saline, Ringer's solutions, dextrose solution, and 5% human serum. Liposomes and non-aqueous vehicles such as fixed oils may also be used.

Excipients may include, for example, encapsulating materials or additives such as absorption accelerators, antioxidants, binders, buffers, coating agents, coloring agents, disintegrating agents, emulsifiers, extenders, fillers, flavoring agents, humectants, lubricants, perfumes, preservatives, propellants, releasing agents, sterilizing agents, sweeteners, solubilizers, wetting agents, and mixtures thereof. The term “excipient” may itself refer to a carrier or diluent.

In some embodiments, the pharmaceutical composition comprises a population of anti-inflammatory EVs provided herein suspended in a sterile buffer. In some embodiments, a pharmaceutical composition provided herein comprises a population of anti-inflammatory EVs in a buffer suitable for administration to a human subject. Examples of buffers suitable for administration to a human subject include saline-containing buffers such as phosphate buffered saline, physiological saline, normal saline or 0.9% saline.

A pharmaceutical composition may be formulated to be compatible with an intended route of administration. For example, pharmaceutical compositions may routinely be formulated to be suitable for administration by routes including intranasal, parenteral (e.g., subcutaneous, intravenous, intramuscular, intraperitoneal, intraarterial, intraventricular, intrathecal, intraurethral, intrasternal, and intrasynovial), intradermal, oral (e.g., ingestion, sublingual), inhalation, nasal, e.g., nasal drip, intracavity, intracranial, ocular, e.g., intraocular, and transdermal (topical).

In certain embodiments, for example, a pharmaceutical composition presented herein that comprises an isolated, cell-free population of anti-inflammatory EVs as described herein has been formulated to be suitable for intranasal administration to a subject, for example, a human subject.

In certain embodiments, a pharmaceutical composition presented herein that comprises an isolated, cell-free population of anti-inflammatory EVs as described herein has been formulated to be suitable for injection, infusion or implantation to a subject, for example, a human subject.

In particular embodiments, a pharmaceutical composition presented herein that comprises an isolated, cell-free population of anti-inflammatory EVs as described herein has been formulated to be suitable for intravenous administration to a subject, for example, a human subject.

In another example, in particular embodiments, a pharmaceutical composition presented herein that comprises an isolated, cell-free population of anti-inflammatory EVs as described herein has been formulated to be suitable for subcutaneous administration to a subject, for example, a human subject.

In yet another example, in particular embodiments, a pharmaceutical composition presented herein that comprises an isolated, cell-free population of anti-inflammatory EVs as described herein has been formulated to be suitable for intramuscular administration to a subject, for example, a human subject.

In certain embodiments, a composition, for example a pharmaceutical composition presented herein that comprises an isolated, cell-free population of anti-inflammatory EVs as described herein has been formulated in solution, suspension, emulsion, micelle, liposome, microsphere, or nanosystem form.

In certain embodiments, a composition, for example a pharmaceutical composition, presented herein that comprises an isolated, cell-free population of anti-inflammatory EVs as described herein may be stored frozen, e.g., may be stored at −20° C. or −80° C. For example, in particular embodiments, such a composition, e.g., pharmaceutical composition, may be stored frozen, for example, frozen at −20° C. or −80° C., for about 1 week, 1 month, about 3 months, about 6 months, about 9 months, about 12 months, about 18 months or about 24 months. In specific embodiments, such a composition, e.g., pharmaceutical composition, may then be thawed and administered to a patient.

In certain embodiments, a composition, for example a pharmaceutical composition, presented herein that comprises an isolated, cell-free population of anti-inflammatory EVs as described herein may be stored frozen, e.g., may be stored at −20° C. or −80° C., thawed, then refrozen. In particular embodiments, such a composition, e.g., pharmaceutical composition, may be thawed then refrozen one, two, three, four, five, six, seven, eight, nine, ten, eleven, twelve, thirteen, fourteen, fifteen, sixteen, seventeen, eighteen, nineteen or twenty times. In specific embodiments, such a composition, e.g., pharmaceutical composition, may then be thawed and administered to a patient.

In certain embodiments, a composition, for example a pharmaceutical composition, presented herein that comprises an isolated, cell-free population of anti-inflammatory EVs as described herein may be stored at about 2° C. to about 8° C. (e.g., at about 4° C.). For example, in particular embodiments, such a composition, e.g., pharmaceutical composition, may be stored at about 2° C. to about 8° C. (e.g., at about 4° C.) for less than about 2 weeks, less than about 1 week, less than about 14 days, less than about 13 days, less than about 12 days, less than about 11 days, less than about 10 days, less than about 9 days, less than about 8 days, less than about 7 days, less than about 6 days, less than about 5 days, less than about 4 days, less than about 3 days, less than about 2 days, less than about 1 day, or about overnight. In specific embodiments, such a composition, e.g., pharmaceutical composition, may be stored at 4° C. prior to administration to a subject, for example, a human subject, e.g., may be thawed after being frozen, then stored at 4° C. prior to administration to a subject, for example to a human subject.

In certain embodiments, provided herein is a cryopreserved composition, for example, pharmaceutical composition, comprising an isolated, cell-free population of anti-inflammatory EVs as described herein. In particular embodiments, the cryopreserved, isolated cell-free population of anti-inflammatory EVs may be cryopreserved for about 1 week, 1 month, about 3 months, about 6 months, about 9 months, about 12 months, about 18 months or about 24 months, then may be thawed and administered to a patient after cryopreservation.

In certain embodiments, provided herein is a composition comprising an isolated, cell-free population of anti-inflammatory EVs as described herein, wherein the population comprises about 1×10⁶ to about 1×10¹⁶ EVs, about 1×10⁷ to about 1×10¹⁶ EVs, 1×10⁸ to about 1×10¹⁶ EVs, about 1×10⁹ to about 1×10¹⁶ EVs, 1×10¹⁰ to about 1×10¹⁶ EVs, about 1×10¹¹ to about 1×10¹⁶ EVs, 1×10¹² to about 1×10¹⁶ EVs, about 1×10¹³ to about 1×10¹⁶ EVs, 1×10⁶ to about 1×10¹⁵ EVs, about 1×10⁷ to about 1×10¹⁵ EVs, 1×10⁸ to about 1×10¹⁵ EVs, about 1×10⁹ to about 1×10¹⁵ EVs, 1×10¹⁰ to about 1×10¹⁵ EVs, about 1×10¹¹ to about 1×10¹⁵ EVs, 1×10¹² to about 1×10¹⁵ EVs, about 1×10¹³ to about 1×10¹⁵ EVs, 1×10⁶ to about 1×10¹⁴ EVs, about 1×10⁷ to about 1×10¹⁴ EVs, 1×10⁸ to about 1×10¹⁴ EVs, about 1×10⁹ to about 1×10¹⁴ EVs, 1×10¹⁰ to about 1×10¹⁴ EVs, about 1×10¹¹ to about 1×10¹⁴ EVs, 1×10¹² to about 1×10¹⁴ EVs, about 1×10¹³ to about 1×10¹⁴ EVs, 1×10⁶ to about 1×10¹³ EVs, about 1×10⁷ to about 1×10¹³ EVs, 1×10⁸ to about 1×10¹³ EVs, about 1×10⁹ to about 1×10¹³ EVs, 1×10¹⁰ to about 1×10¹³ EVs, about 1×10¹¹ to about 1×10¹³ EVs, 1×10¹² to about 1×10¹³ EVs, 1×10⁶ to about 1×10¹² EVs, about 1×10⁷ to about 1×10¹² EVs, 1×10⁸ to about 1×10¹² EVs, about 1×10⁹ to about 1×10¹² EVs, 1×10¹⁰ to about 1×10¹² EVs, about 1×10¹¹ to about 1×10¹² EVs, about 1×10⁶ to about 1×10¹¹ EVs, about 1×10⁷ to about 1×10¹¹ EVs, 1×10⁸ to about 1×10¹¹ EVs, about 1×10⁹ to about 1×10¹¹ EVs, 1×10¹⁰ to about 1×10¹¹ EVs, about 1×10⁶ to about 1×10¹⁰ EVs, about 1×10⁷ to about 1×10¹⁰ EVs, 1×10⁸ to about 1×10¹⁰ EVs, about 1×10⁶ EVs, about 1×10⁷ EVs, 1×10⁸ to about 1×10⁹ EVs, about 1×10¹⁰ to about 1×10¹¹ EVs, 1×10¹² to about 1×10¹³ EVs, about 1×10⁶ EVs, about 1×10⁷ EVs, about 1×10⁸ EVs, about 2×10⁸ EVs, about 3×10⁸ EVs, about 4×10⁸ EVs, about 5×10⁸ EVs, about 6×10⁸ EVs, about 7×10⁸ EVs, about 8×10⁸ EVs, about 9×10⁸ EVs, about 1×10⁹ EVs, about 5×10⁹ EVs, about 1×10¹⁰ EVs, about 1×10¹¹ EVs, about 1×10¹² EVs, about 1×10¹³ EVs, about 1×10¹⁴ EVs, about 1×10¹⁵ EVs, or about 1×10¹⁶ EVs.

In certain embodiments, provided herein is a composition comprising an isolated, cell-free population of anti-inflammatory EVs as described herein, wherein the population comprises 1×10⁶ to about 1×10¹⁶ EVs/ml, about 1×10⁷ to about 1×10¹⁶ EVs/ml, 1×10⁸ to about 1×10¹⁶ EVs/ml, about 1×10⁹ to about 1×10¹⁶ EVs/ml, 1×10¹⁰ to about 1×10¹⁶ EVs/ml, about 1×10¹¹ to about 1×10¹⁶ EVs/ml, 1×10¹² to about 1×10¹⁶ EVs/ml, about 1×10¹³ to about 1×10¹⁶ EVs/ml, 1×10⁶ to about 1×10¹⁵ EVs/ml, about 1×10⁷ to about 1×10¹⁵ EVs/ml, 1×10⁸ to about 1×10¹⁵ EVs/ml, about 1×10⁹ to about 1×10¹⁵ EVs/ml, 1×10¹⁰ to about 1×10¹⁵ EVs/ml, about 1×10¹¹ to about 1×10¹⁵ EVs/ml, 1×10¹² to about 1×10¹⁵ EVs/ml, about 1×10¹³ to about 1×10¹⁵ EVs/ml, about 1×10⁶ to about 1×10¹⁴ EVs/ml, about 1×10⁷ to about 1×10¹⁴ EVs/ml, 1×10⁸ to about 1×10¹⁴ EVs/ml, about 1×10⁹ to about 1×10¹⁴ EVs/ml, 1×10¹⁰ to about 1×10¹⁴ EVs/ml, about 1×10¹¹ to about 1×10¹⁴ EVs/ml, 1×10¹² to about 1×10¹⁴ EVs/ml, about 1×10¹³ to about 1×10¹⁴ EVs/ml, 1×10⁶ to about 1×10¹³ EVs/ml, about 1×10⁷ to about 1×10¹³ EVs/ml, 1×10⁸ to about 1×10¹³ EVs/ml, about 1×10⁹ to about 1×10¹³ EVs/ml, 1×10¹⁰ to about 1×10¹³ EVs/ml, about 1×10¹¹ to about 1×10¹³ EVs/ml, 1×10¹² to about 1×10¹³ EVs,/ml 1×10⁶ to about 1×10¹² EVs/ml, about 1×10⁷ to about 1×10¹² EVs/ml, 1×10⁸ to about 1×10¹² EVs/ml, about 1×10⁹ to about 1×10¹² EVs/ml, 1×10¹⁰ to about 1×10¹² EVs/ml, about 1×10¹¹ to about 1×10¹² EVs/ml, about 1×10⁶ to about 1×10¹¹ EVs/ml, about 1×10⁷ to about 1×10¹¹ EVs/ml, 1×10⁸ to about 1×10¹¹ EVs/ml, about 1×10⁹ to about 1×10¹¹ EVs/ml, 1×10¹⁰ to about 1×10¹¹ EVs/ml, about 1×10⁶ to about 1×10¹⁰ EVs/ml, about 1×10⁷ to about 1×10¹⁰ EVs/ml, 1×10⁸ to about 1×10¹⁰ EVs/ml, about 1×10⁶ EVs/ml, about 1×10⁷ EVs/ml, 1×10⁸ to about 1×10⁹ EVs/ml, about 1×10¹⁰ to about 1×10¹¹ EVs/ml, 1×10¹² to about 1×10¹³ EVs/ml, about 1×10⁶ EVs/ml, about 1×10⁷ EVs/ml, about 1×10⁸ EVs/ml, about 2×10⁸ EVs/ml, about 3×10⁸ EVs/ml, about 4×10⁸ EVs,/ml about 5×10⁸ EVs/ml, about 6×10⁸ EVs/ml, about 7×10⁸ EVs/ml, about 8×10⁸ EVs/ml, about 9×10⁸ EVs/ml, about 1×10⁹ EVs/ml, about 5×10⁹ EVs/ml, about 1×10¹⁰ EVs/ml, about 1×10¹¹ EVs/ml, about 1×10¹² EVs,/ml about 1×10¹³ EVs/ml, about 1×10¹⁴ EVs/ml, about 1×10¹⁵ EVs/ml, or about 1×10¹⁶ EV/mls

In certain embodiments, provided herein is a composition comprising an isolated, cell-free population of anti-inflammatory EVs as described herein, wherein the population comprises about 1 μg to about 200 mg EVs, about 1 μg to about 150 mg EVs, about 1 μg to about 100 mg EVs, about 1 μg to about 75 mg EVs, about 1 μg to about 50 mg EVs, about 1 μg to about 25 mg EVs, about 1 μg to about 20 mg EVs, about 1 μg to about 15 mg EVs, about 1 μg to about 10 mg EVs, about 1 μg to about 5 mg EVs, about 1 μg to about 1 mg EVs, about 1 μg to about 500 μg EVs, about 1 μg to about 250 μg EVs, about 1 μg to about 125 μg EVs, about 1 μg to about 100 μg EVs, about 1 μg to about 50 μg EVs, about 1 μg to about 25 μg EVs, about 1 μg to about 20 μg EVs, about 1 μg to about 10 μg EVs, about 1 μg to about 5 μg EVs, about 10 μg to about 500 μg EVs, about 10 μg to about 250 μg EVs, about 10 μg to about 125 μg EVs, about 10 μg to about 100 μg EVs, about 10 μg to about 50 μg EVs, about 10 μg to about 25 μg EVs, about 10 μg to about 20 μg EVs, about 100 μg to about 500 μg EVs, about 100 μg to about 250 μg EVs, or about 100 μg to about 125 μg EVs.

In certain embodiments, provided herein is a composition comprising an isolated, cell-free population of anti-inflammatory EVs as described herein, wherein the population comprises about 1 μg to about 200 mg EVs/ml, about 1 μg to about 150 mg EVs/ml, about 1 μg to about 100 mg EVs/ml, about 1 μg to about 75 mg EVs/ml, about 1 μg to about 50 mg EVs/ml, about 1 μg to about 25 mg EVs/ml, about 1 μg to about 20 mg EVs/ml, about 1 μg to about 15 mg EVs/ml, about 1 μg to about 10 mg EVs/ml, about 1 μg to about 5 mg EVs/ml, about 1 μg to about 1 mg EVs/ml, about 1 μg to about 500 μg EVs/ml, about 1 μg to about 250 μg EVs/ml, about 1 μg to about 125 μg EVs/ml, about 1 μg to about 100 μg EVs,/ml about 1 μg to about 50 μg EVs/ml, about 1 μg to about 25 μg EVs/ml, about 1 μg to about 20 μg EVs/ml, about 1 μg to about 10 μg EVs,/ml about 1 μg to about 5 μg EVs/ml, about 10 μg to about 500 μg EVs/ml, about 10 μg to about 250 μg EVs/ml, about 10 μg to about 125 μg EVs,/ml about 10 μg to about 100 μg EVs/ml, about 10 μg to about 50 μg EVs/ml, about 10 μg to about 25 μg EVs/ml, about 10 μg to about 20 μg EVs/ml, about 100 μg to about 500 μg EVs/ml, about 100 μg to about 250 μg EVs/ml, or about 100 μg to about 125 μg EVs/ml.

In certain embodiments, the isolated, cell-free populations of anti-inflammatory EVs described herein are present in a composition that is substantially free of other EVs. For example, in certain embodiments, the isolated, cell-free populations of anti-inflammatory EVs described herein are present in a composition that contains less than about 20%, less than about 10%, less than about 5%, or less than about 1% other EVs.

In certain embodiments, an isolated, cell-free population of anti-inflammatory EVs described herein is present in a composition that comprises other EVs, wherein the isolated, cell-free population of anti-inflammatory EVs makes up about 10%, about 20%, about 25%, about 30%, about 35%, about 40%, about 50%, about 60%, about 70%, about 80%, about 90%, about 95%, or greater than about 95% of the EVs in the composition. In specific embodiments, the other EVs are serum EVs, for example, bovine serum EVs or human serum EVs.

In some embodiments, a composition comprising a population of anti-inflammatory EVs provided herein comprises no contaminants. In some embodiments, a composition comprising a population of anti-inflammatory EVs provided herein comprises a sufficiently low level of contaminants as to be suitable for administration, e.g., therapeutic administration, to a subject, for example a human subject. Contaminants include, for example, bacteria, fungus, mycoplasma, endotoxins or residual beads from the Treg expansion culture. In some embodiments, a composition comprising a population of anti-inflammatory EVs provided herein comprises less than about 5 EU/kg endotoxins. In some embodiments, a composition comprising a population of anti-inflammatory EVs provided herein comprises about or less than about 100 beads per 3×10⁶ cells.

In some embodiments, a composition comprising a population of anti-inflammatory EVs provided herein is substantially free of components utilized during the Treg cell expansion process and/or the EV isolation process. For example, in some embodiments, a composition comprising a population of anti-inflammatory EVs provided herein is substantially free of IL2. In particular embodiments, for example, a composition comprising a population of anti-inflammatory EVs provided herein, comprises less than about 10%, 9%, 8%, 7%, 6%, 5%, 4%, 3%, 2% or less of IL2 (as a percentage of the original amount of IL2 in the Treg cell culture).

As discussed herein, in certain embodiments, Treg cells from which Treg EVs are obtained may be cultured in an albumin-containing media. In certain embodiments, a composition comprising a population of anti-inflammatory EVs provided herein is substantially free of albumin. In particular embodiments, for example, a composition comprising a population of anti-inflammatory EVs provided herein, comprises less than about 10%, 9%, 8%, 7%, 6%, 5%, 4%, 3%, 2% or less of albumin (as a percentage of the original amount of albumin in the Treg cell culture).

In some embodiments, a composition comprising a population of anti-inflammatory EVs provided herein is sterile. In some embodiments, isolation or enrichment of the cells is carried out in a closed or sterile environment, for example, to minimize error, user handling and/or contamination. In some embodiments, sterility may be readily accomplished, e.g., by filtration through sterile filtration membranes.

In certain embodiments, presented herein is a pharmaceutical composition comprising a population of anti-inflammatory EVs described herein, wherein the pharmaceutical composition comprises any amount or concentration of anti-inflamatory EVs, e.g., Treg EVs, described herein. For example, in a certain embodiment, presented herein is a pharmaceutical composition comprising about 1×10⁹ Treg EVs. In a particular embodiment, presented herein is a pharmaceutical composition comprising about 1×10⁹ Treg EVs formulated in a unit dose form. In a particular embodiment, presented herein is a pharmaceutical composition comprising about 1×10⁹ Treg EVs formulated into a unit dose in saline, e.g., sterile saline, such as sterile saline for injection. In yet another particular embodiment, presented herein is a pharmaceutical composition comprising about 1×10⁹ Treg EVs formulated into a unit dose in 1 mL, 2 mL, 3 mL. 4 mL or 5 mL saline, e.g., sterile saline, such as sterile saline for injection. In a specific embodiment, presented herein is a pharmaceutical composition comprising about 1×10⁹ Treg EVs formulated into a unit dose in 2 mL saline, e.g., sterile saline, such as sterile saline for injection. Such a pharmaceutical composition may be present, for example present in a vial, such as a sterile vial, as a single unit dose or as multiple unit doses. For example, in a particular embodiment, presented herein is a pharmaceutical composition comprising about 1×10⁹ Treg EVs as a unit dose in 1 mL, 2 mL, 3 mL, 4 mL, or 5 mL sterile saline in a vial. In a specific embodiment, presented herein is a pharmaceutical composition comprising about 1×10⁹ Treg EVs as a unit dose in 2 mL sterile saline in a vial.

In certain embodiments, presented herein is a pharmaceutical composition comprising a population of anti-inflammatory EVs described herein, wherein the pharmaceutical composition comprises any amount or concentration of anti-inflamatory EVs, e.g., Treg EVs, described herein. For example, in a certain embodiment, presented herein is a pharmaceutical composition comprising about 5×10⁹ Treg EVs. In a particular embodiment, presented herein is a pharmaceutical composition comprising about 5×10⁹ Treg EVs formulated in a unit dose form. In a particular embodiment, presented herein is a pharmaceutical composition comprising about 5×10⁹ Treg EVs formulated into a unit dose in saline, e.g., sterile saline, such as sterile saline for injection. In yet another particular embodiment, presented herein is a pharmaceutical composition comprising about 5×10⁹ Treg EVs formulated into a unit dose in 1 mL, 2 mL, 3 mL. 4 mL or 5 mL saline, e.g., sterile saline, such as sterile saline for injection. In a specific embodiment, presented herein is a pharmaceutical composition comprising about 5×10⁹ Treg EVs formulated into a unit dose in 2 mL saline, e.g., sterile saline, such as sterile saline for injection. Such a pharmaceutical composition may be present, for example present in a vial, such as a sterile vial, as a single unit dose or as multiple unit doses. For example, in a particular embodiment, presented herein is a pharmaceutical composition comprising about 5×10⁹ Treg EVs as a unit dose in 1 mL, 2 mL, 3 mL, 4 mL, or 5 mL sterile saline in a vial. In a specific embodiment, presented herein is a pharmaceutical composition comprising about 5×10⁹ Treg EVs as a unit dose in 2 mL sterile saline in a vial.

In certain embodiments, presented herein is a pharmaceutical composition comprising a population of anti-inflammatory EVs described herein, wherein the pharmaceutical composition comprises any amount or concentration of anti-inflamatory EVs, e.g., Treg EVs, described herein. For example, in a certain embodiment, presented herein is a pharmaceutical composition comprising about 1×10¹⁰ Treg EVs. In a particular embodiment, presented herein is a pharmaceutical composition comprising about 1×10¹⁰ Treg EVs formulated in a unit dose form. In a particular embodiment, presented herein is a pharmaceutical composition comprising about 1×10¹⁰ Treg EVs formulated into a unit dose in saline, e.g., sterile saline, such as sterile saline for injection. In yet another particular embodiment, presented herein is a pharmaceutical composition comprising about 1×10¹⁰ Treg EVs formulated into a unit dose in 1 mL, 2 mL, 3 mL. 4 mL or 5 mL saline, e.g., sterile saline, such as sterile saline for injection. In a specific embodiment, presented herein is a pharmaceutical composition comprising about 1×10¹⁰ Treg EVs formulated into a unit dose in 2 mL saline, e.g., sterile saline, such as sterile saline for injection. Such a pharmaceutical composition may be present, for example present in a vial, such as a sterile vial, as a single unit dose or as multiple unit doses. For example, in a particular embodiment, presented herein is a pharmaceutical composition comprising about 1×10¹⁰ Treg EVs as a unit dose in 1 mL, 2 mL, 3 mL, 4 mL, or 5 mL sterile saline in a vial. In a specific embodiment, presented herein is a pharmaceutical composition comprising about 1×10¹⁰ Treg EVs as a unit dose in 2 mL sterile saline in a vial.

In certain embodiments, presented herein is a pharmaceutical composition comprising a population of anti-inflammatory EVs described herein, wherein the pharmaceutical composition comprises any amount or concentration of anti-inflamatory EVs, e.g., Treg EVs, described herein. For example, in a certain embodiment, presented herein is a pharmaceutical composition comprising about 5×10¹⁰ Treg EVs. In a particular embodiment, presented herein is a pharmaceutical composition comprising about 5×10¹⁰ Treg EVs formulated in a unit dose form. In a particular embodiment, presented herein is a pharmaceutical composition comprising about 5×10¹⁰ Treg EVs formulated into a unit dose in saline, e.g., sterile saline, such as sterile saline for injection. In yet another particular embodiment, presented herein is a pharmaceutical composition comprising about 5×10¹⁰ Treg EVs formulated into a unit dose in 1 mL, 2 mL, 3 mL. 4 mL or 5 mL saline, e.g., sterile saline, such as sterile saline for injection. In a specific embodiment, presented herein is a pharmaceutical composition comprising about 5×10¹⁰ Treg EVs formulated into a unit dose in 2 mL saline, e.g., sterile saline, such as sterile saline for injection. Such a pharmaceutical composition may be present, for example present in a vial, such as a sterile vial, as a single unit dose or as multiple unit doses. For example, in a particular embodiment, presented herein is a pharmaceutical composition comprising about 5×10¹⁰ Treg EVs as a unit dose in 1 mL, 2 mL, 3 mL, 4 mL, or 5 mL sterile saline in a vial. In a specific embodiment, presented herein is a pharmaceutical composition comprising about 5×10¹⁰ Treg EVs as a unit dose in 2 mL sterile saline in a vial.

In certain embodiments, presented herein is a pharmaceutical composition comprising a population of anti-inflammatory EVs described herein, wherein the pharmaceutical composition comprises any amount or concentration of anti-inflamatory EVs, e.g., Treg EVs, described herein. For example, in a certain embodiment, presented herein is a pharmaceutical composition comprising about 1×10¹¹ Treg EVs. In a particular embodiment, presented herein is a pharmaceutical composition comprising about 1×10¹¹ Treg EVs formulated in a unit dose form. In a particular embodiment, presented herein is a pharmaceutical composition comprising about 1×10¹¹ Treg EVs formulated into a unit dose in saline, e.g., sterile saline, such as sterile saline for injection. In yet another particular embodiment, presented herein is a pharmaceutical composition comprising about 1×10¹¹ Treg EVs formulated into a unit dose in 1 mL, 2 mL, 3 mL. 4 mL or 5 mL saline, e.g., sterile saline, such as sterile saline for injection. In a specific embodiment, presented herein is a pharmaceutical composition comprising about 1×10¹¹ Treg EVs formulated into a unit dose in 2 mL saline, e.g., sterile saline, such as sterile saline for injection. Such a pharmaceutical composition may be present, for example present in a vial, such as a sterile vial, as a single unit dose or as multiple unit doses. For example, in a particular embodiment, presented herein is a pharmaceutical composition comprising about 1×10¹¹ Treg EVs as a unit dose in 1 mL, 2 mL, 3 mL, 4 mL, or 5 mL sterile saline in a vial. In a specific embodiment, presented herein is a pharmaceutical composition comprising about 1×10¹¹ Treg EVs as a unit dose in 2 mL sterile saline in a vial.

In certain embodiments, presented herein is a pharmaceutical composition comprising a population of anti-inflammatory EVs described herein, wherein the pharmaceutical composition comprises any amount or concentration of anti-inflamatory EVs, e.g., Treg EVs, described herein. For example, in a certain embodiment, presented herein is a pharmaceutical composition comprising about 5×10¹¹ Treg EVs. In a particular embodiment, presented herein is a pharmaceutical composition comprising about 5×10¹¹ Treg EVs formulated in a unit dose form. In a particular embodiment, presented herein is a pharmaceutical composition comprising about 5×10¹¹ Treg EVs formulated into a unit dose in saline, e.g., sterile saline, such as sterile saline for injection. In yet another particular embodiment, presented herein is a pharmaceutical composition comprising about 5×10¹¹ Treg EVs formulated into a unit dose in 1 mL, 2 mL, 3 mL. 4 mL or 5 mL saline, e.g., sterile saline, such as sterile saline for injection. In a specific embodiment, presented herein is a pharmaceutical composition comprising about 5×10¹¹ Treg EVs formulated into a unit dose in 2 mL saline, e.g., sterile saline, such as sterile saline for injection. Such a pharmaceutical composition may be present, for example present in a vial, such as a sterile vial, as a single unit dose or as multiple unit doses. For example, in a particular embodiment, presented herein is a pharmaceutical composition comprising about 5×10¹¹ Treg EVs as a unit dose in 1 mL, 2 mL, 3 mL, 4 mL, or 5 mL sterile saline in a vial. In a specific embodiment, presented herein is a pharmaceutical composition comprising about 5×10¹¹ Treg EVs as a unit dose in 2 mL sterile saline in a vial.

In certain embodiments, presented herein is a pharmaceutical composition comprising a population of anti-inflammatory EVs described herein, wherein the pharmaceutical composition comprises any amount or concentration of anti-inflamatory EVs, e.g., Treg EVs, described herein. For example, in a certain embodiment, presented herein is a pharmaceutical composition comprising about 1×10¹² Treg EVs. In a particular embodiment, presented herein is a pharmaceutical composition comprising about 1×10¹² Treg EVs formulated in a unit dose form. In a particular embodiment, presented herein is a pharmaceutical composition comprising about 1×10¹² Treg EVs formulated into a unit dose in saline, e.g., sterile saline, such as sterile saline for injection. In yet another particular embodiment, presented herein is a pharmaceutical composition comprising about 1×10¹² Treg EVs formulated into a unit dose in 1 mL, 2 mL, 3 mL. 4 mL or 5 mL saline, e.g., sterile saline, such as sterile saline for injection. In a specific embodiment, presented herein is a pharmaceutical composition comprising about 1×10¹² Treg EVs formulated into a unit dose in 2 mL saline, e.g., sterile saline, such as sterile saline for injection. Such a pharmaceutical composition may be present, for example present in a vial, such as a sterile vial, as a single unit dose or as multiple unit doses. For example, in a particular embodiment, presented herein is a pharmaceutical composition comprising about 1×10¹² Treg EVs as a unit dose in 1 mL, 2 mL, 3 mL, 4 mL, or 5 mL sterile saline in a vial. In a specific embodiment, presented herein is a pharmaceutical composition comprising about 1×10¹² Treg EVs as a unit dose in 2 mL sterile saline in a vial.

### 5.4 Methods of Treatment

Provided herein are methods of treatment comprising administering an effective amount of a population of anti-inflammatory EVs as described herein to a subject in need thereof.

In some embodiments, the subject is diagnosed with or is suspected of having a disorder associated with Treg dysfunction. In some embodiments, the subject is diagnosed with or is suspected of having a disorder associated with Treg deficiency. In some embodiments, the subject is diagnosed with or is suspected of having a condition (e.g., an inflammatory condition) driven by a T cell response. In some embodiments, the subject is diagnosed with or is suspected of having a condition (e.g., an inflammatory condition) driven by a myeloid cell response. In some embodiments, the subject is diagnosed with or is suspected of having a condition whose symptoms are contributed to (e.g., brought on or worsened by) a myeloid cell response. Certain embodiments, the condition is an inflammatory, autoimmune or neurodegenerative disorder. In specific embodiments, the myeloid cell is a monocyte, macrophage or microglia. In certain embodiments, the myeloid cells comprise microglia in the brain. In certain embodiments, the myeloid cells comprise monocytes or macrophages in the periphery, outside the central nervous system.

In some embodiments the subject is diagnosed with or is suspected of having a neurodegenerative disease. In some embodiments, the subject is diagnosed with or is suspected of having Alzheimer's disease, Amyotrophic Lateral Sclerosis, Huntington's disease, Parkinson's disease, or frontotemporal dementia.

In some embodiments, the subject is diagnosed with or is suspected of having a disorder that would benefit from downregulation of the immune system.

In some embodiments, the subject is diagnosed with or suspected of having an autoimmune disease. The autoimmune disease may be, for example, systemic sclerosis (scleroderma), polymyositis, ulcerative colitis, inflammatory bowel disease, Crohn's disease, celiac disease, multiple sclerosis (MS), rheumatoid arthritis (RA), Type I diabetes, psoriasis, dermatomyositis, lupus, e.g., systemic lupus erythematosus, or cutaneous lupus, myasthenia gravis, autoimmune nephropathy, autoimmune hemolytic anemia, autoimmune cytopenia, autoimmune encephalitis, autoimmune hepatitis, autoimmune uveitis, alopecia, thyroiditis or pemphigus.

In some embodiments, the subject is diagnosed with or suspected of having heart failure or ischemic cardiomyopathy.

In some embodiments, the subject is diagnosed with or suspected of having graft-versus-host disease, e.g., after undergoing organ transplantation (such as a kidney transplantation or a liver transplantation), or after undergoing stem cell transplantation (such as hematopoietic stem cell transplantation including a bone marrow transplant).

In some embodiments, the subject is diagnosed with or suspected of having neuroinflammation. Neuroinflammation may be associated, for example, with stroke, acute disseminated encephalomyelitis (ADEM), acute optic neuritis, acute inflammatory demyelinating polyradiculoneuropathy, chronic inflammatory demyelinating polyradiculoneuropathy, Guillain-Barre syndrome, transverse myelitis, neuromyelitis optica (NMO), epilepsy, traumatic brain injury, spinal cord injury, encephalitis central nervous system (CNS) vasculitis, neurosarcoidosis, autoimmune or post-infectious encephalitis, or chronic meningitis.

In some embodiments, the subject is diagnosed with or suspected of having a liver disorder. The liver disorder may, for example, be fatty liver, e.g., nonalcoholic fatty liver disease (NAFLD), non-alcoholic steatohepatitus (NASH), primary biliary cholangitis, autoimmune hepatitis, liver cancer, liver inflammation, hepatitis A, hepatitis B, and hepatitis C. In certain embodiments, the liver disorder is NAFLD. In certain embodiments, the liver disorder is NASH.

In some embodiments, the subject is diagnosed with or suspected of having alcoholic hepatitis (AH) or alcoholic steatohepatitis (ASH).

In some embodiments, the subject is diagnosed with or suspected of having a metabolic disorder. The metabolic disorder can be, but is not limited to, fibrosis, metabolic syndrome, NAFLD, and NASH.

In some embodiments, the subject is in need of improving islet graft survival, and the method comprises administering to the subject an effective amount of a population of anti-inflammatory EVs as described herein or a pharmaceutical composition described herein in combination with the islet transplantation.

In some embodiments, the subject is diagnosed with or suspected of having cardo-inflammation, e.g., cardio-inflammation associated with atheroscleorosis, myocardial infarction, ischemic cardiomyopathy, with heart failure.

In some embodiments, the subject is diagnosed with or suspected of having chronic inflammatory demyelinating polyradiculoneuropathy (CIDP). In some embodiments, the subject is diagnosed with or suspected of having acute inflammatory demyelinating polyneuropathy (AIDP). In some embodiments, the subject is diagnosed with or suspected of having Guillain-Barré syndrome (GBS).

In some embodiments, the subject has had a stroke.

In some embodiments, the subject is diagnosed with or suspected of having cancer, e.g., a blood cancer.

In some embodiments, the subject is diagnosed with or suspected of having asthma.

In some embodiments, the subject is diagnosed with or suspected of having eczema.

In some embodiments, the subject is diagnosed with or suspected of having a disorder associated with overactivation of the immune system.

In some embodiments, the subject is diagnosed with or suspected of having Tregopathy. The Tregopathy may be caused by a FOXP3, CD25, cytotoxic T lymphocyte-associated antigen 4 (CTLA4), LPS-responsive and beige-like anchor protein (LRBA), or BTB domain and CNC homolog 2 (BACH2) gene loss-of-function mutation, or a signal transducer and activator of transcription 3 (STAT3) gain-of-function mutation.

In some embodiments, the donor subject is a geriatric subject, e.g., a subject of at least 65, at least 70, at least 75, at least 80, at least 85 or at least 90 years of age.

In some embodiments, about 1×10⁸ to about 1×10¹⁴ EVs, about 1×10⁸ to about 1×10¹² EVs, about 1×10⁸ to about 1×10¹⁰ EVs, about 1×10¹⁰ to about 1×10¹⁴ EVs, about 1×10¹⁰ to about 1×10¹² EVs, about 1×10⁶ to about 1×10⁷ EVs, about 1×10⁷ to about 1×10⁸ EVs, about 1×10⁸ to about 1×10⁹ EVs, about 1×10⁹ to about 1×10¹⁰ EVs, about 1×10¹⁰ to about 1×10¹¹ EVs, about 1×10¹² to about 1×10¹³ EVs, about 1×10¹³ to about 1×10¹⁴ EVs, or about 1×10¹⁴ to about 1×10¹⁵ EVs are administered.

In some embodiments, about 1×10⁸ EVs, about 2×10⁸ EVs, about 3×10⁸ EVs, about 4×10⁸ EVs, about 5×10⁸ EVs, about 6×10⁸ EVs, about 7×10⁸ EVs, about 8×10⁸ EVs, about 9×10⁸ EVs, about 1×10⁹ EVs, about 2×10⁹ EVs, about 3×10⁹ EVs, about 4×10⁹ EVs, about 5×10⁹ EVs, about 6×10⁹ EVs, about 7×10⁹ EVs, about 8×10⁹ EVs, about 9×10⁹ EVs or about 1×10¹⁰ EVs are administered, for example, are administered per dose.

In some embodiments, about 1×10⁸ to about 1×10¹⁴ EVs/mL, about 1×10⁸ to about 1×10¹² EVs/mL, about 1×10⁸ to about 1×10¹⁰ EVs/mL, about 1×10¹⁰ to about 1×10¹⁴ EVs/mL, about 1×10¹⁰ to about 1×10¹² EVs/mL, about 1×10⁶ to about 1×10⁷ EVs/mL, about 1×10⁷ to about 1×10⁸ EVs/mL, about 1×10⁸ to about 1×10⁹ EVs/mL, about 1×10⁹ to about 1×10¹⁰ EVs/mL, about 1×10¹⁰ to about 1×10¹¹ EVs/mL, about 1×10¹² to about 1×10¹³ EVs/mL, about 1×10¹³ to about 1×10¹⁴ EVs/mL, or about 1×10¹⁴ to about 1×10¹⁵ EV/mL are administered.

In some embodiments, about 1×10⁸ EVs/ml, about 2×10⁸ EVs/ml, about 3×10⁸ EVs/ml, about 4×10⁸ EVs/ml, about 5×10⁸ EVs/ml, about 6×10⁸ EVs/ml, about 7×10⁸ EVs/ml, about 8×10⁸ EVs/ml, about 9×10⁸ EVs/ml, about 1×10⁹ EVs/ml, about 2×10⁹ EVs/ml, about 3×10⁹ EVs/ml, about 4×10⁹ EVs/ml, about 5×10⁹ EVs/ml, about 6×10⁹ EVs/ml, about 7×10⁹ EVs/ml, about 8×10⁹ EVs/ml, about 9×10⁹ EVs/ml or about 1×10¹⁰ EVs/ml are administered, for example, are administered per dose.

In some embodiments, about 1 μg to about 100 μg EVs, about 100 μg to about 200 μg EVs, about 200 μg to about 300 μg EVs, about 300 μg to about 400 μg EVs, about 400 μg to about 500 μg EVs, about 500 μg to about 600 μg EVs, about 600 μg to about 700 μg EVs, about 700 μg to about 800 μg EVs, about 800 μg to about 900 μg EVs, about 900 μg to about 1 mg EVs, about 1 mg to about 10 mg EVs, about 10 mg to about 20 mg EVs, about 20 mg to about 30 mg EVs, about 30 mg to about 40 mg EVs, about 40 mg to about 50 mg EVs, about 50 mg to about 60 mg EVs, about 60 mg to about 70 mg EVs, about 70 mg to about 80 mg EVs, about 80 mg to about 90 mg EVs, about 90 mg to about 100 mg EVs, about 100 mg to about 110 mg EVs, about 110 mg to about 120 mg EVs, about 120 mg to about 130 mg EVs, about 130 mg to about 140 mg EVs, about 150 mg to about 160 mg EVs, about 160 mg to about 170 mg EVs, about 170 mg to about 180 mg EVs, about 180 mg to about 190 mg EVs, or about 190 mg to about 200 mg EVs are administered.

In some embodiments, about 1 μg to about 100 μg EVs/mL, about 100 μg to about 200 μg EVs/mL, about 200 μg to about 300 μg EVs/mL, about 300 μg to about 400 μg EVs/mL, about 400 μg to about 500 μg EVs/mL, about 500 μg to about 600 μg EVs/mL, about 600 μg to about 700 μg EVs/mL, about 700 μg to about 800 μg EVs/mL, about 800 μg to about 900 μg EVs/mL, about 900 μg to about 1 mg EVs/mL, about 1 mg to about 10 mg EVs/mL, about 10 mg to about 20 mg EVs/mL, about 20 mg to about 30 mg EVs/mL, about 30 mg to about 40 mg EVs/mL, about 40 mg to about 50 mg EVs/mL, about 50 mg to about 60 mg EVs/mL, about 60 mg to about 70 mg EVs/mL, about 70 mg to about 80 mg EVs/mL, about 80 mg to about 90 mg EVs/mL, about 90 mg to about 100 mg EVs/mL, about 100 mg to about 110 mg EVs/mL, about 110 mg to about 120 mg EVs/mL, about 120 mg to about 130 mg EVs/mL, about 130 mg to about 140 mg EVs/mL, about 150 mg to about 160 mg EVs/mL, about 160 mg to about 170 mg EVs/mL, about 170 mg to about 180 mg EVs/mL, about 180 mg to about 190 mg EVs/mL, or about 190 mg to about 200 mg EVs/mL are administered.

A population of anti-inflammatory EVs may be administered to the subject by any suitable route. For example, a population of anti-inflammatory EVs may be administered to the subject by routes including intranasal, parenteral (e.g., subcutaneous, intravenous, intramuscular, intraperitoneal, intraarterial, intraventricular, intrathecal, intraurethral, intrasternal, and intrasynovial), intradermal, oral (e.g., ingestion, sublingual), inhalation, nasal, e.g., nasal drip, intracavity, intracranial, ocular, e.g., intraocular, and transdermal (topical). In particular embodiments, for example, a population of anti-inflammatory EVs may be administered to the subject in a pharmaceutical composition formulated for administration by a route that includes including intranasal, parenteral (e.g., subcutaneous, intravenous, intramuscular, intraperitoneal, intraarterial, intraventricular, intrathecal, intraurethral, intrasternal, and intrasynovial), intradermal, oral (e.g., ingestion, sublingual), inhalation, nasal, e.g., nasal drip, intracavity, intracranial, ocular, e.g., intraocular, and transdermal (topical).

In certain embodiments, for example, a method of treatment presented herein comprises administering to a subject in need of treatment a pharmaceutical composition that comprises an effective amount of an isolated, cell-free population of anti-inflammatory EVs as described herein and has been formulated to be suitable for intranasal administration to a subject, for example, a human subject.

In certain embodiments, for example, a method of treatment presented herein comprises administering to a subject in need of treatment a pharmaceutical composition that comprises an effective amount of an isolated, cell-free population of anti-inflammatory EVs as described herein and has been formulated to be suitable for injection, infusion or implantation to a subject, for example, a human subject.

In certain embodiments, for example, a method of treatment presented herein comprises administering to a subject in need of treatment a pharmaceutical composition that comprises an effective amount of an isolated, cell-free population of anti-inflammatory EVs as described herein and has been formulated to be suitable for intravenous administration to a subject, for example, a human subject.

In certain embodiments, for example, a method of treatment presented herein comprises administering to a subject in need of treatment a pharmaceutical composition that comprises an effective amount of an isolated, cell-free population of anti-inflammatory EVs as described herein and has been formulated to be suitable for subcutaneous administration to a subject, for example, a human subject.

In certain embodiments, for example, a method of treatment presented herein comprises administering to a subject in need of treatment a pharmaceutical composition that comprises an effective amount of an isolated, cell-free population of anti-inflammatory EVs as described herein and has been formulated to be suitable for intramuscular administration to a subject, for example, a human subject.

A population of anti-inflammatory EVs may be administered to the subject more than once. For example, a population of anti-inflammatory EVs may be administered to the subject every week, every other week, every three weeks, once a month, every other month, every 3 months, every 6 months, ever 12 months, every 18 months, every year, every other year, every 3 years, or every 5 years.

The population of anti-inflammatory EVs administered to the subject may be autologous to the subject. The population of anti-inflammatory EVs administered to the subject may be allogeneic to the subject. The population of anti-inflammatory EVs administered to the subject may be derived from human suppressive immune cells, e.g., Tregs, from more than one individual. For example, the population of anti-inflammatory EVs administered to the subject may be a pooled population of anti-inflammatory EVs, wherein some or all of the population of anti-inflammatory EVs is allogeneic to the subject.

In certain embodiments, a population of anti-inflammatory EVs may be administered to the subject more than once. For example, a population of anti-inflammatory EVs may be administered to the subject every week, every other week, every three weeks, once a month, every other month, every 3 months, every 6 months, ever 12 months, every 18 months, every year, every other year, every 3 years, or every 5 years.

### 5.4.1. Additional Therapies

In some embodiments, a subject treated in accordance with the method of treatment described herein further received one or more additional therapy or additional therapies.

In some embodiments, the subject is additionally administered an effective amount of an ex vivo-expanded population of Tregs. In some embodiments, the population of Tregs has been cryopreserved. In some embodiments, the cryopreserved population of Tregs is thawed and administered to the subject without further expansion.

In some embodiments, the population of Tregs or cryopreserved population of Tregs may be the population, or one of the populations, of Tregs from which the EVs being administered to the subject have been isolated. In some embodiments, the population of Tregs or the cryopreserved population of Tregs is a population of Tregs described in International Patent Application No. PCT/US2020/63378, or is produced by a method described in International Patent Application No. PCT/US2020/63378, which is incorporated by reference herein in its entirety.

In some embodiments, about 1×10⁶ to about 2×10⁶, about 2×10⁶ to about 3×10⁶, about 3×10⁶ to about 4×10⁶, about 4×10⁶ to about 5×10⁶, about 5×10⁶ to about 6×10⁶, about 6×10⁶ to about 7×10⁶, about 7×10⁶ to about 8×10⁶, about 8×10⁶ to about 9×10⁶, about 9×10⁶ to about 1×10⁷, about 1×10⁷ to about 2×10⁷, about 2×10⁷ to about 3×10⁷, about 3×10⁷ to about 4×10⁷, about 4×10⁷ to about 5×10⁷, about 5×10⁷ to about 6×10⁷, about 6×10⁷ to about 7×10⁷, about 7×10⁷ to about 8×10⁷, about 8×10⁷ to about 9×10⁷, about 9×10⁷ to about 1×10⁸, about 1×10⁸ to about 2×10⁸, about 2×10⁸ to about 3×10⁸, about 3×10⁸ to about 4×10⁸, about 4×10⁸ to about 5×10⁸, about 5×10⁸ to about 6×10⁸, about 6×10⁸ to about 7×10⁸, about 7×10⁸ to about 8×10⁸, about 8×10⁸ to about 9×10⁸, about 9×10⁸ to about 1×10⁹ CD4+CD25+ cells per kg of body weight of the subject are administered. In some embodiments, 1×10⁶ Tregs, e.g., CD4+CD25+ cells (+/−10%) per kg of body weight of the subject are administered.

In some embodiments, about 1×10⁶ to about 2×10⁶, about 2×10⁶ to about 3×10⁶, about 3×10⁶ to about 4×10⁶, about 4×10⁶ to about 5×10⁶, about 5×10⁶ to about 6×10⁶, about 6×10⁶ to about 7×10⁶, about 7×10⁶ to about 8×10⁶, about 8×10⁶ to about 9×10⁶, about 9×10⁶ to about 1×10⁷, about 1×10⁷ to about 2×10⁷, about 2×10⁷ to about 3×10⁷, about 3×10⁷ to about 4×10⁷, about 4×10⁷ to about 5×10⁷, about 5×10⁷ to about 6×10⁷, about 6×10⁷ to about 7×10⁷, about 7×10⁷ to about 8×10⁷, about 8×10⁷ to about 9×10⁷, about 9×10⁷ to about 1×10⁸, about 1×10⁸ to about 2×10⁸, about 2×10⁸ to about 3×10⁸, about 3×10⁸ to about 4×10⁸, about 4×10⁸ to about 5×10⁸, about 5×10⁸ to about 6×10⁸, about 6×10⁸ to about 7×10⁸, about 7×10⁸ to about 8×10⁸, about 8×10⁸ to about 9×10⁸, about 9×10⁸ to about 1×10⁹ Tregs, e.g., CD4 CD25 cells are administered to a patient.

In some embodiments, about 1×10⁶ to about 2×10⁶, about 2×10⁶ to about 3×10⁶, about 3×10⁶ to about 4×10⁶, about 4×10⁶ to about 5×10⁶, about 5×10⁶ to about 6×10⁶, about 6×10⁶ to about 7×10⁶, about 7×10⁶ to about 8×10⁶, about 8×10⁶ to about 9×10⁶, about 9×10⁶ to about 1×10⁷, about 1×10⁷ to about 2×10⁷, about 2×10⁷ to about 3×10⁷, about 3×10⁷ to about 4×10⁷, about 4×10⁷ to about 5×10⁷, about 5×10⁷ to about 6×10⁷, about 6×10⁷ to about 7×10⁷, about 7×10⁷ to about 8×10⁷, about 8×10⁷ to about 9×10⁷, about 9×10⁷ to about 1×10⁸, about 1×10⁸ to about 2×10⁸, about 2×10⁸ to about 3×10⁸, about 3×10⁸ to about 4×10⁸, about 4×10⁸ to about 5×10⁸, about 5×10⁸ to about 6×10⁸, about 6×10⁸ to about 7×10⁸, about 7×10⁸ to about 8×10⁸, about 8×10⁸ to about 9×10⁸, about 9×10⁸ to about 1×10⁹ Tregs, e.g., CD4 CD25 cells are administered to a patient in one infusion.

In some embodiments, a cryopreserved composition comprising a population of Tregs is administered within about 30 minutes, about 1h, about 2-3h, about 3-4h, about 4-5h, about 5-6, about 6-7h, about 7-8h, about 8-9h, or about 9-10h of thawing the cryopreserved composition comprising a population of Tregs. The cryopreserved composition comprising a population of Tregs may be stored at about 2° C. to about 8° C. (e.g., at about 4°) between thawing and administration.

In some embodiments, one dose of a population of Tregs or a composition comprising a population of Tregs is administered to a subject. In some embodiments, a population of Tregs or a composition comprising a population of Tregs is administered more than once. In some embodiments, a population of Tregs or a composition comprising a population of Tregs is administered two or more times. In some embodiments, a population of Tregs or a composition comprising a population of Tregs is administered every 1-2 weeks, 2-3 weeks, 3-4 weeks, 4-5 weeks, 5-6 weeks, 6-7 weeks, 7-8 weeks, 8-9 weeks, 9-10 weeks, 10-11 weeks, 11-12 weeks, every 1-2 months, 2-3 months, 3-4 months, 4-5 months, 5-6 months, 6-7 months, 7-8 months, 8-9 months, 9-10 months, 10-11 months, 11-12 months, 13-14 months, 14-15 months, 15-16 months, 16-17 months, 17-18 months, 18-19 months, 19-20 months, 20-21 months, 21-22 months, 22-23 months, 23-24 months, every 1-2 years, 2-3 years, 3-4 years or 4-5 years.

In some embodiments, about 1×10⁶ Tregs per kg of body weight of the subject are administered in the first administration and the number of Tregs administered is increased in the second third and subsequent administration. In some embodiments, about 1×10⁶ Tregs per kg of body weight of the subject are administered in the first two administrations, and the number of Tregs administered is increased in every other administration thereafter (e.g., the 4th, 6th, 8th and 10th administration). Thus, for example, about 1×10⁶ Tregs per kg of body weight of the subject may be administered per month for the first and second month, and about 2×10⁶ Tregs per kg of body weight of the subject may be administered per month for the third and fourth month, and/or about 3×10⁶ cells per kg of body weight of the subject are administered per month for the fifth and sixth month.

In some embodiments, a method of treatment provide herein comprises administering a population of autologous Tregs or a composition comprising a population of autologous Tregs to the subject. In other embodiments, a method of treating a neurodegenerative disorder in a subject comprises administering a population of allogeneic Tregs or a composition comprising a population of allogeneic Tregs to the subject.

In some embodiments, the subject is additionally administered IL-2. The dose of IL-2 may be about 0.5-1×10⁵ IU/m², about 1-1.5×10⁵ IU/m², about 1.5-2×10⁵ IU/m², about 2-2.5×10⁵ IU/m², about 2.5-3×10⁵IU/m², about 3-3.5×10⁵ IU/m², about 3.5-4×10⁵ IU/m², about 4-4.5×10⁵ IU/m², about 4.5-5×10⁵ IU/m², about 5-6×10⁵IU/m², about 6-7×10⁵ IU/m², about 7-8×10⁵ IU/m², about 8-9×10⁵ IU/m², about 9-10×10⁵ IU/m², about 10-15×10⁵ IU/m², about 15-20×10⁵ IU/m², about 20-25×10⁵ IU/m², about 25-30×10⁵ IU/m², about 30-35×10⁵ IU/m², about 35-40×10⁵ IU/m², about 40-45×10⁵ IU/m², about 45-50×10⁵ IU/m², about 50-60×10⁵ IU/m², about 60-70×10⁵ IU/m², about 70-80×10⁵ IU/m², about 80-90×10⁵ IU/m², or about 90-100×10⁵ IU/m². In specific embodiments, the subject is administered 2×10⁵ IU/m² of IL-2.

The IL-2 may be administered one, two or more times a month. In some embodiments, the IL-2 is administered three times a month. In some embodiments, the IL-2 is administered subcutaneously. The IL-2 may be administered at least 2 weeks, at least 3 weeks, or at least 4 weeks prior to the population of anti-inflammatory EVs.

In some embodiments, the subjected treated in accordance with the methods described herein receives one or more additional therapies are for the treatment of Alzheimer's. Addition therapies for the treatment of Alzheimer's may include acetylcholinesterase inhibitors (e.g., donepezil (Aricept®), galantamine (Razadyne®), or rivastigmine (Exelon®)) or NMDA receptor antagonists (e.g., Memantine (Akatinol®, Axura®, Ebixa®/Abixa®, Memox® and Namenda®). Additional therapies may also include anti-inflammatory agents (e.g., nonsteroidal anti-inflammatory drugs (NSAID) such as ibuprofen, indomethacin, and sulindac sulfide)), neuronal death associated protein kinase (DAPK) inhibitors such as derivatives of 3-amino pyridazine, Cyclooxygenases (COX-1 and 2) inhibitors, or antioxidants such as vitamins C and E.

In some embodiments, a subject treated in accordance with the methods described herein receives on or more additional therapies for the treatment of ALS. Additional therapies for the treatment of ALS may include Riluzole (Rilutek®) or Riluzole (Rilutek®).

### 5.4.2. Methods of Determining Treatment Effect

The effect of a method of treatment provided herein may be assessed by monitoring clinical signs and symptoms of the disease to be treated.

The efficacy of a method of treatment described herein may be assessed at about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, about 72 weeks, about 76 weeks, about 80 weeks, about 84 weeks, about 88 weeks, about 92 weeks, about 96 weeks, about 100 weeks, at about 2-3 months, 3-4 months, 4-5 months, 5-6 months, 6-7 months, 7-8 months, 8-9 months, about 9-10 months, about 10-11 months, about 11-12 months, about 12-18 months, about 18-24 months, about 1-2 years, about 2-3 years, about 3-4 years, about 4-5 years, about 5-6 years, about 6-7 years, about 7-8 years, about 8-9 years, or about 9-10 years after initiation of treatment in accordance with the method described herein.

In some embodiments, method of treatment provided herein results in a change in the Appel ALS score compared to baseline. In the context of an assessment of the effect of a method of treatment, the term “baseline” refers to a measurement pre-treatment. The Appel ALS score measures overall progression of disability or altered function. In some embodiments, the Appel ALS score decreases in a subject treated in accordance with a method provided herein compared to baseline, indicating an improvement of symptoms. In other embodiments, the Appel ALS score remains unchanged ins a subject treated in accordance with a method provided herein compared to baseline.

In some embodiments, a method of treatment provided herein results in a change in the Amyotrophic Lateral Sclerosis Functional Rating Scale-revised (ALSFRS-R) score compared to baseline. The ALSFRS-R score assesses the progression of disability or altered function. In some embodiments, the ALSFRS-R score increases in a subject treated in accordance with a method provided herein compared to baseline, indicating an improvement of symptoms. In other embodiments, the Appel ALSFRS-R score remains unchanged in a subject treated in accordance with a method provided herein compared to baseline.

In some embodiments, a method of treatment provided herein results in a change in forced vital capacity (FVC; strength of muscles used with expiration) compared to baseline, where the highest number is the strongest measurement. In some embodiments, FVC increases in a subject treated in accordance with a method provided herein compared to baseline. In other embodiments, FVC remains unchanged in a subject treated in accordance with a method provided herein compared to baseline.

In some embodiments, a method of treatment provided herein results in a change in Maximum Inspiratory Pressure (MIP; strength of muscles used with inspiration) compared where the highest number is the strongest measurement. In some embodiments, MIP increases in a subject treated in accordance with a method provided herein compared to baseline. In other embodiments, MIP remains unchanged in a subject treated in accordance with a method provided herein compared to baseline.

In some embodiments, a method of treatment provided herein results in a change in Neuropsychiatric Inventory Questionnaire (NPI-Q) compared to baseline. The NPI-Q provides symptom Severity and Distress ratings for each symptom reported, and total Severity and Distress scores reflecting the sum of individual domain scores. In some embodiments, the NPI-Q score decreases in a subject treated in accordance with a method provided herein compared to baseline. In other embodiments, NPI-Q score remains unchanged in a subject treated in accordance with a method provided herein compared to baseline.

In some embodiments, a method of treatment provided herein results in a decrease in the frequency of GI symptoms, anaphylaxis or seizures compared to baseline.

In some embodiments, a method of treatment provided herein results in a change in a change in CSF amyloid and/or CSF tau protein (CSF-tau) compared to baseline. In some embodiments, the levels of CSF amyloid and/or CSF tau protein decreases in a subject treated in accordance with a method provided herein compared to baseline. In other embodiments, the levels of CSF amyloid and/or CSF tau protein remains unchanged in a subject treated in accordance with a method provided herein compared to baseline.

In some embodiments, a method of treatment provided herein results in a change in Clinical Dementia Rating (CDR) compared to baseline. The CDR rates memory, orientation, judgment and problem-solving, community affairs, home and hobbies, and personal care, and a global rating is then generated, ranging from 0-no impairment to 3-severe impairment. In some embodiments, the CDR decreases in a subject treated in accordance with the methods provided herein compared to baseline. In other embodiments, the CDR remains unchanged in a subject treated in accordance with a method provided herein compared to baseline.

In some embodiments, a method of treatment provided herein results in a change in Alzheimer's Disease Assessment Scale (ADAS)-cog13 score compared to baseline. ADAS-cog tests cognitive performance and has an upper limit is 85 (poor performance) and lower limit is zero (best performance). In some embodiments, the ADAS-cog13 score decreases in a subject treated in accordance with a method provided herein compared to baseline. In other embodiments, the ADAS-cog13 score remains unchanged in a subject treated in accordance with a method provided herein.

In some embodiments, wherein the method of treatment comprises administration of a population of anti-inflammatory EVs as well as administration of a population of Tregs or a cryopreserved population of Tregs, the method results in an increase in the Treg suppressive function in the blood from baseline. In some embodiments, a method of treatment provided herein results in an increase in the Treg suppressive function in the blood from baseline to week 4, week 8, week 16, week 24, week 30 or week 36. In some embodiments, a method of treatment provided herein results in an increase in the Treg suppressive function in the blood from baseline to week 24. In some embodiments, a method of treatment provided herein results in an increase in the Treg numbers in the blood from baseline. In some embodiments, a method of treatment provided herein results in an increase in the Treg numbers in the blood from baseline to week 4, week 8, week 16, week 24, week 30 or week 36. In some embodiments, a method of treatment provided herein results in an increase in the Treg numbers in the blood from baseline to week 24.

### 5.5 Kits

Provided herein are kits comprising a therapeutic composition of a population of anti-inflammatory EVs provided herein or a composition comprising a population of anti-inflammatory EVs provided herein.

In some embodiments, a kit provided herein comprises instructions for use, additional reagents (e.g., sterilized water or saline solutions for dilution of the compositions), or components, such as tubes, containers or syringes for collection of biological samples, processing of biological samples, and/or reagents for quantitating the amount of one or more surface markers in a sample (e.g., detection reagents, such as antibodies).

In some embodiments, the kits contain one or more containers containing a population of anti-inflammatory EVs provided herein or a composition comprising a population of anti-inflammatory EVs provided herein. The one or more containers holding the composition may be a single-use vial or a multi-use vial. In some embodiments, the article of manufacture or kit may further comprise a second container comprising a suitable diluent. In some embodiments, the kit contains instruction for use (e.g., dilution and/or administration) of a population of anti-inflammatory EVs provided herein or a composition comprising a population of Tregs provided herein.

In certain embodiments, a kit provided herein comprise one or more unit doses of anti-inflammatory EV as described herein, in one or more containers, e.g., one or more vials. Such a kit may, for example, comprise a single unit dose per container, for example, a single unit dose per vial, or may comprise multiple unit doses per container, for example, multiple unit doses per vial.

### 6. EXAMPLES

### 6.1 Example 1: An Improved Treg Manufacturing Protocol

### 6.1.1. Details of an Improved Treg Manufacturing Protocol

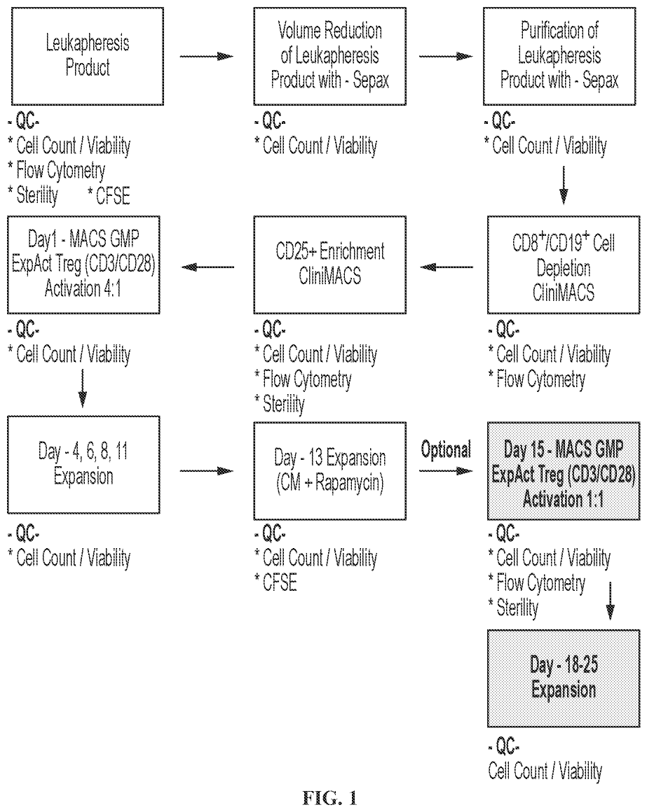

The following is a list of representative steps illustrates an embodiment of the improved Treg ex-vivo expansion protocol. The exemplary protocol may, for example, be used as part of a method for producing an isolated, cell-free population of anti-inflammatory EVs, as presented herein. A more detailed summary of such a representative Treg manufacturing protocol is presented at FIG. 1 and a more detailed protocol is presented in 6.1.2, below. As noted in the description of FIG. 1, the improved Treg manufacturing method may be used to expand Tregs from ALS patients. It is to be understood that the Treg production methods presented herein may also be applied to expand Tregs from other starting materials, including from cell samples from subjects with other disorders, e.g., other neurodegenerative disorders, or from healthy donor subjects. Exemplary methods of producing obtaining, enriching for and ex-vivo expanding a population of Tregs utilizing improved methods such as this are also described in International Patent Application No. PCT/US2020/63378, which is incorporated by reference herein in its entirety.

### Donor Cell Isolation.

1) Fresh leukapheresis (or blood sample) products are used immediately after isolation on Day 0. They are not stored at 4° C. overnight.

### Treg Enrichment.

2) CD25+ cells are obtained following leukapheresis (or blood sample draw); enrichment includes volume reduction, purification, then CD8+/CD19+ depletion, followed by CD25+ enrichment.

### Expansion.

3) Freshly isolated CD25+ cells are immediately (within approximately 30 minutes) placed into culture media. They are not cryopreserved first.

4) IL-2 is administered every 2-3 days on Days 6, 8 and 11. Cells are not discarded during expansion and thus, the scale is much larger.

5) During media changes, half the media is removed and replenished on Day 1, Day 4 and Day 6. Cells removed during these media changes are not returned to the culture flasks.

6) Cells are generally not centrifuged during media changes.
EVs may made be isolated at any point during the expansion process. For example, if desired, EVs may be isolated from the culture media removed in 5), above.
   6.1.2. Exemplary Protocol for Isolation and Expansion of Regulatory T Cells (Tregs) from a leukapheresis or blood sample product

This protocol may be applied to isolation and expansion of leukapheresis or blood sample products from, e.g., ALS patients, Alzheimer's Disease patients, or patients exhibiting a different disorder, for example a different neurodegenerative disorder, or from healthy subjects.

### 6.1.2.1 Step 1: Patient Leukapheresis/Blood Sample Product Processing

Leukapheresis products should be processed within 24 hours. The total volume of the leukapheresis product should be between 100 mL and 840 mL. If the leukapheresis product is less than 100 mL, an equal volume of CliniMACS Buffer with 1% human serum albumin (HAS) should be added.

Volume reduction of the leukapheresis product is carried out using the GE Healthcare/Biosafe Sepax 2 RM with the PeriCell Protocol and CS490.1 kit (PeriCell).

Leukapheresis Products are purified using the GE Healthcare-Biosafe Sepax 2 RM NeatCell Protocol and CS900.2 kit.

6.1.2.2 Step 2: Treg enrichment

CD8+ and CD19+ Cells are depleted using CliniMACS kit according to manufacturer's instructions, which includes labeling cells with CD8+ and CD19+ micro beads for depletion and then using automatic cell separation using the CliniMACS® Plus Instrument in combination with CliniMACS PBS/EDTA Buffer in 1% HSA, the CliniMACS Tubing Set LS and software sequence DEPLETION 2.1.

Subsequently, the population is enriched for CD25+ Tregs by positive selection using CliniMACS, which includes labeling cells with CD25 for Enrichment CD25 Micro-Beads and then using automatic cell separation using the CliniMACS® Plus Instrument in combination with CliniMACS PBS/EDTA Buffer in 1% HSA, the CliniMACS Tubing Set LS and software sequence ENRICHMENT 3.2.

### 6.1.2.3 Step 3: Treg Expansion

Treg expansion is initiated on Day 0 from CD25+ enriched leukapheresis/blood sample product.

Cell Culture and Harvest Parameter:
Slow-growing expansion: 25 days of cell culture with an estimated cell number <2×10⁹ cells-second leukapheresis/blood sample may be required.
Normal expansion: 25 days of cell culture with an estimated cell number ≥2×10⁹ cells.
Fast-growing expansion: estimated cell number ≥2×10⁹ cells in ≤15 days of cell culture.
All cell culture expansions should maintain a purity of 70% CD4+CD25+ cells with viability above 70% prior to sterile vialing and cryopreservation.

The CD25+ enriched leukapheresis/blood sample product is centrifuged, the pellet washed in TexMACS Medium with 5% Human AB Serum, centrifuged again and the resulting pellet is resuspended in TexMACS media with 5% Human AB Serum at a density of 0.8-1.0×10⁶ cells/mL. The cells are transferred in to flasks and incubated for 16-18 hours at 37° C. in a humidified mixture of 95% air and 5% CO₂.

The cell concentration should be maintained between 0.5×10⁶ cells/mL and 1.2×10⁶ cells/mL after each medium change. EVs may be isolated from the medium which is removed from the Treg culture at one or more media changes. The medium may be frozen before EVs are isolated.

For cell culture medium removal, flasks are stood upright for at least 20 minutes without disturbing them, and then 50% of the total medium volume is removed.

Viability is assessed by trypan blue. If cell viability is over 90%, cells are expanded by changing the cell culture media to obtain 0.5×10⁶ cells/mL-1.2×10⁶ cells/mL.

On Day 1, the cells are stimulated with CD3/CD28 beads using the MACS GMP ExpAct Treg Kit. This kit contains 3.5 μm particles, which are preloaded with CD28 antibodies, anti-biotin antibodies and CD3-Biotin. Each vial contains 1×10⁹ ExpAct Treg Beads (2×10⁵/μL). MACS GMP ExpAct Treg Beads and Treg cells should be at a bead-to-cell ratio of 4:1 for initial stimulation. For activation, the cell concentration should be about 0.5-0.7×10⁶ cells/mL for MACS GMP ExpAct Treg Kit (CD3/CD28 Beads). Activation is carried out on Day 1 and again on Day 15.

The cells are expanded in TexMACS Medium with 5% Human AB Serum supplemented with 100 nmol/L rapamycin and 500 IU/ml IL-2.

The culture medium is changed and rapamycin is replenished on Day 4, Day 6, Day 8, Day 11, Day 13, Day 15, Day 18, Day 20, and Day 22. The IL-2 is replenished on Day 6, Day 8, Day 11, Day 15, Day 18, and Day 20. EVs may be isolated from the medium which is removed from the Treg culture at the time of harvesting. The medium may be frozen before EVs are isolated.

6.1.2.4 Step 4: Treg Harvesting (Optional if Treg Culture is used for EV isolation)

Optionally, the expanded Tregs may be harvested. For example, the expanded Tregs may be harvested on Day 25. The MACS GMP Activation Beads may be removed using CliniMACS Depletion Tubing Set LS (168-01) and software DEPLETION 2.1. according to manufacturer's instructions.

If the final harvested cell product is contemplated for therapeutic use, it should satisfy the release criteria shown in Table 2.

EVs may be isolated from the medium on the day the Tregs would be harvested (whether or not Treg harvesting is performed). For example, EVs may be isolated from media removed from a Treg culture at the time of harvesting or at the time the Treg would be harvested. The medium may be frozen before EVs are isolated.

TABLE 2
Release Criteria

| Test | Specification |
|---|---|
| Visual Inspection | No evidence of contamination |
| Viability | ≥70% |
| Endotoxin (LAL) | <5 EU/kg |
| Gram Stain | Negative |
| Flow Analysis: CD8+ | <20% |
| Flow Analysis: CD4+CD25+ | ≥70% |
| Residual CD3/CD28 beads | ≤100 beads/3 × 10⁶ cells |
| Non-Release Testing | Aerobic: No growth |
| Sterility - 14 days | Anaerobic: No growth |
| (Aerobic and Anaerobic cultures) | |

### 6.2 Example 2: Characterization of Ex Vivo-Expanded Treg Cell Populations by Proteomics

The experiments presented in this example describe a proteomic analysis of baseline Tregs and Tregs that have been ex-vivo expanded using the improved expansion method described in Example 1, above, and in Section 5.2.1.1, above. The results of these experiments demonstrate that the Tregs produced via these methods constitute a unique, non-naturally occurring Treg population. Likewise, therefore, anti-inflammatory populations of EVs derived from such ex-vivo expanded Tregs also constitute a unique, non-naturally occurring EV population. As discussed below, these signatures differ substantially from the baseline Treg gene product signature and are indicative of the health and potency of the expanded Tregs from which EV populations described herein may be derived.

### 6.2.1. Groups Analyzed in the Experiment

Baseline Tregs—Tregs at baseline levels derived from two ALS patients.

Expanded Tregs—The same patients' Tregs following the expansion described herein, in particular, the protocols described in Section 5.2.1.1 and Example 1, above.

### 6.2.2. Proteomic Profiling Methods

Proteomic profiling via single-shot proteomic analysis was performed on Treg Baseline and Treg Expanded cells. Cell pellets were lysed by RIPA buffer. For each sample, 5 μg of protein supernatant was mixed with NuPAGE LDS Sample Buffer (Thermo, NP0007) and boiled at 90° C. for 10 minutes. The proteins were separated on pre-cast NuPAGE Bis-Tris 10% protein gel (Invitrogen, NP0301BOX). For staining, the gels were first fixed with Destain I (40% MeOH, 7% AcOH) for 15 minutes, stained with Coomassie (0.025% Brilliant Blue R-250, 40% MeOH, 7% AcOH) for 5 minutes, de-stained with Destain I buffer 2 times for 30 minutes, and left in water overnight. Four band regions were cut for each sample. The bands were further de-stained completely with Destain II (40% MeOH, 50 mM NH₄CO₃), equilibrated in water, dehydrated with 75% ACN, and incubated in 50 mM Ammonium bicarbonate solution for 1 hr. Then each band was crushed and digested with 22 μl of Trypsin solution (20 μl 50 mM Ammonium Bicarbonate and 2 μl of 100 ng/μl Trypsin (GenDepot: T9600) overnight at 37° C. Next, the digest was acidified by adding 20 μl 2% Formic acid (Thermo, 85178). The peptide from gel was extracted by adding 350 μl of 100% ACN for 15 min and collected by centrifugation at 21,000 rpm for 5 min. The extracted peptides were dried down in a SpeedVac (Thermo Scientific SC210). For MS runs, peptides from all 4 Bands were re-suspended in 20 μl 5% methanol+0.1% FA solution, pooled together, and measured on an Exploris Orbitrap 480 mass spectrometer (Thermo Fisher Scientific, San Jose, CA) with online separation with Thermo Scientific™ EASY-nLC™ 1200 Liquid Chromatography system. Online separation was performed on 1 μg with a 20 cm long, 100 μm inner diameter packed column with sub-2 μm C18 beads (Reprosil-Pur Basic C18, Catalogue #r119.b9.0003, Dr. Maisch GmbH). A linear reverse phase gradient from 2-30% B (100% ACN) was run for 90 minutes.

Raw mass spectrometry data was processed Proteome Discoverer (PD version 2.0.0.802; Thermo Fisher Scientific). Spectra were matched to peptides from the Human RefSeq protein database (downloaded through RefProtDB on 2020-03-24) within 350-10,000 Da mass range and trypsin/P in silico digestion and up to 2 missed cleavages. Mass error was set to 20 ppm for precursor mass, and 0.02 Da for fragment mass. The following dynamic modifications: Acetyl (Protein N-term), Oxidation (M), Carbamidomethyl (C), DeStreak (C), and Deamidated (NQ). Peptide-Spectral Matches (PSMs) were validated with Percolator (v2.05) (Käll et al 2007, PMID 17952086). The target strict and relaxed FDR levels for Percolator were set at 0.01 and 0.05 (1% and 5%), respectively. Label-free quantification of PSMs was made using Proteome Discoverer's Area Detector Module.

Protein inference and quantitation was performed by gpGrouper (v1.0.040) with shared peptide iBAQ area distribution (Saltzman et al 2018 PMID 30093420). Resulting protein values were median normalized and log transformed for downstream analyses. For statistical assessment, missing value imputation was employed through sampling a normal distribution N(μ-1.8 σ, 0.8 σ), where μ, σ are the mean and standard deviation of the quantified values. To assess differences between groups, the moderated t-test was used as implemented in the R package limma (Ritchie et al., 2015). Multiple-hypothesis testing correction was performed with the Benjamini-Hochberg procedure (Benjamini and Hochberg, 1995). Pathway analyses for phenotype associations were examined using Reactome, Kyoto Encyclopedia of Genes and Genome (KEGG), and Gene Set Enrichment Analysis (GSEA).

### 6.2.3. Proteomic Study Finding

In this example, an unbiased single-shot proteomic analysis via mass spectrometry identified gene products from the expanded Treg cell populations as well as from baseline Tregs (freshly isolated, enriched Tregs pre-expansion, here, from ALS patient cell samples). The following results were obtained:

1. A baseline Treg gene product signature was identified that is resolved following the Treg expansion process. This signature suggests, e.g., dysfunctional epigenetic/methylation mechanisms in the baseline Tregs. A methylation gene product signature was also identified within this baseline signature.

2. A unique proteomic gene product signature of the expanded Tregs was identified, indicating that the Treg cell population produced via the methods presented herein is new and that the expanded Treg cell population represents a robust, potent population. Of importance, this signature is remarkably conserved among the different ALS patient Tregs that went through the expansion process.

3. The enhanced Treg gene product signature following expansion can be further defined by at least the following unique gene product signatures:
a. Treg-associated gene product signature.
b. Mitochondria gene product signature.
c. Cell proliferation gene product signature (cell division, cell cycle, and DNA replication/repair).
d. Highest protein expression gene product signature following expansion process.

### 6.2.4. Results

6.2.4.1 Dysfunctional baseline Tregs display proteomic signature that is ameliorated following expansion.

Single-shot proteomic profiling identified peptide sequences that map to 82 gene products out of the 3,709 total found that were increased in the baseline samples but were subsequently significantly reduced or lost during the expansion process (Table 3; dysfunctional baseline gene produce signature). Analysis of the signature included gene products that had a p value of p<0.05 after correction for false discovery rate and multiple hypothesis testing while also having a fold change of at least 4 (log 2 FC>2). Pathway analysis of these significant gene product sets reveals a dysfunctional Treg phenotype including dysregulated calcium dynamics (p=0.0278), loss of MECP2 binding ability to 5 hmC-DNA (p=6.96e-6), dysregulation of MECP2 expression and activity (p=0.0166), and loss of MECP2 regulation (p-0.0303), phosphorylation (p=0.037), and binding abilities (p=0.0456). It has been previously shown that proper MECP2 expression and function are pivotal for the expression of Treg health and function marker FOXP3 (PMID: 24958888).

Additionally, multiple gene products in this signature are also mapped to histone proteins and other proteins that are modified and play a role in the control of the unwinding of DNA to enable epigenetic changes, particularly methylation of DNA that directly affects transcription. Expression of these dysfunctional epigenetic/methylation-associated gene products is decreased in the population of Tregs after expansion relative to that observed for baseline Tregs. See Table 4 (methylation gene product signature).

TABLE 3
Genes products that were increased in the baseline samples but were subsequently
significantly reduced or lost during the expansion process.

| | | | Log2 of | |
|---|---|---|---|---|
| | | | fold-change | |
| | NCBI | | of baseline | Adjusted |
| Gene Symbol | Gene ID | Gene Description | vs. expanded | p-value |
| HIST1H2BC | 8347 | histone cluster 1 H2B family member c | −14.362 | <0.001 |
| HIST1H2BE | 8344 | histone cluster 1 H2B family member e | −14.199 | 0.005 |
| HIST1H2BG | 8339 | histone cluster 1 H2B family member g | −14.091 | 0.002 |
| HIST1H2BI | 8346 | histone cluster 1 H2B family member i | −13.849 | 0.004 |
| HIST1H2BF | 8343 | histone cluster 1 H2B family member f | −11.737 | 0.020 |
| RAB3C | 115827 | RAB3C, member RAS oncogene family | −9.908 | 0.004 |
| CLC | 1178 | Charcot-Leyden crystal galectin | −8.446 | 0.023 |
| TUBA1A | 7846 | tubulin alpha 1a | −8.424 | 0.027 |
| HBB | 3043 | hemoglobin subunit beta | −8.120 | <0.001 |
| HBA1 | 3039 | hemoglobin subunit alpha 1 | −8.068 | 0.001 |
| HBA2 | 3040 | hemoglobin subunit alpha 2 | −7.585 | 0.002 |
| CDK3 | 1018 | cyclin dependent kinase 3 | −7.110 | 0.007 |
| MECP2 | 4204 | methyl-CpG binding protein 2 | −6.924 | <0.001 |
| MTHFS | 10588 | methenyltetrahydrofolate synthetase | −6.566 | 0.001 |
| PLIN3 | 10226 | perilipin 3 | −6.556 | <0.001 |
| H1F0 | 3005 | H1 histone family member 0 | −6.342 | 0.006 |
| ANXA1 | 301 | annexin A1 | −6.131 | <0.001 |
| RAB3D | 9545 | RAB3D, member RAS oncogene family | −5.629 | 0.017 |
| GIMAP1 | 170575 | GTPase, IMAP family member 1 | −5.611 | 0.009 |
| MPO | 4353 | myeloperoxidase | −5.571 | 0.012 |
| LRRD1 | 401387 | leucine rich repeats and death domain | −5.326 | 0.030 |
| | | containing 1 | | |
| HARS2 | 23438 | histidyl-tRNA synthetase 2, | −5.091 | 0.030 |
| | | mitochondrial | | |
| RAB3A | 5864 | RAB3A, member RAS oncogene family | −5.019 | 0.006 |
| GIMAP1- | 1E+08 | GIMAP1-GIMAP5 readthrough | −4.978 | <0.001 |
| GIMAP5 | | | | |
| PPM1F | 9647 | protein phosphatase, Mg2+/Mn2+ | −4.905 | 0.004 |
| | | dependent 1F | | |
| PRG2 | 5553 | proteoglycan 2, pro eosinophil major | −4.903 | 0.004 |
| | | basic protein | | |
| CA5B | 11238 | carbonic anhydrase 5B | −4.877 | 0.007 |
| NAAA | 27163 | N-acylethanolamine acid amidase | −4.764 | 0.023 |
| ELANE | 1991 | elastase, neutrophil expressed | −4.762 | 0.026 |
| A1BG | 1 | alpha-1-B glycoprotein | −4.649 | 0.006 |
| PRPF38B | 55119 | pre-mRNA processing factor 38B | −4.580 | 0.007 |
| CFAP99 | 402160 | cilia and flagella associated protein 99 | −4.512 | 0.020 |
| AFM | 173 | afamin | −4.357 | 0.003 |
| PCYOX1 | 51449 | prenylcysteine oxidase 1 | −4.301 | 0.042 |
| HK3 | 3101 | hexokinase 3 | −4.219 | 0.020 |
| ARFGAP3 | 26286 | ADP ribosylation factor GTPase | −4.168 | 0.048 |
| | | activating protein 3 | | |
| CRIP2 | 1397 | cysteine rich protein 2 | −4.156 | 0.009 |
| HMGN4 | 10473 | high mobility group nucleosomal | −4.035 | 0.007 |
| | | binding domain 4 | | |
| SGSH | 6448 | N-sulfoglucosamine sulfohydrolase | −4.027 | <0.001 |
| RASSF2 | 9770 | Ras association domain family member 2 | −3.973 | 0.002 |
| CRLF3 | 51379 | cytokine receptor like factor 3 | −3.923 | 0.035 |
| HRNR | 388697 | hornerin | −3.801 | 0.001 |
| DPP7 | 29952 | dipeptidyl peptidase 7 | −3.626 | 0.032 |
| WDHD1 | 11169 | WD repeat and HMG-box DNA binding | −3.497 | 0.049 |
| | | protein 1 | | |
| KPRP | 448834 | keratinocyte proline rich protein | −3.431 | 0.004 |
| SKAP1 | 8631 | src kinase associated phosphoprotein 1 | −3.366 | 0.003 |
| RIPOR2 | 9750 | RHO family interacting cell polarization | −3.356 | 0.045 |
| | | regulator 2 | | |
| ATG5 | 9474 | autophagy related 5 | −3.344 | 0.046 |
| SERPINB9 | 5272 | serpin family B member 9 | −3.250 | <0.001 |
| ALOX5AP | 241 | arachidonate 5-lipoxygenase activating | −3.232 | 0.001 |
| | | protein | | |
| GIMAP4 | 55303 | GTPase, IMAP family member 4 | −3.187 | 0.003 |
| SIGIRR | 59307 | single Ig and TIR domain containing | −3.181 | 0.025 |
| WDR37 | 22884 | WD repeat domain 37 | −3.143 | 0.045 |
| HDDC3 | 374659 | HD domain containing 3 | −3.143 | 0.029 |
| HPX | 3263 | hemopexin | −3.094 | 0.001 |
| RASGRP2 | 10235 | RAS guanyl releasing protein 2 | −3.055 | 0.007 |
| IL16 | 3603 | interleukin 16 | −3.035 | <0.001 |
| VAT1 | 10493 | vesicle amine transport 1 | −3.020 | 0.002 |
| DSG1 | 1828 | desmoglein 1 | −3.003 | 0.031 |
| UBASH3A | 53347 | ubiquitin associated and SH3 domain | −2.984 | 0.010 |
| | | containing A | | |
| AAK1 | 22848 | AP2 associated kinase 1 | −2.966 | 0.008 |
| HRG | 3273 | histidine rich glycoprotein | −2.815 | 0.034 |
| ERGIC1 | 57222 | endoplasmic reticulum-golgi | −2.777 | 0.003 |
| | | intermediate compartment 1 | | |
| PIK3R1 | 5295 | phosphoinositide-3-kinase regulatory | −2.706 | 0.026 |
| | | subunit 1 | | |
| PGM2 | 55276 | phosphoglucomutase 2 | −2.698 | <0.001 |
| EML4 | 27436 | EMAP like 4 | −2.694 | <0.001 |
| GCA | 25801 | grancalcin | −2.624 | 0.036 |
| SH3KBP1 | 30011 | SH3 domain containing kinase binding | −2.604 | 0.001 |
| | | protein 1 | | |
| DCXR | 51181 | dicarbonyl and L-xylulose reductase | −2.581 | <0.001 |
| AHNAK | 79026 | AHNAK nucleoprotein | −2.573 | 0.001 |
| FYB1 | 2533 | FYN binding protein 1 | −2.551 | 0.001 |
| HP1BP3 | 50809 | heterochromatin protein 1 binding | −2.529 | <0.001 |
| | | protein 3 | | |
| HP | 3240 | haptoglobin | −2.527 | 0.003 |
| APOH | 350 | apolipoprotein H | −2.468 | <0.001 |
| PDP1 | 54704 | pyruvate dehyrogenase phosphatase | −2.386 | 0.008 |
| | | catalytic subunit 1 | | |
| KRT5 | 3852 | keratin 5 | −2.384 | 0.023 |
| GRK6 | 2870 | G protein-coupled receptor kinase 6 | −2.333 | 0.009 |
| CYB5R1 | 51706 | cytochrome b5 reductase 1 | −2.168 | 0.004 |
| FLNA | 2316 | filamin A | −2.121 | <0.001 |
| PIP4K2A | 5305 | phosphatidylinositol-5-phosphate 4- | −2.094 | 0.001 |
| | | kinase type 2 alpha | | |
| SRSF9 | 8683 | serine and arginine rich splicing factor 9 | −2.088 | 0.028 |
| ALB | 213 | albumin | −2.001 | <0.001 |

TABLE 4
Dysfunctional epigentic/methylation signature in baseline Tregs

| | | | log2 of fold- | |
|---|---|---|---|---|
| | | | change of | |
| | NCBI | | baseline | Adjusted |
| GeneSymbol | GeneID | GeneDescription | vs. expanded | p-value |
| HIST1H2BC | 8347 | histone cluster 1 H2B family member c | −14.362 | <0.001 |
| HIST1H2BE | 8344 | histone cluster 1 H2B family member e | −14.199 | 0.005 |
| HIST1H2BG | 8339 | histone cluster 1 H2B family member g | −14.091 | 0.002 |
| HIST1H2BI | 8346 | histone cluster 1 H2B family member i | −13.849 | 0.004 |
| HIST1H2BF | 8343 | histone cluster 1 H2B family member f | −11.737 | 0.020 |
| MECP2 | 4204 | methyl-CpG binding protein 2 | −6.924 | <0.001 |
| H1F0 | 3005 | H1 histone family member 0 | −6.342 | 0.006 |
| HP1BP3 | 50809 | heterochromatin protein 1 binding | −2.529 | <0.001 |
| | | protein 3 | | |

6.2.4.2 Enriched proteomic signature following expansion of patient Tregs that is observed across patient groups.

The proteomic analysis of expanded Tregs compared to baseline Tregs identified peptide sequences that mapped back to 391 unique gene products out of 3,709 total that were found that are enriched in the expanded Tregs compared to the baseline patient Tregs (Table 5) These genes are a compilation of all significant gene products that had a p value of p<0.05 after correction for false discovery rate and multiple hypothesis testing while also having a fold change of at least 4 (log 2 FC>2).

TABLE 5
Gene products that were enriched in the expanded
Tregs compared to the baseline patient Tregs.

| | | | Log2 of fold- | |
|---|---|---|---|---|
| | | | change of | |
| Gene | NCBI | | baseline | Adjusted |
| Symbol | Gene ID | Gene Description | vs. expanded | p-value |
| NME1 | 4830 | NME/NM23 nucleoside diphosphate | 13.947 | 0.006 |
| | | kinase 1 | | |
| HIST1H2BJ | 8970 | histone cluster 1 H2B family member j | 13.792 | <0.001 |
| NQO1 | 1728 | NAD(P)H quinone dehydrogenase 1 | 9.019 | <0.001 |
| TUBB8 | 347688 | tubulin beta 8 class VIII | 8.661 | 0.028 |
| TUBB4A | 10382 | tubulin beta 4A class IVa | 8.606 | 0.026 |
| AK4 | 205 | adenylate kinase 4 | 8.292 | 0.001 |
| PTMS | 5763 | parathymosin | 8.181 | 0.005 |
| CD70 | 970 | CD70 molecule | 8.139 | <0.001 |
| IL1RN | 3557 | interleukin 1 receptor antagonist | 7.715 | <0.001 |
| PGRMC1 | 10857 | progesterone receptor membrane | 7.555 | 0.002 |
| | | component 1 | | |
| FAH | 2184 | fumarylacetoacetate hydrolase | 7.513 | <0.001 |
| TFRC | 7037 | transferrin receptor | 7.114 | <0.001 |
| MRPL46 | 26589 | mitochondrial ribosomal protein L46 | 6.991 | 0.006 |
| BST2 | 684 | bone marrow stromal cell antigen 2 | 6.904 | <0.001 |
| ARL6IP1 | 23204 | ADP ribosylation factor like GTPase | 6.827 | <0.001 |
| | | 6 interacting protein 1 | | |
| HLA-DQB1 | 3119 | major histocompatibility complex, | 6.779 | 0.001 |
| | | class II, DQ beta 1 | | |
| COX17 | 10063 | cytochrome c oxidase copper | 6.680 | 0.002 |
| | | chaperone COX17 | | |
| MZB1 | 51237 | marginal zone B and B1 cell specific | 6.647 | <0.001 |
| | | protein | | |
| CDK1 | 983 | cyclin dependent kinase 1 | 6.615 | 0.007 |
| MCM5 | 4174 | minichromosome maintenance | 6.473 | <0.001 |
| | | complex component 5 | | |
| CD38 | 952 | CD38 molecule | 6.393 | 0.007 |
| HMOX1 | 3162 | heme oxygenase 1 | 6.359 | <0.001 |
| CDK6 | 1021 | cyclin dependent kinase 6 | 6.206 | 0.006 |
| MCM2 | 4171 | minichromosome maintenance | 6.203 | 0.010 |
| | | complex component 2 | | |
| ENO3 | 2027 | enolase 3 | 6.086 | 0.001 |
| PITRM1 | 10531 | pitrilysin metallopeptidase 1 | 6.046 | <0.001 |
| MCM4 | 4173 | minichromosome maintenance | 6.021 | <0.001 |
| | | complex component 4 | | |
| TBL2 | 26608 | transducin beta like 2 | 5.989 | <0.001 |
| CDK5 | 1020 | cyclin dependent kinase 5 | 5.954 | <0.001 |
| DHRS2 | 10202 | dehydrogenase/reductase 2 | 5.867 | 0.001 |
| TOMM34 | 10953 | translocase of outer mitochondrial | 5.836 | 0.007 |
| | | membrane 34 | | |
| ADI1 | 55256 | acireductone dioxygenase 1 | 5.825 | <0.001 |
| SLC25A10 | 1468 | solute carrier family 25 member 10 | 5.776 | <0.001 |
| APOBEC3D | 140564 | apolipoprotein B mRNA editing | 5.672 | 0.001 |
| | | enzyme catalytic subunit 3D | | |
| GK | 2710 | glycerol kinase | 5.635 | <0.001 |
| MCM3 | 4172 | minichromosome maintenance | 5.613 | <0.001 |
| | | complex component 3 | | |
| DHFR | 1719 | dihydrofolate reductase | 5.566 | 0.001 |
| HLA-DRB1 | 3123 | major histocompatibility complex, | 5.489 | <0.001 |
| | | class II, DR beta 1 | | |
| DHCR24 | 1718 | 24-dehydrocholesterol reductase | 5.432 | 0.005 |
| ITGB7 | 3695 | integrin subunit beta 7 | 5.421 | <0.001 |
| MMGT1 | 93380 | membrane magnesium transporter 1 | 5.355 | 0.002 |
| ATOX1 | 475 | antioxidant 1 copper chaperone | 5.355 | 0.007 |
| SELPLG | 6404 | selectin P ligand | 5.317 | 0.019 |
| USP10 | 9100 | ubiquitin specific peptidase 10 | 5.286 | <0.001 |
| CTSH | 1512 | cathepsin H | 5.282 | 0.012 |
| HM13 | 81502 | histocompatibility minor 13 | 5.277 | 0.001 |
| MRPL22 | 29093 | mitochondrial ribosomal protein L22 | 5.268 | 0.007 |
| SPTLC1 | 10558 | serine palmitoyltransferase long chain | 5.205 | 0.001 |
| | | base subunit 1 | | |
| TST | 7263 | thiosulfate sulfurtransferase | 5.170 | <0.001 |
| APOA1 | 335 | apolipoprotein A1 | 5.165 | 0.022 |
| CHP1 | 11261 | calcineurin like EF-hand protein 1 | 5.113 | 0.001 |
| SLC25A4 | 291 | solute carrier family 25 member 4 | 5.089 | 0.030 |
| STMN2 | 11075 | stathmin 2 | 5.041 | 0.023 |
| ATP2A2 | 488 | ATPase sarcoplasmic/endoplasmic | 5.017 | 0.005 |
| | | reticulum Ca2+ transporting 2 | | |
| CTLA4 | 1493 | cytotoxic T-lymphocyte associated | 4.993 | 0.002 |
| | | protein 4 | | |
| CD59 | 966 | CD59 molecule (CD59 blood group) | 4.979 | 0.007 |
| GLA | 2717 | galactosidase alpha | 4.978 | 0.005 |
| PYDC1 | 260434 | pyrin domain containing 1 | 4.931 | 0.049 |
| MYCBP | 26292 | MYC binding protein | 4.922 | 0.005 |
| TNFRSF18 | 8784 | TNF receptor superfamily member 18 | 4.896 | 0.001 |
| IRF4 | 3662 | interferon regulatory factor 4 | 4.896 | 0.003 |
| MTHFD2 | 10797 | methylenetetrahydrofolate | 4.894 | 0.022 |
| | | dehydrogenase (NADP+ dependent) | | |
| | | 2, methenyltetrahydrofolate | | |
| | | cyclohydrolase | | |
| GNA15 | 2769 | G protein subunit alpha 15 | 4.877 | 0.002 |
| CCDC124 | 115098 | coiled-coil domain containing 124 | 4.855 | 0.006 |
| TMEM97 | 27346 | transmembrane protein 97 | 4.848 | 0.000 |
| ACP5 | 54 | acid phosphatase 5, tartrate resistant | 4.822 | 0.014 |
| TIGAR | 57103 | TP53 induced glycolysis regulatory | 4.808 | <0.001 |
| | | phosphatase | | |
| MED20 | 9477 | mediator complex subunit 20 | 4.788 | 0.004 |
| SLC3A2 | 6520 | solute carrier family 3 member 2 | 4.787 | <0.001 |
| ARMC1 | 55156 | armadillo repeat containing 1 | 4.781 | 0.044 |
| MRPL14 | 64928 | mitochondrial ribosomal protein L14 | 4.773 | 0.029 |
| PAIP2 | 51247 | poly(A) binding protein interacting | 4.770 | 0.020 |
| | | protein 2 | | |
| MCM7 | 4176 | minichromosome maintenance | 4.743 | <0.001 |
| | | complex component 7 | | |
| RPL22L1 | 200916 | ribosomal protein L22 like 1 | 4.733 | 0.002 |
| ITPK1 | 3705 | inositol-tetrakisphosphate 1-kinase | 4.723 | 0.002 |
| HLA-DRA | 3122 | major histocompatibility complex, | 4.722 | <0.001 |
| | | class II, DR alpha | | |
| BSG | 682 | basigin (Ok blood group) | 4.721 | 0.001 |
| OCIAD2 | 132299 | OCIA domain containing 2 | 4.678 | <0.001 |
| CHMP6 | 79643 | charged multivesicular body protein 6 | 4.644 | 0.029 |
| RALB | 5899 | RAS like proto-oncogene B | 4.644 | 0.008 |
| MAOA | 4128 | monoamine oxidase A | 4.637 | 0.002 |
| HMBS | 3145 | hydroxymethylbilane synthase | 4.635 | 0.005 |
| SLC25A19 | 60386 | solute carrier family 25 member 19 | 4.627 | 0.001 |
| FOXP3 | 50943 | forkhead box P3 | 4.589 | 0.001 |
| SLC16A1 | 6566 | solute carrier family 16 member 1 | 4.588 | 0.016 |
| ACOT7 | 11332 | acyl-CoA thioesterase 7 | 4.566 | <0.001 |
| RBX1 | 9978 | ring-box 1 | 4.562 | 0.010 |
| DDB2 | 1643 | damage specific DNA binding protein 2 | 4.516 | 0.004 |
| DYNLL1 | 8655 | dynein light chain LC8-type 1 | 4.503 | <0.001 |
| RRAS2 | 22800 | RAS related 2 | 4.486 | 0.006 |
| SMC2 | 10592 | structural maintenance of | 4.463 | 0.002 |
| | | chromosomes 2 | | |
| VAMP8 | 8673 | vesicle associated membrane protein 8 | 4.459 | 0.024 |
| CDK2 | 1017 | cyclin dependent kinase 2 | 4.455 | 0.005 |
| HYPK | 25764 | huntingtin interacting protein K | 4.434 | 0.004 |
| BPGM | 669 | bisphosphoglycerate mutase | 4.434 | 0.005 |
| RBM38 | 55544 | RNA binding motif protein 38 | 4.433 | 0.004 |
| AKR1C3 | 8644 | aldo-keto reductase family 1 member C3 | 4.415 | 0.014 |
| MCM6 | 4175 | minichromosome maintenance | 4.394 | <0.001 |
| | | complex component 6 | | |
| AUH | 549 | AU RNA binding methylglutaconyl- | 4.352 | 0.003 |
| | | CoA hydratase | | |
| SAP30 | 8819 | Sin3A associated protein 30 | 4.350 | 0.016 |
| EMC7 | 56851 | ER membrane protein complex | 4.338 | 0.037 |
| | | subunit 7 | | |
| NRBP1 | 29959 | nuclear receptor binding protein 1 | 4.333 | 0.011 |
| ICOS | 29851 | inducible T cell costimulator | 4.331 | 0.001 |
| HSPH1 | 10808 | heat shock protein family H (Hsp110) | 4.329 | 0.009 |
| | | member 1 | | |
| PPP1R8 | 5511 | protein phosphatase 1 regulatory | 4.321 | 0.042 |
| | | subunit 8 | | |
| TCAF2 | 285966 | TRPM8 channel associated factor 2 | 4.305 | <0.001 |
| DAP3 | 7818 | death associated protein 3 | 4.303 | 0.002 |
| MRPS27 | 23107 | mitochondrial ribosomal protein S27 | 4.293 | 0.003 |
| MRPS14 | 63931 | mitochondrial ribosomal protein S14 | 4.291 | 0.023 |
| CTPS1 | 1503 | CTP synthase 1 | 4.290 | <0.001 |
| LAMP2 | 3920 | lysosomal associated membrane | 4.288 | 0.011 |
| | | protein 2 | | |
| ERI1 | 90459 | exoribonuclease 1 | 4.285 | 0.007 |
| RHEB | 6009 | Ras homolog, mTORC1 binding | 4.259 | 0.006 |
| MAEA | 10296 | macrophage erythroblast attacher | 4.259 | 0.007 |
| MRPL17 | 63875 | mitochondrial ribosomal protein L17 | 4.252 | 0.002 |
| MRPL43 | 84545 | mitochondrial ribosomal protein L43 | 4.246 | 0.030 |
| REXO2 | 25996 | RNA exonuclease 2 | 4.242 | <0.001 |
| DCTN3 | 11258 | dynactin subunit 3 | 4.232 | 0.001 |
| CASP3 | 836 | caspase 3 | 4.228 | 0.017 |
| APOL2 | 23780 | apolipoprotein L2 | 4.176 | 0.001 |
| ACSL4 | 2182 | acyl-CoA synthetase long chain | 4.164 | 0.029 |
| | | family member 4 | | |
| ERMP1 | 79956 | endoplasmic reticulum | 4.149 | 0.025 |
| | | metallopeptidase 1 | | |
| PPME1 | 51400 | protein phosphatase methylesterase 1 | 4.122 | 0.001 |
| IWS1 | 55677 | IWS1, SUPT6H interacting protein | 4.113 | 0.017 |
| BNIP1 | 662 | BCL2 interacting protein 1 | 4.112 | 0.022 |
| PPID | 5481 | peptidylprolyl isomerase D | 4.107 | <0.001 |
| MRPS2 | 51116 | mitochondrial ribosomal protein S2 | 4.105 | 0.023 |
| MAIP1 | 79568 | matrix AAA peptidase interacting | 4.099 | 0.004 |
| | | protein 1 | | |
| RIOX2 | 84864 | ribosomal oxygenase 2 | 4.095 | 0.004 |
| BCL2L1 | 598 | BCL2 like 1 | 4.094 | 0.031 |
| ALDH3A2 | 224 | aldehyde dehydrogenase 3 family | 4.083 | 0.001 |
| | | member A2 | | |
| NAMPT | 10135 | nicotinamide | 4.070 | <0.001 |
| | | phosphoribosyltransferase | | |
| SEC63 | 11231 | SEC63 homolog, protein translocation | 4.052 | 0.010 |
| | | regulator | | |
| UBAP2L | 9898 | ubiquitin associated protein 2 like | 4.024 | <0.001 |
| GCLM | 2730 | glutamate-cysteine ligase modifier | 4.023 | <0.001 |
| | | subunit | | |
| TMEM14C | 51522 | transmembrane protein 14C | 4.016 | 0.013 |
| BCCIP | 56647 | BRCA2 and CDKNIA interacting | 4.012 | <0.001 |
| | | protein | | |
| LGMN | 5641 | legumain | 4.004 | 0.001 |
| RFC5 | 5985 | replication factor C subunit 5 | 4.000 | 0.006 |
| TGFBI | 7045 | transforming growth factor beta | 3.982 | 0.003 |
| | | induced | | |
| APOD | 347 | apolipoprotein D | 3.982 | 0.007 |
| MAD2L1 | 4085 | mitotic arrest deficient 2 like 1 | 3.978 | 0.001 |
| TXN | 7295 | thioredoxin | 3.977 | <0.001 |
| GGH | 8836 | gamma-glutamyl hydrolase | 3.970 | 0.008 |
| HLA-DQA1 | 3117 | major histocompatibility complex, | 3.944 | <0.001 |
| | | class II, DQ alpha 1 | | |
| EIF2B2 | 8892 | eukaryotic translation initiation factor | 3.931 | 0.012 |
| | | 2B subunit beta | | |
| TRABD | 80305 | TraB domain containing | 3.922 | <0.001 |
| GGCT | 79017 | gamma-glutamylcyclotransferase | 3.913 | 0.001 |
| MVD | 4597 | mevalonate diphosphate | 3.908 | 0.037 |
| | | decarboxylase | | |
| LRRC59 | 55379 | leucine rich repeat containing 59 | 3.907 | <0.001 |
| TM9SF3 | 56889 | transmembrane 9 superfamily | 3.905 | 0.007 |
| | | member 3 | | |
| PTRH2 | 51651 | peptidy1-tRNA hydrolase 2 | 3.904 | <0.001 |
| CUL4B | 8450 | cullin 4B | 3.896 | 0.034 |
| ACP2 | 53 | acid phosphatase 2, lysosomal | 3.894 | 0.032 |
| SEC11C | 90701 | SEC11 homolog C, signal peptidase | 3.892 | 0.014 |
| | | complex subunit | | |
| HPGD | 3248 | 15-hydroxyprostaglandin | 3.890 | 0.022 |
| | | dehydrogenase | | |
| ACOT8 | 10005 | acyl-CoA thioesterase 8 | 3.848 | 0.014 |
| CD82 | 3732 | CD82 molecule | 3.842 | 0.034 |
| L2HGDH | 79944 | L-2-hydroxyglutarate dehydrogenase | 3.842 | 0.030 |
| HUWE1 | 10075 | HECT, UBA and WWE domain | 3.829 | 0.005 |
| | | containing 1, E3 ubiquitin protein | | |
| | | ligase | | |
| ARG2 | 384 | arginase 2 | 3.827 | 0.010 |
| SLC29A1 | 2030 | solute carrier family 29 member 1 | 3.825 | 0.048 |
| | | (Augustine blood group) | | |
| SATB1 | 6304 | SATB homeobox 1 | 3.823 | <0.001 |
| FCHO1 | 23149 | FCH domain only 1 | 3.804 | 0.007 |
| MRPL4 | 51073 | mitochondrial ribosomal protein L4 | 3.799 | 0.014 |
| CD28 | 940 | CD28 molecule | 3.789 | 0.009 |
| MRGBP | 55257 | MRG domain binding protein | 3.778 | 0.004 |
| TMA16 | 55319 | translation machinery associated 16 | 3.767 | 0.015 |
| | | homolog | | |
| PPIF | 10105 | peptidylprolyl isomerase F | 3.731 | 0.020 |
| SMS | 6611 | spermine synthase | 3.726 | 0.004 |
| PGP | 283871 | phosphoglycolate phosphatase | 3.718 | 0.001 |
| WARS | 7453 | tryptophanyl-tRNA synthetase | 3.715 | <0.001 |
| CPOX | 1371 | coproporphyrinogen oxidase | 3.711 | <0.001 |
| SCPEP1 | 59342 | serine carboxypeptidase 1 | 3.689 | 0.020 |
| MFSD10 | 10227 | major facilitator superfamily domain | 3.684 | 0.019 |
| | | containing 10 | | |
| MCMBP | 79892 | minichromosome maintenance | 3.680 | 0.013 |
| | | complex binding protein | | |
| GBE1 | 2632 | 1,4-alpha-glucan branching enzyme 1 | 3.672 | 0.012 |
| RFC3 | 5983 | replication factor C subunit 3 | 3.663 | 0.012 |
| TRUB1 | 142940 | TruB pseudouridine synthase family | 3.653 | 0.047 |
| | | member 1 | | |
| BAG6 | 7917 | BCL2 associated athanogene 6 | 3.651 | 0.026 |
| MRPL48 | 51642 | mitochondrial ribosomal protein L48 | 3.649 | 0.007 |
| MRPS11 | 64963 | mitochondrial ribosomal protein S11 | 3.623 | <0.001 |
| RSU1 | 6251 | Ras suppressor protein 1 | 3.606 | 0.001 |
| THOC6 | 79228 | THO complex 6 | 3.593 | 0.004 |
| GTF3C3 | 9330 | general transcription factor IIIC | 3.571 | 0.028 |
| | | subunit 3 | | |
| MRPL44 | 65080 | mitochondrial ribosomal protein L44 | 3.569 | 0.003 |
| NMI | 9111 | N-myc and STAT interactor | 3.565 | 0.008 |
| LIG1 | 3978 | DNA ligase 1 | 3.564 | 0.001 |
| RFC4 | 5984 | replication factor C subunit 4 | 3.546 | <0.001 |
| MANF | 7873 | mesencephalic astrocyte derived | 3.543 | <0.001 |
| | | neurotrophic factor | | |
| CELF1 | 10658 | CUGBP Elav-like family member 1 | 3.543 | 0.048 |
| ACY1 | 95 | aminoacylase 1 | 3.530 | 0.003 |
| MRPS31 | 10240 | mitochondrial ribosomal protein S31 | 3.520 | 0.039 |
| EIF4E2 | 9470 | eukaryotic translation initiation factor | 3.502 | 0.013 |
| | | 4E family member 2 | | |
| POLD2 | 5425 | DNA polymerase delta 2, accessory | 3.491 | 0.003 |
| | | subunit | | |
| FASN | 2194 | fatty acid synthase | 3.483 | <0.001 |
| NADSYN1 | 55191 | NAD synthetase 1 | 3.462 | 0.010 |
| KPNA2 | 3838 | karyopherin subunit alpha 2 | 3.456 | 0.006 |
| RNASEH2A | 10535 | ribonuclease H2 subunit A | 3.447 | 0.037 |
| HAT1 | 8520 | histone acetyltransferase 1 | 3.441 | <0.001 |
| STAT1 | 6772 | signal transducer and activator of | 3.425 | <0.001 |
| | | transcription 1 | | |
| UAPIL1 | 91373 | UDP-N-acetylglucosamine | 3.401 | 0.007 |
| | | pyrophosphorylase 1 like 1 | | |
| PYCR2 | 29920 | pyrroline-5-carboxylate reductase 2 | 3.399 | 0.021 |
| PLEKHA2 | 59339 | pleckstrin homology domain | 3.397 | 0.020 |
| | | containing A2 | | |
| NCF4 | 4689 | neutrophil cytosolic factor 4 | 3.388 | <0.001 |
| RNF213 | 57674 | ring finger protein 213 | 3.383 | <0.001 |
| MAN1A1 | 4121 | mannosidase alpha class 1A member 1 | 3.356 | 0.018 |
| POFUT1 | 23509 | protein O-fucosyltransferase 1 | 3.347 | 0.001 |
| CSDE1 | 7812 | cold shock domain containing E1 | 3.347 | 0.025 |
| IDE | 3416 | insulin degrading enzyme | 3.324 | 0.003 |
| HELLS | 3070 | helicase, lymphoid specific | 3.317 | 0.037 |
| ATXN2L | 11273 | ataxin 2 like | 3.315 | 0.004 |
| CALU | 813 | calumenin | 3.310 | 0.048 |
| KTN1 | 3895 | kinectin 1 | 3.306 | <0.001 |
| FAS | 355 | Fas cell surface death receptor | 3.302 | <0.001 |
| TBCD | 6904 | tubulin folding cofactor D | 3.300 | 0.016 |
| JPT1 | 51155 | Jupiter microtubule associated | 3.295 | 0.004 |
| | | homolog 1 | | |
| OAT | 4942 | ornithine aminotransferase | 3.292 | 0.010 |
| BRK1 | 55845 | BRICK1, SCAR/WAVE actin | 3.292 | 0.022 |
| | | nucleating complex subunit | | |
| TAOK3 | 51347 | TAO kinase 3 | 3.285 | 0.026 |
| MSI2 | 124540 | musashi RNA binding protein 2 | 3.285 | 0.030 |
| VPS28 | 51160 | VPS28, ESCRT-I subunit | 3.279 | 0.041 |
| MRPL12 | 6182 | mitochondrial ribosomal protein L12 | 3.274 | 0.009 |
| LACTB2 | 51110 | lactamase beta 2 | 3.255 | 0.012 |
| SFXN2 | 118980 | sideroflexin 2 | 3.240 | 0.032 |
| FAM160B1 | 57700 | family with sequence similarity 160 | 3.240 | 0.001 |
| | | member B1 | | |
| EXOSC5 | 56915 | exosome component 5 | 3.237 | 0.011 |
| HMGB3 | 3149 | high mobility group box 3 | 3.236 | 0.002 |
| ZC2HC1A | 51101 | zinc finger C2HC-type containing 1A | 3.233 | 0.008 |
| PLSCR3 | 57048 | phospholipid scramblase 3 | 3.226 | 0.005 |
| DTD1 | 92675 | D-tyrosyl-tRNA deacylase 1 | 3.198 | 0.034 |
| NAPG | 8774 | NSF attachment protein gamma | 3.185 | <0.001 |
| LSM1 | 27257 | LSM1 homolog, mRNA degradation | 3.172 | 0.018 |
| | | associated | | |
| TIMM13 | 26517 | translocase of inner mitochondrial | 3.159 | 0.017 |
| | | membrane 13 | | |
| S1PR4 | 8698 | sphingosine-1-phosphate receptor 4 | 3.145 | 0.022 |
| IPO9 | 55705 | importin 9 | 3.122 | 0.029 |
| MFSD1 | 64747 | major facilitator superfamily domain | 3.077 | 0.027 |
| | | containing 1 | | |
| MSH2 | 4436 | mutS homolog 2 | 3.075 | 0.020 |
| CPT1A | 1374 | carnitine palmitoyltransferase 1A | 3.069 | <0.001 |
| FAM192A | 80011 | family with sequence similarity 192 | 3.055 | 0.023 |
| | | member A | | |
| AP3D1 | 8943 | adaptor related protein complex 3 | 3.047 | 0.037 |
| | | subunit delta 1 | | |
| RCL1 | 10171 | RNA terminal phosphate cyclase like 1 | 3.042 | 0.019 |
| PTGES3L- | 1E+08 | PTGES3L-AARSD1 readthrough | 3.038 | 0.023 |
| AARSD1 | | | | |
| GBP5 | 115362 | guanylate binding protein 5 | 3.034 | <0.001 |
| MRPL13 | 28998 | mitochondrial ribosomal protein L13 | 3.030 | <0.001 |
| NPM3 | 10360 | nucleophosmin/nucleoplasmin 3 | 3.030 | 0.019 |
| PPP2R5D | 5528 | protein phosphatase 2 regulatory | 3.022 | 0.003 |
| | | subunit B'delta | | |
| CYB5B | 80777 | cytochrome b5 type B | 3.021 | 0.043 |
| MRPS35 | 60488 | mitochondrial ribosomal protein S35 | 3.003 | 0.040 |
| POLD1 | 5424 | DNA polymerase delta 1, catalytic | 2.998 | 0.001 |
| | | subunit | | |
| AGMAT | 79814 | agmatinase | 2.994 | 0.007 |
| PTDSS1 | 9791 | phosphatidylserine synthase 1 | 2.977 | 0.036 |
| IPO7 | 10527 | importin 7 | 2.974 | 0.014 |
| ARL3 | 403 | ADP ribosylation factor like GTPase 3 | 2.973 | <0.001 |
| MRPS9 | 64965 | mitochondrial ribosomal protein S9 | 2.970 | 0.033 |
| PBXIP1 | 57326 | PBX homeobox interacting protein 1 | 2.957 | 0.001 |
| SLC16A3 | 9123 | solute carrier family 16 member 3 | 2.954 | 0.000 |
| EIF2B3 | 8891 | eukaryotic translation initiation factor | 2.954 | 0.022 |
| | | 2B subunit gamma | | |
| NUDT1 | 4521 | nudix hydrolase 1 | 2.947 | <0.001 |
| WDR61 | 80349 | WD repeat domain 61 | 2.944 | 0.041 |
| MPST | 4357 | mercaptopyruvate sulfurtransferase | 2.938 | <0.001 |
| ASF1A | 25842 | anti-silencing function 1A histone | 2.937 | 0.001 |
| | | chaperone | | |
| HTRA2 | 27429 | HtrA serine peptidase 2 | 2.929 | 0.017 |
| SLC2A3 | 6515 | solute carrier family 2 member 3 | 2.923 | 0.042 |
| HSPB1 | 3315 | heat shock protein family B (small) | 2.921 | 0.004 |
| | | member 1 | | |
| LPXN | 9404 | leupaxin | 2.904 | 0.001 |
| GLRX3 | 10539 | glutaredoxin 3 | 2.885 | <0.001 |
| GCLC | 2729 | glutamate-cysteine ligase catalytic | 2.881 | 0.009 |
| | | subunit | | |
| TF | 7018 | transferrin | 2.873 | 0.001 |
| CARM1 | 10498 | coactivator associated arginine | 2.872 | 0.005 |
| | | methyltransferase 1 | | |
| RNASEH2B | 79621 | ribonuclease H2 subunit B | 2.858 | <0.001 |
| AIMP2 | 7965 | aminoacyl tRNA synthetase complex | 2.852 | 0.003 |
| | | interacting multifunctional protein 2 | | |
| TOMM40 | 10452 | translocase of outer mitochondrial | 2.848 | 0.005 |
| | | membrane 40 | | |
| EMC2 | 9694 | ER membrane protein complex | 2.828 | 0.013 |
| | | subunit 2 | | |
| CD2 | 914 | CD2 molecule | 2.826 | 0.001 |
| UROD | 7389 | uroporphyrinogen decarboxylase | 2.825 | 0.007 |
| ADAM10 | 102 | ADAM metallopeptidase domain 10 | 2.816 | 0.012 |
| HTATIP2 | 10553 | HIV-1 Tat interactive protein 2 | 2.804 | <0.001 |
| MTX1 | 4580 | metaxin 1 | 2.803 | 0.014 |
| RPL7L1 | 285855 | ribosomal protein L7 like 1 | 2.779 | 0.038 |
| ERBIN | 55914 | erbb2 interacting protein | 2.778 | 0.001 |
| CASP6 | 839 | caspase 6 | 2.778 | 0.001 |
| MRPL37 | 51253 | mitochondrial ribosomal protein L37 | 2.762 | 0.002 |
| EIF4G1 | 1981 | eukaryotic translation initiation factor | 2.759 | 0.004 |
| | | 4 gamma 1 | | |
| CACYBP | 27101 | calcyclin binding protein | 2.754 | <0.001 |
| DNAJB11 | 51726 | DnaJ heat shock protein family | 2.750 | 0.001 |
| | | (Hsp40) member B11 | | |
| TRAF1 | 7185 | TNF receptor associated factor 1 | 2.735 | 0.017 |
| RRP1B | 23076 | ribosomal RNA processing 1B | 2.734 | 0.023 |
| RFC2 | 5982 | replication factor C subunit 2 | 2.731 | 0.001 |
| PPP2R5C | 5527 | protein phosphatase 2 regulatory | 2.696 | 0.004 |
| | | subunit B'gamma | | |
| RAB1A | 5861 | RAB1A, member RAS oncogene | 2.680 | 0.001 |
| | | family | | |
| IFI35 | 3430 | interferon induced protein 35 | 2.675 | 0.002 |
| RFTN1 | 23180 | raftlin, lipid raft linker 1 | 2.675 | 0.038 |
| RMND1 | 55005 | required for meiotic nuclear division 1 | 2.669 | 0.019 |
| | | homolog | | |
| RRM1 | 6240 | ribonucleotide reductase catalytic | 2.657 | 0.010 |
| | | subunit M1 | | |
| TBL1XR1 | 79718 | transducin beta like 1 X-linked | 2.649 | 0.017 |
| | | receptor 1 | | |
| RAP2B | 5912 | RAP2B, member of RAS oncogene | 2.647 | 0.017 |
| | | family | | |
| CORO1C | 23603 | coronin 1C | 2.643 | 0.003 |
| DPP4 | 1803 | dipeptidyl peptidase 4 | 2.628 | 0.001 |
| CD74 | 972 | CD74 molecule | 2.625 | <0.001 |
| PCNA | 5111 | proliferating cell nuclear antigen | 2.615 | <0.001 |
| TXNDC5 | 81567 | thioredoxin domain containing 5 | 2.612 | 0.001 |
| MRPL39 | 54148 | mitochondrial ribosomal protein L39 | 2.610 | 0.038 |
| B4GALT5 | 9334 | beta-1,4-galactosyltransferase 5 | 2.582 | 0.028 |
| FIBP | 9158 | FGF1 intracellular binding protein | 2.575 | 0.003 |
| ACADVL | 37 | acyl-CoA dehydrogenase very long | 2.567 | <0.001 |
| | | chain | | |
| POLDIP3 | 84271 | DNA polymerase delta interacting | 2.561 | 0.024 |
| | | protein 3 | | |
| LGALS3 | 3958 | galectin 3 | 2.523 | <0.001 |
| KIF5B | 3799 | kinesin family member 5B | 2.519 | <0.001 |
| ACAA1 | 30 | acetyl-CoA acyltransferase 1 | 2.519 | 0.006 |
| ATXN10 | 25814 | ataxin 10 | 2.517 | 0.003 |
| GMDS | 2762 | GDP-mannose 4,6-dehydratase | 2.514 | 0.001 |
| ATP13A1 | 57130 | ATPase 13A1 | 2.506 | 0.042 |
| RBPJ | 3516 | recombination signal binding protein | 2.481 | 0.003 |
| | | for immunoglobulin kappa J region | | |
| ECI1 | 1632 | enoyl-CoA delta isomerase 1 | 2.477 | 0.012 |
| ACSL5 | 51703 | acyl-CoA synthetase long chain | 2.457 | 0.003 |
| | | family member 5 | | |
| BCLAF1 | 9774 | BCL2 associated transcription factor 1 | 2.455 | 0.032 |
| EIF1 | 10209 | eukaryotic translation initiation factor 1 | 2.454 | 0.037 |
| DUT | 1854 | deoxyuridine triphosphatase | 2.436 | <0.001 |
| STMN1 | 3925 | stathmin 1 | 2.411 | <0.001 |
| MTHFD1 | 4522 | methylenetetrahydrofolate | 2.410 | <0.001 |
| | | dehydrogenase, cyclohydrolase and | | |
| | | formyltetrahydrofolate synthetase 1 | | |
| ADPRH | 141 | ADP-ribosylarginine hydrolase | 2.387 | <0.001 |
| CD84 | 8832 | CD84 molecule | 2.379 | 0.043 |
| TRMT112 | 51504 | tRNA methyltransferase subunit 11-2 | 2.378 | 0.031 |
| BYSL | 705 | bystin like | 2.375 | 0.040 |
| SNAP23 | 8773 | synaptosome associated protein 23 | 2.371 | 0.013 |
| ICMT | 23463 | isoprenylcysteine carboxyl | 2.357 | 0.010 |
| | | methyltransferase | | |
| ZC3H12D | 340152 | zinc finger CCCH-type containing | 2.357 | 0.007 |
| | | 12D | | |
| IPO5 | 3843 | importin 5 | 2.350 | <0.001 |
| ACSL1 | 2180 | acyl-CoA synthetase long chain | 2.348 | 0.002 |
| | | family member 1 | | |
| TUBAL3 | 79861 | tubulin alpha like 3 | 2.336 | <0.001 |
| GET4 | 51608 | golgi to ER traffic protein 4 | 2.336 | 0.033 |
| LCP2 | 3937 | lymphocyte cytosolic protein 2 | 2.314 | 0.040 |
| TUBG1 | 7283 | tubulin gamma 1 | 2.304 | 0.001 |
| MRI1 | 84245 | methylthioribose-1-phosphate | 2.300 | 0.001 |
| | | isomerase 1 | | |
| TXNRD1 | 7296 | thioredoxin reductase 1 | 2.299 | 0.000 |
| SERPINH1 | 871 | serpin family H member 1 | 2.288 | 0.042 |
| TTC1 | 7265 | tetratricopeptide repeat domain 1 | 2.287 | 0.021 |
| ERG28 | 11161 | ergosterol biosynthesis 28 homolog | 2.280 | 0.042 |
| LIMS1 | 3987 | LIM zinc finger domain containing 1 | 2.270 | 0.003 |
| ARL2 | 402 | ADP ribosylation factor like GTPase 2 | 2.250 | 0.020 |
| YBX1 | 4904 | Y-box binding protein 1 | 2.249 | 0.013 |
| FEN1 | 2237 | flap structure-specific endonuclease 1 | 2.245 | 0.000 |
| NCSTN | 23385 | nicastrin | 2.234 | 0.001 |
| AGK | 55750 | acylglycerol kinase | 2.232 | 0.001 |
| RNMT | 8731 | RNA guanine-7 methyltransferase | 2.230 | 0.002 |
| OTULIN | 90268 | OTU deubiquitinase with linear | 2.226 | 0.007 |
| | | linkage specificity | | |
| NDUFA8 | 4702 | NADH:ubiquinone oxidoreductase | 2.224 | 0.004 |
| | | subunit A8 | | |
| TUBA1B | 10376 | tubulin alpha 1b | 2.224 | 0.001 |
| RMDN1 | 51115 | regulator of microtubule dynamics 1 | 2.219 | <0.001 |
| ACAA2 | 10449 | acetyl-CoA acyltransferase 2 | 2.217 | <0.001 |
| TMCO1 | 54499 | transmembrane and coiled-coil | 2.204 | <0.001 |
| | | domains 1 | | |
| LRPPRC | 10128 | leucine rich pentatricopeptide repeat | 2.193 | <0.001 |
| | | containing | | |
| DTYMK | 1841 | deoxythymidylate kinase | 2.186 | <0.001 |
| FDXR | 2232 | ferredoxin reductase | 2.169 | 0.006 |
| IGBP1 | 3476 | immunoglobulin binding protein 1 | 2.169 | 0.001 |
| FAHD2A | 51011 | fumarylacetoacetate hydrolase domain | 2.146 | 0.029 |
| | | containing 2A | | |
| HTATSF1 | 27336 | HIV-1 Tat specific factor 1 | 2.143 | 0.001 |
| GRSF1 | 2926 | G-rich RNA sequence binding factor 1 | 2.142 | 0.031 |
| DNMIL | 10059 | dynamin 1 like | 2.141 | 0.010 |
| AP3M1 | 26985 | adaptor related protein complex 3 | 2.133 | 0.005 |
| | | subunit mu 1 | | |
| UBE2L6 | 9246 | ubiquitin conjugating enzyme E2 L6 | 2.129 | 0.001 |
| CISD2 | 493856 | CDGSH iron sulfur domain 2 | 2.128 | 0.002 |
| HSPBP1 | 23640 | HSPA (Hsp70) binding protein 1 | 2.127 | 0.001 |
| MRPL1 | 65008 | mitochondrial ribosomal protein L1 | 2.126 | <0.001 |
| PMPCA | 23203 | peptidase, mitochondrial processing | 2.125 | 0.025 |
| | | alpha subunit | | |
| ACADM | 34 | acyl-CoA dehydrogenase medium | 2.116 | 0.001 |
| | | chain | | |
| BDH1 | 622 | 3-hydroxybutyrate dehydrogenase 1 | 2.108 | 0.002 |
| DCAF8 | 50717 | DDB1 and CUL4 associated factor 8 | 2.108 | 0.018 |
| STRAP | 11171 | serine/threonine kinase receptor | 2.108 | <0.001 |
| | | associated protein | | |
| TIMM23 | 1E+08 | translocase of inner mitochondrial | 2.107 | 0.027 |
| | | membrane 23 | | |
| FKBP3 | 2287 | FKBP prolyl isomerase 3 | 2.105 | <0.001 |
| SEC61A1 | 29927 | Sec61 translocon alpha 1 subunit | 2.097 | 0.012 |
| TM9SF2 | 9375 | transmembrane 9 superfamily | 2.096 | 0.011 |
| | | member 2 | | |
| TMEM65 | 157378 | transmembrane protein 65 | 2.089 | 0.002 |
| ENOPH1 | 58478 | enolase-phosphatase 1 | 2.087 | 0.026 |
| CPT2 | 1376 | carnitine palmitoyltransferase 2 | 2.079 | 0.019 |
| CIAPIN1 | 57019 | cytokine induced apoptosis inhibitor 1 | 2.074 | 0.003 |
| IDI1 | 3422 | isopentenyl-diphosphate delta | 2.065 | <0.001 |
| | | isomerase 1 | | |
| POLDIP2 | 26073 | DNA polymerase delta interacting | 2.050 | 0.011 |
| | | protein 2 | | |
| SUMF2 | 25870 | sulfatase modifying factor 2 | 2.046 | <0.001 |
| NUDC | 10726 | nuclear distribution C, dynein | 2.046 | <0.001 |
| | | complex regulator | | |
| PKN1 | 5585 | protein kinase N1 | 2.044 | 0.013 |
| NAA50 | 80218 | N(alpha)-acetyltransferase 50, NatE | 2.037 | 0.010 |
| | | catalytic subunit | | |
| CHDH | 55349 | choline dehydrogenase | 2.029 | 0.029 |
| PPP4R3A | 55671 | protein phosphatase 4 regulatory | 2.025 | 0.022 |
| | | subunit 3A | | |
| KRR1 | 11103 | KRR1, small subunit processome | 2.023 | 0.020 |
| | | component homolog | | |
| TOMM22 | 56993 | translocase of outer mitochondrial | 2.022 | 0.002 |
| | | membrane 22 | | |
| FKBP8 | 23770 | FKBP prolyl isomerase 8 | 2.014 | 0.001 |
| LGALS1 | 3956 | galectin 1 | 2.014 | <0.001 |
| AIMP1 | 9255 | aminoacyl tRNA synthetase complex | 2.012 | 0.002 |
| | | interacting multifunctional protein 1 | | |

6.2.4.3 Enhanced Treg proteomic signatures following expansion.

The phenotypic analysis reveals that the expanded Treg gene product signature includes gene products from prominent pathways that are associated with functional processes, specifically pathways enriched in Treg immune signatures, mitochondria activation and energetics, and cellular proliferation including cell division, cell cycle, and DNA replication/repair. The proteomics data has also been stratified to present the highest expression signatures found in the patient Tregs following expansion. Each of these gene product signatures of the expanded Treg cell population is described below.

6.2.4.3.1 Treg-associated gene product signature.

The proteomic analysis reveals a number of gene products involved in immunological pathways that are enriched in the expanded Treg cell populations, as evidenced by their increased expression relative to that observed in baseline Tregs. These pathways include, for example, adaptive immune pathways (p=0.00726), innate immune pathways (p=0.09), cytokine signaling in the immune system (p=0.0338), MHC class II antigen presentation (p=9.33e-13), PD-1 signaling (p=7.66e-11), costimulation by the CD28 family (p=9.12e-11), generation of second messenger molecules (p=7.69e-13), Interferon signaling (p=1.31e-7), downstream TCR signaling (p=1.31e-7), and RUNX1 and FOXP3 control of the development of regulatory T lymphocytes (p=1.05e-3). Table 6 (Treg-associated gene product signature) lists gene products whose expression is increased relative to baseline Tregs, wherein the gene products are documented in the literature as being important to the proliferation, health, identification, and/or mechanism of Treg cells.

As shown in Table 6, the Treg-associated gene product signature includes, for example: ADAM10, AIMP1, AIMP2, ARG2, BCL2L1, BSG, CD2, CD28, CD38, CD74, CD84, CTLA4, FAS, FOXP3, GCLC, HAT1, HLA-DQA1, HLA-DQB1, HLA-DRA, HLA-DRB1, HPGD, ICOS, ILIRN, IRF4, KPNA2, LGALS1, LGMN, PCNA, POFUTI, SATB1, SELPLG, STAT1, TFRC, and TNFRSF18. PMID: Pubmed ID.

TABLE 6
Gene products in expanded Tregs that are documented in the literature as being important
to the proliferation, health, identification, and/or mechanism of Treg cells.

| | | | | log2 of | | log2 of | |
|---|---|---|---|---|---|---|---|
| | NCBI | | PMID | fold-change | | fold-change | |
| Gene | Gene | | cross | of baseline | Adjusted | of expanded | Adjusted |
| Symbol | ID | Gene Description | reference | vs. expanded | p-value | vs. freeze-thaw | p-value |
| IL1RN | 3557 | interleukin 1 receptor | 24770649 | 7.715 | <0.001 | 0.218 | 0.911 |
| | | antagonist | | | | | |
| TFRC | 7037 | transferrin receptor | 29311383 | 7.114 | <0.001 | 0.261 | 0.902 |
| HLA- | 3119 | major histocompatibility | 16585553 | 6.779 | 0.001 | 0.367 | 0.942 |
| DQB1 | | complex, class II, DQ beta 1 | | | | | |
| CD38 | 952 | CD38 molecule | 28249894 | 6.393 | 0.007 | −0.897 | 0.873 |
| HLA- | 3123 | major histocompatibility | 16585553 | 5.489 | <0.001 | 0.347 | 0.685 |
| DRB1 | | complex, class II, DR beta 1 | | | | | |
| SELPLG | 6404 | selectin P ligand | 24174617 | 5.317 | 0.019 | −1.004 | 0.848 |
| CTLA4 | 1493 | cytotoxic T-lymphocyte | 23849743 | 4.993 | 0.002 | −0.610 | 0.856 |
| | | associated protein 4 | | | | | |
| TNFRSF18 | 8784 | TNF receptor superfamily | 25961057 | 4.896 | 0.001 | −0.185 | 0.964 |
| | | member 18 | | | | | |
| IRF4 | 3662 | interferon regulatory factor | 32125291 | 4.896 | 0.003 | 0.478 | 0.907 |
| | | 4 | | | | | |
| HLA-DRA | 3122 | major histocompatibility | 16585553 | 4.722 | 0.000 | 0.353 | 0.793 |
| | | complex, class II, DR alpha | | | | | |
| BSG | 682 | basigin (Ok blood group) | 21937704 | 4.721 | 0.001 | 0.042 | 0.993 |
| FOXP3 | 50943 | forkhead box P3 | 25683611 | 4.589 | 0.001 | 0.522 | 0.946 |
| ICOS | 29851 | inducible T cell | 32983168 | 4.331 | 0.001 | 0.629 | 0.798 |
| | | costimulator | | | | | |
| BCL2L1 | 598 | BCL2 like 1 | 31068951 | 4.094 | 0.031 | −0.344 | 0.954 |
| LGMN | 5641 | legumain | 19453521 | 4.004 | 0.001 | 0.528 | 0.819 |
| HLA- | 3117 | major histocompatibility | 16585553 | 3.944 | <0.001 | 0.215 | 0.932 |
| DQA1 | | complex, class II, DQ alpha | | | | | |
| | | 1 | | | | | |
| HPGD | 3248 | 15-hydroxyprostaglandin | 31027998 | 3.890 | 0.022 | −0.067 | 0.993 |
| | | dehydrogenase | | | | | |
| ARG2 | 384 | arginase 2 | 31852848 | 3.827 | 0.010 | 1.012 | 0.722 |
| SATB1 | 6304 | SATB homeobox 1 | 27992401 | 3.823 | <0.001 | 0.567 | 0.739 |
| CD28 | 940 | CD28 molecule | 18684917 | 3.789 | 0.009 | 0.770 | 0.805 |
| KPNA2 | 3838 | karyopherin subunit alpha 2 | 31597697 | 3.456 | 0.006 | 0.657 | 0.804 |
| HAT1 | 8520 | histone acetyltransferase 1 | 24315995 | 3.441 | <0.001 | −0.868 | 0.279 |
| STAT1 | 6772 | signal transducer and | 19337996 | 3.425 | <0.001 | −0.104 | 0.882 |
| | | activator of transcription 1 | | | | | |
| POFUT1 | 23509 | protein O-fucosyltransferase | 26437242 | 3.347 | 0.001 | 0.941 | 0.543 |
| | | 1 | | | | | |
| FAS | 355 | Fas cell surface death | 32294156 | 3.302 | <0.001 | 0.368 | 0.543 |
| | | receptor | | | | | |
| GCLC | 2729 | glutamate-cysteine ligase | 32213345 | 2.881 | 0.009 | −1.135 | 0.560 |
| | | catalytic subunit | | | | | |
| AIMP2 | 7965 | aminoacyl tRNA synthetase | 32709848 | 2.852 | 0.003 | −0.485 | 0.805 |
| | | complex interacting | | | | | |
| | | multifunctional protein 2 | | | | | |
| CD2 | 914 | CD2 molecule | 22539784 | 2.826 | 0.001 | 1.083 | 0.345 |
| ADAM10 | 102 | ADAM metallopeptidase | 31269441 | 2.816 | 0.012 | 0.725 | 0.743 |
| | | domain 10 | | | | | |
| CD74 | 972 | CD74 molecule | 27760760 | 2.625 | <0.001 | 0.366 | 0.357 |
| PCNA | 5111 | proliferating cell nuclear | 29166588 | 2.615 | <0.001 | 0.183 | 0.655 |
| | | antigen | | | | | |
| CD84 | 8832 | CD84 molecule | 26371251 | 2.379 | 0.043 | 0.538 | 0.848 |
| LGALS1 | 3956 | galectin 1 | 16836768 | 2.014 | <0.001 | 0.096 | 0.877 |
| AIMP1 | 9255 | aminoacyl tRNA synthetase | 31084930 | 2.012 | 0.002 | 0.123 | 0.946 |
| | | complex interacting | | | | | |
| | | multifunctional protein 1 | | | | | |

6.2.4.3.2 Mitochondria gene product signature.

Mitochondria play a large role in Treg health and function. The proteomic study revealed a large, enriched gene product signature of mitochondria-related genes in the ex vivo-expanded Treg cell populations whose expression is increased relative to that seen in baseline Tregs (p=2.96e-29). See Table 7. The literature describes the importance of mitochondrial fitness and energetics in Treg function and mitochondrial dysfunction inevitably leads to Treg dysfunction (ex. PMID: 30320604). Targeting and restoring mitochondrial function is currently looked at as a way to revive dysfunctional Tregs (PMID: 30473188). This mitochondria gene product signature is, for example, highly enriched with pathways involved in mitochondria replication (p=1.12e-14) and mitochondrial energy metabolism (p=1.83e-2). This gene product signature indicates that mitochondrial activation, function, and restoration is an important product of the Treg expansion processes described herein.

As shown in Table 7, the mitochondria gene product signature includes, for example: ACAA2, ACADM, ACADVL, ACOT7, ACSL1, ACSL4, ACSL5, AGK, AGMAT, AK4, ARG2, ARL2, AUH, BCL2L1, BDH1, BNIP1, CDK1, CHDH, CIAPINI, CISD2, COX17, CPOX, CPTIA, CPT2, CYB5B, DAP3, DHRS2, DNMIL, DUT, DYNLLI, ECI1, FDXR, FEN1, FKBP8, GK, GRSF1, HTRA2, L2HGDH, LACTB2, LRPPRC, MAIP1, MAOA, MPST, MRPL1, MRPL12, MRPL13, MRPL14, MRPL17, MRPL22, MRPL37, MRPL39, MRPL4, MRPL43, MRPL44, MRPL46, MRPL48, MRPS11, MRPS14, MRPS2, MRPS27, MRPS31, MRPS35, MRPS9, MTHFD2, MTX1, MYCBP, NDUFA8, NUDTI, OAT, PITRMI, PLSCR3, PMPCA, PPIF, PTRH2, PYCR2, REXO2, RMNDI, SFXN2, SLC25A10, SLC25A19, SLC25A4, TIGAR, TIMM13, TIMM23, TMEM14C, TOMM22, TOMM34, TOMM40, and TST.

TABLE 6
Enriched signature of mitochondria-related genes products

| | | | log2 of | | log2 of | |
|---|---|---|---|---|---|---|
| | | | fold-change | | fold-change | |
| | NCBI | | of baseline | Adjusted | of expanded | Adjusted |
| GeneSymbol | GeneID | GeneDescription | vs. expanded | p-value | vs. freeze-thaw | p-value |
| MRPL46 | 26589 | mitochondrial ribosomal protein | 6.991 | 0.006 | −0.041 | 0.997 |
| | | L46 | | | | |
| COX17 | 10063 | cytochrome c oxidase copper | 6.680 | 0.002 | 0.654 | 0.897 |
| | | chaperone COX17 | | | | |
| CDK1 | 983 | cyclin dependent kinase 1 | 6.615 | 0.007 | 1.621 | 0.502 |
| PITRM1 | 10531 | pitrilysin metallopeptidase 1 | 6.046 | <0.001 | 0.611 | 0.805 |
| DHRS2 | 10202 | dehydrogenase/reductase 2 | 5.867 | 0.001 | 0.562 | 0.893 |
| TOMM34 | 10953 | translocase of outer mitochondrial | 5.836 | 0.007 | −0.161 | 0.984 |
| | | membrane 34 | | | | |
| SLC25A10 | 1468 | solute carrier family 25 member 10 | 5.776 | <0.001 | 0.960 | 0.568 |
| GK | 2710 | glycerol kinase | 5.635 | <0.001 | 0.636 | 0.826 |
| MRPL22 | 29093 | mitochondrial ribosomal protein | 5.268 | 0.007 | 0.605 | 0.904 |
| | | L22 | | | | |
| TST | 7263 | thiosulfate sulfurtransferase | 5.170 | <0.001 | 1.050 | 0.566 |
| SLC25A4 | 291 | solute carrier family 25 member 4 | 5.089 | 0.030 | 0.730 | 0.903 |
| MYCBP | 26292 | MYC binding protein | 4.922 | 0.005 | −1.834 | 0.526 |
| MTHFD2 | 10797 | methylenetetrahydrofolate | 4.894 | 0.022 | 0.301 | 0.969 |
| | | dehydrogenase (NADP+ | | | | |
| | | dependent) 2, | | | | |
| | | methenyltetrahydrofolate | | | | |
| | | cyclohydrolase | | | | |
| TIGAR | 57103 | TP53 induced glycolysis regulatory | 4.808 | <0.001 | −0.569 | 0.819 |
| | | phosphatase | | | | |
| MRPL14 | 64928 | mitochondrial ribosomal protein | 4.773 | 0.029 | −0.961 | 0.855 |
| | | L14 | | | | |
| MAOA | 4128 | monoamine oxidase A | 4.637 | 0.002 | 0.210 | 0.960 |
| SLC25A19 | 60386 | solute carrier family 25 member 19 | 4.627 | 0.001 | 0.924 | 0.703 |
| ACOT7 | 11332 | acyl-CoA thioesterase 7 | 4.566 | <0.001 | −0.075 | 0.973 |
| DYNLL1 | 8655 | dynein light chain LC8-type 1 | 4.503 | <0.001 | 0.610 | 0.712 |
| AUH | 549 | AU RNA binding | 4.352 | 0.003 | 0.422 | 0.908 |
| | | methylglutaconyl-CoA hydratase | | | | |
| DAP3 | 7818 | death associated protein 3 | 4.303 | 0.002 | 1.091 | 0.655 |
| MRPS27 | 23107 | mitochondrial ribosomal protein | 4.293 | 0.003 | 0.400 | 0.908 |
| | | S27 | | | | |
| MRPS14 | 63931 | mitochondrial ribosomal protein | 4.291 | 0.023 | 1.703 | 0.645 |
| | | S14 | | | | |
| MRPL17 | 63875 | mitochondrial ribosomal protein | 4.252 | 0.002 | 0.756 | 0.769 |
| | | L17 | | | | |
| MRPL43 | 84545 | mitochondrial ribosomal protein | 4.246 | 0.030 | 0.905 | 0.847 |
| | | L43 | | | | |
| REXO2 | 25996 | RNA exonuclease 2 | 4.242 | 0.000 | 0.213 | 0.940 |
| ACSL4 | 2182 | acyl-CoA synthetase long chain | 4.164 | 0.029 | 0.596 | 0.911 |
| | | family member 4 | | | | |
| BNIP1 | 662 | BCL2 interacting protein 1 | 4.112 | 0.022 | −0.296 | 0.960 |
| MRPS2 | 51116 | mitochondrial ribosomal protein S2 | 4.105 | 0.023 | 0.593 | 0.904 |
| MAIP1 | 79568 | matrix AAA peptidase interacting | 4.099 | 0.004 | −0.022 | 0.997 |
| | | protein 1 | | | | |
| BCL2L1 | 598 | BCL2 like 1 | 4.094 | 0.031 | −0.344 | 0.954 |
| TMEM14C | 51522 | transmembrane protein 14C | 4.016 | 0.013 | 0.699 | 0.848 |
| PTRH2 | 51651 | peptidyl-tRNA hydrolase 2 | 3.904 | <0.001 | 0.699 | 0.512 |
| L2HGDH | 79944 | L-2-hydroxyglutarate | 3.842 | 0.030 | −0.072 | 0.993 |
| | | dehydrogenase | | | | |
| ARG2 | 384 | arginase 2 | 3.827 | 0.010 | 1.012 | 0.722 |
| MRPL4 | 51073 | mitochondrial ribosomal protein L4 | 3.799 | 0.014 | 1.653 | 0.552 |
| PPIF | 10105 | peptidylprolyl isomerase F | 3.731 | 0.020 | 0.930 | 0.792 |
| CPOX | 1371 | coproporphyrinogen oxidase | 3.711 | <0.001 | 0.318 | 0.714 |
| MRPL48 | 51642 | mitochondrial ribosomal protein | 3.649 | 0.007 | 1.113 | 0.662 |
| | | L48 | | | | |
| MRPS11 | 64963 | mitochondrial ribosomal protein | 3.623 | <0.001 | 1.272 | 0.301 |
| | | S11 | | | | |
| MRPL44 | 65080 | mitochondrial ribosomal protein | 3.569 | 0.003 | 1.303 | 0.496 |
| | | L44 | | | | |
| MRPS31 | 10240 | mitochondrial ribosomal protein | 3.520 | 0.039 | 0.148 | 0.982 |
| | | S31 | | | | |
| PYCR2 | 29920 | pyrroline-5-carboxylate reductase 2 | 3.399 | 0.021 | 1.046 | 0.722 |
| OAT | 4942 | ornithine aminotransferase | 3.292 | 0.010 | 0.508 | 0.865 |
| MRPL12 | 6182 | mitochondrial ribosomal protein | 3.274 | 0.009 | 0.857 | 0.722 |
| | | L12 | | | | |
| LACTB2 | 51110 | lactamase beta 2 | 3.255 | 0.012 | −0.091 | 0.986 |
| SFXN2 | 118980 | sideroflexin 2 | 3.240 | 0.032 | 0.731 | 0.840 |
| PLSCR3 | 57048 | phospholipid scramblase 3 | 3.226 | 0.005 | 0.094 | 0.982 |
| TIMM13 | 26517 | translocase of inner mitochondrial | 3.159 | 0.017 | 0.257 | 0.950 |
| | | membrane 13 | | | | |
| CPT1A | 1374 | carnitine palmitoyltransferase 1A | 3.069 | <0.001 | 0.247 | 0.834 |
| MRPL13 | 28998 | mitochondrial ribosomal protein | 3.030 | <0.001 | 0.581 | 0.607 |
| | | L13 | | | | |
| CYB5B | 80777 | cytochrome b5 type B | 3.021 | 0.043 | −0.428 | 0.920 |
| MRPS35 | 60488 | mitochondrial ribosomal protein | 3.003 | 0.040 | 1.467 | 0.588 |
| | | S35 | | | | |
| AGMAT | 79814 | agmatinase | 2.994 | 0.007 | 0.588 | 0.805 |
| MRPS9 | 64965 | mitochondrial ribosomal protein S9 | 2.970 | 0.033 | 1.633 | 0.518 |
| NUDT1 | 4521 | nudix hydrolase 1 | 2.947 | <0.001 | −1.222 | 0.227 |
| MPST | 4357 | mercaptopyruvate sulfurtransferase | 2.938 | <0.001 | 0.621 | 0.552 |
| HTRA2 | 27429 | HtrA serine peptidase 2 | 2.929 | 0.017 | 1.077 | 0.652 |
| TOMM40 | 10452 | translocase of outer mitochondrial | 2.848 | 0.005 | 0.692 | 0.710 |
| | | membrane 40 | | | | |
| MTX1 | 4580 | metaxin 1 | 2.803 | 0.014 | 0.075 | 0.989 |
| MRPL37 | 51253 | mitochondrial ribosomal protein | 2.762 | 0.002 | 0.667 | 0.657 |
| | | L37 | | | | |
| RMND1 | 55005 | required for meiotic nuclear | 2.669 | 0.019 | −0.033 | 0.994 |
| | | division 1 homolog | | | | |
| MRPL39 | 54148 | mitochondrial ribosomal protein | 2.610 | 0.038 | 0.254 | 0.950 |
| | | L39 | | | | |
| ACADVL | 37 | acyl-CoA dehydrogenase very long | 2.567 | <0.001 | 0.411 | 0.070 |
| | | chain | | | | |
| ECI1 | 1632 | enoyl-CoA delta isomerase 1 | 2.477 | 0.012 | −2.003 | 0.185 |
| ACSL5 | 51703 | acyl-CoA synthetase long chain | 2.457 | 0.003 | 0.610 | 0.684 |
| | | family member 5 | | | | |
| DUT | 1854 | deoxyuridine triphosphatase | 2.436 | <0.001 | 0.116 | 0.847 |
| ACSL1 | 2180 | acyl-CoA synthetase long chain | 2.348 | 0.002 | 0.254 | 0.873 |
| | | family member 1 | | | | |
| ARL2 | 402 | ADP ribosylation factor like | 2.250 | 0.020 | 0.579 | 0.781 |
| | | GTPase 2 | | | | |
| FEN1 | 2237 | flap structure-specific endonuclease | 2.245 | <0.001 | 0.235 | 0.566 |
| | | 1 | | | | |
| AGK | 55750 | acylglycerol kinase | 2.232 | 0.001 | −0.159 | 0.938 |
| NDUFA8 | 4702 | NADH:ubiquinone oxidoreductase | 2.224 | 0.004 | 0.276 | 0.873 |
| | | subunit A8 | | | | |
| ACAA2 | 10449 | acetyl-CoA acyltransferase 2 | 2.217 | <0.001 | 0.313 | 0.260 |
| LRPPRC | 10128 | leucine rich pentatricopeptide | 2.193 | <0.001 | 0.207 | 0.810 |
| | | repeat containing | | | | |
| FDXR | 2232 | ferredoxin reductase | 2.169 | 0.006 | 0.407 | 0.806 |
| GRSF1 | 2926 | G-rich RNA sequence binding | 2.142 | 0.031 | 1.055 | 0.566 |
| | | factor 1 | | | | |
| DNM1L | 10059 | dynamin 1 like | 2.141 | 0.010 | −0.193 | 0.938 |
| CISD2 | 493856 | CDGSH iron sulfur domain 2 | 2.128 | 0.002 | −0.021 | 0.993 |
| MRPL1 | 65008 | mitochondrial ribosomal protein L1 | 2.126 | 0.000 | 0.537 | 0.497 |
| PMPCA | 23203 | peptidase, mitochondrial processing | 2.125 | 0.025 | 1.008 | 0.565 |
| | | alpha subunit | | | | |
| ACADM | 34 | acyl-CoA dehydrogenase medium | 2.116 | 0.001 | 0.518 | 0.581 |
| | | chain | | | | |
| BDH1 | 622 | 3-hydroxybutyrate dehydrogenase 1 | 2.108 | 0.002 | 0.589 | 0.566 |
| TIMM23 | 1E+08 | translocase of inner mitochondrial | 2.107 | 0.027 | 0.411 | 0.858 |
| | | membrane 23 | | | | |
| CPT2 | 1376 | carnitine palmitoyltransferase 2 | 2.079 | 0.019 | 0.652 | 0.714 |
| CIAPIN1 | 57019 | cytokine induced apoptosis | 2.074 | 0.003 | −0.691 | 0.540 |
| | | inhibitor 1 | | | | |
| CHDH | 55349 | choline dehydrogenase | 2.029 | 0.029 | 1.195 | 0.465 |
| TOMM22 | 56993 | translocase of outer mitochondrial | 2.022 | 0.002 | 0.624 | 0.546 |
| | | membrane 22 | | | | |
| FKBP8 | 23770 | FKBP prolyl isomerase 8 | 2.014 | 0.001 | 1.093 | 0.138 |

6.2.4.3.3 Cell proliferation gene product signature (cell division, cell cycle, and DNA replication/repair)

The proteomics study has revealed that the expression of a number of gene products associated with cell proliferation pathways is increased relative to their expression in baseline Tregs. See Table 8. These enriched gene products include, for example, ones associated with cell cycle (p=0.0014), cell division (p=0.0478), DNA replication (p=5.05e-13), and DNA Repair (p=0.0496) pathways.

As shown in Table 8, the cell proliferation gene product signature includes, for example: ARL2, ARL3, BCCIP, CCDCl24, CDK1, CDK2, CDK5, CDK6, CUL4B, DCTN3, FEN1, HELLS, LIG1, MAD2L1, MAEA, MCM2, MCM2, MCM3, MCM4, MCM5, MCM6, MCM7, MCMBP, NUDC, PCNA, POLD1, POLD2, RALB, RBM38, RFC2, RFC3, RFC4, RFC5, RNASEH2A, RNASEH2B, SMC2.

TABLE 7
Cell proliferation gene product signature

| | | | log2 of | | log2 of | |
|---|---|---|---|---|---|---|
| | | | fold-change | | fold-change | |
| | NCBI | | of baseline | Adjusted | of expanded | Adjusted |
| GeneSymbol | GeneID | GeneDescription | vs. expanded | p-value | vs. freeze-thaw | p-value |
| CDK1 | 983 | cyclin dependent kinase 1 | 6.615 | 0.007 | −0.077 | 0.993 |
| MCM5 | 4174 | minichromosome maintenance | 6.473 | <0.001 | −0.031 | 0.993 |
| | | complex component 5 | | | | |
| CDK6 | 1021 | cyclin dependent kinase 6 | 6.206 | 0.006 | −1.043 | 0.831 |
| MCM2 | 4171 | minichromosome maintenance | 6.203 | 0.010 | 1.548 | 0.735 |
| | | complex component 2 | | | | |
| MCM4 | 4173 | minichromosome maintenance | 6.021 | <0.001 | 0.213 | 0.839 |
| | | complex component 4 | | | | |
| CDK5 | 1020 | cyclin dependent kinase 5 | 5.954 | <0.001 | 0.197 | 0.949 |
| MCM3 | 4172 | minichromosome maintenance | 5.613 | <0.001 | 0.122 | 0.966 |
| | | complex component 3 | | | | |
| CCDC124 | 115098 | coiled-coil domain containing 124 | 4.855 | 0.006 | 0.277 | 0.960 |
| MCM7 | 4176 | minichromosome maintenance | 4.743 | 0.000 | 0.060 | 0.924 |
| | | complex component 7 | | | | |
| RALB | 5899 | RAS like proto-oncogene B | 4.644 | 0.008 | −0.199 | 0.975 |
| SMC2 | 10592 | structural maintenance of | 4.463 | 0.002 | 0.600 | 0.616 |
| | | chromosomes 2 | | | | |
| CDK2 | 1017 | cyclin dependent kinase 2 | 4.455 | 0.005 | −0.124 | 0.982 |
| RBM38 | 55544 | RNA binding motif protein 38 | 4.433 | 0.004 | −0.435 | 0.912 |
| MCM6 | 4175 | minichromosome maintenance | 4.394 | <0.001 | 0.152 | 0.946 |
| | | complex component 6 | | | | |
| MAEA | 10296 | macrophage erythroblast attacher | 4.259 | 0.007 | −1.045 | 0.722 |
| DCTN3 | 11258 | dynactin subunit 3 | 4.232 | 0.001 | 0.377 | 0.902 |
| BCCIP | 56647 | BRCA2 and CDKN1A interacting | 4.012 | <0.001 | 0.194 | 0.908 |
| | | protein | | | | |
| RFC5 | 5985 | replication factor C subunit 5 | 4.000 | 0.006 | −0.243 | 0.954 |
| MAD2L1 | 4085 | mitotic arrest deficient 2 like 1 | 3.978 | 0.001 | −0.209 | 0.975 |
| CUL4B | 8450 | cullin 4B | 3.896 | 0.034 | −0.871 | 0.844 |
| MCMBP | 79892 | minichromosome maintenance | 3.680 | 0.013 | 0.344 | 0.938 |
| | | complex binding protein | | | | |
| RFC3 | 5983 | replication factor C subunit 3 | 3.663 | 0.012 | −0.308 | 0.946 |
| LIG1 | 3978 | DNA ligase 1 | 3.564 | 0.001 | −0.547 | 0.770 |
| RFC4 | 5984 | replication factor C subunit 4 | 3.546 | <0.001 | 0.130 | 0.960 |
| POLD2 | 5425 | DNA polymerase delta 2, | 3.491 | 0.003 | 0.289 | 0.920 |
| | | accessory subunit | | | | |
| RNASEH2A | 10535 | ribonuclease H2 subunit A | 3.447 | 0.037 | 0.751 | 0.848 |
| HELLS | 3070 | helicase, lymphoid specific | 3.317 | 0.037 | 0.966 | 0.783 |
| POLD1 | 5424 | DNA polymerase delta 1, catalytic | 2.998 | 0.001 | −0.228 | 0.915 |
| | | subunit | | | | |
| ARL3 | 403 | ADP ribosylation factor like | 2.973 | <0.001 | 0.034 | 0.990 |
| | | GTPase 3 | | | | |
| RNASEH2B | 79621 | ribonuclease H2 subunit B | 2.858 | <0.001 | 0.037 | 0.990 |
| RFC2 | 5982 | replication factor C subunit 2 | 2.731 | 0.001 | −0.203 | 0.911 |
| PCNA | 5111 | proliferating cell nuclear antigen | 2.615 | <0.001 | 0.183 | 0.655 |
| ARL2 | 402 | ADP ribosylation factor like | 2.250 | 0.020 | 0.579 | 0.781 |
| | | GTPase 2 | | | | |
| FEN1 | 2237 | flap structure-specific | 2.245 | <0.001 | 0.235 | 0.566 |
| | | endonuclease 1 | | | | |
| NUDC | 10726 | nuclear distribution C, dynein | 2.046 | <0.001 | −0.212 | 0.742 |
| | | complex regulator | | | | |

6.2.4.3.4 Highest protein expression gene product signature (following expansion)

Next, gene products obtained from the proteomics study were stratified based on highest protein expression observed in the ex vivo-expanded Tregs produced by the methods presented herein, as quantified using intensity based absolute quantification (iBAQ) which is a measure of protein abundance in the proteomics assay. The top 40 expressed gene products obtained from the study are compiled at Table 9. As noted in Table 9, the expression of each of the members of this highest protein expression gene product signature is increased relative to the expression seen in baseline Tregs.

The gene products making up the highest expressing protein signatures include, for example: ACAA2, ACADM, ACADVL, ACOT7, BSG, CACYBP, CD74, CDK1, CPOX, DUT, ECI1, ENO3, FEN1, FKBP3, HIST1H2BJ, HLA-DQA1, HLA-DRA, HLA-DRB1, LGALS1, LGALS3, MCM5, MCM6, MCM7, MTHFD1, NAMPT, NME1, NQ01, PCNA, RABIA, RALB, SLC25A4, STAT1, STMN1, STMN2, TUBA1B, TUBB4A, TUBB8, TXN, TXNRD1, and WARS.

TABLE 8
Highest protein expression gene product signature

| | | | log2 of | | log2 of | |
|---|---|---|---|---|---|---|
| | | | fold-change | | fold-change | |
| | NCBI | | of baseline | Adjusted | of expanded | Adjusted |
| GeneSymbol | GeneID | GeneDescription | vs. expanded | p-value | vs. freeze-thaw | p-value |
| HIST1H2BJ | 8970 | histone cluster 1 H2B family | 13.792 | <0.001 | −2.190 | 0.000 |
| | | member j | | | | |
| TXN | 7295 | thioredoxin | 3.977 | <0.001 | 0.064 | 0.908 |
| TUBA1B | 10376 | tubulin alpha 1b | 2.224 | 0.001 | 0.111 | 0.946 |
| LGALS3 | 3958 | galectin 3 | 2.523 | <0.001 | −0.108 | 0.940 |
| NME1 | 4830 | NME/NM23 nucleoside | 13.947 | 0.006 | −3.549 | 0.693 |
| | | diphosphate kinase 1 | | | | |
| TUBB8 | 347688 | tubulin beta 8 class VIII | 8.661 | 0.028 | −3.253 | 0.672 |
| STMN1 | 3925 | stathmin 1 | 2.411 | <0.001 | −0.029 | 0.975 |
| TUBB4A | 10382 | tubulin beta 4A class IVa | 8.606 | 0.026 | 1.514 | 0.874 |
| STAT1 | 6772 | signal transducer and activator of | 3.425 | <0.001 | −0.104 | 0.882 |
| | | transcription 1 | | | | |
| LGALS1 | 3956 | galectin 1 | 2.014 | <0.001 | 0.096 | 0.877 |
| CACYBP | 27101 | calcyclin binding protein | 2.754 | <0.001 | −0.007 | 0.994 |
| WARS | 7453 | tryptophanyl-tRNA synthetase | 3.715 | <0.001 | 0.025 | 0.982 |
| PCNA | 5111 | proliferating cell nuclear antigen | 2.615 | <0.001 | 0.183 | 0.655 |
| ACAA2 | 10449 | acetyl-CoA acyltransferase 2 | 2.217 | <0.001 | 0.313 | 0.260 |
| CDK1 | 983 | cyclin dependent kinase 1 | 6.615 | 0.007 | 1.621 | 0.502 |
| ECI1 | 1632 | enoyl-CoA delta isomerase 1 | 2.477 | 0.012 | −2.003 | 0.185 |
| ACADVL | 37 | acyl-CoA dehydrogenase very long | 2.567 | <0.001 | 0.411 | 0.070 |
| | | chain | | | | |
| SLC25A4 | 291 | solute carrier family 25 member 4 | 5.089 | 0.030 | −2.206 | 0.621 |
| RAB1A | 5861 | RAB1A, member RAS oncogene | 2.680 | 0.001 | −0.017 | 0.996 |
| | | family | | | | |
| DUT | 1854 | deoxyuridine triphosphatase | 2.436 | <0.001 | 0.116 | 0.847 |
| HLA-DRA | 3122 | major histocompatibility complex, | 4.722 | <0.001 | 0.353 | 0.353 |
| | | class II, DR alpha | | | | |
| FKBP3 | 2287 | FKBP prolyl isomerase 3 | 2.105 | <0.001 | 0.039 | 0.979 |
| NAMPT | 10135 | nicotinamide | 4.070 | <0.001 | 0.255 | 0.385 |
| | | phosphoribosyltransferase | | | | |
| FEN1 | 2237 | flap structure-specific endonuclease | 2.245 | <0.001 | 0.235 | 0.566 |
| | | 1 | | | | |
| STMN2 | 11075 | stathmin 2 | 5.041 | 0.023 | 0.073 | 0.993 |
| NQO1 | 1728 | NAD(P)H quinone dehydrogenase | 9.019 | <0.001 | −0.188 | 0.882 |
| | | 1 | | | | |
| HLA-DRB1 | 3123 | major histocompatibility complex, | 5.489 | <0.001 | 0.347 | 0.685 |
| | | class II, DR beta 1 | | | | |
| TXNRD1 | 7296 | thioredoxin reductase 1 | 2.299 | <0.001 | 0.080 | 0.940 |
| MCM7 | 4176 | minichromosome maintenance | 4.743 | <0.001 | 0.060 | 0.924 |
| | | complex component 7 | | | | |
| HLA-DQA1 | 3117 | major histocompatibility complex, | 3.944 | <0.001 | 0.215 | 0.932 |
| | | class II, DQ alpha 1 | | | | |
| CD74 | 972 | CD74 molecule | 2.625 | <0.001 | 0.366 | 0.357 |
| MTHFD1 | 4522 | methylenetetrahydrofolate | 2.410 | <0.001 | −0.065 | 0.938 |
| | | dehydrogenase, cyclohydrolase and | | | | |
| | | formyltetrahydrofolate synthetase 1 | | | | |
| MCM5 | 4174 | minichromosome maintenance | 6.473 | <0.001 | −0.031 | 0.993 |
| | | complex component 5 | | | | |
| CPOX | 1371 | coproporphyrinogen oxidase | 3.711 | <0.001 | 0.318 | 0.714 |
| RALB | 5899 | RAS like proto-oncogene B | 4.644 | 0.008 | −0.199 | 0.975 |
| BSG | 682 | basigin (Ok blood group) | 4.721 | 0.001 | 0.042 | 0.993 |
| ACADM | 34 | acyl-CoA dehydrogenase medium | 2.116 | 0.001 | 0.518 | 0.581 |
| | | chain | | | | |
| MCM6 | 4175 | minichromosome maintenance | 4.394 | <0.001 | 0.152 | 0.946 |
| | | complex component 6 | | | | |
| ACOT7 | 11332 | acyl-CoA thioesterase 7 | 4.566 | <0.001 | −0.075 | 0.973 |
| ENO3 | 2027 | enolase 3 | 6.086 | 0.001 | −0.296 | 0.950 |

### 6.3 Example 3: Extracellular Vesicles Derived from Suppressive Immune Cells Modulate In Vitro and In Vivo Inflammation

### 6.3.1. Methods

### 6.3.1.1 Ex-Vivo Treg Expansion

Tregs were enriched and ex vivo-expanded following the protocols set out in Example 1, above. The Tregs were either cultured in human AB serum as described in Example 1, or, to isolate a pure population of Treg EVs, Tregs were cultured with the same expansion protocols but with the serum substituted for exosome-depleted fetal bovine serum (FBS).

The Tregs were obtained from either healthy subjects or ALS patients as indicated below.

### 6.3.1.2 Extracellular Vesicle Isolation Using PEG

Extracellular vesicles (EVs) were isolated according to manufacturer's protocols using a polyethylene glycol precipitation (PEG) method and ExoQuick-TC reagent (System Biosciences, SBI). Briefly, media from Treg expansion cultures were centrifuged at 3000×g for 15 minutes to remove cells and debris. PEG reagent was added to spun supernatant at 1:5 ratio of PEG: TC Media, mixed thoroughly, and refrigerated overnight at 4° C. The mixture was then centrifuged at 1500×g for 30 minutes, the supernatant aspirated, spun again at 1500×g for 10 minutes, and the supernatant aspirated again. The resulting EV pellet was resuspended in sterile PBS and diluted for Nanosight EV size/concentration analysis and for future use. EVs were stored at −20° C. while limiting freeze/thaw cycles.

### 6.3.1.3 Extracellular Vesicle Isolation Using TFF

EV populations isolated using tangential flow filtration (TFF) techniques were isolated using the protocol summarized herein.

Briefly, the isolation utilized a Repligen KR2i TFF system that allows for isolation, concentration, and diafiltration of Treg EV populations using a buffer appropriate for therapeutic use.

First, media from the Treg expansion culture was circulated using TFF and a Midi 20 cm 0.65 μm Spectrum mPES 0.75 mm Hollow Fiber filter (D02-E65U-07-N) with a membrane area of 85 cm² and fiber diameter of 0.75 mm to filter out cells and debris. This process utilized a flow rate of 100-200 mL/min that resulted in a shear rate of about 2,000-5,000 s⁻¹ while maintaining a variable transmembrane pressure (TMP) driven by a retentate pressure of 5 psi.

The permeate of this step was then subjected to a process designed to concentrate and diafiltrate the EV population into the retentate with continuous circulation. This process utilized the TFF system and a Midi 20 cm 500 kD Spectrum mPES 0.5 mm Hollow Fiber filter (D02-E500-05-N) with a membrane area of 115 cm² and fiber diameter of 0.5 mm to retain/concentrate all particles greater than approximately 60-80 nm into the retentate. This process utilized a flow rate of 80-200 mL/min that resulted in a shear rate of 2,000-7,500 s⁻¹ while maintaining and driving the filtration at 10 psi TMP. The final volume after concentration was targeted to be around 20 mLs.

Sterile PBS was incorporated into the circulation, resulting in 10× diafiltration and replacement of the existing solution. 10× diafiltration effectively resulted in a full buffer volume exchange of 10 times and was performed to eliminate 99% + of the soluble material or impurities that would remain.

### 6.3.1.4 Nanosight EV Size/Concentration Readings

EV readings were obtained using Nanosight NS300 (Malvern Panalytical) particle analyzer. EV samples were diluted for readings and analyzed using continual measurement at 50 μl per minute speed with 3 analysis recordings of 60 seconds each with the following parameters: camera level (12-15), temperature (22° C.), and detection threshold (5). Concentration was recorded as particles/ml and size statistics were recorded as mean and mode.

6.3.1.5 iPS-Derived M1 Myeloid Cultures

Myeloid cells were generated using protocols previously developed/described (Thome et al., 2018, Molecular Neurodegeneration 13.1:1-11; Zhao et al., 2020, Iscience 23.6:101192).

Briefly, myeloid cells were produced using a 4-step culturing process that allows for the generation of CD14+ cells from control iPSC lines. CD14+ myeloid cells are isolated using positive, magnetic selection with Miltenyi Biotec CD14 beads, isolation columns, and magnet setup.

For M1 cells: CD14+ cells were cultured in complete RPMI media (10% fetal bovine serum, 25 mM HEPES, 1 mM sodium pyruvate, 1× nonessential amino acids, 55 μM 2-mercaptoethanol, 100 units/ml penicillin, and 100 μg/ml streptomycin) supplemented with 50 ng/ml GMCSF (R&D systems) for 7 days to create M0 cells for M1 use. M0 cells were then primed with 0.1 ng/ml lipopolysaccharides (LPS) (Sigma) and 0.2 ng/ml IFNγ (Invitrogen) to polarize myeloid cells to be pro-inflammatory, M1 cells.

### 6.3.1.6 MSC EVs

Human mesenchymal stem cells (MSCs) were obtained from bone marrow and were cultured in a T75 flask utilizing culture media containing 10% FBS in Minimal Essential Media supplemented with antibiotic/antimycotic solution. The cells were allowed to become 80% confluent in the flask followed by media aspiration, PBS washing, and replacement with the same media but with 10% exosome free FBS in place of normal FBS. T75 flask was cultured another 48 hours and EVs were isolated from the tissue culture media using PEG isolation.

6.3.1.7 EV Suppression Assays with Myeloid Cells and Tresp Proliferation Assays

M0 (GM-CSF) cells were lifted with enzyme-free dissociation buffer, pelleted, and replated at 50,000 cells/well in 24 well plates.

M1 cells were primed with 0.1 ng/ml LPS (Sigma) and 0.2 ng/ml IFNγ (Invitrogen) for 1 hour to polarize to M1 cells. Treg EVs (1×10⁸ particles) were spiked into the cultures following M1 polarization for overnight time point followed by collection of M1 cells for RNA analysis and cultured media for protein analysis.

For responder T cell (Tresp) proliferation assays, control Tresp were isolated using Miltenyi Biotec reagents and protocols to isolated CD4+CD25-T cells from peripheral blood. Tresps were plated at 50,000 cells per well in 96 well, round-bottom plates and stimulated with CD3/28 beads (Miltenyi Biotec).

Treg EVs were added to the cultures in escalating doses and remained in Tresp culture for the entire experiment. After 4 days in culture, Tresps were pulsed with tritium and proliferation was determined by examining tritium incorporation 18 hours after tritium pulsing.

### 6.3.1.8 LPS-Induced Neuroinflammation Mouse Model and SOD1 Mouse Model of ALS

For the acute LPS-induced neuroinflammation mouse model, C57B16 WT mice were injected intraperitoneally (IP) with 2 mg/kg LPS (Sigma; O111:B4) followed by intranasal administration Treg EV (1×10⁹ particles) 2 hours after LPS injection. Mice were then sacrificed at 12 hours or 22 hours post-intranasal administration and organs harvested for RNA and protein analyses, specifically brain components (hippocampus and cortex) and spleen. Myeloid cells were isolated from spleen by extracting single cells through a 40 μm cell strainer and using mouse CD11b beads and magnetic columns (Miltenyi Biotec).

Transgenic mice harboring the SOD1-G93A mutation (see Gurney et al., 1994, Science 264.5166:1772-1775) were used as a motor neuron degeneration model for ALS. Phenotype analysis of SOD1 mice began at day 70 and intranasal injections of Treg EVs (1×10⁹ particles) began at day 90 and continued every two weeks until the mice reached their ethical endpoint, requiring them to be sacrificed. Mouse phenotype was assessed using a modified “BASH scoring system” whereby SOD1 mice gain a degenerative point from 0-6 as phenotype worsens with disease progression. The phenotypes were assessed and points added as follows (but not necessarily in this order): +1 Tremulousness, +1 Gait abnormalities, +1 Hind limb weakness/paresis, +1 Weight loss of more than 10% adult weight, +1 Spasticity to one or both hindlimbs, +1 Paralysis, which is terminal stage resulting in sacrificing the mouse and harvesting organs for RNA and protein analysis.

### 6.3.1.9 RNA Purification, RT-PCR Analysis, and Protein ELISAs

RNA was isolated from cells and tissues using Trizol reagent followed by Direct-zol RNA MiniPrep Plus Kit (Zymo Research) according to manufacturer's recommendations. Quantitative RT-PCR was performed using one-step RT-PCR kit with SYBR Green (Bio-Rad) and an iQ5 Multicolor Real-Time PCR detection system (Bio-Rad). Primers for RT-PCR (IL-6, IL-1β, TNFα, IL-10, Arg-1, IFN-γ, FOXP3, and CD206) were acquired from Bio-Rad and run according to the manufacturer's protocols. The relative expression level of each mRNA was assessed using the AACt method and normalized to B-actin/controls. Supernatants were collected from co-culture paradigms and IL-6 protein amounts were assessed using ELISA-based immunoassays (Invitrogen).

### 6.3.2. Results

6.3.2.1 Treg-Derived EVs Suppress Tresp Proliferation and Pro-Inflammatory iPSC-Derived M1 Cells.

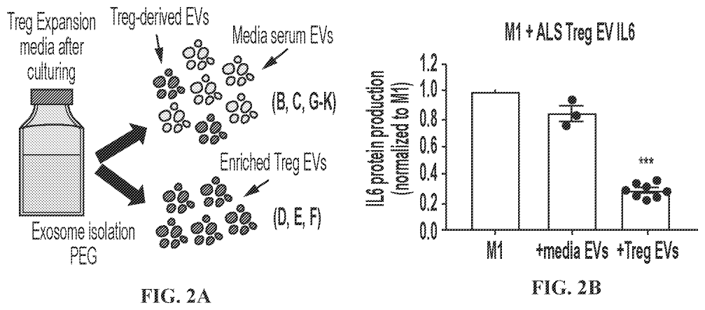

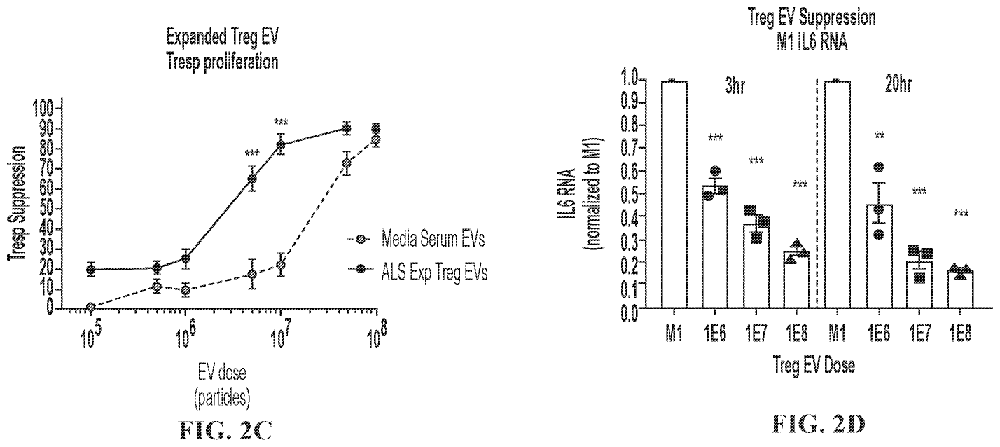

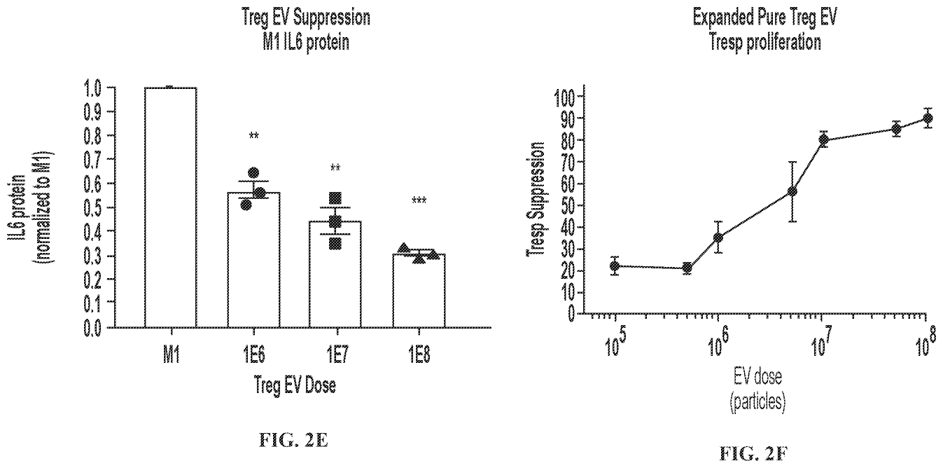

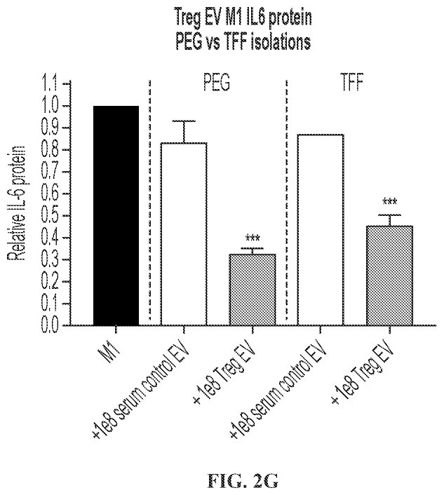

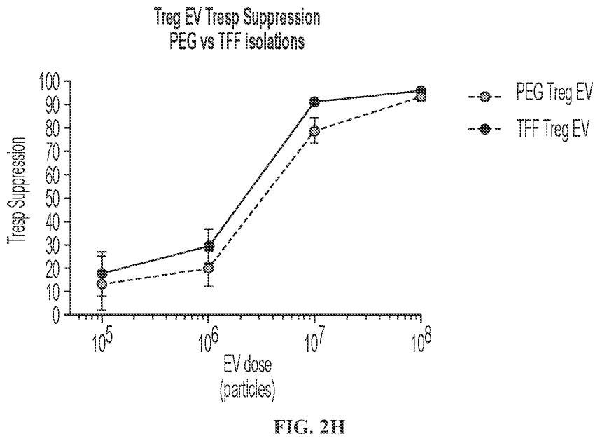

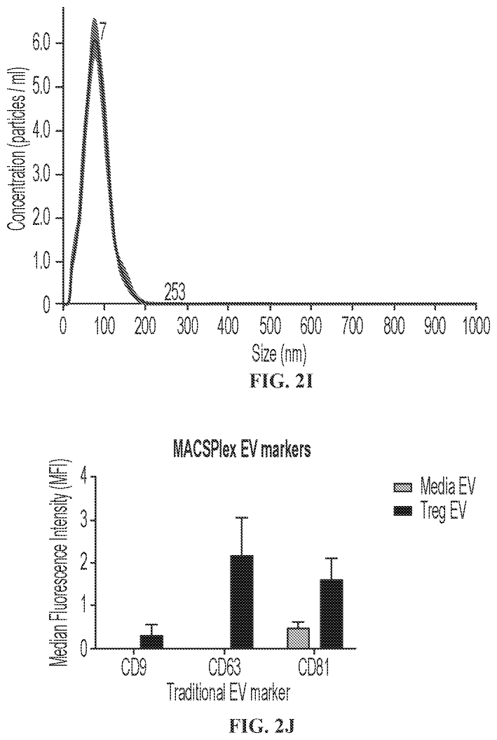

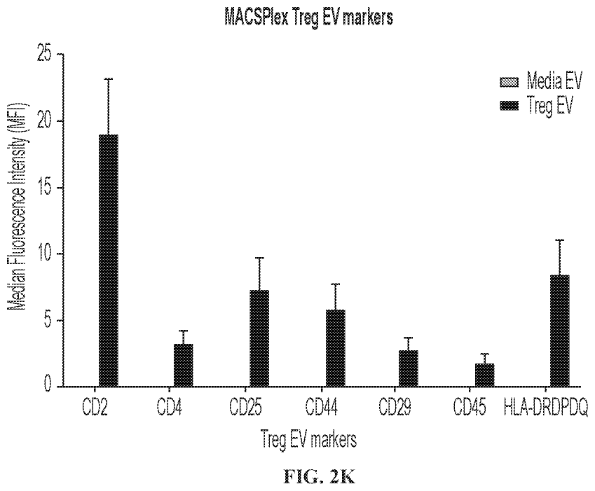

Treg expansion media was saved from expansion cultures of ALS patient-derived Tregs and EVs were isolated using PEG methods described above. This results in a final mixture of EVs containing approximately 70% media serum-derived EVs and about 30% Treg-derived EVs (FIG. 2A).

Treg/media EV mixtures showed the ability to suppress M1 IL-6 protein production by roughly 70% when given at a dose of 1×10⁸ particles per 50,000 M1 cells. In contrast, media EVs alone showed only minimal effect (less than 20%). FIG. 2B.

When the Treg EV mixture was added to Tresp proliferation assays at escalating doses, a dose-dependent inhibition of Tresp proliferation was observed, becoming prominent at 65% suppression at a fixed dose of 5×10⁷ particles per 50,000 M1 cells and increasing to 82% and 91% suppression at fixed doses of 1×10⁷ and 5×10⁷ particles per 50,000 M1 cells, respectively (FIG. 2C).

To isolate a pure population of Treg EVs, Tregs were cultured with the same expansion protocols but with the serum substituted for exosome-depleted fetal bovine serum (FBS). This yields a population of Treg EVs that has the same purity as the purity of the population of Tregs from with the EVs are derived, since there will be no EVs from serum. The pure Tregs EVs were tested in the same fashion as described above to examine their suppressive capacity on pro-inflammatory M1 cells and Tresp proliferation. The Treg EVs exhibited significant, dose-dependent suppressive abilities when examining LPS-induced IL-6 RNA production by M1 cells after both 3 hours and 20 hours (FIG. 2D). These data were confirmed by assessing inhibition of LPS-induced IL-6 protein production by M1 cells following use of escalating doses of pure Treg EVs; all of which showed to be significantly effective (FIG. 2E). The suppressive profile of pure Treg EVs on Tresp proliferation was consistent with the profile of the mixed populations, with escalating doses of Treg EV conferring increased suppression of proliferation (FIG. 2F).

When Treg mixed EVs were isolated using TFF, the EV populations retained the suppressive activities observed using PEG isolation. In particular, Treg mixed EVs isolated using TFF reduced iPSC-derived M1 IL-6 protein in a dose-dependent manner similar to that observed when the EVs were isolated using PEG (FIG. 2G). Also, mixed Treg EVs isolated using TFF were able to suppress Tresp proliferation at escalated dosing in a manner similar to that observed when the EVs were isolated using PEG (FIG. 2H).

FIG. 2I depicts an exemplary size profile of Treg mixed EVs produced and PEG isolated as described in this example, which demonstrates a single peak indicating a diameter size distribution between about 50-150 nm. The size range was verified using scanning electron microscopy (SEM). It is noted that nanoparticle analysis of diverse populations of Treg EVs obtained a described herein have demonstrated a consistent size distribution between about 50 nm and about 150 nm (e.g., exhibiting mean=94.5 nm, mode=76.8 nm, D10=56.6 nm, D50 (median)=86.4 nm, D90=146.9 nm).

Using Miltenyi MACSPlex Exosome Kit (Miltenyi Biotec) analysis, mixed EVs produced and isolated using such a process were found to express a combination of exosome markers including CD9, CD63, and CD81 whereas, in contrast, of these markers, media EVs only expressed CD81 (FIG. 2J). Additionally, Miltenyi MACSPlex Exosome Kit (Miltenyi Biotec) analysis also demonstrated that Treg EVs were positive for CD2, CD4, CD25, CD44, CD29, CD45, and HLA-DRDPDQ, whereas, in contrast, media EVs did not express any of these markers (FIG. 2K).

Overall, Treg EVs derived from ex vivo expanded Treg cells demonstrated a unique and Treg-conserved signature along with suppressive function in vitro similar to that of expanded Treg cells.

### 6.3.2.2 Treg EVs Suppress Inflammation in LPS-Induced Mouse Model of Neuroinflammation

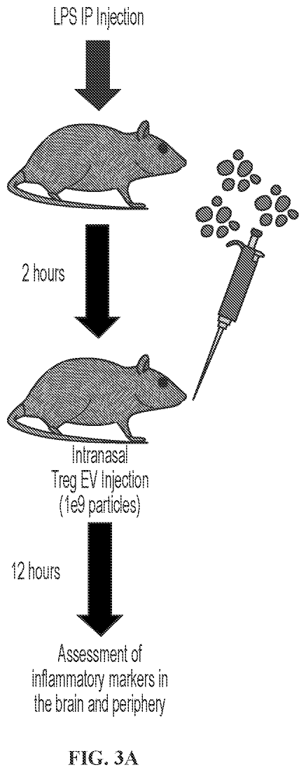

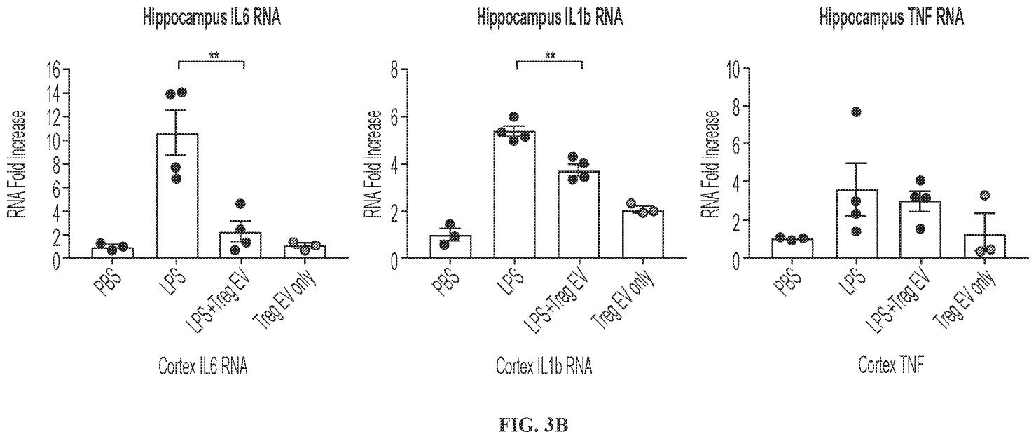

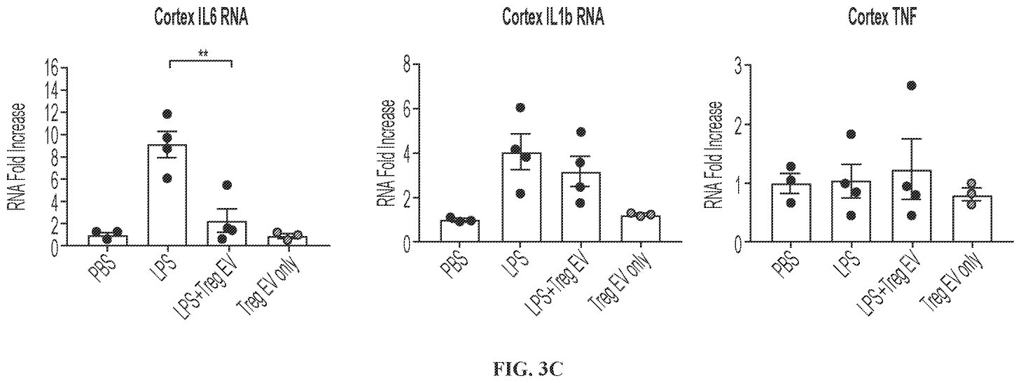

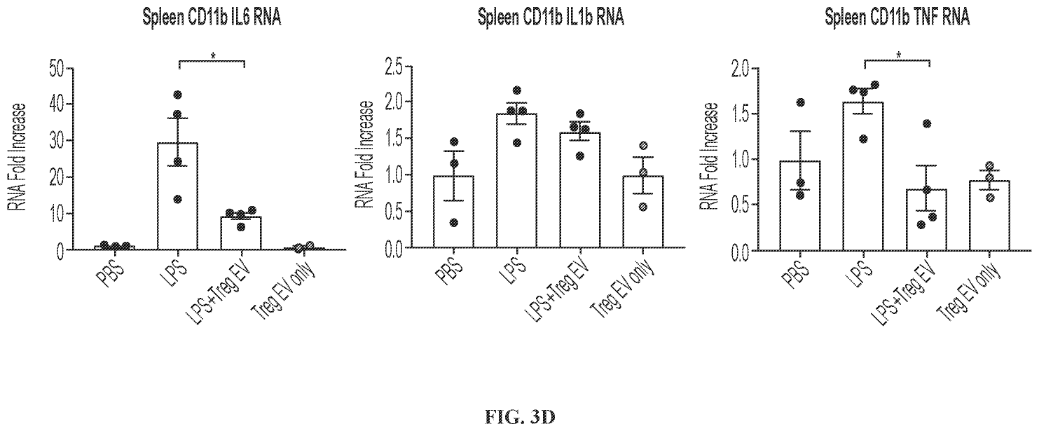

Mice were injected with LPS peripherally to induce neuroinflammatory mechanisms in the brain to test the in vivo suppressive effects of Treg EVs when administered intranasally. One ×10⁹ Treg EVs were delivered intranasally 2 hours post 2 mg/kg IP injection of LPS and the mice were sacrificed after an additional 12 hours to examine neuroinflammatory parameters (FIG. 3A). The Treg EVs utilized for these experiments were pure Treg EV populations from cultures of ex vivo-expanded Tregs from healthy human subjects. The Tregs were cultured in exosome-free FBS, and the EVs were isolated using the PEG process described above.

For the neuroinflammatory analysis, the hippocampus and cortex were isolated from sacrificed mice and RNA was extracted for RT-PCR analysis. Treg EVs demonstrated the ability to significantly reduce hippocampal IL-6 and IL-1β transcripts generated by the LPS injections, while there were no significant changes in hippocampal TNFα transcripts following treatment (FIG. 3B).

When examining the cortex, a Treg EV treatment-specific reduction in IL-6 transcripts was observed, while IL-1β and TNF transcripts stayed elevated (FIG. 3C). Peripheral inflammation was examined by analyzing inflammatory transcript changes in CD11b+ myeloid cells isolated from spleens following LPS IP injection and Treg intranasal treatments.

A robust increase in myeloid pro-inflammatory activation was observed in spleens as a result of the peripheral LPS injections (FIG. 3D). Intranasal Treg EV treatments decreased peripheral, myeloid cell-derived IL-6 and TNF transcripts 14 hours post-LPS injection.

Thus, Treg EV administration in an LPS-induced in vivo model of neuroinflammation suppressed pro-inflammatory mechanisms centrally and peripherally.

### 6.3.2.3 Treg EVs Suppress Inflammation, Extend Survival and Slow Later Stage Disease Progression in a SOD1 Mouse Model of ALS.

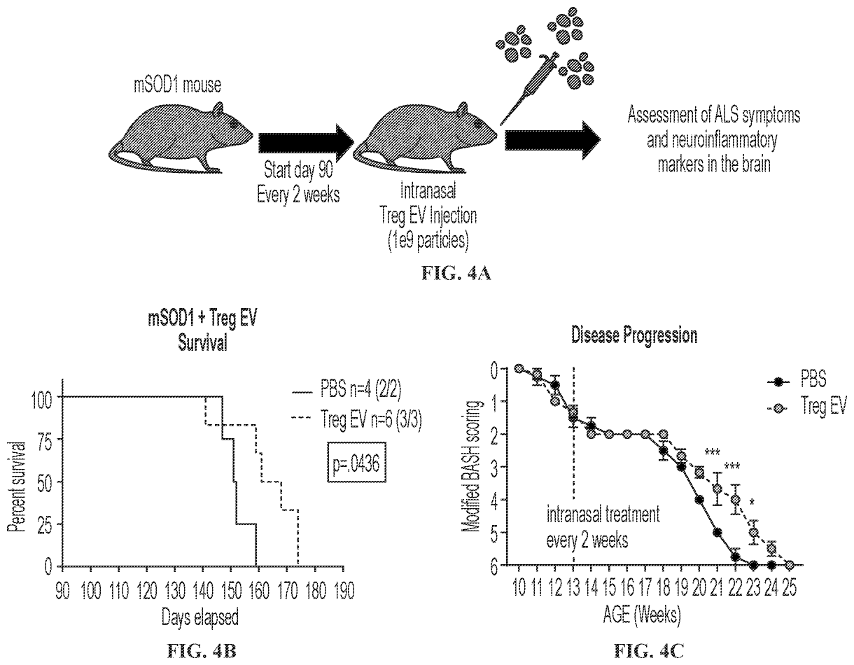

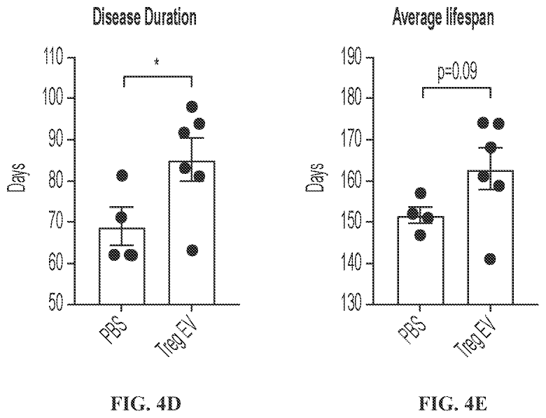

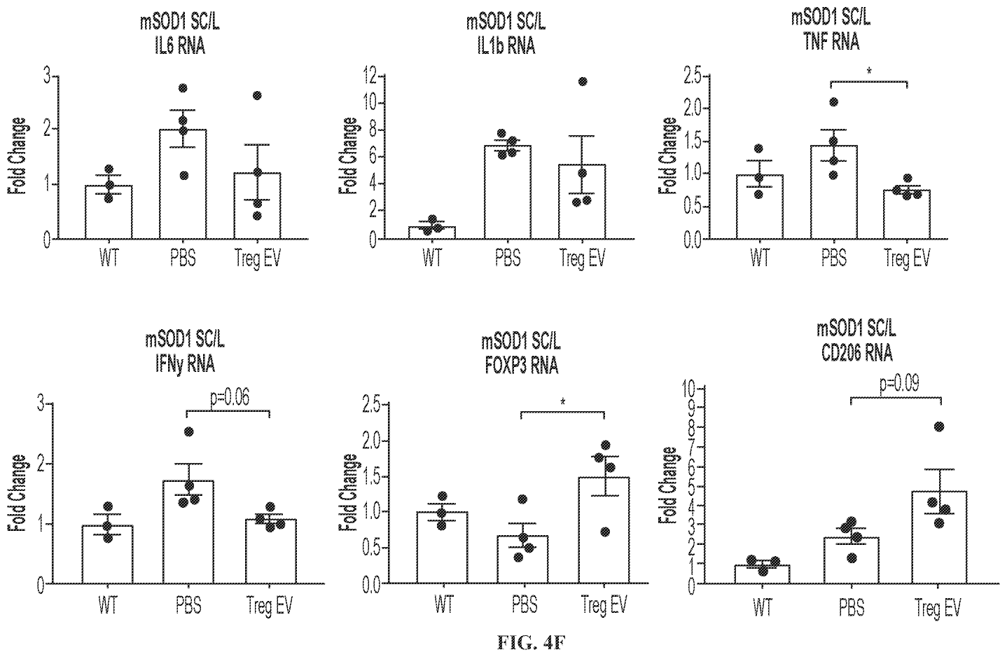

The SOD1 motor neuron degeneration mouse model of ALS was used to examine the effects of Treg EVs in a neurodegenerative model driven by inflammatory mechanisms. Intranasal treatments with 1×10⁹ particles of Treg EVs began at day 90 when the animals were already showing symptoms of degeneration, and these treatments were continued every 2 weeks until the animals were sacrificed and tissue was harvested for analysis (FIG. 4A). The Treg EVs utilized for these experiments were pure Treg EV populations from cultures of ex vivo-expanded Tregs from healthy human subjects. The Tregs were cultured in exosome-free FBS, and the EVs were isolated using the PEG process described above.

The two week interval treatments of Treg EVs significantly increased the probability of survival in the treated group compared to PBS injected controls (FIG. 4B). Additionally, the Treg EV treatments slowed disease progression in the later stages of motor neuron disease as measured by a modified scoring system detailing motor dysfunction phenotypes in the mice (FIG. 4C).

The average disease duration from first symptom was statistically increased from 85 days in the Treg EV-treated animals to only 69 days in the PBS treated mice (FIG. 4D). The average lifespan tended to be increased in the Treg EV-treated animals compared to PBS controls at 162.8 days vs. 151.7 days, respectively (FIG. 4E).

Following animal sacrifice, RNA was extracted from the lumbar portions of the spinal cord to evaluate treatment-associated inflammatory changes. Treg EVs had the ability to reduce TNF transcripts in the SOD1 spinal cords along with beneficial reductions in IL6, IL1β, and IFNγ transcripts. Additionally, levels of anti-inflammatory, Treg-associated FOXP3 RNA were increased with Treg EV treatment. Anti-inflammatory myeloid-specific CD206 transcripts were also increasing in Treg EV treated SOD1 animals compared with controls (FIG. 4F).

The results preented in this Example indicate that adminstering a Treg EV population, such as a pure (no media EV) Treg EV population, intranasally, produces a CNS benefit through reductions in pro-inflammatory transcripts in multiple areas of the brain. Further, the increase in anti-inflammatory transcripts in these same cells is indicative of a Treg EV-induced repolarization of the peripheral myeloid cells.

### 6.4 Example 4: Treg EVs have a Greater Suppressive Effect on Pro-Inflammatory M1 Cells Compared to MSC EVs

EVs isolated from Tregs (“Treg EVs”) and EVs isolated from mesenchymal stem cells (“MSC EVs”) were tested for their ability to suppress immune cells. As demonstrated herein, the Treg EVs exhibit greater suppression of pro-inflammatory M1 cells compared to MSC EVs. The Treg EVs utilized for these experiments were pure Treg EV populations from cultures of ex vivo-expanded Tregs from healthy human subjects. The Tregs were cultured in exosome-free FBS, and the EVs were isolated using the PEG process described above. The MSC EVs utilized for these experiments were isolated from cultures of human bone marrow MSCs utilizing exosome-free FBS, and were isolated using the PEG process described above.

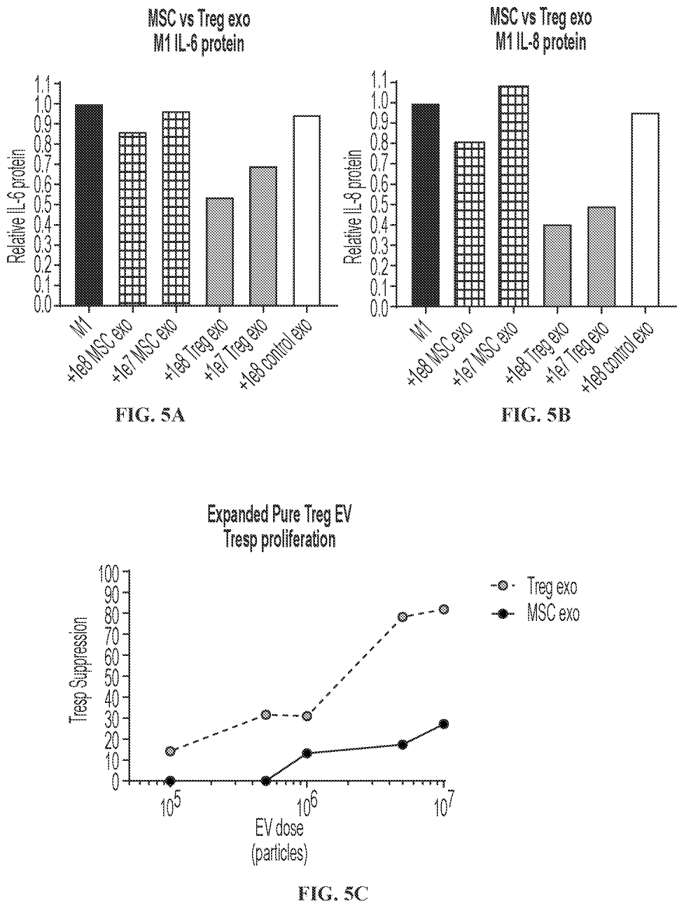

Treg EVs were able to suppress M1 pro-inflammatory IL-6 protein by 46% at a dose of 1×10⁸ EVs and 30.6% at a dose of 1×10⁷ EV compared to MSC EVs that suppressed 13.7% and 3.3%, respectively (FIG. 5A). Treg EVs suppressed M1-derived pro-inflammatory IL-8 protein by 60% at a dose of 1×10⁸ and 50% at a dose of 1×10⁷ dose compared to MSC EV that showed a 20% suppression at a dose of 1×10⁸ dose (FIG. 5B). Treg EVs suppressed T cell proliferation more than MSC EVs in a comparison study (FIG. 5C).

These experiments are also presented, below, in Example 9, which includes, e.g., figures demonstrating the statistical significance of these results.

### 6.5 Example 5: Stability and Immune Cell Suppression of EVs

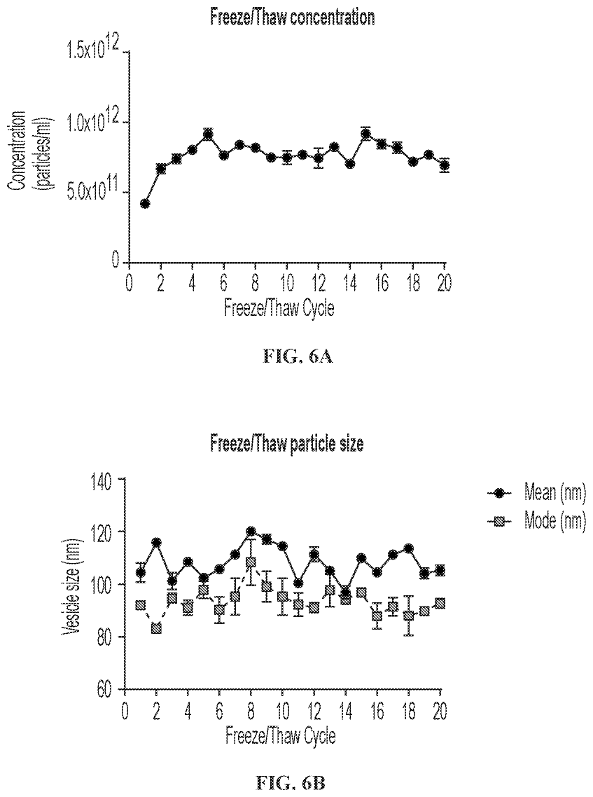

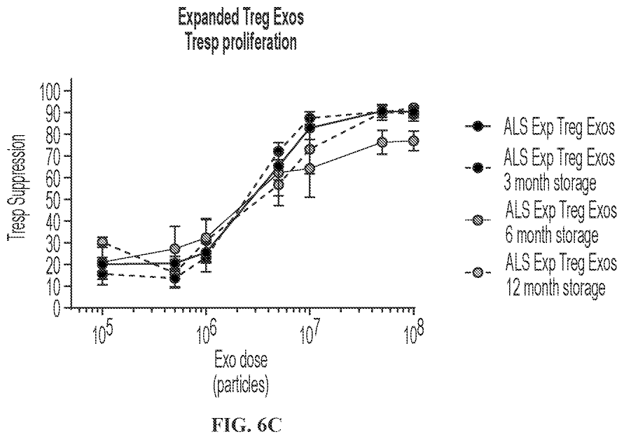

Treg EV stability and function were evaluated after 1 to 20 freeze/thaw cycles and after storage at −20° for 3 months, 6 months, or 12 months. No loss in Treg EV particle number (FIG. 6A) or significant deviation in particle size (FIG. 6B) was observed following multiple freeze/thaw cycles. Treg EV suppression of T cell proliferation did not decrease over time in frozen-20° C. storage (FIG. 6C). The Treg EVs utilized for these experiments were from cultures of ex vivo-expanded Tregs from ALS patients. The Tregs were cultured in human serum (not exosome-depleted), and the EVs were isolated using the PEG process described above.

### 6.6 Example 6: EV Yield

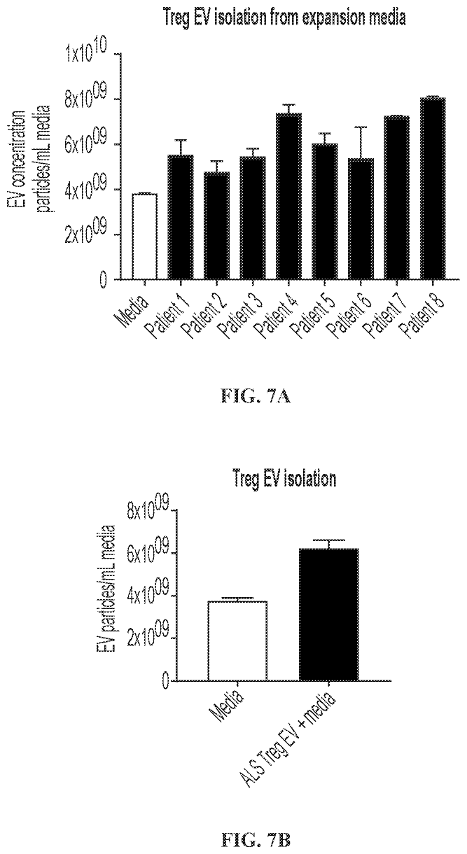

ALS Treg EV concentrations were measured after EV isolations (using PEG described above) from expansion media and from media alone. FIG. 7A shows the EV particle yield/ml of media. The number of EVs increased 1.61 fold in the expansion media compared to media alone (FIG. 7B). Therefore, the increase in EVs likely stems from the expanded Treg populations. It is estimated that about 30% of the total EV population from these samples are derived from expanded Treg cells.

### 6.7 Example 7: Particle Size of EVs Isolated by TFF

Treg EVs derived from ex vivo-expanded ALS patient Tregs cultured in human serum (not exosome-depleted) were isolated using tangential flow filtration (TFF) techniques. Briefly, the isolation utilized a Repligen KR2i TFF system that allows for isolation, concentration, and diafiltration of Treg EV populations using a PBS buffer.

First, media from the Treg expansion culture was circulated using TFF and a 0.65 μm Spectrum mPES Hollow Fiber 85 cm² filter to filter out cells and debris.

The permeate of this step was then subjected to a process designed to concentrate and diafiltrate the EV population into the retentate with continuous circulation. This process utilized the TFF system and a Spectrum mPES Hollow Fiber 115 cm² filter (500 kD) to retain/concentrate all particles greater than approximately 60-80 nm into the retentate.

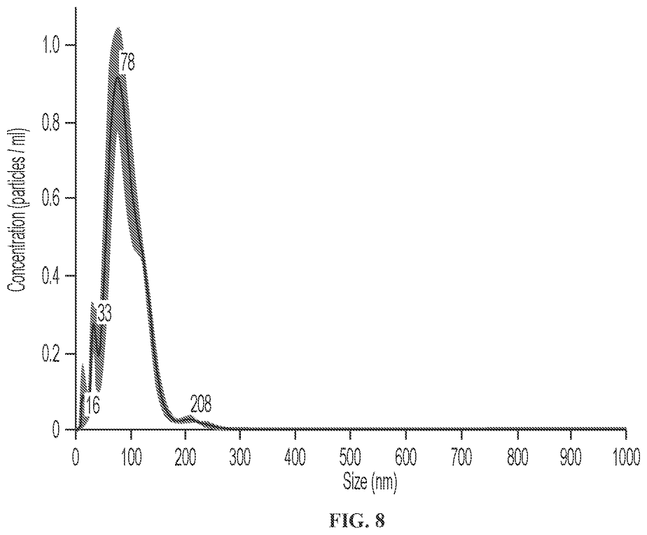

Sterile PBS was incorporated into the circulation, resulting in diafiltration and replacement of the existing solution. The Treg EVs isolated using the TFF protocol showed a size profile with a mean of 92.1 nm+4.2 nm and a mode of 73.3 nm+6.1 nm (FIG. 8).

### 6.8 Example 8: Treg EVs May Induce Conversion of Pro-Inflammatory M1 Cells to Anti-Inflammatory M2 Cells

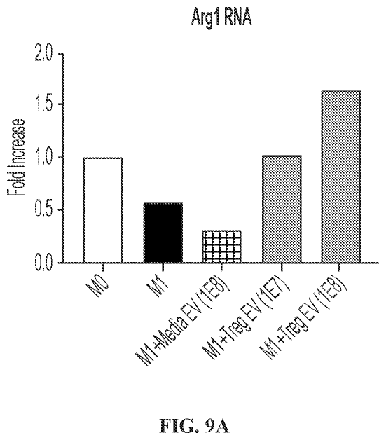

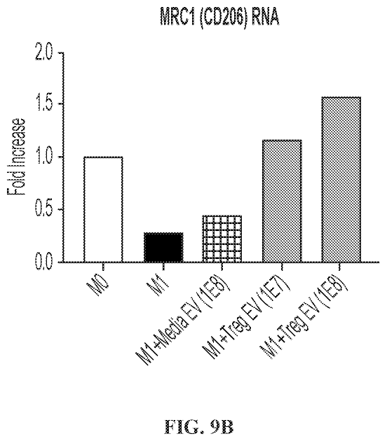

Treg EVs derived from ALS patient Tregs (in particular, ALS patient Tregs ex vivo-expanded in media containing serum not depleted for exosomes) were added to M1 cell cultures at different doses for an overnight timepoint (18 hr). The Treg EVs were found to be able to induce Arg1 and CD206 mRNA (see FIG. 9A and FIG. 9B, respectively), suggesting a conversion of the M1 cells to anti-inflammatory M2 cells.

The Treg EVs utilized for these experiments were from cultures of ex vivo-expanded Tregs from ALS patients. The Tregs were cultured in human serum (not exosome-depleted), and the EVs were isolated using the PEG process described above. Pro-inflammatory M1 cells were polarized as described above.

### 6.9 Example 9: Treg EVs Have a Greater Suppressive Effect on Pro-Inflammatory M1 Cells Compared to MSC EVs

As explained in Example 4, above, EVs isolated from Tregs (“Treg EVs”) and EVs isolated from mesenchymal stem cells (“MSC EVs”) were tested for their ability to suppress immune cells. As demonstrated herein, the Treg EVs exhibit greater suppression of pro-inflammatory M1 cells compared to MSC EVs. The Treg EVs utilized for these experiments were pure Treg EV populations from cultures of ex vivo-expanded Tregs from healthy human subjects. The Tregs were cultured in exosome-free FBS, and the EVs were isolated using the PEG process described above. The MSC EVs utilized for these experiments were isolated from cultures of human bone marrow MSCs and were isolated using the PEG process described above.

In particular, MSCs were obtained from a collaboration whereby MSCs were obtained and grown from bone marrow and passaged 3-4 times before being grown to 80% confluency in flasks in 10% FBS supplemented 1640 media. Then the 10% FBS supplemented 1640 media was replaced with serum-free media for 48 hours. Following 48 hours in serum-free MSC media, the EVs were harvested from the tissue culture media.

Pro-inflammatory myeloid studies and T cell proliferation studies were performed using iPSC-derived pro-inflammatory myeloid cell protocols. The T cell proliferation assays used T cells isolated from the same control patient for reproducibility. Control EVs were isolated from media containing 5% human AB serum that were never used in culture (i.e., effectively serum EVs). Control T cells were isolated from human blood. All assays were performed in triplicate.

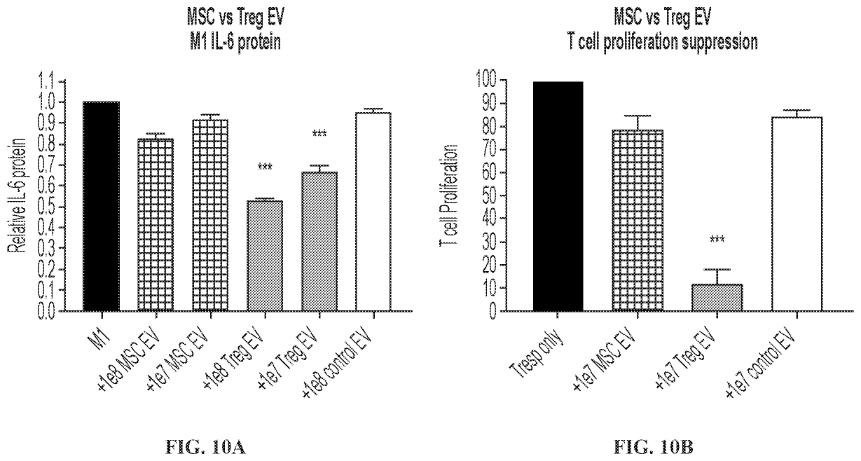

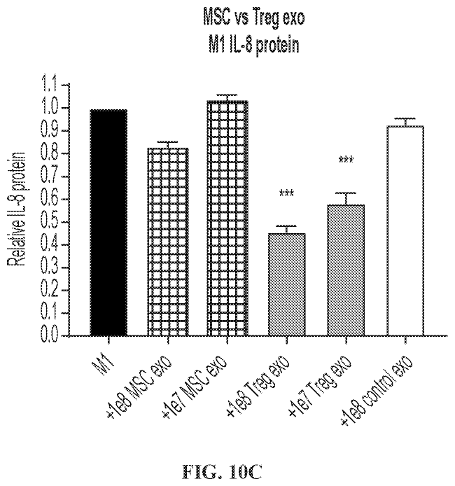

The data shows that Treg EVs were significantly more potent than MSC EVs in suppressing M1 pro-inflammatory IL-6 protein production (FIG. 10A), T cell proliferation (FIG. 10B) and M1 pro-inflammatory IL-8 protein production (FIG. 10C).

### 6.10 Example 10: Particle Size of EVs Isolated by TFF

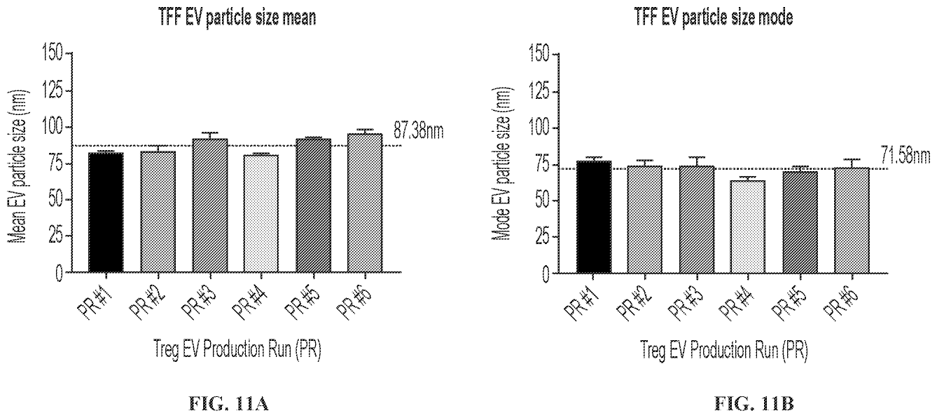

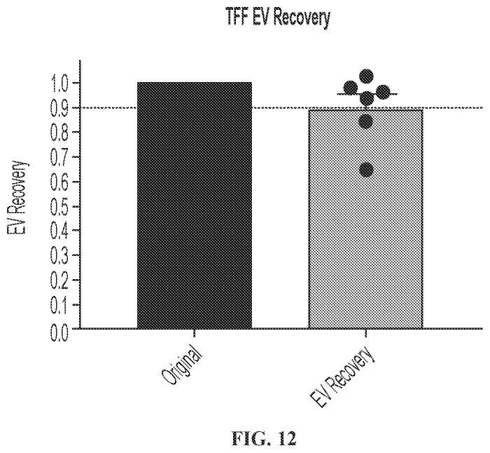

EVs derived from the cultured media from patient Treg expansions were isolated using tangential flow filtration (TFF) techniques. TFF was run utilizing the two-step protocol described above in Example 3. The size profiles of the isolated EVs were measured and data is shown in FIG. 11A and FIG. 11B. Each column in the figures represents the EVs isolated from a different patient's Treg cultured media from the expansion process.

FIG. 11A shows the mean value of particle size. FIG. 11B shows the mode value of particle size. The data shows that Treg EVs isolated using the TFF protocol showed a size profile with a mean of 87.38 nm (FIG. 11A) and a mode of 71.58 nm (FIG. 11B).

For patients #1-4, Treg expansion was performed using the flask production method as described in Example 1. For patients #5 and #6, Treg expansion was performed using a bioreactor (a Terumo BCT Quantum R Cell Expansion System). The data shows that EV size was not significantly different between the two expansion methods.

### 6.11 Example 11: TFF EV Recovery

Recovery of EVs after TFF isolation was calculated via Nanosight particle analysis, as described in Section 6.3.1.4, above. Total EV numbers were calculated using particles/mL values and multiplied by the solution volumes of the original media and the total isolated product, respectively. The level of recovery of EVs recovered from the original medium by the TFF isolation is shown in FIG. 12.

### 6.12 Example 12: Automated Treg Expansion

Following isolation and enrichment (CD25* enrichment/CD8+CD19+ depletion, for example, via CliniMACSR Plus or CliniMACS Prodigy®), the CD25*-enriched cells are incubated in a Quantum Cell Expansion System (Terumo BCT).

Within 24 hours of the initiation of the culturing of the isolated and enriched cells (preferably within 30 minutes following isolation and enrichment) (Day 0), the CD25™ cells are activated with anti-CD3/anti-CD28 beads at a 4:1 beads-to-cell ratio in the bioreactor. IL-2 and rapamycin are also added on Day 0 within 24 hours of the initiation of the culturing of the isolated and enriched cells (preferably within 30 minutes of isolation and enrichment).

The culture medium is replenished with IL-2 every 3-4 days, and IL-2 concentration is adjusted depending on cell number (i.e., the number of all cells in culture, including the enriched Treg cells). Specifically, the cells are cultured in a culture medium containing about 200 IU/mL IL-2 until the cell number reaches 600×10⁶, and then are cultured in a culture medium containing about 250 IU/mL IL-2. The culture medium also contains human AB serum (e.g., 1% or 0.5% human AB serum).

The flow rate of the extracapillary (EC) medium is also adjusted depending on cell number (i.e., the number of all cells in culture, including the enriched Treg cells). Specifically, the flow rate of the EC medium is maintained at 0 until the cell number reaches 500×10⁶, then is increased to about 0.2 mL/min and maintained at about 0.2 mL/min until the cell number reaches 750×10⁶, then is increased to about 0.4 mL/min and maintained at about 0.4 mL/min until the cell number reaches about 1,000×10⁶, then is increased to about 0.6 mL/min and maintained at about 0.6 mL/min until the cell number reaches about 1,500×10⁶, and then is increased to about 0.8 mL/min and maintained at about 0.8 mL/min. The EC medium contains rapamycin. The cells are expanded in the Quantum bioreactor from Day 1. Cell counts and viability are determined each day. Glucose and lactate levels in the culture media are also measured daily.

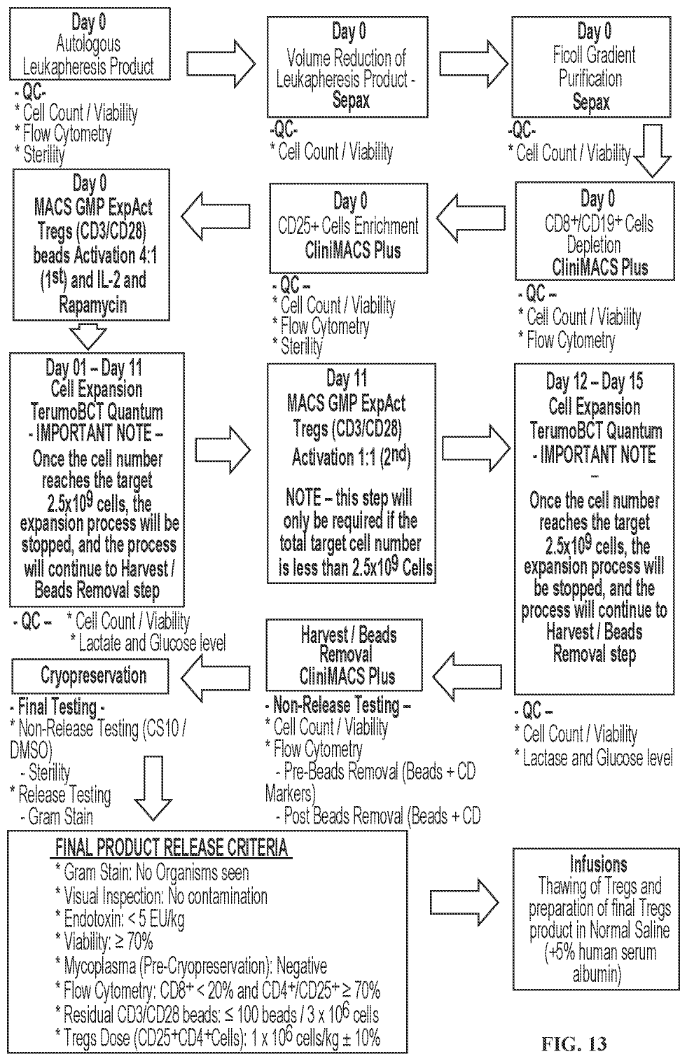

Before or on Day 11, if the cell expansion yields the dose of cells required (≥2.5×10⁹ cells), then the cells are harvested and cryopreserved following bead removal. If the cell expansion process has not reached dose by Day 11, then the cells are reactivated on Day 11 with anti-CD3/anti-CD28 beads at a 1:1 beads-to-cell ratio in the bioreactor. The expansion process may continue in the bioreactor from Day 12 to Day 15, as necessary. Cell counts and viability are measured each day. Once the cell expansion process yields the dose of cells needed (occurring any day between Days 12 and 15), then the cells are immediately harvested and cryopreserved following bead removal. See FIG. 13 for a corresponding process flow diagram.

### 6.13 Example 13: Characterization of TFF Isolated EVs by Proteomics

A proteomic analysis was done on three TFF isolated Treg EV samples. These samples had a mix of serum EVs that came from the 5% human AB supplemented media that was utilized in the GMP manufacturing. Two independent samples of the 5% human AB serum supplemented media were characterized in order to subtract the serum EV background and to obtain a unique signature for Treg EVs. The serum EVs (also referred to as the “media EVs”) also were isolated using TFF.

The proteomic signature of Treg EVs was compared to that of the media serum EVs, effectively generating the proteomic profile of the Treg EVs by subtracting the background. Data is presented in Table 10 below, which shows the top gene products enriched in the Treg EVs compared to background media EVs, which top gene products met the adjusted p-value cutoff of less than 0.1. A positive “Log 2 of fold-change of media EV vs. Treg EV” value in the table represents enrichment of the corresponding gene product in Treg EVs compared to background media EVs. The value shown is the log 2 fold change. Also presented in the table are the adjusted p-values, and the intensity-based absolute quantification (iBAQ) values of the gene products from the mass spectrometry run for the three Treg EV samples and the two media EV samples.

TABLE 9
Gene products enriched in Treg EVs compared to media EVs

| | | Log2 of | |
|---|---|---|---|
| | | fold-change | |
| | | of media EV | Adjusted |
| Gene Symbol | Gene Description | vs. Treg EV | p-value |
| HIST1H2BB | histone cluster 1, H2bb | 16.786 | 0.000 |
| HIST1H3I | histone cluster 1, H3i | 16.295 | 0.001 |
| KRT8 | keratin 8, type II | 16.100 | 0.000 |
| KRT7 | keratin 7, type II | 15.907 | 0.000 |
| HIST1H1B | histone cluster 1, H1b | 14.144 | 0.003 |
| LOC102724334 | histone H2B type F-S-like | 14.069 | 0.017 |
| HIST1H2BH | histone cluster 1, H2bh | 13.746 | 0.003 |
| HIST1H2BD | histone cluster 1, H2bd | 13.597 | 0.000 |
| HIST1H3C | histone cluster 1, H3c | 13.527 | 0.002 |
| ARHGDIB | Rho GDP dissociation | 13.216 | 0.002 |
| | inhibitor (GDI) beta | | |
| HIST2H3A | histone cluster 2, H3a | 13.173 | 0.001 |
| XPNPEP3 | X-prolyl aminopeptidase 3, | 13.037 | 0.003 |
| | mitochondrial | | |
| PFN1 | profilin 1 | 13.036 | 0.019 |
| HIST1H2BE | histone cluster 1, H2be | 12.946 | 0.001 |
| HIST1H3F | histone cluster 1, H3f | 12.741 | 0.004 |
| PSMA5 | proteasome subunit alpha 5 | 12.657 | 0.006 |
| HIST1H2BC | histone cluster 1, H2bc | 12.022 | 0.000 |
| PSMA3 | proteasome subunit alpha 3 | 11.962 | 0.002 |
| HIST4H4 | histone cluster 4, H4 | 11.947 | 0.019 |
| KRT73 | keratin 73, type II | 11.774 | 0.019 |
| HIST1H2BM | histone cluster 1, H2bm | 11.764 | 0.002 |
| HIST1H4B | histone cluster 1, H4b | 11.678 | 0.017 |
| HIST1H4I | histone cluster 1, H4i | 11.629 | 0.016 |
| HIST2H2AB | histone cluster 2, H2ab | 11.605 | 0.000 |
| RAC2 | ras-related C3 botulinum | 11.508 | 0.000 |
| | toxin substrate 2 (rho | | |
| | family, small GTP binding | | |
| | protein Rac2) | | |
| CFL1 | cofilin 1 (non-muscle) | 11.470 | 0.007 |
| HIST2H3D | histone cluster 2, H3d | 11.398 | 0.007 |
| HIST1H4F | histone cluster 1, H4f | 11.322 | 0.013 |
| HIST1H3G | histone cluster 1, H3g | 11.311 | 0.000 |
| HIST1H2BL | histone cluster 1, H2bl | 11.284 | 0.003 |
| HIST1H3A | histone cluster 1, H3a | 11.183 | 0.006 |
| HIST2H2BE | histone cluster 2, H2be | 11.109 | 0.023 |
| HIST1H4D | histone cluster 1, H4d | 11.069 | 0.010 |
| HIST1H2BJ | histone cluster 1, H2bj | 10.978 | 0.001 |
| HIST1H2BI | histone cluster 1, H2bi | 10.960 | 0.043 |
| HIST1H3H | histone cluster 1, H3h | 10.941 | 0.006 |
| HIST1H1C | histone cluster 1, Hlc | 10.891 | 0.007 |
| HIST1H2BK | histone cluster 1, H2bk | 10.840 | 0.005 |
| GSTP1 | glutathione S-transferase pi | 10.801 | 0.094 |
| | 1 | | |
| HIST1H3J | histone cluster 1, H3j | 10.794 | 0.074 |
| RAB35 | RAB35, member RAS | 10.786 | 0.003 |
| | oncogene family | | |
| LGALS1 | lectin, galactoside-binding, | 10.733 | 0.022 |
| | soluble, 1 | | |
| HIST1H4A | histone cluster 1, H4a | 10.630 | 0.007 |
| HIST1H4C | histone cluster 1, H4c | 10.617 | 0.007 |
| CLIC1 | chloride intracellular | 10.612 | 0.002 |
| | channel 1 | | |
| RPS20 | ribosomal protein S20 | 10.448 | 0.019 |
| HIST1H3D | histone cluster 1, H3d | 10.423 | 0.006 |
| HIST2H2BF | histone cluster 2, H2bf | 10.418 | 0.007 |
| FTH1 | ferritin, heavy polypeptide | 10.407 | 0.006 |
| | 1 | | |
| ARPC4 | actin related protein 2/3 | 10.398 | 0.003 |
| | complex, subunit 4, 20 kDa | | |
| HIST1H3B | histone cluster 1, H3b | 10.377 | 0.000 |
| APOF | apolipoprotein F | 10.356 | 0.006 |
| MSN | moesin | 10.311 | 0.003 |
| RHOG | ras homolog family | 10.252 | 0.006 |
| | member G | | |
| PPIA | peptidylprolyl isomerase A | 10.063 | 0.018 |
| | (cyclophilin A) | | |
| HIST1H2AE | histone cluster 1, H2ae | 9.971 | 0.000 |
| HIST1H2AC | histone cluster 1, H2ac | 9.971 | 0.000 |
| HIST1H2AB | histone cluster 1, H2ab | 9.971 | 0.000 |
| HIST3H2A | histone cluster 3, H2a | 9.971 | 0.000 |
| ANXA5 | annexin A5 | 9.887 | 0.007 |
| HIST1H4H | histone cluster 1, H4h | 9.848 | 0.003 |
| PSMA7 | proteasome subunit alpha 7 | 9.847 | 0.069 |
| RPS18 | ribosomal protein S18 | 9.843 | 0.074 |
| HIST1H2BN | histone cluster 1, H2bn | 9.827 | 0.009 |
| HIST2H3C | histone cluster 2, H3c | 9.786 | 0.003 |
| HIST1H1D | histone cluster 1, H1d | 9.764 | 0.006 |
| MYL6 | myosin, light chain 6, | 9.753 | 0.022 |
| | alkali, smooth muscle and | | |
| | non-muscle | | |
| GGH | gamma-glutamyl hydrolase | 9.699 | 0.006 |
| | (conjugase, folylpolygammaglutamyl | | |
| | hydrolase) | | |
| RAN | RAN, member RAS | 9.609 | 0.007 |
| | oncogene family | | |
| TRAP1 | TNF receptor-associated | 9.418 | 0.020 |
| | protein 1 | | |
| S100A4 | S100 calcium binding | 9.347 | 0.006 |
| | protein A4 | | |
| STMN1 | stathmin 1 | 9.303 | 0.030 |
| HSP90AA1 | heat shock protein 90 kDa | 9.237 | 0.003 |
| | alpha (cytosolic), class A | | |
| | member 1 | | |
| HIST1H4L | histone cluster 1, H41 | 9.080 | 0.001 |
| YWHAZ | tyrosine 3- | 9.063 | 0.007 |
| | monooxygenase/tryptophan | | |
| | 5-monooxygenase | | |
| | activation protein, zeta | | |
| MIF | macrophage migration | 8.954 | 0.002 |
| | inhibitory factor | | |
| | (glycosylation-inhibiting | | |
| | factor) | | |
| HIST3H3 | histone cluster 3, H3 | 8.857 | 0.035 |
| YWHAQ | tyrosine 3- | 8.836 | 0.017 |
| | monooxygenase/tryptophan | | |
| | 5-monooxygenase | | |
| | activation protein, theta | | |
| HIST2H4B | histone cluster 2, H4b | 8.835 | 0.000 |
| HIST1H2BF | histone cluster 1, H2bf | 8.748 | 0.000 |
| RAB11A | RAB11A, member RAS | 8.510 | 0.061 |
| | oncogene family | | |
| YWHAB | tyrosine 3- | 8.459 | 0.042 |
| | monooxygenase/tryptophan | | |
| | 5-monooxygenase | | |
| | activation protein, beta | | |
| HIST1H2AD | histone cluster 1, H2ad | 8.441 | 0.000 |
| HIST1H2AI | histone cluster 1, H2ai | 8.441 | 0.000 |
| HIST1H2AK | histone cluster 1, H2ak | 8.441 | 0.000 |
| HIST1H2AJ | histone cluster 1, H2aj | 8.441 | 0.000 |
| HIST1H2AL | histone cluster 1, H2al | 8.441 | 0.000 |
| HIST1H2AM | histone cluster 1, H2am | 8.441 | 0.000 |
| HIST2H2AA3 | histone cluster 2, H2aa3 | 8.441 | 0.000 |
| HIST2H2AC | histone cluster 2, H2ac | 8.441 | 0.000 |
| HIST1H2AG | histone cluster 1, H2ag | 8.441 | 0.000 |
| H2AFJ | H2A histone family, | 8.441 | 0.000 |
| | member J | | |
| HIST1H2AH | histone cluster 1, H2ah | 8.441 | 0.000 |
| HIST2H2AA4 | histone cluster 2, H2aa4 | 8.441 | 0.000 |
| ENO1 | enolase 1, (alpha) | 8.349 | 0.057 |
| GNA13 | guanine nucleotide binding | 8.340 | 0.074 |
| | protein (G protein), alpha | | |
| | 13 | | |
| LGALS3 | lectin, galactoside-binding, | 8.319 | 0.053 |
| | soluble, 3 | | |
| UTS2 | urotensin 2 | 8.244 | 0.006 |
| HIST1H4E | histone cluster 1, H4e | 8.227 | 0.000 |
| PSMB9 | proteasome subunit beta 9 | 8.191 | 0.047 |
| H2BFS | H2B histone family, | 8.079 | 0.095 |
| | member S (pseudogene) | | |
| RPL11 | ribosomal protein L11 | 8.011 | 0.079 |
| VCP | valosin containing protein | 7.997 | 0.049 |
| TPM3 | tropomyosin 3 | 7.973 | 0.003 |
| NME2 | NME/NM23 nucleoside | 7.898 | 0.014 |
| | diphosphate kinase 2 | | |
| RPS15A | ribosomal protein S15a | 7.758 | 0.092 |
| CALM3 | calmodulin 3 | 7.757 | 0.008 |
| | (phosphorylase kinase, | | |
| | delta) | | |
| HIST1H2BG | histone cluster 1, H2bg | 7.683 | 0.031 |
| RPS19 | ribosomal protein S19 | 7.669 | 0.072 |
| RAP1A | RAPIA, member of RAS | 7.657 | 0.049 |
| | oncogene family | | |
| CALM2 | calmodulin 2 | 7.472 | 0.013 |
| | (phosphorylase kinase, | | |
| | delta) | | |
| PSMA6 | proteasome subunit alpha 6 | 7.447 | 0.003 |
| EZR | ezrin | 7.436 | 0.019 |
| HIST2H4A | histone cluster 2, H4a | 7.419 | 0.003 |
| TUBA1A | tubulin, alpha 1a | 7.384 | 0.033 |
| MDH1 | malate dehydrogenase 1, | 7.270 | 0.031 |
| | NAD (soluble) | | |
| TAS2R42 | taste receptor, type 2, | 7.258 | 0.090 |
| | member 42 | | |
| HIST1H4K | histone cluster 1, H4k | 7.202 | 0.006 |
| LDHA | lactate dehydrogenase A | 7.146 | 0.026 |
| PSMB2 | proteasome subunit beta 2 | 7.033 | 0.019 |
| PGK1 | phosphoglycerate kinase 1 | 6.949 | 0.074 |
| PSMA4 | proteasome subunit alpha 4 | 6.918 | 0.074 |
| NME1-NME2 | NME1-NME2 readthrough | 6.820 | 0.026 |
| HIST1H1E | histone cluster 1, H1e | 6.673 | 0.006 |
| RPL38 | ribosomal protein L38 | 6.666 | 0.007 |
| PSMA2 | proteasome subunit alpha 2 | 6.592 | 0.006 |
| GNAI1 | guanine nucleotide binding | 6.574 | 0.073 |
| | protein (G protein), alpha | | |
| | inhibiting activity | | |
| | polypeptide 1 | | |
| TUBA1B | tubulin, alpha 1b | 6.432 | 0.046 |
| F5 | coagulation factor V | 6.377 | 0.007 |
| | (proaccelerin, labile factor) | | |
| PARK7 | parkinson protein 7 | 6.334 | 0.022 |
| HIST1H4J | histone cluster 1, H4j | 6.262 | 0.034 |
| RPL22 | ribosomal protein L22 | 6.150 | 0.074 |
| LPCAT2 | lysophosphatidylcholine | 6.053 | 0.086 |
| | acyltransferase 2 | | |
| KRT19 | keratin 19, type I | 5.940 | 0.040 |
| HLA-A | major histocompatibility | 5.631 | 0.095 |
| | complex, class I, A | | |
| KRT15 | keratin 15, type I | 5.550 | 0.074 |
| TPI1 | triosephosphate isomerase | 5.523 | 0.002 |
| | 1 | | |
| SPARCL1 | SPARC-like 1 (hevin) | 5.420 | 0.059 |
| NUP155 | nucleoporin 155 kDa | 5.407 | 0.076 |
| KATNAL2 | katanin p60 subunit A-like | 5.359 | 0.096 |
| | 2 | | |
| KRT6B | keratin 6B, type II | 5.351 | 0.014 |
| CD14 | CD14 molecule | 5.244 | 0.009 |
| PSMB3 | proteasome subunit beta 3 | 5.225 | 0.068 |
| CDC42 | cell division cycle 42 | 5.197 | 0.060 |
| HABP2 | hyaluronan binding protein | 4.966 | 0.010 |
| | 2 | | |
| GNAT2 | guanine nucleotide binding | 4.956 | 0.090 |
| | protein (G protein), alpha | | |
| | transducing activity | | |
| | polypeptide 2 | | |
| ARF1 | ADP-ribosylation factor 1 | 4.851 | 0.033 |
| RAB11B | RAB11B, member RAS | 4.672 | 0.038 |
| | oncogene family | | |
| ACTB | actin, beta | 4.591 | 0.001 |
| ACTG1 | actin gamma 1 | 4.591 | 0.001 |
| UBC | ubiquitin C | 4.518 | 0.035 |
| UBB | ubiquitin B | 4.518 | 0.019 |
| RPS27A | ribosomal protein S27a | 4.518 | 0.013 |
| UBA52 | ubiquitin A-52 residue | 4.518 | 0.014 |
| | ribosomal protein fusion | | |
| | product 1 | | |
| PKM | pyruvate kinase, muscle | 4.083 | 0.017 |
| LDHB | lactate dehydrogenase B | 4.046 | 0.028 |
| KRT77 | keratin 77, type II | 3.988 | 0.026 |
| CALM1 | calmodulin 1 | 3.855 | 0.092 |
| | (phosphorylase kinase, | | |
| | delta) | | |
| PIGR | polymeric immunoglobulin | 3.793 | 0.007 |
| | receptor | | |
| MYL12B | myosin, light chain 12B, | 3.718 | 0.041 |
| | regulatory | | |
| B2M | beta-2-microglobulin | 3.678 | 0.017 |
| FTL | ferritin, light polypeptide | 3.456 | 0.011 |
| C9 | complement component 9 | 3.370 | 0.026 |
| LBP | lipopolysaccharide binding | 3.350 | 0.007 |
| | protein | | |
| GANAB | glucosidase, alpha; neutral | 3.242 | 0.062 |
| | AB | | |
| SERPINA10 | serpin peptidase inhibitor, | 3.135 | 0.013 |
| | clade A (alpha-1 | | |
| | antiproteinase, antitrypsin), | | |
| | member 10 | | |
| PCYOX1 | prenylcysteine oxidase 1 | 2.908 | 0.038 |
| RIN1 | Ras and Rab interactor 1 | 2.899 | 0.013 |
| ARPC3 | actin related protein 2/3 | 2.873 | 0.019 |
| | complex, subunit 3, 21 kDa | | |
| KRT6A | keratin 6A, type II | 2.798 | 0.053 |
| HSPA5 | heat shock 70 kDa protein 5 | 2.624 | 0.074 |
| | (glucose-regulated protein, | | |
| | 78 kDa) | | |
| LAMP2 | lysosomal-associated | 2.437 | 0.081 |
| | membrane protein 2 | | |
| PRDX1 | peroxiredoxin 1 | 2.262 | 0.052 |
| SERPIND1 | serpin peptidase inhibitor, | 2.113 | 0.005 |
| | clade D (heparin cofactor), | | |
| | member 1 | | |
| C4BPA | complement component 4 | 1.492 | 0.029 |
| | binding protein, alpha | | |
| APOE | apolipoprotein E | 1.488 | 0.033 |
| APOH | apolipoprotein H (beta-2- | 1.420 | 0.026 |
| | glycoprotein I) | | |
| PGLYRP2 | peptidoglycan recognition | 1.264 | 0.019 |
| | protein 2 | | |
| PROS1 | protein S (alpha) | 1.202 | 0.096 |
| HPX | hemopexin | 1.149 | 0.023 |
| C5 | complement component 5 | 1.131 | 0.099 |
| CPN1 | carboxypeptidase N, | 1.102 | 0.096 |
| | polypeptide 1 | | |
| HYI | hydroxypyruvate isomerase | 1.098 | 0.047 |
| | (putative) | | |
| C1RL | complement component 1, | 1.033 | 0.047 |
| | r subcomponent-like | | |
| PON3 | paraoxonase 3 | 1.002 | 0.060 |
| C4B | complement component 4B | 0.999 | 0.092 |
| | (Chido blood group) | | |
| F2 | coagulation factor II | 0.998 | 0.096 |
| | (thrombin) | | |
| PON1 | paraoxonase 1 | 0.977 | 0.042 |
| SERPINC1 | serpin peptidase inhibitor, | 0.798 | 0.085 |
| | clade C (antithrombin), | | |
| | member 1 | | |
| C3 | complement component 3 | 0.789 | 0.089 |
| VTN | vitronectin | 0.741 | 0.074 |

| | iBAQ | iBAQ | iBAQ | iBAQ | iBAQ |
|---|---|---|---|---|---|
| | Treg EV | Treg EV | Treg EV | Media EV | Media EV |
| Gene Symbol | sample 1 | sample 2 | sample 3 | sample 1 | sample 2 |
| HIST1H2BB | 69.742 | 79.297 | 104.170 | 0.000 | 0.000 |
| HIST1H3I | 17.106 | 13.684 | 15.612 | 0.000 | 0.000 |
| KRT8 | 84.821 | 48.527 | 64.742 | 0.000 | 0.000 |
| KRT7 | 67.281 | 52.529 | 50.531 | 0.000 | 0.000 |
| HIST1H1B | 3.915 | 5.896 | 7.176 | 0.000 | 0.000 |
| LOC102724334 | 20.812 | 23.719 | 30.980 | 0.000 | 0.000 |
| HIST1H2BH | 20.812 | 23.719 | 30.980 | 0.000 | 0.000 |
| HIST1H2BD | 19.424 | 22.138 | 28.915 | 0.000 | 0.000 |
| HIST1H3C | 17.106 | 13.684 | 15.612 | 0.000 | 0.000 |
| ARHGDIB | 2.048 | 3.601 | 4.813 | 0.000 | 0.000 |
| HIST2H3A | 17.106 | 13.684 | 15.612 | 0.000 | 0.000 |
| XPNPEP3 | 1.384 | 0.955 | 1.773 | 0.000 | 0.000 |
| PFN1 | 13.561 | 17.296 | 18.494 | 0.000 | 0.000 |
| HIST1H2BE | 20.812 | 23.719 | 30.980 | 0.000 | 0.000 |
| HIST1H3F | 17.106 | 13.684 | 15.612 | 0.000 | 0.000 |
| PSMA5 | 1.885 | 1.568 | 1.976 | 0.000 | 0.000 |
| HIST1H2BC | 20.812 | 23.719 | 30.980 | 0.000 | 0.000 |
| PSMA3 | 2.043 | 2.025 | 2.454 | 0.000 | 0.000 |
| HIST4H4 | 10.916 | 9.556 | 10.598 | 0.055 | 0.000 |
| KRT73 | 46.688 | 30.090 | 33.280 | 0.219 | 0.000 |
| HIST1H2BM | 20.812 | 23.719 | 30.980 | 0.000 | 0.000 |
| HIST1H4B | 10.916 | 9.556 | 10.598 | 0.055 | 0.000 |
| HIST1H4I | 10.916 | 9.556 | 10.598 | 0.055 | 0.000 |
| HIST2H2AB | 109.304 | 148.877 | 184.610 | 0.095 | 0.070 |
| RAC2 | 11.594 | 10.403 | 11.627 | 0.000 | 0.000 |
| CFL1 | 9.681 | 13.235 | 13.171 | 0.000 | 0.000 |
| HIST2H3D | 17.106 | 13.684 | 15.612 | 0.000 | 0.000 |
| HIST1H4F | 10.916 | 9.556 | 10.598 | 0.055 | 0.000 |
| HIST1H3G | 17.106 | 13.684 | 15.612 | 0.000 | 0.000 |
| HIST1H2BL | 20.812 | 23.719 | 30.980 | 0.000 | 0.000 |
| HIST1H3A | 17.106 | 13.684 | 15.612 | 0.000 | 0.000 |
| HIST2H2BE | 69.742 | 79.297 | 104.170 | 0.000 | 0.000 |
| HIST1H4D | 10.916 | 9.556 | 10.598 | 0.055 | 0.000 |
| HIST1H2BJ | 69.742 | 79.297 | 104.170 | 0.000 | 0.000 |
| HIST1H2BI | 20.812 | 23.719 | 30.980 | 0.000 | 0.000 |
| HIST1H3H | 17.106 | 13.684 | 15.612 | 0.000 | 0.000 |
| HIST1H1C | 3.714 | 6.087 | 8.729 | 0.000 | 0.000 |
| HIST1H2BK | 20.812 | 23.719 | 30.980 | 0.000 | 0.000 |
| GSTP1 | 2.437 | 2.601 | 2.691 | 0.000 | 0.110 |
| HIST1H3J | 17.106 | 13.684 | 15.612 | 0.000 | 0.000 |
| RAB35 | 1.471 | 1.441 | 1.742 | 0.000 | 0.000 |
| LGALS1 | 0.215 | 1.534 | 0.560 | 0.000 | 0.000 |
| HIST1H4A | 10.916 | 9.556 | 10.598 | 0.055 | 0.000 |
| HIST1H4C | 10.916 | 9.556 | 10.598 | 0.055 | 0.000 |
| CLIC1 | 1.683 | 6.223 | 4.093 | 0.000 | 0.000 |
| RPS20 | 1.666 | 2.284 | 2.358 | 0.000 | 0.000 |
| HIST1H3D | 17.106 | 13.684 | 15.612 | 0.000 | 0.000 |
| HIST2H2BF | 19.424 | 22.138 | 28.915 | 0.000 | 0.000 |
| FTH1 | 2.440 | 0.567 | 2.656 | 0.000 | 0.000 |
| ARPC4 | 8.936 | 6.375 | 8.387 | 0.000 | 0.000 |
| HIST1H3B | 17.106 | 13.684 | 15.612 | 0.000 | 0.000 |
| APOF | 1.620 | 0.646 | 0.505 | 0.000 | 0.000 |
| MSN | 5.395 | 10.165 | 14.425 | 0.000 | 0.000 |
| RHOG | 0.882 | 0.883 | 0.828 | 0.000 | 0.000 |
| PPIA | 13.898 | 14.412 | 16.305 | 0.000 | 0.000 |
| HIST1H2AE | 33.728 | 49.475 | 60.131 | 0.095 | 0.070 |
| HIST1H2AC | 33.728 | 49.475 | 60.131 | 0.095 | 0.070 |
| HIST1H2AB | 33.728 | 49.475 | 60.131 | 0.095 | 0.070 |
| HIST3H2A | 33.728 | 49.475 | 60.131 | 0.095 | 0.070 |
| ANXA5 | 0.511 | 0.473 | 1.337 | 0.000 | 0.000 |
| HIST1H4H | 10.916 | 9.556 | 10.598 | 0.055 | 0.000 |
| PSMA7 | 0.407 | 0.570 | 0.663 | 0.000 | 0.000 |
| RPS18 | 0.577 | 5.905 | 0.931 | 0.000 | 0.000 |
| HIST1H2BN | 20.812 | 23.719 | 30.980 | 0.000 | 0.000 |
| HIST2H3C | 17.106 | 13.684 | 15.612 | 0.000 | 0.000 |
| HIST1H1D | 3.376 | 5.534 | 7.936 | 0.000 | 0.000 |
| MYL6 | 2.575 | 3.032 | 3.364 | 0.000 | 0.000 |
| GGH | 0.482 | 0.784 | 1.169 | 0.000 | 0.000 |
| RAN | 5.762 | 4.642 | 7.515 | 0.000 | 0.000 |
| TRAP1 | 0.998 | 0.725 | 1.199 | 0.000 | 0.000 |
| S100A4 | 1.100 | 3.476 | 4.542 | 0.000 | 0.000 |
| STMN1 | 0.217 | 1.607 | 0.480 | 0.000 | 0.000 |
| HSP90AA1 | 3.443 | 1.658 | 4.886 | 0.000 | 0.000 |
| HIST1H4L | 10.916 | 9.556 | 10.598 | 0.055 | 0.000 |
| YWHAZ | 12.433 | 13.667 | 14.473 | 0.000 | 0.000 |
| MIF | 4.408 | 4.318 | 3.645 | 0.000 | 0.000 |
| HIST3H3 | 17.106 | 13.684 | 15.612 | 0.000 | 0.000 |
| YWHAQ | 6.372 | 1.428 | 2.108 | 0.000 | 0.000 |
| HIST2H4B | 10.916 | 9.556 | 10.598 | 0.055 | 0.000 |
| HIST1H2BF | 20.812 | 23.719 | 30.980 | 0.000 | 0.000 |
| RAB11A | 0.259 | 0.290 | 0.270 | 0.000 | 0.000 |
| YWHAB | 8.020 | 2.034 | 1.659 | 0.000 | 0.000 |
| HIST1H2AD | 11.630 | 17.025 | 21.078 | 0.095 | 0.070 |
| HIST1H2AI | 11.630 | 17.025 | 21.078 | 0.095 | 0.070 |
| HIST1H2AK | 11.630 | 17.025 | 21.078 | 0.095 | 0.070 |
| HIST1H2AJ | 11.630 | 17.025 | 21.078 | 0.095 | 0.070 |
| HIST1H2AL | 11.630 | 17.025 | 21.078 | 0.095 | 0.070 |
| HIST1H2AM | 11.630 | 17.025 | 21.078 | 0.095 | 0.070 |
| HIST2H2AA3 | 11.630 | 17.025 | 21.078 | 0.095 | 0.070 |
| HIST2H2AC | 11.630 | 17.025 | 21.078 | 0.095 | 0.070 |
| HIST1H2AG | 11.630 | 17.025 | 21.078 | 0.095 | 0.070 |
| H2AFJ | 11.630 | 17.025 | 21.078 | 0.095 | 0.070 |
| HIST1H2AH | 11.630 | 17.025 | 21.078 | 0.095 | 0.070 |
| HIST2H2AA4 | 11.630 | 17.025 | 21.078 | 0.095 | 0.070 |
| ENO1 | 4.159 | 2.691 | 3.018 | 0.204 | 0.000 |
| GNA13 | 0.000 | 0.248 | 0.136 | 0.000 | 0.000 |
| LGALS3 | 1.202 | 5.134 | 2.980 | 0.142 | 0.000 |
| UTS2 | 3.330 | 2.390 | 2.832 | 0.000 | 0.000 |
| HIST1H4E | 10.916 | 9.556 | 10.598 | 0.055 | 0.000 |
| PSMB9 | 0.570 | 0.522 | 1.047 | 0.000 | 0.000 |
| H2BFS | 20.812 | 23.719 | 30.980 | 0.000 | 0.000 |
| RPL11 | 0.530 | 0.484 | 0.894 | 0.000 | 0.000 |
| VCP | 0.189 | 0.000 | 1.203 | 0.000 | 0.000 |
| TPM3 | 3.050 | 2.451 | 3.478 | 0.000 | 0.000 |
| NME2 | 0.665 | 0.942 | 2.729 | 0.000 | 0.000 |
| RPS15A | 0.563 | 0.000 | 0.874 | 0.000 | 0.000 |
| CALM3 | 0.440 | 0.847 | 1.242 | 0.000 | 0.000 |
| HIST1H2BG | 20.812 | 23.719 | 30.980 | 0.000 | 0.000 |
| RPS19 | 0.239 | 0.085 | 0.000 | 0.000 | 0.000 |
| RAP1A | 1.660 | 1.945 | 2.043 | 0.000 | 0.000 |
| CALM2 | 0.387 | 0.745 | 1.092 | 0.000 | 0.000 |
| PSMA6 | 1.791 | 2.304 | 3.758 | 0.000 | 0.000 |
| EZR | 1.164 | 1.176 | 3.560 | 0.000 | 0.000 |
| HIST2H4A | 10.916 | 9.556 | 10.598 | 0.055 | 0.000 |
| TUBA1A | 1.062 | 0.158 | 0.840 | 0.000 | 0.000 |
| MDH1 | 0.170 | 0.348 | 0.536 | 0.000 | 0.000 |
| TAS2R42 | 3.639 | 0.000 | 4.466 | 0.000 | 0.000 |
| HIST1H4K | 10.916 | 9.556 | 10.598 | 0.055 | 0.000 |
| LDHA | 7.136 | 15.831 | 16.037 | 0.753 | 0.000 |
| PSMB2 | 0.737 | 0.259 | 0.364 | 0.000 | 0.000 |
| PGK1 | 2.463 | 1.177 | 1.582 | 0.000 | 0.000 |
| PSMA4 | 1.086 | 0.964 | 1.364 | 0.000 | 0.000 |
| NME1-NME2 | 0.280 | 0.397 | 1.149 | 0.000 | 0.000 |
| HIST1H1E | 3.714 | 6.087 | 8.729 | 0.000 | 0.000 |
| RPL38 | 1.326 | 1.091 | 1.610 | 0.000 | 0.000 |
| PSMA2 | 1.480 | 1.332 | 2.803 | 0.000 | 0.000 |
| GNAI1 | 0.077 | 0.066 | 0.036 | 0.000 | 0.000 |
| TUBA1B | 1.040 | 0.155 | 0.823 | 0.000 | 0.000 |
| F5 | 32.241 | 7.483 | 22.408 | 0.262 | 0.528 |
| PARK7 | 0.663 | 0.661 | 0.577 | 0.000 | 0.000 |
| HIST1H4J | 10.916 | 9.556 | 10.598 | 0.055 | 0.000 |
| RPL22 | 1.316 | 1.888 | 2.027 | 0.000 | 0.000 |
| LPCAT2 | 0.218 | 0.000 | 0.977 | 0.000 | 0.000 |
| KRT19 | 21.061 | 132.885 | 114.393 | 6.833 | 0.565 |
| HLA-A | 2.164 | 0.097 | 2.146 | 0.000 | 0.000 |
| KRT15 | 12.190 | 128.515 | 112.434 | 7.873 | 0.565 |
| TPI1 | 3.767 | 6.187 | 4.321 | 0.163 | 0.195 |
| SPARCL1 | 0.257 | 0.179 | 0.333 | 0.000 | 0.000 |
| NUP155 | 4.759 | 4.651 | 0.000 | 0.000 | 0.000 |
| KATNAL2 | 0.232 | 0.000 | 0.696 | 0.000 | 0.000 |
| KRT6B | 72.280 | 28.473 | 167.757 | 3.610 | 2.544 |
| CD14 | 9.408 | 5.139 | 4.186 | 0.564 | 0.132 |
| PSMB3 | 0.919 | 1.232 | 1.704 | 0.000 | 0.000 |
| CDC42 | 0.085 | 0.588 | 0.482 | 0.000 | 0.000 |
| HABP2 | 15.185 | 5.201 | 4.370 | 0.304 | 0.515 |
| GNAT2 | 0.000 | 0.054 | 0.030 | 0.000 | 0.000 |
| ARF1 | 0.347 | 0.450 | 0.336 | 0.000 | 0.000 |
| RAB11B | 0.216 | 0.242 | 0.225 | 0.000 | 0.000 |
| ACTB | 51.649 | 48.090 | 62.849 | 3.620 | 4.283 |
| ACTG1 | 51.649 | 48.090 | 62.849 | 3.620 | 4.283 |
| UBC | 0.316 | 1.121 | 0.566 | 0.069 | 0.029 |
| UBB | 0.992 | 3.523 | 1.777 | 0.216 | 0.093 |
| RPS27A | 2.314 | 8.221 | 4.147 | 0.504 | 0.216 |
| UBA52 | 1.984 | 7.047 | 3.555 | 0.432 | 0.185 |
| PKM | 1.892 | 1.176 | 2.575 | 0.143 | 0.243 |
| LDHB | 4.234 | 5.389 | 8.201 | 1.431 | 0.261 |
| KRT77 | 58.661 | 38.578 | 156.125 | 5.706 | 10.807 |
| CALM1 | 0.400 | 0.771 | 1.131 | 0.000 | 0.000 |
| PIGR | 13.786 | 9.950 | 12.247 | 1.072 | 2.131 |
| MYL12B | 0.558 | 0.403 | 0.702 | 0.000 | 0.000 |
| B2M | 29.805 | 8.869 | 13.004 | 0.000 | 2.097 |
| FTL | 5.191 | 3.423 | 5.086 | 0.559 | 0.930 |
| C9 | 166.416 | 41.607 | 77.896 | 13.321 | 14.463 |
| LBP | 3.508 | 3.029 | 2.153 | 0.478 | 0.504 |
| GANAB | 0.319 | 0.468 | 0.536 | 0.109 | 0.000 |
| SERPINA10 | 7.172 | 3.513 | 3.812 | 0.902 | 0.937 |
| PCYOX1 | 1.321 | 1.619 | 1.123 | 0.547 | 0.181 |
| RIN1 | 1.997 | 2.406 | 3.064 | 0.660 | 0.508 |
| ARPC3 | 1.546 | 1.560 | 1.693 | 0.000 | 0.000 |
| KRT6A | 81.923 | 38.407 | 153.606 | 18.411 | 21.493 |
| HSPA5 | 3.752 | 0.950 | 2.106 | 0.569 | 0.550 |
| LAMP2 | 1.493 | 0.668 | 1.313 | 0.278 | 0.456 |
| PRDX1 | 3.959 | 4.264 | 6.821 | 1.297 | 2.465 |
| SERPIND1 | 385.872 | 361.825 | 439.632 | 171.512 | 150.608 |
| C4BPA | 475.207 | 598.814 | 708.432 | 340.376 | 396.587 |
| APOE | 542.263 | 679.252 | 775.551 | 359.475 | 476.351 |
| APOH | 34.674 | 32.106 | 31.520 | 20.757 | 22.419 |
| PGLYRP2 | 140.794 | 130.457 | 111.302 | 93.465 | 92.899 |
| PROS1 | 111.191 | 171.076 | 192.747 | 103.902 | 134.276 |
| HPX | 1794.675 | 1877.294 | 1956.196 | 1385.543 | 1603.165 |
| C5 | 304.074 | 314.130 | 393.013 | 214.791 | 338.022 |
| CPN1 | 28.625 | 23.166 | 15.732 | 18.360 | 17.534 |
| HYI | 121.984 | 129.599 | 138.786 | 99.113 | 115.505 |
| C1RL | 74.732 | 73.140 | 59.096 | 57.680 | 60.602 |
| PON3 | 25.631 | 27.264 | 23.412 | 23.952 | 20.843 |
| C4B | 1588.465 | 1953.768 | 2080.454 | 1369.356 | 1969.134 |
| F2 | 286.633 | 306.290 | 307.298 | 215.054 | 325.753 |
| PON1 | 259.394 | 288.143 | 202.505 | 257.122 | 190.753 |
| SERPINC1 | 912.432 | 1018.188 | 1092.449 | 952.570 | 1089.933 |
| C3 | 2649.853 | 2630.572 | 2926.007 | 2518.199 | 3084.285 |
| VTN | 754.623 | 777.828 | 811.568 | 805.130 | 842.739 |

The 191 gene products in Table 10 were further analyzed using the Reactome pathway database. The top 200 pathways from the analysis are presented in Table 11. Selected immune pathways are presented in Table 12.

TABLE 11
Top 200 Reactome pathways

| | Entities | Entities | Entities |
|---|---|---|---|
| Pathway name | found | p-value | FDR |
| Packaging Of Telomere Ends | 20 | 1.11E−16 | 1.33E−15 |
| DNA methylation | 21 | 1.11E−16 | 1.33E−15 |
| PRC2 methylates histones and DNA | 21 | 1.11E−16 | 1.33E−15 |
| Ub-specific processing proteases | 35 | 1.11E−16 | 1.33E−15 |
| HATs acetylate histones | 25 | 1.11E−16 | 1.33E−15 |
| Metalloprotease DUBs | 14 | 1.11E−16 | 1.33E−15 |
| Antigen Presentation: Folding, | 23 | 1.11E−16 | 1.33E−15 |
| assembly and peptide loading of class I MHC | | | |
| Condensation of Prophase Chromosomes | 22 | 1.11E−16 | 1.33E−15 |
| HDACs deacetylate histones | 24 | 1.11E−16 | 1.33E−15 |
| Deubiquitination | 37 | 1.11E−16 | 1.33E−15 |
| Inhibition of DNA recombination at telomere | 20 | 1.11E−16 | 1.33E−15 |
| Nucleosome assembly | 19 | 1.11E−16 | 1.33E−15 |
| Deposition of new CENPA-containing nucleosomes at the | 19 | 1.11E−16 | 1.33E−15 |
| centromere | | | |
| Endosomal/Vacuolar pathway | 22 | 1.11E−16 | 1.33E−15 |
| Activated PKN1 stimulates transcription of AR (androgen | 21 | 1.11E−16 | 1.33E−15 |
| receptor) regulated genes KLK2 and KLK3 | | | |
| Activation of anterior HOX genes in hindbrain development | 21 | 1.11E−16 | 1.33E−15 |
| during early embryogenesis | | | |
| Activation of HOX genes during differentiation | 21 | 1.11E−16 | 1.33E−15 |
| B-WICH complex positively regulates rRNA expression | 22 | 1.11E−16 | 1.33E−15 |
| Meiotic synapsis | 20 | 1.11E−16 | 1.33E−15 |
| HCMV Early Events | 27 | 1.11E−16 | 1.33E−15 |
| UCH proteinases | 25 | 1.11E−16 | 1.33E−15 |
| RUNX1 regulates genes involved in megakaryocyte | 21 | 1.11E−16 | 1.33E−15 |
| differentiation and platelet function | | | |
| SIRT1 negatively regulates rRNA expression | 21 | 1.11E−16 | 1.33E−15 |
| Class I MHC mediated antigen processing & presentation | 38 | 1.11E−16 | 1.33E−15 |
| Positive epigenetic regulation of rRNA expression | 22 | 1.11E−16 | 1.33E−15 |
| Meiosis | 22 | 1.11E−16 | 1.33E−15 |
| RNA Polymerase I Promoter Opening | 21 | 1.11E−16 | 1.33E−15 |
| ERCC6 (CSB) and EHMT2 (G9a) positively regulate rRNA | 21 | 1.11E−16 | 1.33E−15 |
| expression | | | |
| RNA Polymerase I Promoter Escape | 21 | 1.11E−16 | 1.33E−15 |
| RHO GTPases activate PKNs | 26 | 1.11E−16 | 1.33E−15 |
| Negative epigenetic regulation of rRNA expression | 21 | 1.11E−16 | 1.33E−15 |
| ER-Phagosome pathway | 37 | 1.11E−16 | 1.33E−15 |
| Meiotic recombination | 22 | 1.11E−16 | 1.33E−15 |
| RHO GTPase Effectors | 38 | 1.11E−16 | 1.33E−15 |
| NoRC negatively regulates rRNA expression | 21 | 1.11E−16 | 1.33E−15 |
| Transcriptional regulation by small RNAs | 23 | 1.11E−16 | 1.33E−15 |
| Antigen processing-Cross presentation | 37 | 1.11E−16 | 1.33E−15 |
| Defective pyroptosis | 21 | 1.11E−16 | 1.33E−15 |
| RUNX1 regulates transcription of genes involved in | 35 | 1.11E−16 | 1.33E−15 |
| differentiation of HSCs | | | |
| Reproduction | 22 | 1.11E−16 | 1.33E−15 |
| M Phase | 41 | 1.11E−16 | 1.33E−15 |
| Mitotic Prophase | 23 | 1.11E−16 | 1.33E−15 |
| TCF dependent signaling in response to WNT | 37 | 1.11E−16 | 1.33E−15 |
| Amyloid fiber formation | 26 | 1.11E−16 | 1.33E−15 |
| Signaling by WNT | 41 | 1.11E−16 | 1.33E−15 |
| Cleavage of the damaged purine | 20 | 1.11E−16 | 1.33E−15 |
| Depurination | 20 | 1.11E−16 | 1.33E−15 |
| Transcriptional regulation by RUNX1 | 37 | 1.11E−16 | 1.33E−15 |
| Recognition and association of DNA glycosylase with site | 20 | 1.11E−16 | 1.33E−15 |
| containing an affected purine | | | |
| RNA Polymerase I Promoter Clearance | 21 | 1.11E−16 | 1.33E−15 |
| Oxidative Stress Induced Senescence | 26 | 1.11E−16 | 1.33E−15 |
| Cellular responses to stress | 58 | 1.11E−16 | 1.33E−15 |
| Immune System | 95 | 1.11E−16 | 1.33E−15 |
| Cell Cycle Checkpoints | 30 | 1.11E−16 | 1.33E−15 |
| Cellular responses to external stimuli | 58 | 1.11E−16 | 1.33E−15 |
| G2/M Checkpoints | 30 | 1.11E−16 | 1.33E−15 |
| HCMV Late Events | 25 | 1.11E−16 | 1.33E−15 |
| Developmental Biology | 68 | 1.11E−16 | 1.33E−15 |
| Formation of the beta-catenin:TCF transactivating complex | 22 | 1.11E−16 | 1.33E−15 |
| Infectious disease | 67 | 1.11E−16 | 1.33E−15 |
| Cellular Senescence | 31 | 1.11E−16 | 1.33E−15 |
| RNA Polymerase I Transcription | 21 | 1.11E−16 | 1.33E−15 |
| Senescence-Associated Secretory Phenotype (SASP) | 25 | 1.11E−16 | 1.33E−15 |
| Gene Silencing by RNA | 24 | 1.11E−16 | 1.33E−15 |
| DNA Damage/Telomere Stress Induced Senescence | 24 | 1.11E−16 | 1.33E−15 |
| Signaling by NOTCH | 35 | 1.11E−16 | 1.33E−15 |
| Pre-NOTCH Transcription and Translation | 21 | 1.11E−16 | 1.33E−15 |
| Base-Excision Repair, AP Site Formation | 20 | 1.11E−16 | 1.33E−15 |
| Pre-NOTCH Expression and Processing | 21 | 1.11E−16 | 1.33E−15 |
| Diseases of programmed cell death | 22 | 1.11E−16 | 1.33E−15 |
| Base Excision Repair | 20 | 1.11E−16 | 1.33E−15 |
| Cytokine Signaling in Immune system | 58 | 1.11E−16 | 1.33E−15 |
| Cleavage of the damaged pyrimidine | 20 | 1.11E−16 | 1.33E−15 |
| Depyrimidination | 20 | 1.11E−16 | 1.33E−15 |
| Recognition and association of DNA glycosylase with site | 20 | 1.11E−16 | 1.33E−15 |
| containing an affected pyrimidine | | | |
| Transcriptional regulation of granulopoiesis | 21 | 1.11E−16 | 1.33E−15 |
| Cell Cycle, Mitotic | 41 | 2.22E−16 | 2.66E−15 |
| Epigenetic regulation of gene expression | 22 | 3.33E−16 | 4.00E−15 |
| Recruitment and ATM-mediated phosphorylation of repair | 17 | 4.44E−16 | 5.33E−15 |
| and signaling proteins at DNA double strand breaks | | | |
| Telomere Maintenance | 20 | 7.77E−16 | 9.33E−15 |
| Signaling by Rho GTPases, Miro GTPases and RHOBTB3 | 44 | 9.99E−16 | 1.20E−14 |
| Cell Cycle | 44 | 1.89E−15 | 2.26E−14 |
| E3 ubiquitin ligases ubiquitinate target proteins | 16 | 2.22E−15 | 2.44E−14 |
| Signaling by Rho GTPases | 43 | 2.22E−15 | 2.44E−14 |
| Estrogen-dependent gene expression | 22 | 2.78E−15 | 3.05E−14 |
| DNA Double Strand Break Response | 17 | 3.11E−15 | 3.42E−14 |
| HCMV Infection | 27 | 8.88E−15 | 9.77E−14 |
| The role of GTSE1 in G2/M progression after G2 checkpoint | 17 | 1.55E−14 | 1.71E−13 |
| Chromatin organization | 26 | 2.02E−14 | 2.22E−13 |
| Chromatin modifying enzymes | 26 | 2.02E−14 | 2.22E−13 |
| Chromosome Maintenance | 20 | 4.14E−14 | 4.14E−13 |
| Hh mutants are degraded by ERAD | 15 | 4.27E−14 | 4.27E−13 |
| Regulation of activated PAK-2p34 by proteasome mediated | 14 | 5.43E−14 | 5.43E−13 |
| degradation | | | |
| Processing of DNA double-strand break ends | 17 | 5.61E−14 | 5.61E−13 |
| Hh mutants abrogate ligand secretion | 15 | 8.45E−14 | 8.45E−13 |
| Regulation of expression of SLITs and ROBOs | 22 | 8.58E−14 | 8.58E−13 |
| Adaptive Immune System | 49 | 8.88E−14 | 8.88E−13 |
| RMTs methylate histone arginines | 14 | 1.18E−13 | 1.18E−12 |
| Vpu mediated degradation of CD4 | 14 | 1.18E−13 | 1.18E−12 |
| Defective CFTR causes cystic fibrosis | 15 | 1.31E−13 | 1.18E−12 |
| Interferon alpha/beta signaling | 22 | 1.46E−13 | 1.31E−12 |
| p53-Independent DNA Damage Response | 14 | 1.51E−13 | 1.36E−12 |
| p53-Independent G1/S DNA damage checkpoint | 14 | 1.51E−13 | 1.36E−12 |
| Ubiquitin Mediated Degradation of Phosphorylated Cdc25A | 14 | 1.51E−13 | 1.36E−12 |
| Autodegradation of the E3 ubiquitin ligase COP1 | 14 | 1.51E−13 | 1.36E−12 |
| Ubiquitin-dependent degradation of Cyclin D | 14 | 1.51E−13 | 1.36E−12 |
| Regulation of Apoptosis | 14 | 1.51E−13 | 1.36E−12 |
| G2/M DNA damage checkpoint | 16 | 1.61E−13 | 1.45E−12 |
| Host Interactions of HIV factors | 20 | 1.88E−13 | 1.55E−12 |
| Signaling by ROBO receptors | 24 | 1.92E−13 | 1.55E−12 |
| FBXL7 down-regulates AURKA during mitotic entry and in | 14 | 1.93E−13 | 1.55E−12 |
| early mitosis | | | |
| SCF-beta-TrCP mediated degradation of Emi1 | 14 | 1.93E−13 | 1.55E−12 |
| Apoptosis | 22 | 2.20E−13 | 1.76E−12 |
| Vif-mediated degradation of APOBEC3G | 14 | 2.46E−13 | 1.97E−12 |
| AUF1 (hnRNP D0) binds and destabilizes mRNA | 14 | 2.46E−13 | 1.97E−12 |
| Protein ubiquitination | 16 | 2.78E−13 | 2.22E−12 |
| Degradation of DVL | 14 | 3.11E−13 | 2.49E−12 |
| Degradation of AXIN | 14 | 3.11E−13 | 2.49E−12 |
| Negative regulation of NOTCH4 signaling | 14 | 3.11E−13 | 2.49E−12 |
| Hedgehog ligand biogenesis | 15 | 4.46E−13 | 3.57E−12 |
| Axon guidance | 36 | 4.47E−13 | 3.58E−12 |
| CDT1 association with the CDC6:ORC:origin complex | 14 | 4.91E−13 | 3.93E−12 |
| Stabilization of p53 | 14 | 4.91E−13 | 3.93E−12 |
| Post-translational protein modification | 62 | 5.48E−13 | 4.39E−12 |
| ROS sensing by NFE2L2 | 14 | 6.13E−13 | 4.90E−12 |
| Interferon gamma signaling | 24 | 7.02E−13 | 4.91E−12 |
| NIK-->noncanonical NF-kB signaling | 14 | 7.63E−13 | 5.34E−12 |
| Downstream signaling events of B Cell Receptor (BCR) | 16 | 9.14E−13 | 6.40E−12 |
| Degradation of GLI1 by the proteasome | 14 | 9.45E−13 | 6.62E−12 |
| GLI3 is processed to GLI3R by the proteasome | 14 | 9.45E−13 | 6.62E−12 |
| Degradation of GLI2 by the proteasome | 14 | 9.45E−13 | 6.62E−12 |
| ESR-mediated signaling | 24 | 1.15E−12 | 8.04E−12 |
| Regulation of mRNA stability by proteins that bind AU-rich | 16 | 1.26E−12 | 8.83E−12 |
| elements | | | |
| Innate Immune System | 55 | 1.63E−12 | 1.14E−11 |
| Interferon Signaling | 29 | 1.64E−12 | 1.15E−11 |
| Programmed Cell Death | 23 | 1.90E−12 | 1.33E−11 |
| Asymmetric localization of PCP proteins | 14 | 2.15E−12 | 1.51E−11 |
| Dectin-1 mediated noncanonical NF-kB signaling | 14 | 2.15E−12 | 1.51E−11 |
| Nervous system development | 36 | 2.47E−12 | 1.73E−11 |
| PCP/CE pathway | 16 | 3.18E−12 | 2.22E−11 |
| APC/C:Cdc20 mediated degradation of Securin | 14 | 3.19E−12 | 2.23E−11 |
| Assembly of the pre-replicative complex | 14 | 3.19E−12 | 2.23E−11 |
| Metabolism of proteins | 76 | 3.63E−12 | 2.54E−11 |
| p53-Dependent G1/S DNA damage checkpoint | 14 | 4.67E−12 | 2.80E−11 |
| p53-Dependent G1 DNA Damage Response | 14 | 4.67E−12 | 2.80E−11 |
| Regulation of RUNX3 expression and activity | 13 | 5.51E−12 | 3.31E−11 |
| Nonhomologous End-Joining (NHEJ) | 13 | 5.51E−12 | 3.31E−11 |
| Regulation of RAS by GAPs | 14 | 5.62E−12 | 3.37E−11 |
| Disease | 76 | 6.31E−12 | 3.79E−11 |
| Oxygen-dependent proline hydroxylation of Hypoxia- | 14 | 6.74E−12 | 4.05E−11 |
| inducible Factor Alpha | | | |
| G1/S DNA Damage Checkpoints | 14 | 6.74E−12 | 4.05E−11 |
| Activation of NF-kappaB in B cells | 14 | 6.74E−12 | 4.05E−11 |
| Cdc20:Phospho-APC/C mediated degradation of Cyclin A | 14 | 8.08E−12 | 4.85E−11 |
| Orc1 removal from chromatin | 14 | 8.08E−12 | 4.85E−11 |
| HDR through Homologous Recombination (HRR) or Single | 17 | 8.42E−12 | 5.05E−11 |
| Strand Annealing (SSA) | | | |
| APC/C:Cdh1 mediated degradation of Cdc20 and other | 14 | 9.65E−12 | 5.79E−11 |
| APC/C:Cdh1 targeted proteins in late mitosis/early G1 | | | |
| APC:Cdc20 mediated degradation of cell cycle proteins prior | 14 | 9.65E−12 | 5.79E−11 |
| to satisfation of the cell cycle checkpoint | | | |
| Regulation of PTEN stability and activity | 14 | 9.65E−12 | 5.79E−11 |
| CDK-mediated phosphorylation and removal of Cdc6 | 14 | 1.15E−11 | 6.90E−11 |
| Regulation of HMOX1 expression and activity | 14 | 1.15E−11 | 6.90E−11 |
| PC/C:Cdc20 mediated degradation of mitotic proteins | 14 | 1.37E−11 | 8.19E−11 |
| SCF(Skp2)-mediated degradation of p27/p21 | 13 | 1.54E−11 | 9.26E−11 |
| Activation of APC/C and APC/C:Cdc20 mediated | 14 | 1.62E−11 | 9.71E−11 |
| degradation of mitotic proteins | | | |
| Homology Directed Repair | 17 | 1.74E−11 | 1.04E−10 |
| Autodegradation of Cdh1 by Cdh1:APC/C | 13 | 2.27E−11 | 1.14E−10 |
| ABC transporter disorders | 15 | 3.25E−11 | 1.63E−10 |
| Regulation of RUNX2 expression and activity | 14 | 4.29E−11 | 2.14E−10 |
| Regulation of APC/C activators between G1/S and early | 14 | 4.29E−11 | 2.14E−10 |
| anaphase | | | |
| Cellular response to hypoxia | 14 | 5.83E−11 | 2.91E−10 |
| DNA Repair | 26 | 6.49E−11 | 3.25E−10 |
| Immunoregulatory interactions between a Lymphoid and a | 24 | 8.39E−11 | 4.19E−10 |
| non-Lymphoid cell | | | |
| Hedgehog ‘off’ state | 16 | 8.45E−11 | 4.23E−10 |
| Beta-catenin independent WNT signaling | 18 | 8.78E−11 | 4.39E−10 |
| DNA Replication Pre-Initiation | 14 | 9.11E−11 | 4.56E−10 |
| MAPK6/MAPK4 signaling | 15 | 9.53E−11 | 4.76E−10 |
| Degradation of beta-catenin by the destruction complex | 14 | 1.40E−10 | 7.01E−10 |
| Signaling by Nuclear Receptors | 26 | 1.51E−10 | 7.56E−10 |
| APC/C-mediated degradation of cell cycle proteins | 14 | 1.61E−10 | 8.07E−10 |
| Regulation of mitotic cell cycle | 14 | 1.61E−10 | 8.07E−10 |
| Signaling by NOTCH4 | 14 | 1.61E−10 | 8.07E−10 |
| Hedgehog ‘on’ state | 14 | 1.61E−10 | 8.07E−10 |
| Switching of origins to a post-replicative state | 14 | 1.85E−10 | 9.26E−10 |
| CLEC7A (Dectin-1) signaling | 15 | 5.12E−10 | 2.56E−09 |
| Signaling by Interleukins | 33 | 5.25E−10 | 2.63E−09 |
| ABC-family proteins mediated transport | 15 | 6.39E−10 | 3.19E−09 |
| TNFR2 non-canonical NF-KB pathway | 14 | 7.69E−10 | 3.85E−09 |
| DNA Double-Strand Break Repair | 17 | 8.35E−10 | 4.18E−09 |
| Signaling by Hedgehog | 17 | 8.35E−10 | 4.18E−09 |
| Transcriptional regulation by RUNX2 | 16 | 9.57E−10 | 4.78E−09 |
| Cyclin E associated events during G1/S transition | 13 | 1.05E−09 | 5.24E−09 |
| Cyclin A:Cdk2-associated events at S phase entry | 13 | 1.37E−09 | 6.84E−09 |
| Interleukin-1 signaling | 14 | 1.39E−09 | 6.96E−09 |
| Gene and protein expression by JAK-STAT signaling after | 12 | 1.44E−09 | 7.18E−09 |
| Interleukin-12 stimulation | | | |
| HIV Infection | 20 | 3.55E−09 | 1.78E−08 |
| Regulation of ornithine decarboxylase (ODC) | 10 | 6.68E−09 | 3.34E−08 |
| Interleukin-12 signaling | 12 | 6.72E−09 | 3.36E−08 |
| Downstream TCR signaling | 14 | 6.98E−09 | 3.49E−08 |
| Cross-presentation of soluble exogenous antigens | 10 | 9.56E−09 | 3.83E−08 |
| (endosomes) | | | |
| Synthesis of DNA | 14 | 1.66E−08 | 6.63E−08 |
| G2/M Transition | 17 | 2.48E−08 | 9.90E−08 |

TABLE 12
Selected immune pathways

| | Entities | Entities | Entities |
|---|---|---|---|
| Pathway name | found | p-value | FDR |
| Immune System | 95 | 1.11E−16 | 1.33E−15 |
| Cytokine Signaling in Immune system | 58 | 1.11E−16 | 1.33E−15 |
| Adaptive Immune System | 49 | 8.88E−14 | 8.88E−13 |
| Innate Immune System | 55 | 1.63E−12 | 1.14E−11 |
| Immunoregulatory interactions | 24 | 8.39E−11 | 4.19E−10 |
| between a Lymphoid and a | | | |
| non-Lymphoid cell | | | |
| TCR signaling | 14 | 5.62E−08 | 2.25E−07 |
| Rap1 signalling | 3 | 5.61E−03 | 8.22E−03 |

### 6.14 Example 14: In Vivo Immune Cell Effects of Systemically (IV) Administered Treg EVs in LPS-Induced Mouse Model of Neuroinflammation

The experiments described in this example were designed to assess the effects of systemic (intravenous) administration of Treg EVs in an LPS-induced mouse model of neuroinflammation (as described above).

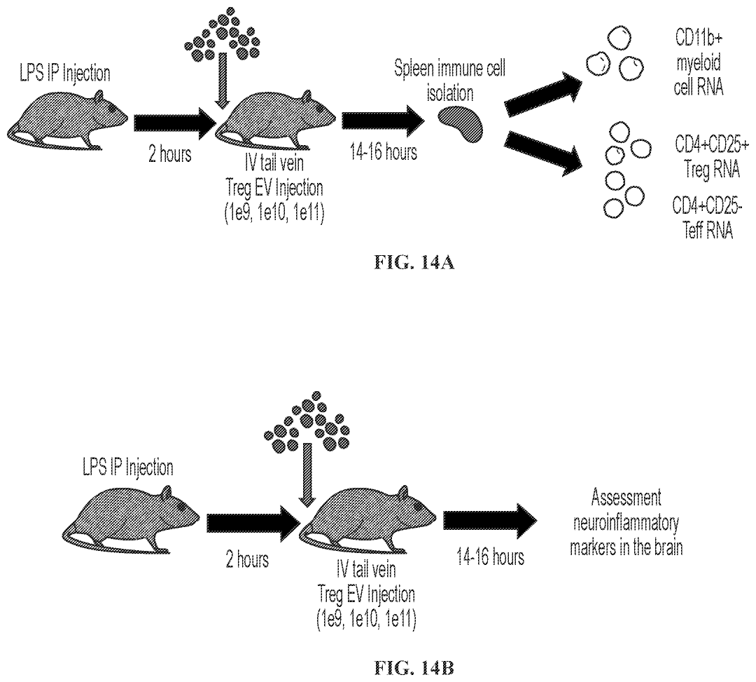

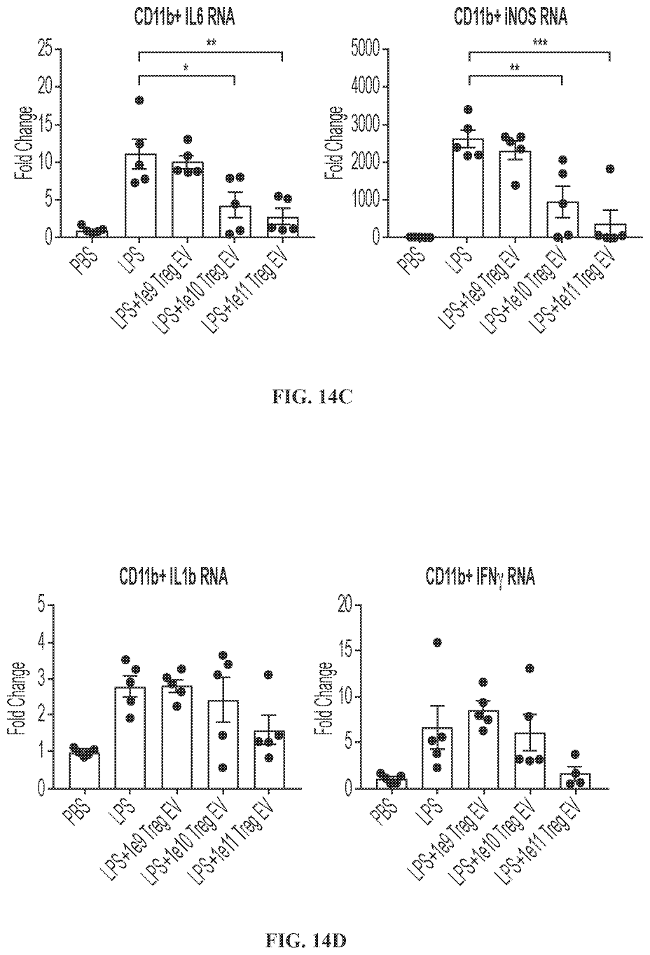

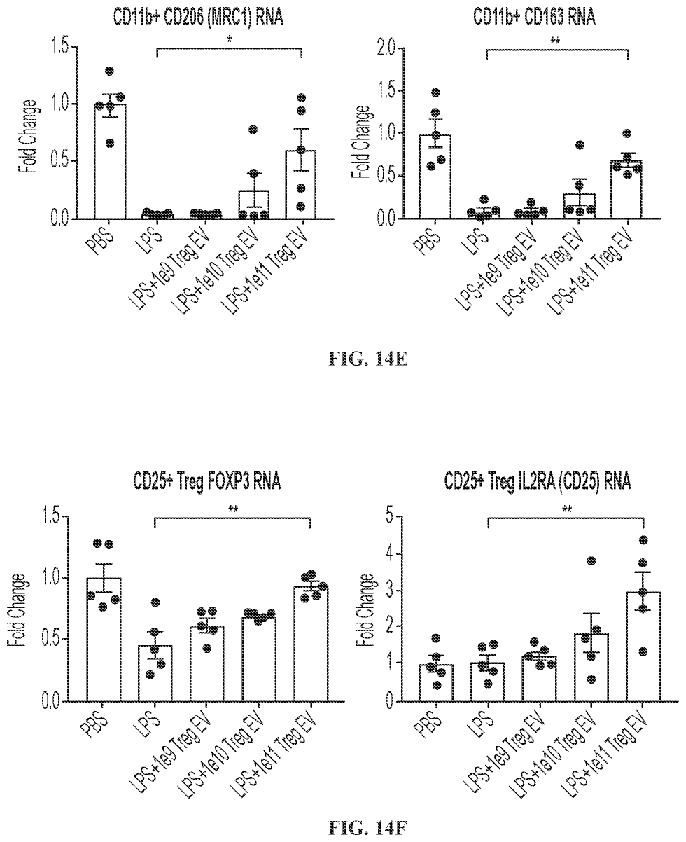

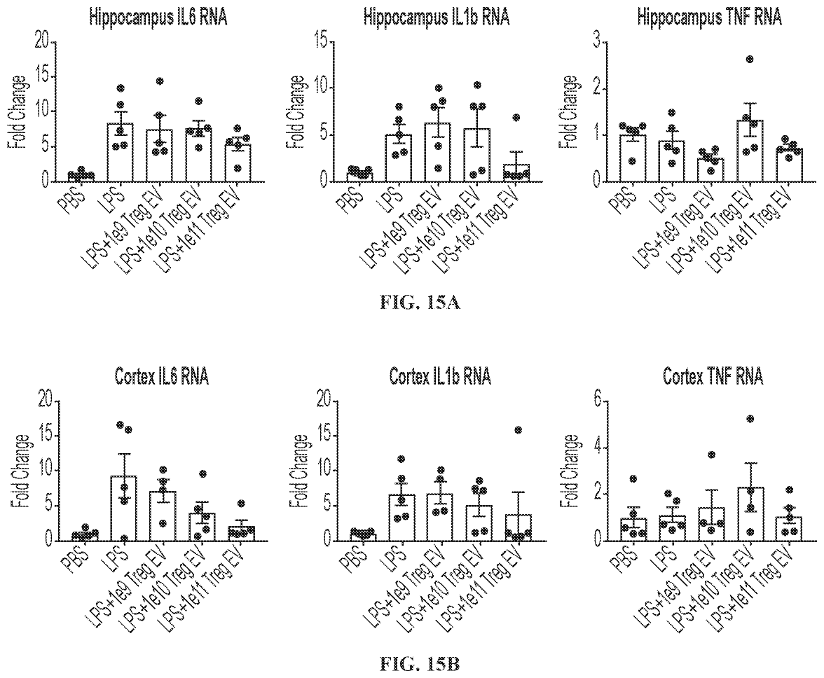

The Treg EVs utilized were produced from bioreactor cultured Tregs healthy subjects and TFF isolated. See Example 15, below (in particular, the Treg used in these experiments were from Example 15 bioreactor run #4). Mice were injected with LPS peripherally to induce neuroinflammatory mechanisms in the brain to test the in vivo suppressive effects of Treg EVs when administered intravenously. Treg EVs were administered via tail vein injection (IV) at different doses (1×10⁹, 1×10¹⁰ and 1×10¹¹) to mice following 2 mg/kg IP injection of LPS for an inflammation mouse model. Following overnight treatment, mice were sacrificed. Spleens were dissected to isolate immune cells, including CD11b+ myeloid cells, CD4+CD25+ Treg cells, and CD4+CD25-effector T cells. See, FIGS. 14A-14B.

Effect of IV-administered Treg EVs on peripheral immune cell signatures. Transcript analysis of spleen-derived CD11b+ myeloid cells showed an induction of pro-inflammatory transcripts such as IL-6, iNOS, IL-1b, and IFNγ following LPS inflammatory induction in the mice (FIG. 14C). Increasing doses of IV-administered Treg EVs demonstrated a corresponding, significant reduction in myeloid pro-inflammatory IL-6 transcripts at doses of 1×10¹⁰ (61%) and 1×10¹¹ (75%) and iNOS transcripts in the same cells at these same doses (1×10¹⁰ (64%) and 1×10¹¹ (85%) (FIG. 14C). Trending decreases in IL-1β and IFNγ transcripts were observed, with the best results at the higher doses administered (FIG. 14D). Anti-inflammatory transcripts from these myeloid cells following LPS activation and subsequent IV Treg EV administration were then analyzed. At higher Treg EV doses, the pro-inflammatory, activated myeloid cells exhibited increased anti-inflammatory transcripts such as MRC1 (mannose receptor/CD206) and CD163 compared to the LPS-injection only control animals (FIG. 14E). These results demonstrate that IV-administered Treg EVs both reduce pro-inflammatory transcripts in activated myeloid cells in the LPS mouse model of inflammation and increase anti-inflammatory transcripts in these same activated cells. These results are indicative of a shift towards the M2, or anti-inflammatory, myeloid phenotype.

Transcript analysis of spleen-derived CD4+CD25+(Treg) and CD4+CD25+(T effector; Teff) cell populations was performed to assess the immune-modulating effects of the Treg EVs. Transcript analysis of the spleen-derived CD4+CD25+ Treg cell population demonstrates a decrease in Treg health and function marker FOXP3 expression following LPS-induced inflammation (FIG. 14F). Treatment with Treg EVs increased FOXP3 transcript levels in a dose-dependent fashion with a significant increase in expression at 1×10¹¹ (FIG. 14F). Further, treatment with Treg EVs increased Treg health and function marker IL2RA transcripts (CD25) in a dose-dependent fashion, with a significant increase in expression at 1×10¹¹ (FIG. 14F). Measurement at the same time point of multiple inflammatory transcripts (TNF, IFNγ, IL-10 and IL-2) from the spleen-derived CD4+CD25-T effector (Teff) cell population indicated that LPS treatment did not result in significant Teff cell activation. There were also no significant alterations in these transcripts observed in the Teff cell population following Treg EV treatment alone, indicating that administration of the human Treg EVs alone to the mice was not sufficient to drive a T cell immune response.

Effect of IV-administered Treg EVs on neuroinflammatory changes in the brain. The results presented above demonstrate that intravenous administration of Treg EVs significantly modulates peripheral immune cell signatures in the LPS-induced model of inflammation. Next, effects of IV-administered Teg EVs on neuroinflammatory changes in the central nervous system (brain) was assessed. Hippocampal and cortex brain tissues were isolated from the same treated animals whose peripheral tissue was assessed above. Transcript analysis from these brain tissues was performed to assess the potential of the peripherally (IV)-administered Treg EVs to reduce neuroinflammation. In the hippocampus region of the LPS-treated mice, IV-delivered Treg EVs produced a reduction in pro-inflammatory transcripts of IL-6 and IL-1β at the highest dose of 1×10¹¹, with the reduction in IL-6 being a modest one (FIG. 15A). In the cortex, there was a trend for a dose-dependent increase in suppression of IL-6 and IL-1β was observed (FIG. 15B). LPS treatment alone did not significantly increase TNF transcript levels in either the hippocampus or cortex relative to PBS control. Likewise, Treg EV administrated did not affect TNF transcript levels in these tissues.

### 6.15 Example 15: Size Distribution of Bioreactor Generated, TFF-Isolated Treg EVs

A series of EV bioreactor production runs (BioR1-BioR6) from healthy patient Treg expansions were performed according to the protocols described herein (see, e.g., Example 12) in media containing 1% human AB serum, where EVs were isolated using a TFF techniques described (see, e.g., Example 3, above) and utilizing the particular parameters set out in Table 13, below.

TABLE 13

| | | IL-2 con. during | Day to add |
|---|---|---|---|
| | | Expansion | activation |
| | Isolation | (IU/ml) | beads and IL-2 |
| BioR1 | CliniMACS Plus | 200 | Day 1 |
| BioR2 | CliniMACS Plus | 200 | Day 1 |
| BioR3 | CliniMACS Plus | 250 | Day 0 |
| BioR4 | CliniMACS Plus | 350 | Day 0 |
| BioR5 | CliniMACS Plus | 500 | Day 0 |
| BioR6 | Prodigy | 500 | Day 0 |

Nanoparticle tracking analysis using the Nanosight NS300 (see Section 6.3.1.4, above) of TFF isolated Treg EVs demonstrated a remarkably consistent population of ˜20-200 nm.

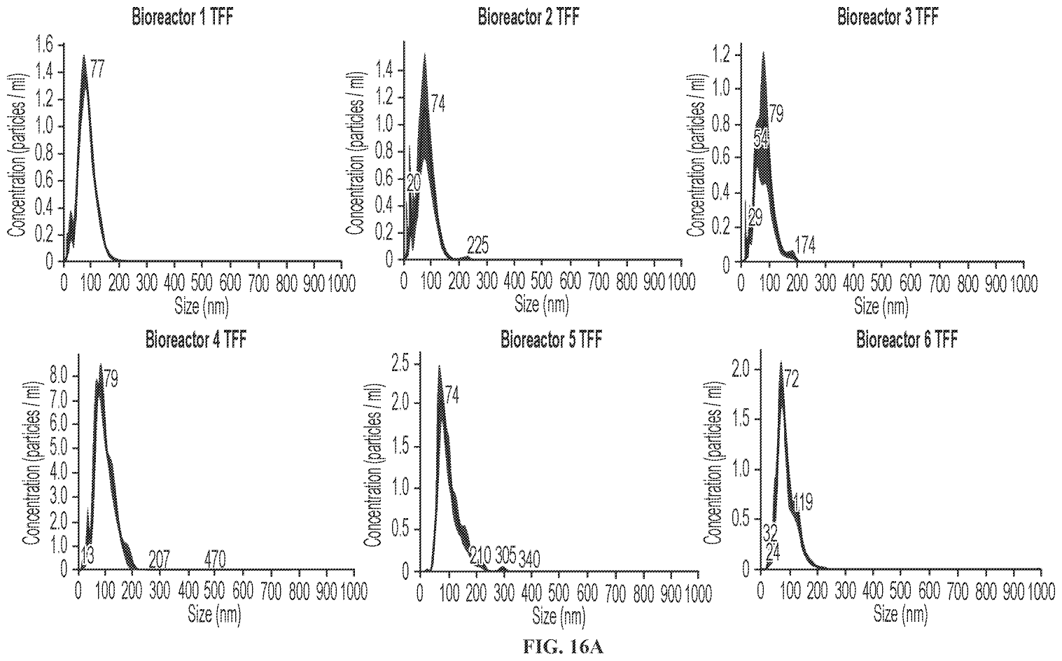

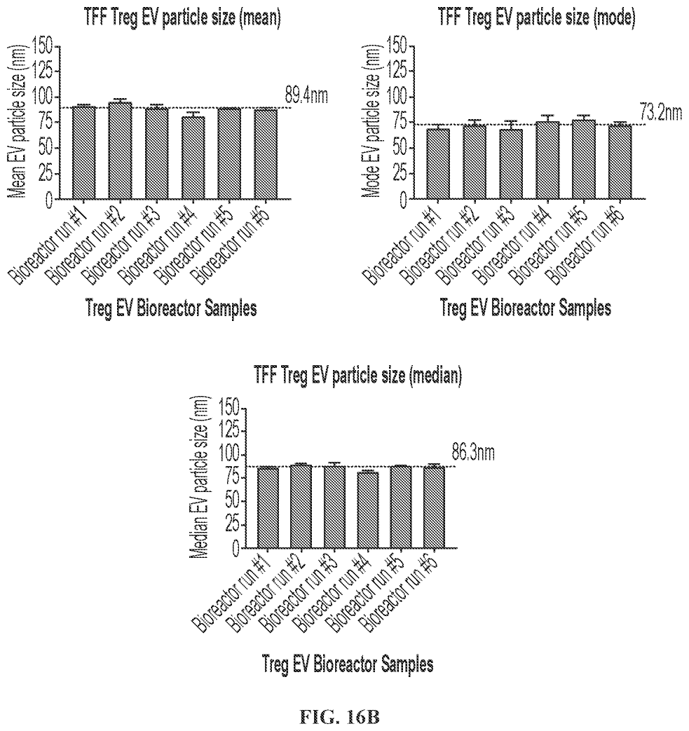

As shown in FIG. 16A, the single peaks produced in the nanoparticle analysis describes a homogeneous EV population (as opposed to a heterogenous population that would have been signified by multiple strong peaks within the distribution. Additionally, the population size parameters are remarkably reproducible across the six runs, for both the mean and mode particle size. In particular, Treg EVs showed a size profile with a mean of 89.4 nm, a median of 86.3 nm and a mode of 73.2 nm (FIG. 16B).

### 6.16 Example 16: Treg EVs Suppress T Cell Proliferation In Vitro

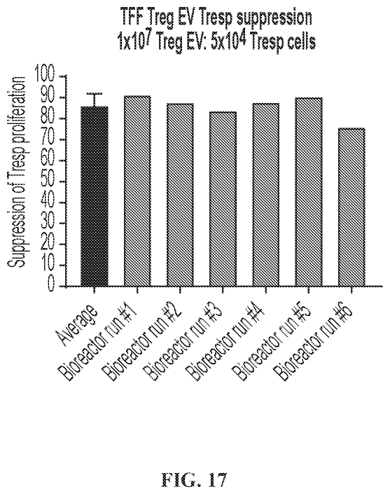

Bioactivity B assays on the Treg EVs from bioreactor runs described in Example 15 were performed to examine Treg EV suppression in T cell proliferation assays. In vitro dose response studies indicated that 1×10⁷ Treg EVs was equivalent to 50,000 Treg cells in their ability to suppress T responder (Tresp) proliferation once activated with CD3/CD28 beads. Treg EV suppression was observed to be 86.12%+5.17%, n=6 (FIG. 17).

### 6.17 Example 17: Quantification of Functional Proteins in Treg EVs

Treg EVs from the bioreactor runs described in Example 15, above were further characterized as described in this example.

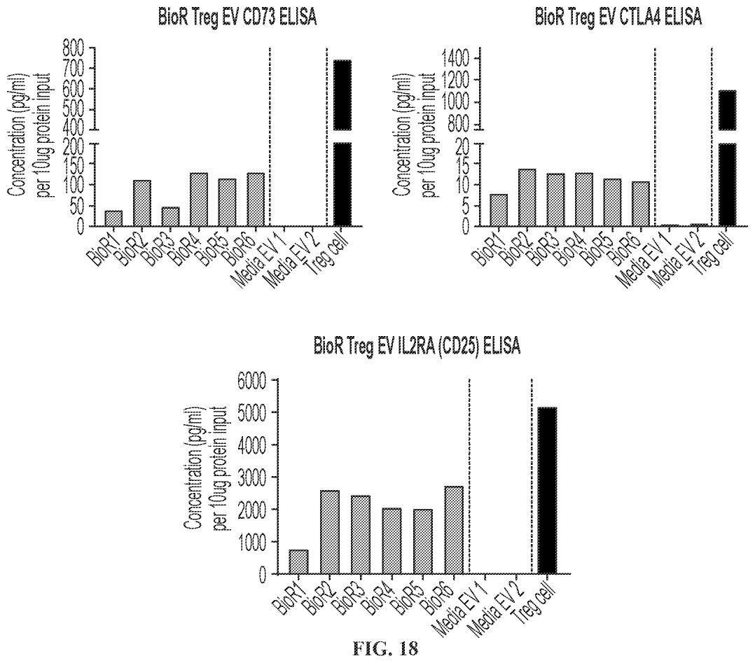

Enzyme-linked immunoassays (ELISA) were utilized to quantify Treg-conserved functional proteins in the Treg EVs. It was found that each of CD73, CTLA4 and CD25 were present in the Treg EVs while virtually absent in the media EVs alone (FIG. 18). As also shown at FIG. 18, each of these markers is also present at substantial levels in the Treg cells from which the Treg EVs are obtained. Interestingly, CD73 and CTLA4 play major roles in mechanisms of Treg suppressive function, and CD25 (IL2RA) is a known marker of Treg cell health and function.

### 6.18 Example 18: Quantification of Residual IL2 and Albumin

Impurity profiles of the Treg EVs produced as described in Example 15, above, were generated by quantification of residual IL2 and albumin (as a percent of the original amounts in culture).

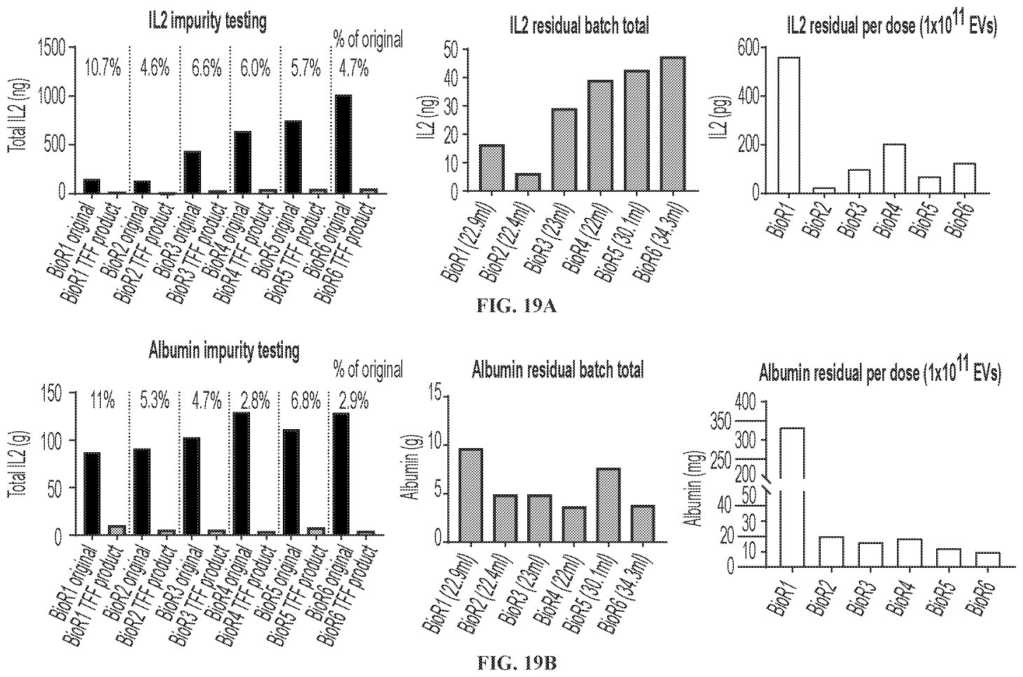

Following TFF, it was determined that only an average of 6.38% of total IL2 remained in the concentrated retentate (n=6) (FIG. 19A). This corresponds to less than 50 ng total IL2. When diluted to a dose of 1×10¹¹ Treg EVs in 2 mL of sterile saline per vial, for example, this would amount to less than 200 pg IL2.

Following TFF, it was determined that only an average of 5.58% of total albumin remained following TFF processing, which equates to less than 10 grams in total concentration Treg EV product (FIG. 19B). When diluted to a dose of 1×10¹¹ Treg EVs in 2 mL of sterile saline per vial, for example, this would amount to less than 20 mg albumin. It is noted that both IL2 and albumin are typically used as a direct injection into a patient or used in cell therapy administration, and both are known to be well tolerated at much higher amount than this.

### 6.19 Example 19: Stability and Particle Size Distribution of Treg EVs at Room Temperature, 4° C., −20° C. and −80° C.

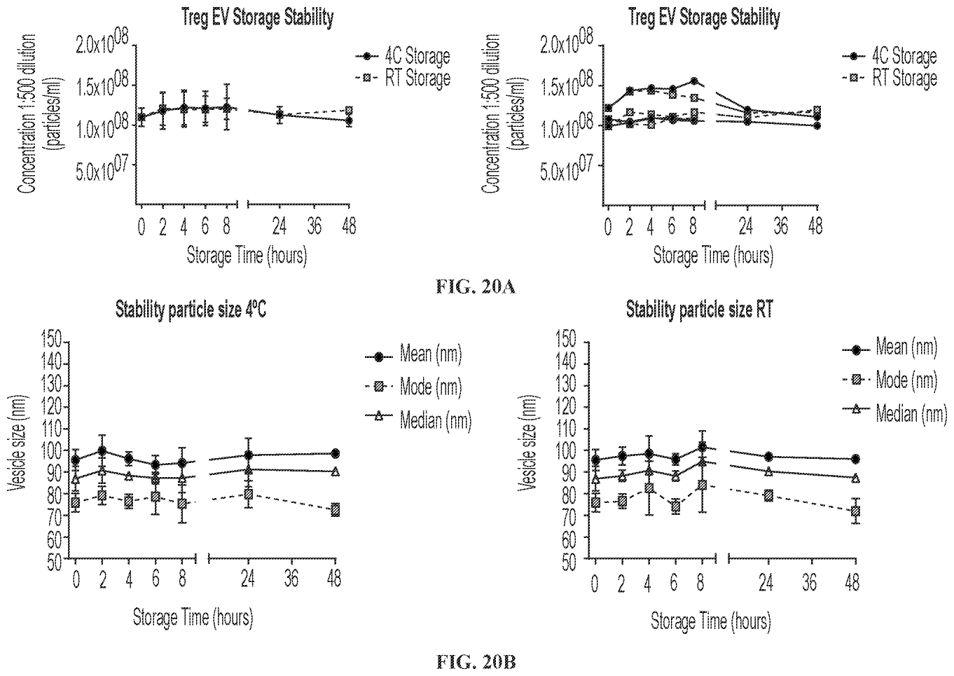

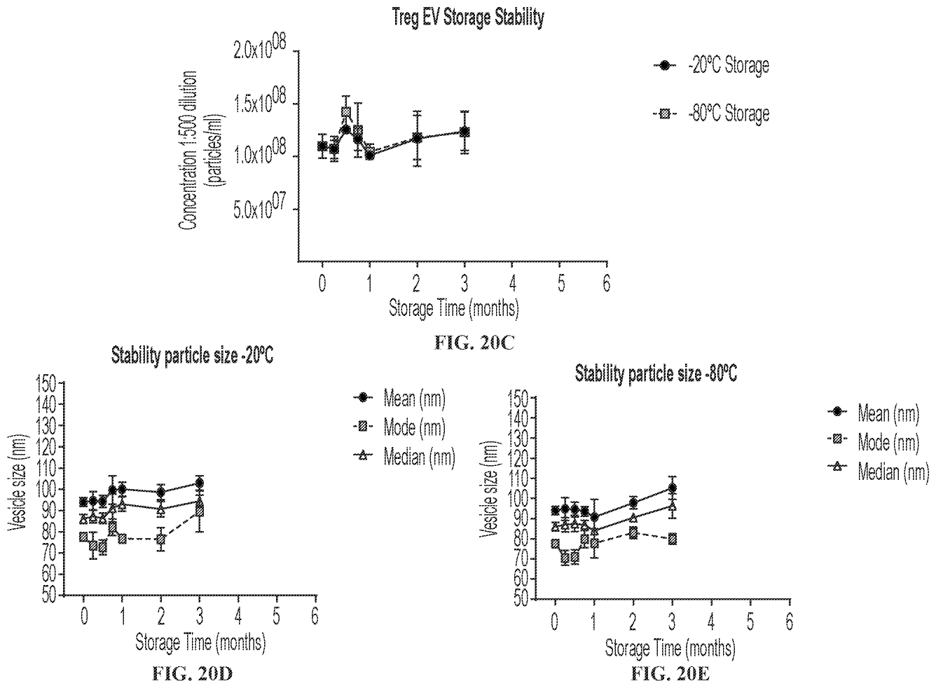

Treg EVs produced from biotreactor cultured Tregs of healthy patients (see Example 15, above, in particular, the EVs from BioR4-6) and isolated via the TFF techniques described herein (see, e.g., Example 3, above) were used for these stability experiments, diluted to an exemplary unit dose of 1×10¹¹ EVs per dose/vial in 2 mL of 0.9% sodium chloride solution (sterile saline solution for injection). Examination of Treg EV particle concentration and size parameters with prolonged periods of time at both room temperature (RT) and 4° C. were done to assess stability of the Treg EV product. Treg EVs were stable at the exemplary unit dose up to the 48 hour upper limit of the examination, 48 hours (FIG. 20A-20B). Additionally, particle size distribution remained stable at both 4° C. and room temperature storage at dose with regard to mean, mode, and median particle size as assessed via nanoparticle analysis (FIG. 20C). Moreover, both the stability of the Tregs as well as the stability of the Treg particle size remains stable after prolonged storage (up to 3 months, the latest timepoint tested) at −20° C. and −80° C. (FIG. 20D-20E).

### 6.20 Example 20: Stability and Particle Size Distribution of Treg EVs at Room Temperature, 4° C., −20° C. and −80° C.

This example was performed with the TFF-isolated Treg EVs from the bioreactor runs described in Example 15.

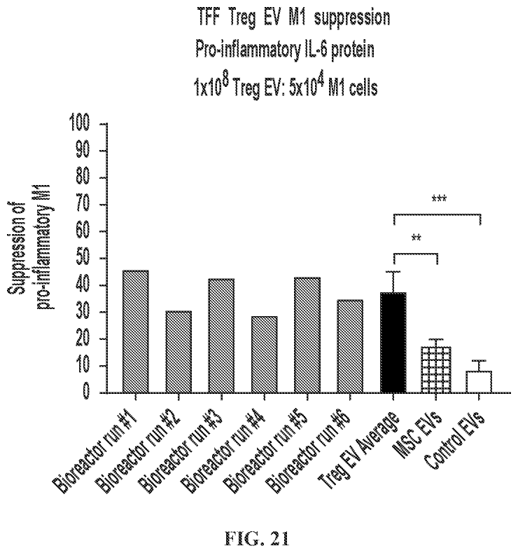

Treg EVs were co-cultured overnight with iPSC-derived, pro-inflammatory myeloid M1 cells that had been activated with GM-CSF and LPS/IFNy. The Treg EVs suppressed the pro-inflammatory IL-6 cytokine output of M1 cells by approximately 40% (FIG. 21), which was significantly greater than the suppressive activity of mesenchymal stem cell (MSC) EVs or the minimal suppression mediated by a control population of serum EVs, thereby further demonstrating the potential of the Treg EVs described herein as therapeutics for the treatment of autoimmune and inflammatory disorders.

### 6.21 Example 21: Treg EV Surface Marker Profile

To further characterize the Treg EV populations described herein, an EV surface marker analysis of the Treg EVs from the six bioreactor runs described in Example 15 and TFF isolated (see, e.g., Example 3) was performed. As noted therein, the Treg EVs were from Tregs obtained from healthy subjects and ex vivo expanded, and the Treg EVs were TFF isolated. The resulting Treg EV surface marker profile is discussed herein.

Treg EV surface proteins were assessed using a Miltenyi MACSPlex Exosome Kit (Miltenyi Biotec) according to manufacturer's instructions and analyzed on MACSQuant Analyzer flow cytometer (Miltenyi Biotec). Briefly, EV populations were incubated overnight with a cocktail of various fluorescently labeled bead populations coated with specific antibodies targeting different surface epitopes. EV detection reagents were used to form sandwich complexes on the beads that were then analyzed based on their unique fluorescent characteristics. Distinct positive populations were then measured with the MACSQuant flow cytometer.

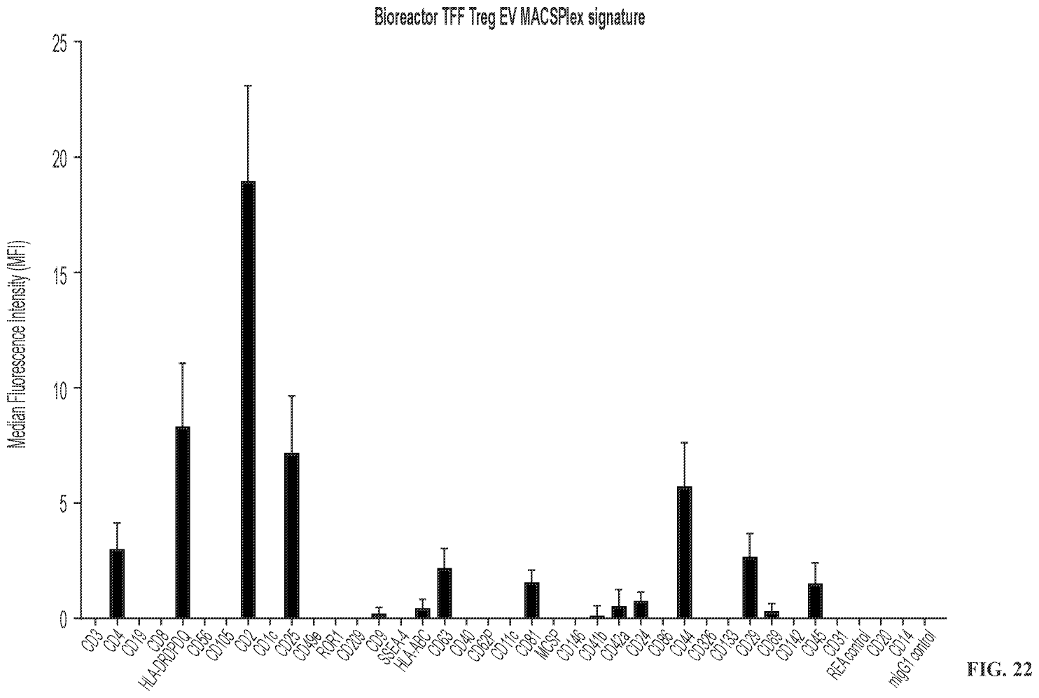

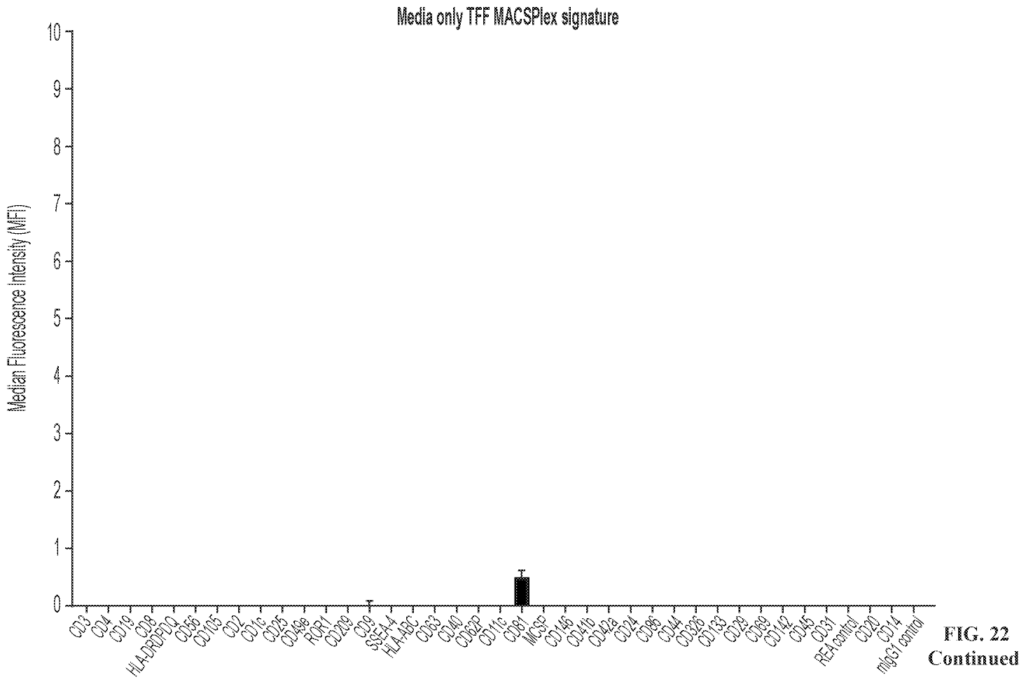

The Treg EVs assayed were from the six bioreactor runs described in Example 15. As noted therein, the Treg EVs were from Tregs obtained from healthy subjects and ex vivo expanded, and the Treg EVs were TFF isolated. The resulting Treg EV signature of these populations is shown at the upper panel of FIG. 22, which is the averaged data of the 6 Treg EV populations. The control “media EVs” are derived from the 1% serum supplemented bioreactor media (no cell culture). This provides a control population for experiments but also allows for the differential characterization of the Treg EVs from the media EVs alone (FIG. 22, lower panel). For example, the Treg EV results shown in FIG. 22, upper panel have had the potential contribution from the media EVs alone removed.

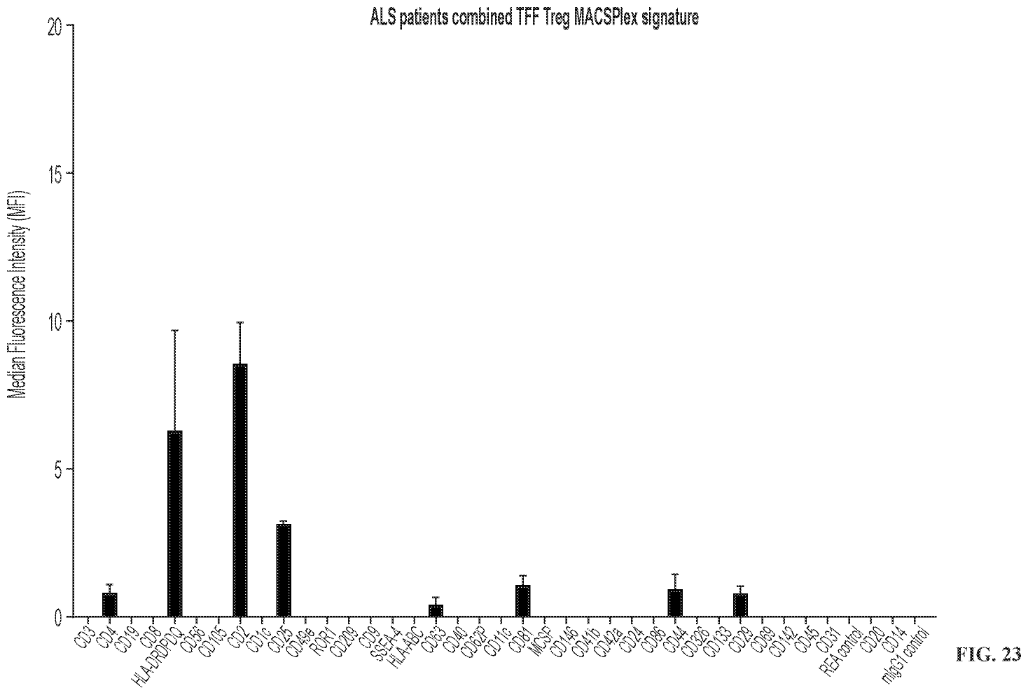

ALS patient Treg EVs were prepared following Treg expansion according to the protocol described in Example 1 above using the TFF protocol described in Example 3 above. The signature of ALS patient-derived Treg EVs is shown in FIG. 23. Because these Treg EVs were also cultured in serum, the media EV panel shown at FIG. 22, lower panel also allows for differential characterization of these Treg EVs, as well.

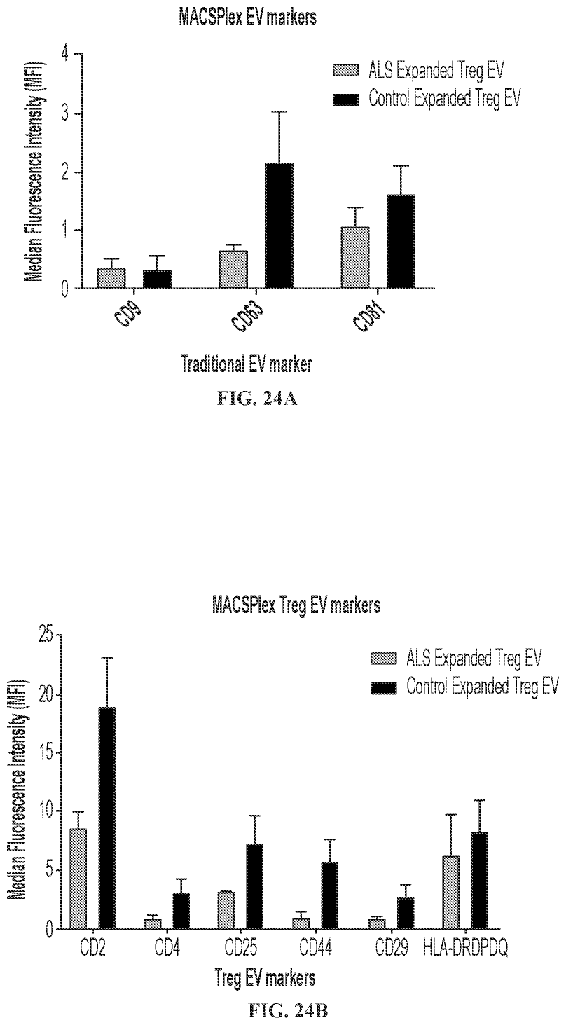

The signature of Treg EVs from healthy subjects (FIG. 22, upper panel) is similar to that of ALS patient Treg EVs (FIG. 23). For example, CD9, CD63, and CD81, which are commonly used as exosome markers, were positive in both the Treg EVs isolated from ALS patients as well as healthy subjects (see each of FIG. 22, upper panel, FIG. 23 and FIG. 24A). Markers that are associated specifically with Treg EVs as compared to media EVs (see, also, e.g. FIGS. 2J and 2K), were found be positively associated with Tregs from both ALS patients and healthy subjects (see each of FIG. 22, FIG. 23 and FIG. 24B). Note: healthy subjects are referred to as “control” in FIG. 24A-B. Interestingly, CD2 is present at a particularly high abundance in Tregs obtained from both healthy and ALS starting material. Such surface markers as HLA-DRPDQ, CD25, CD44, CD45, CD29, CD4, and CD125 were also observed at particularly appreciable levels. It is noted that “HLA-DRDPDQ” refers to HLA-Class II molecules HLA-DR, HLA-DP and HLA-DQ.

To verify that the signatures being observed were unique to the Treg EVs and not, for example, the result of an artifact of the assay, EVs derived from a different cell type (CD14+ cells) were also assessed for surface markers using the Miltenyi MACSPlex assay. As expected, the signature obtained from these EVs was distinct from the signature obtained from the Treg EVs.

### 6.22 Example 22: Treg EV RNA Profile

To further characterize the Treg EV populations described herein, an analysis of the RNA components, in particular, micro-RNA components, of the Treg EVs obtained from the six bioreactor runs described in Example 15, above, was performed. As a control, a media only Ev analysis was also performed. Described herein is the Treg EV RNA profile obtained through the analysis.

As noted in Example 15, the Treg EVs were from Tregs obtained from healthy subjects and ex vivo expanded, and the Treg EVs were TFF isolated.

RNA extraction. EVs were lysed using 3 ml of TRI reagent added to 1 ml of EV sample. Samples were mixed and then centrifuged at 12,000×g at 4° C. for 5 min. Cleared supernatant was transferred into new tubes and incubated at room temperature for 5 min. BAN Phase Separation Reagent was added to the supernatant (0.05 ml of reagent per 1 ml of supernatant), followed by vigorous shaking and 5 min incubation at room temperature. To separate aqueous phase from organic phase, samples were centrifuged at 12,000×g at 4° C. for 15 min and then aqueous phase containing RNA was transferred into a new tube. To clean up RNA from the aqueous phase and enrich for small RNA species (those approximately 17-200 nucleotides in length), RNA Clean & Concentrator™-5 Kit from Zymo Research (cat. #R1015) was used. The manufacturer's protocol was followed with the modification of 1.5 volumes of ethanol being added to the aqueous phase.

microRNA-Seq Library preparation (Zymo Research): microRNA-Seq libraries were constructed from 100 ng of RNA. In brief, RNA was ligated to the miRNA adapter. Excess adapters were blocked to prevent interference with subsequent steps. The miRNA-adapter ligation product was circularized, and the blocked adapter dimer was removed. The circularized miRNA-adapter product was then reverse transcribed. The resulting cDNA was amplified using Indexing PCR primers which added on Illumina-compatible adapter and index sequences. microRNA-Seq libraries were sequenced on an Illumina NovaSeq sequener to a sequencing depth of at least ˜5-10 million read pairs per sample.

Sequence Data Alignments: Illumina NovaSeq reads from microRNA-Seq data files were first adaptor trimmed using Trim Galore! (v0.6.6) and then quality analyzed using a custom pipeline integrating Bowtie (v1.3.0), Samtools (v1.11), and FASTX (v0.0.14) with alignment of short reads to the reference genome of interest. Further miRNA-specific computational analysis was performed using the software programs miR Trace (v1.0.1), mirtop (v0.4.23), and isomiRs (v1.16.0). A sequencing read was considered a miRNA count if the sequence corresponded to a known miRNA sequence. Table 14, below, presents the total number of miRNA read counts obtained from each Treg EV population tested (Treg_EV_1-6), the average of the six totals (Treg EV AVG) and the standard deviation (SD). As the Treg EVs were obtained from Treg cells cultured in serum (1% AV serum), a media only EV population was also obtained, and miRNA from the this population was sequenced.

TABLE 14

| # of small RNA | |
|---|---|
| reads in EVs | miRNA |
| Treg_EV_1 | 689554 |
| Treg_EV_2 | 1646852 |
| Treg_EV_3 | 953235 |
| Treg_EV_4 | 1171101 |
| Treg_EV_5 | 2337624 |
| Treg_EV_6 | 1211914 |
| Treg EV AVG | 1335046.7 |
| Treg EV SD | 533364.3 |
| media EV | 9811 |

Table 15, below, presents the top 50 most abundant miRNAs present in the Treg EVs tested, as determined based on the number of miRNA read counts obtained for each sequence, averaged for the six sets of Treg EVs tested (Treg EV AVG). The table also presents the miRNA read counts for each of the individual Treg populations (Treg_EV_1-6). SD=standard deviation. As shown in Table 14 (media EV), the media only EVs contained very little miRNA and, as such, did not substantially contribute to the Treg EV miRNA results shown herein.

TABLE 15

| | | | | | | | | Treg EV | |
|---|---|---|---|---|---|---|---|---|---|
| Rank | miRNA | Treg_EV_1 | Treg_EV_2 | Treg_EV_3 | Treg_EV_4 | Treg_EV_5 | Treg_EV_6 | AVG | SD |
| 1 | hsa-miR- | 225964 | 276761 | 527230 | 379685 | 440434 | 235302 | 347562.7 | 111309.5 |
| | 1290 | | | | | | | | |
| 2 | hsa-miR- | 105164 | 387840 | 141676 | 177746 | 329422 | 180907 | 220459.2 | 102288.6 |
| | 146a-5p | | | | | | | | |
| 3 | hsa-miR- | 49648 | 73026 | 73383 | 58541 | 171730 | 87369 | 85616.17 | 40318.73 |
| | 155-5p | | | | | | | | |
| 4 | hsa-let-7a-5p | 49644 | 111100 | 46789 | 75462 | 90178 | 90330 | 77250.5 | 23011.6 |
| 5 | hsa-miR-21- | 13060 | 51134 | 36819 | 67616 | 151688 | 45282 | 60933.17 | 43771.03 |
| | 5p | | | | | | | | |
| 6 | hsa-miR- | 34793 | 70234 | 39306 | 43463 | 73005 | 50960 | 51960.17 | 14747.09 |
| | 191-5p | | | | | | | | |
| 7 | hsa-miR- | 39543 | 82505 | 27853 | 69259 | 58090 | 28442 | 50948.67 | 20624.09 |
| | 1246 | | | | | | | | |
| 8 | hsa-let-7b- | 26611 | 53240 | 20077 | 31318 | 56861 | 41872 | 38329.83 | 13527.3 |
| | 5p | | | | | | | | |
| 9 | hsa-miR- | 10597 | 30539 | 24040 | 39684 | 70003 | 35686 | 35091.5 | 18180.11 |
| | 29a-3p | | | | | | | | |
| 10 | hsa-miR- | 20979 | 42404 | 19116 | 29203 | 65103 | 28631 | 34239.33 | 15708.79 |
| | 320a-3p | | | | | | | | |
| 11 | hsa-miR- | 22844 | 45861 | 15488 | 29891 | 50578 | 27383 | 32007.5 | 12379.5 |
| | 423-5p | | | | | | | | |
| 12 | hsa-let-7f-5p | 16960 | 41878 | 20580 | 31898 | 42293 | 34824 | 31405.5 | 9712.57 |
| 13 | hsa-let-7i-5p | 14617 | 36461 | 19384 | 27633 | 46769 | 33244 | 29684.67 | 10706.77 |
| 14 | hsa-miR-16- | 12997 | 26984 | 22582 | 24238 | 55555 | 24120 | 27746 | 13188.22 |
| | 5p | | | | | | | | |
| 15 | hsa-miR- | 7165 | 22018 | 18540 | 20744 | 46691 | 22392 | 22925 | 11820.12 |
| | 26a-5p | | | | | | | | |
| 16 | hsa-let-7g- | 11785 | 26661 | 15114 | 22318 | 34387 | 25209 | 22579 | 7475.66 |
| | 5p | | | | | | | | |
| 17 | hsa-miR- | 4615 | 17737 | 16876 | 14968 | 55060 | 13114 | 20395 | 16086.14 |
| | 221-3p | | | | | | | | |
| 18 | hsa-miR- | 4730 | 18271 | 13680 | 19182 | 43109 | 12379 | 18558.5 | 11947.19 |
| | 222-3p | | | | | | | | |
| 19 | hsa-miR- | 6429 | 16513 | 17398 | 15244 | 33814 | 19502 | 18150 | 8126.71 |
| | 26b-5p | | | | | | | | |
| 20 | hsa-miR- | 11649 | 12181 | 6737 | 17591 | 21880 | 14466 | 14084 | 4774.09 |
| | 342-3p | | | | | | | | |
| 21 | hsa-miR- | 4406 | 11832 | 12107 | 12469 | 27391 | 14395 | 13766.67 | 6853.47 |
| | 146b-5p | | | | | | | | |
| 22 | hsa-miR- | 8414 | 21060 | 8048 | 9686 | 18565 | 10778 | 12758.5 | 5116.26 |
| | 92a-3p | | | | | | | | |
| 23 | hsa-miR-93- | 7869 | 12620 | 10724 | 9248 | 19071 | 12800 | 12055.33 | 3588.68 |
| | 5p | | | | | | | | |
| 24 | hsa-miR- | 5329 | 8050 | 6675 | 4083 | 22705 | 9991 | 9472.17 | 6210.65 |
| | 23a-3p | | | | | | | | |
| 25 | hsa-miR- | 7913 | 15837 | 6309 | 8734 | 9905 | 6883 | 9263.5 | 3165.5 |
| | 181a-5p | | | | | | | | |
| 26 | hsa-miR- | 4094 | 5781 | 5579 | 8699 | 18892 | 9827 | 8812 | 4908.3 |
| | 150-5p | | | | | | | | |
| 27 | hsa-miR- | 3980 | 7853 | 9773 | 5314 | 16557 | 6769 | 8374.33 | 4091.39 |
| | 20a-5p | | | | | | | | |
| 28 | hsa-miR- | 2593 | 3863 | 7896 | 4380 | 21847 | 8087 | 8111 | 6469.66 |
| | 27a-3p | | | | | | | | |
| 29 | hsa-let-7d- | 4012 | 8492 | 4062 | 6817 | 9430 | 6252 | 6510.83 | 2035.04 |
| | 5p | | | | | | | | |
| 30 | hsa-miR-17- | 3047 | 5612 | 6495 | 4234 | 11836 | 5393 | 6102.83 | 2786.83 |
| | 5p | | | | | | | | |
| 31 | hsa-miR- | 2693 | 4714 | 4466 | 4615 | 10942 | 6996 | 5737.67 | 2641.46 |
| | 142-3p | | | | | | | | |
| 32 | hsa-miR- | 2639 | 8094 | 3474 | 3559 | 10995 | 3443 | 5367.33 | 3085.1 |
| | 130b-3p | | | | | | | | |
| 33 | hsa-miR-25- | 2522 | 6404 | 2583 | 3345 | 6939 | 3450 | 4207.17 | 1783.41 |
| | 3p | | | | | | | | |
| 34 | hsa-miR- | 625 | 1567 | 2535 | 2646 | 11280 | 2808 | 3576.83 | 3526.91 |
| | 142-5p | | | | | | | | |
| 35 | hsa-miR- | 1537 | 3856 | 2314 | 3385 | 5337 | 4627 | 3509.33 | 1293.84 |
| | 103a-3p | | | | | | | | |
| 36 | hsa-miR-28- | 2133 | 6239 | 1967 | 1864 | 4820 | 3839 | 3477 | 1645.78 |
| | 3p | | | | | | | | |
| 37 | hsa-let-7e-5p | 1391 | 4677 | 1555 | 2679 | 5074 | 4177 | 3258.83 | 1464.99 |
| 38 | hsa-miR- | 1884 | 3911 | 2579 | 3347 | 4712 | 2910 | 3223.83 | 913.92 |
| | 425-5p | | | | | | | | |
| 39 | hsa-miR- | 846 | 2804 | 1545 | 2312 | 7318 | 1774 | 2766.5 | 2124.84 |
| | 186-5p | | | | | | | | |
| 40 | hsa-miR- | 2365 | 3673 | 2135 | 1777 | 3708 | 2728 | 2731 | 734.98 |
| | 625-5p | | | | | | | | |
| 41 | hsa-miR- | 2410 | 2887 | 1403 | 2329 | 2801 | 2373 | 2367.17 | 481.56 |
| | 4516 | | | | | | | | |
| 42 | hsa-miR-22- | 320 | 2245 | 1353 | 2091 | 6521 | 1384 | 2319 | 1979.8 |
| | 3p | | | | | | | | |
| 43 | hsa-miR-24- | 692 | 2509 | 1919 | 1892 | 4663 | 2012 | 2281.17 | 1197.61 |
| | 3p | | | | | | | | |
| 44 | hsa-miR- | 1076 | 3183 | 1514 | 3275 | 3595 | 703 | 2224.33 | 1157.54 |
| | 486-5p | | | | | | | | |
| 45 | hsa-miR-98- | 1727 | 2888 | 1333 | 1900 | 3084 | 2215 | 2191.17 | 621.82 |
| | 5p | | | | | | | | |
| 46 | hsa-miR- | 2159 | 3419 | 1494 | 1368 | 3046 | 1491 | 2162.83 | 805.03 |
| | 181b-5p | | | | | | | | |
| 47 | hsa-miR- | 1384 | 2068 | 2223 | 1105 | 4228 | 1058 | 2011 | 1086.7 |
| | 378a-3p | | | | | | | | |
| 48 | hsa-miR- | 658 | 2062 | 953 | 2664 | 3702 | 1437 | 1912.67 | 1041.63 |
| | 30d-5p | | | | | | | | |
| 49 | hsa-miR- | 886 | 2674 | 1681 | 1618 | 2554 | 1905 | 1886.33 | 602.96 |
| | 454-3p | | | | | | | | |
| 50 | hsa-miR- | 1245 | 2075 | 825 | 1816 | 3513 | 1611 | 1847.5 | 845.68 |
| | 342-5p | | | | | | | | |

The most abundant miRNA identified as hsa-miR-1290. On average, based on the miRNA read counts, the abundance of this miRNA was approximately 26%.

Interestingly, a large number of the most abundant miRNAs in the Treg EV RNA profile are inflammatory- or immune-related miRNAs, based on the Tam2.0 miRbase (Kozomara and Griffiths-Jones (2011) Nuc. Acids Res. 39:D152-D157. See Table 16, below.

TABLE 16

| Associated Function | Count | P-value | miRNA |
|---|---|---|---|
| Immune Response | 30 | 3.48E−25 | hsa-mir-186, hsa-mir-320a, hsa-mir-103a-2, hsa- |
| | | | mir-181a-1, hsa-mir-92a-1, hsa-mir-486-1, hsa- |
| | | | mir-16-2, hsa-mir-146b, hsa-mir-181b-1, hsa-mir- |
| | | | 155, hsa-mir-181a-2, hsa-mir-16-1, hsa-mir-103a- |
| | | | 1, hsa-mir-92a-2, hsa-mir-150, hsa-let-7g, hsa- |
| | | | mir-98, hsa-mir-22, hsa-mir-146a, hsa-mir- |
| | | | 93, hsa-mir-486-2, hsa-mir-342, hsa-let-7i, hsa- |
| | | | mir-20a, hsa-mir-17, hsa-mir-25, hsa-mir-21, hsa- |
| | | | mir-181b-2, hsa-mir-29a, hsa-mir-27a |
| Innate Immunity | 21 | 5.91E−22 | hsa-let-7a-2, hsa-let-7b, hsa-mir-24-1, hsa-let-7f- |
| | | | 1, hsa-let-7f-2, hsa-mir-181a-1, hsa-let-7e, hsa-let- |
| | | | 7d, hsa-mir-146a, hsa-mir-146b, hsa-mir-24- |
| | | | 2, hsa-mir-26a-1, hsa-mir-21, hsa-mir-181b- |
| | | | 2, hsa-mir-181b-1, hsa-mir-142, hsa-let-7a-1, hsa- |
| | | | mir-26a-2, hsa-mir-155, hsa-mir-181a-2, hsa-let- |
| | | | 7g |
| Inflammation | 28 | 9.45E−20 | hsa-mir-24-1, hsa-mir-320a, hsa-mir-181a-1, hsa- |
| | | | let-7d, hsa-mir-146b, hsa-mir-181b-1, hsa-mir- |
| | | | 155, hsa-mir-181a-2, hsa-mir-222, hsa-mir- |
| | | | 150, hsa-let-7g, hsa-mir-221, hsa-let-7f-2, hsa- |
| | | | mir-98, hsa-mir-22, hsa-mir-146a, hsa-mir- |
| | | | 93, hsa-mir-342, hsa-mir-20a, hsa-mir-17, hsa- |
| | | | mir-130b, hsa-mir-25, hsa-mir-24-2, hsa-mir- |
| | | | 21, hsa-mir-181b-2, hsa-mir-29a, hsa-mir- |
| | | | 142, hsa-mir-27a |
| Immune System | 11 | 5.66E−12 | hsa-mir-181a-1, hsa-mir-17, hsa-mir-181a-2, hsa- |
| | | | mir-146a, hsa-mir-25, hsa-mir-181b-1, hsa-mir- |
| | | | 93, hsa-mir-155, hsa-mir-20a, hsa-mir-150, hsa- |
| | | | mir-181b-2 |

Among these inflammatory miRNAs are two of the most abundant miRNAs present in the Treg EV populations, hsa-miR-146-5p and hsa-miR-155-5p. Table 17, below, presents abundance of these two miRNAs in the Treg populations tested (as assessed by read counts), the hsa-miR-146-5p/hsa-miR-155-5p ratio in each of the 6 Treg EV populations tested, and the range of ratios (1.93-5.31) among the six populations. Based on these results, the average hsa-miR-146-5p/hsa-miR-155-5p ratio for the 6 Treg EV populations tested is about 2.7. Table 18, below, depicts the proporation of total miRNA reads that were identified as hsa-miR-146-5p, hsa-miR-155-5p, or hsa-miR-146-5p and hsa-miR-155-5p.

TABLE 17

| miRNA | Treg_EV_1 | Treg_EV_2 | Treg_EV_3 | Treg_EV_4 | Treg_EV_5 | Treg_EV_6 | Range |
|---|---|---|---|---|---|---|---|
| hsa-miR-146a-5p | 105164 | 387840 | 141676 | 177746 | 329422 | 180907 | |
| hsa-miR-155-5p | 49648 | 73026 | 73383 | 58541 | 171730 | 87369 | |
| Ratio of miR-146a/miR-155 | 2.12 | 5.31 | 1.93 | 3.04 | 1.92 | 2.07 | 1.93-5.31 |

TABLE 18

| | Treg | Treg | Treg | Treg | Treg | Treg | Treg EV |
|---|---|---|---|---|---|---|---|
| miRNA | EV 1 | EV 2 | EV 3 | EV 4 | EV 5 | EV 6 | AVG |
| Total miRNA | 689554 | 1646852 | 953235 | 1171101 | 2337624 | 1211914 | 1335047 |
| reads | | | | | | | |
| hsa-miR-146a-5p | 105164 | 387840 | 141676 | 177746 | 329422 | 180907 | 220459 |
| % hsa-miR-146a- | 15.25 | 23.55 | 14.86 | 15.18 | 14.09 | 14.93 | 16.51 |
| 5p of total | | | | | | | |
| miRNA reads | | | | | | | |
| hsa-miR-155-5p | 49648 | 73026 | 73383 | 58541 | 171730 | 87369 | 85616 |
| % hsa-miR-155- | 7.20 | 4.43 | 7.70 | 5.00 | 7.35 | 7.21 | 6.41 |
| 5p of total | | | | | | | |
| miRNA reads | | | | | | | |
| % hsa-miR-146a- | 22.45 | 27.98 | 22.56 | 20.18 | 21.44 | 22.14 | 22.93 |
| 5p and hsa-miR- | | | | | | | |
| 155-5p of total | | | | | | | |
| miRNA reads | | | | | | | |

All publications, patents and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

What is claimed is:

1. An isolated, cell-free population of anti-inflammatory extracellular vesicles (EVs), wherein the anti-inflammatory EVs are derived from ex vivo-expanded human suppressive immune cells,
wherein:
i) the population exhibits a size diameter distribution of about 50 nm to about 150 nm;
ii) the population comprises EV surface CD2, CD25 and HLA-DRDPDQ;
iii) the population comprises hsa-miR-1290, hsa-miR-146a-5p, and hsa-miR-155-5p micro-RNAs (miRNAs); and
iv) the population exhibits an ability to suppress myeloid cells, as measured by an ability to reduce pro-inflammatory cytokine production by the myeloid cells and an ability to increase the expression of one or more anti-inflammatory markers in the myeloid cells, or as measured by an ability to suppress proliferation of responder T cells; and
wherein the human suppressive immune cells are regulatory T cells (Tregs).

2. The population of anti-inflammatory EVs of claim 1, wherein at least about 90% of the EVs in the population exhibit a size diameter of about 50 nm to about 150 nm.

3. The population of anti-inflammatory EVs of claim 1 or 2, wherein the population exhibits a mean size diameter of about 80 nm to about 110 nm.

4. The population of anti-inflammatory EVs of any one of claims 1-3, wherein the population exhibits a median size diameter of about 70 nm to about 110 nm.

5. The population of anti-inflammatory EVs of any one of claims 1-4, wherein the population exhibits a mode size diameter of about 65 nm to about 95 nm.

6. The population of anti-inflammatory EVs of claim 1, wherein at least about 90% of the EVs in the population exhibit a size diameter of about 50 to about 150 nm, and the population exhibits a mean size diameter of about 80 nm to about 110 nm, a median size diameter of about 70 nm to about 110 nm, and a mode size diameter of about 65 nm to about 95 nm.

7. The population of anti-inflammatory EVs of any one of claims 1-6, wherein the population further comprises EV surface CD44, CD29, CD4 and CD45.

8. The population of anti-inflammatory EVs of any one of claims 1-7, wherein the population further comprises EV surface CD9, CD63 and CD81.

9. The population of anti-inflammatory EVs of any one of claims 1-8, wherein the population substantially lacks EV surface CD3, CD19, CD8, CD56, CD105, CD1c, CD49e, ROR1, CD209, SSEA-4, CD40, CD62P, CD11c, CD40, MSCP, CD146, CD86, CD326, CD133, CD142, CD31 and CD14.

10. The population of anti-inflammatory EVs of claim 1 or 6, wherein the population further comprises EV surface CD44, CD29, CD4, CD45, CD9, CD63 and CD81, and wherein the population substantially lacks EV surface CD3, CD19, CD8, CD56, CD105, CD1c, CD49e, ROR1, CD209, SSEA-4, CD40, CD62P, CD11c, CD40, MSCP, CD146, CD86, CD326, CD133, CD142, CD31 and CD14.

11. The population of anti-inflammatory EVs of any one of claims 1-10, wherein the ratio of hsa-miR-146a-5p to hsa-miR-155-5p in the population is about 2 to about 3.

12. The population of anti-inflammatory EVs of any one of claims 1-11, the abundance of hsa-miR-1290 is at least 2-fold that of hsa-mir-155-5p.

13. The population of anti-inflammatory EVs of any one of claims 1-12, wherein the Tregs are from a healthy human subject.

14. The population of anti-inflammatory EVs of any one of claims 1-12, wherein the Tregs are from a human subject diagnosed with or suspected of having Amyotrophic Lateral Sclerosis (ALS).

15. The population of anti-inflammatory EVs of any one of claims 1-14, wherein the anti-inflammatory EVs exhibit an ability to increase the expression of IL-10, Arg1 and/or CD206 in the myeloid cells.

16. The population of anti-inflammatory EVs of any one of claims 1-15, wherein the anti-inflammatory EVs exhibit an ability to decrease the expression of IL-6, IL-8, IL1β or Interferon-γ in the myeloid cells.

17. The population of anti-inflammatory EVs of claim 1, wherein the proliferation of responder T cells is determined by flow cytometry or thymidine incorporation.

18. The population of anti-inflammatory EVs of any one of claims 1-17, wherein the population is a saline-containing population of anti-inflammatory EVs.

19. An isolated, cell-free population of anti-inflammatory extracellular vesicles (EVs), wherein the anti-inflammatory EVs are derived from ex vivo-expanded human suppressive immune cells.

20. The population of anti-inflammatory EVs of claim 19, wherein the human suppressive immune cells are regulatory T cells (Tregs).

21. The population of anti-inflammatory EVs of claim 20, wherein the Tregs are from a healthy human subject.

22. The population of anti-inflammatory EVs of claim 21, wherein the Tregs are from a human subject diagnosed with or suspected of having a neurodegenerative disorder.

23. The population of anti-inflammatory EVs of claim 22, wherein the neurodegenerative disorder is Alzheimer's disease.

24. The population of anti-inflammatory EVs of claim 22, wherein the neurodegenerative disorder is Amyotrophic Lateral Sclerosis (ALS).

25. The population of anti-inflammatory EVs of claim 22, wherein the neurodegenerative disease is multiple sclerosis (MS).

26. The population of anti-inflammatory EVs of claim 22, wherein the neurodegenerative disease is Parkinson's Disease.

27. The population of anti-inflammatory EVs of claim 20, wherein the Tregs are from a human subject who is diagnosed as having, or suspected of having had, a stroke.

28. The population of anti-inflammatory EVs of claim 20 wherein the Tregs are from a geriatric human subject.

29. The population of anti-inflammatory EVs of any one of claims 20-28, wherein the Tregs are from multiple human subjects.

30. The population of anti-inflammatory EVs of claim 29, wherein the Tregs are from multiple unrelated human subjects.

31. The population of anti-inflammatory EVs of any one of claims 19-30, wherein the anti-inflammatory EVs exhibit an ability to increase the expression of one or more anti-inflammatory markers in inflammatory cells.

32. The population of anti-inflammatory EVs of claim 31, wherein the inflammatory cells are myeloid cells.

33. The population of anti-inflammatory EVs of claim 31 or 32, wherein the anti-inflammatory EVs exhibit an ability to increase the expression of IL-10, Arg1 and/or CD206 in inflammatory cells.

34. The population of anti-inflammatory EVs of any one of claims 19-33, wherein the anti-inflammatory EVs exhibits an ability to suppress inflammatory cells, as measured by pro-inflammatory cytokine production by the inflammatory cells.

35. The method of claim 34, wherein the inflammatory cells are myeloid cells.

36. The population of anti-inflammatory EVs of claim 35, wherein the myeloid cells are monocytes, macrophages, or microglia.

37. The population of anti-inflammatory EVs of claim 36, wherein the macrophages are M1 macrophages.

38. The population of anti-inflammatory EVs of claim 37, wherein the M1 macrophages are induced pluripotent stem cell (iPSC)-derived M1 macrophages.

39. The population of anti-inflammatory EVs of any one of claims 31-38, wherein the ability to suppress inflammatory cells is measured by IL-6, IL-8, TNFα, IL1β and/or Interferon-γ production by the inflammatory cells.

40. The population of anti-inflammatory EVs of any one of claims 19-39 wherein the anti-inflammatory EVs exhibit a suppressive function, as determined by suppression of proliferation of responder T cells.

41. The population of anti-inflammatory EVs of claim 40, wherein the proliferation of responder T cells is determined by flow cytometry or thymidine incorporation.

42. The population of anti-inflammatory EVs of any one of claims 19-41, wherein the population is a saline-containing population of anti-inflammatory EVs.

43. The population of anti-inflammatory EVs of any one of claims 19-41, wherein the population is a physiological saline-containing population of anti-inflammatory EVs.

44. The population of anti-inflammatory EVs of any one of claims 19-41, wherein the population is a phosphate-buffered saline-containing population of anti-inflammatory EVs.

45. The population of anti-inflammatory EVs of any one of any one of claims 19-44, wherein the population of anti-inflammatory EVs comprises exosomes and microvesicles.

46. The population of anti-inflammatory EVs of claim 45, wherein the majority of the EVs are exosomes.

47. The population of anti-inflammatory EVs of claim 46, wherein at least about 80%, about 90%, or about 95% of the EVs are exosomes.

48. The population of anti-inflammatory EVs of claim 47 wherein the majority of the EVs are microvesicles.

49. The population of anti-inflammatory EVs of claim 48, wherein at least about 80%, about 90%, or about 95% of the EVs are microvesicles.

50. The population of anti-inflammatory EVs of claim 45, wherein the majority of the EVs have diameters from about 30 nm to about 1000 nm.

51. The population of anti-inflammatory EVs of claim 45, wherein the majority of the EVs have diameters from about 30 nm to about 100 nm, about 30 nm to about 150 nm, about 30 to about 200 nm, about 40 to about 100 nm, about 80 to about 100 nm, about 80 to about 110 nm, about 80 to about 125 nm, or about 100 to about 120 nm.

52. The population of anti-inflammatory EVs of claim 25 wherein the majority of the EVs have diameters from about 60 nm to about 1000 nm, about 70 nm to about 1000 nm, about 80 nm to about 1000 nm, 100 to about 1000 nm, about 200 to about 1000 nm, or about 300 to about 1000 nm.

53. A pharmaceutical composition comprising an isolated, cell-free population of anti-inflammatory EVs of any one of claims 1-52.

54. The pharmaceutical composition of claim 53, wherein the population of anti-inflammatory EVs comprises about 1×10⁶ to about 1×10¹⁴ EVs, about 1×10⁸ to about 1×10¹⁴ EVs, about 1×10⁸ to about 1×10¹² EVs, about 1×10⁸ to about 1×10¹⁰ EVs, about 1×10¹⁰ to about 1×10¹⁴ EVs, or about 1×10¹⁰ to about 1×10¹² EVs.

55. The pharmaceutical composition of claim 53, wherein the population of anti-inflammatory EVs comprises about 1×10⁶ to about 1×10¹⁴ EVs/ml, about 1×10⁸ to about 1×10¹⁴ EVs/ml, about 1×10⁸ to about 1×10¹² EVs/ml, about 1×10⁸ to about 1×10¹⁰ EVs/ml, about 1×10¹⁰ to about 1×10¹⁴ EVs/ml, or about 1×10¹⁰ to about 1×10¹² EVs/ml.

56. The pharmaceutical composition of claim 53, wherein the population of anti-inflammatory EVs comprises about 1 μg to about 200 mg EVs.

57. The pharmaceutical composition of claim 53, wherein the population of anti-inflammatory EVs comprises about 1 μg to about 15 mg EVs.

58. The pharmaceutical composition of claim 53, wherein the population of anti-inflammatory EVs comprises about 1 μg to about 15 mg EV/ml.

59. The pharmaceutical composition of any one of claims 53-58, wherein the pharmaceutical composition is a cryopreserved pharmaceutical composition.

60. The pharmaceutical composition of any one of claims 53-58, wherein the pharmaceutical composition had previously been cryopreserved.

61. A cryopreserved composition comprising an isolated, cell-free population of anti-inflammatory EVs of any one of claims 1-53.

62. A method of producing an isolated, cell-free population of anti-inflammatory extracellular vesicles (EVs), said method comprising the steps of:
a. ex-vivo expanding a human suppressive immune cell population in culture media to produce a culture comprising the cells, the culture media and anti-inflammatory EVs; and
b. isolating the anti-inflammatory EVs from the culture.

63. The method of claim 62, wherein the human suppressive immune cell population is a population of regulatory T cells (Tregs).

64. The method of claim 62 or 63 wherein step b) comprises removing cells from the culture, followed by polyethylene glycol precipitation of the culture.

65. The method of claim 62 or 63, wherein step b) comprises:
i) removing the cells from the culture to produce a cell-free, anti-inflammatory EV-containing solution; and
ii) isolating the anti-inflammatory EVs from the cell-free, anti-inflammatory EV-containing solution of i).

66. The method of claim 65, wherein step i) comprises passing the culture through a filter such that the cells are retained by the filter, and thereby removed from the culture.

67. The method of claim 65 or 66, wherein step i) comprises microfiltration.

68. The method of any one of claims 65-67, wherein step ii) comprises step ii-a): passing the cell-free, anti-inflammatory EV-containing solution through a filter such that the anti-inflammatory EVs are retained by the filter.

69. The method of claim 68, wherein the filter has a molecular weight cut-off (MWCO) of about 200 kilodaltons (kDa) to about 600 kDa.

70. The method of claim 69, wherein the filter has an MWCO of about 500 kDa.

71. The method of any one of claims 65-70, wherein step ii) comprises ultrafiltration.

72. The method of any one of claims 68-71, wherein step ii) further comprises step ii-b):
performing buffer exchange such that the isolated, cell-free population of anti-inflammatory EVs produced is a buffer-containing isolated, cell-free population of anti-inflammatory EVs.

73. The method of claim 72, wherein the buffer is a saline-containing buffer.

74. The method of claim 73, wherein the saline-containing buffer is physiological saline.

75. The method of claim 74, wherein the saline-containing buffer is PBS.

76. The method of any one of claims 73-75, wherein step ii-b) comprises diafiltration.

77. The method of any one of claim 73-76 wherein steps ii-a) and ii-b) are performed simultaneously.

78. The method of any one of claims 62-77, wherein step b) comprises tangential flow filtration.

79. The method of any one of claims 62-78, wherein the culture media in step a) is serum-free.

80. The method of any one of claims 62-79, wherein the culture media in step a) comprises serum.

81. The method of claim 80, wherein the serum is human AB serum.

82. The method of claim 80 or 81, wherein the serum is depleted for serum-derived EVs.

83. The method of any one of claims 62-82 further comprising, prior to step a), the step of enriching Tregs from a cell sample suspected of containing Tregs, to produce a baseline Treg cell population that is the population of Tregs that is then expanded in a).

84. The method of claim 83, wherein the cell sample is a leukapheresis cell sample.

85. The method of claim 83 or 84, wherein the method further comprises obtaining the cell sample from a donor by leukapheresis.

86. The method of any one of claims 83-85, wherein the cell sample is not stored overnight or frozen before carrying out the enriching step.

87. The method of any one of claims 83-86, wherein the cell sample is obtained within 30 minutes before initiation of enriching step.

88. The method of any one of claims 82-87, wherein the enriching step comprises depleting CD8+/CD19+ cells then enriching for CD25+ cells.

89. The method of any one of claims 62-88, wherein step a) is carried out within 30 minutes of the enriching step.

90. The method of any one of claims 62-89, wherein step a) comprises culturing the Tregs in a culture media that comprises beads coated with anti-CD3 antibodies and anti-CD28 antibodies.

91. The method of claim 90, wherein the beads are first added to the culture media within about 24 hours of the initiation of the culturing.

92. The method of claim 90 or 91, wherein beads coated with anti-CD3 antibodies and anti-CD28 antibodies are added to the culture media about 14 days after beads coated with anti-CD3 antibodies and anti-CD28 antibodies were first added to the culture medium.

93. The method of any one of claims 90-92, wherein step a) further comprises adding IL-2 to the culture medium within about 6 days of the initiation of culturing.

94. The method of claim 93, wherein step a) further comprises replenishing the culture medium with IL-2 about every 2-3 days after IL-2 is first added to the culture medium.

95. The method of any one of claims 90-94, wherein step a) further comprises adding rapamycin to the culture medium within about 24 hours of the initiation of the culturing.

96. The method of claim 95, wherein step a) further comprises replenishing the culture medium with rapamycin every 2-3 days after the rapamycin is first added to the culture medium.

97. The method of any one of claims 62-96, wherein step a) is automated.

98. The method of any one of claims 62-97, wherein step a) takes place in a bioreactor.

99. The method of any one of claims 62-98, wherein step b) may commence at any point during step a).

100. The method of any one of claims 63-99, wherein the Tregs are from a healthy human subject.

101. The method of any one of claims 63-99, wherein the Tregs are from a human subject diagnosed with or suspected of having a neurodegenerative disorder.

102. The method of claim 101, wherein the neurodegenerative disorder is Alzheimer's disease, Amyotrophic Lateral Sclerosis (ALS), multiple sclerosis (MS), or Parkinson's Disease.

103. The method of any one of claims 63-102, wherein the Tregs are from a human subject who is diagnosed as having, or suspected of having had, a stroke.

104. The method of any one of claims 63-102, wherein the Tregs are from a geriatric human subject.

105. The method of any one of claims 63-104, wherein the Tregs are from multiple human subjects.

106. The method of claim 62, wherein the human suppressive immune cell population is a genetically engineered human suppressive immune cell population.

107. The method of any one of claims 63-106, wherein the population of Tregs is a genetically engineered population of Tregs.

108. A pharmaceutical composition comprising an isolated, cell-free population of anti-inflammatory EVs, wherein the population is made by any one of the methods of claim 62-107.

109. The method of any one of claims 62-107, further comprising: c) cryopreserving the isolated, cell-free population of anti-inflammatory EVs, thereby producing a cryopreserved, isolated, cell-free population of anti-inflammatory EVs.

110. The method of claim 109, further comprises thawing the cryopreserved, isolated cell-free population of anti-inflammatory EVs after cryopreservation for about 1 week, 1 month, about 3 months, about 6 months, about 9 months, about 12 months, about 18 months or about 24 months.

111. A pharmaceutical composition comprising the isolated, cell-free population of anti-inflammatory EVs of claim 110.

112. An isolated, cell-free population of anti-inflammatory EVs, wherein the anti-inflammatory EVs are derived from an ex vivo-expanded Treg cell population that exhibits an ability to suppress inflammatory cells, as measured by pro-inflammatory cytokine production by the inflammatory cells, wherein the inflammatory cells are macrophages or monocytes from human donors or generated from induced pluripotent stem cells, wherein the ex vivo-expanded Treg cell population has been expanded from baseline Tregs, and wherein, in the ex vivo-expanded Treg cell population:
a) expression of one or more dysfunctional baseline signature gene products listed in Table 3 and/or Table 4 is decreased relative to the expression of the one or more gene products in baseline Tregs;
b) expression of one or more dysfunctional baseline signature gene products listed in Table 5 is decreased relative to the expression of the one or more gene products in baseline Tregs;
c) expression of one or more Treg-associated signature gene products listed in Table 6 is increased relative to the expression of the one or more gene products in baseline Tregs;
d) expression of one or more mitochondria signature gene products listed in Table 7 is increased relative to the expression of the one or more gene products in baseline Tregs;
e) expression of one or more cell proliferation signature gene products listed in Table 8 is increased relative to the expression of the one or more gene products in baseline Tregs; or
f) expression of one or more highest protein expression signature gene products listed in Table 9 is increased relative to the expression of the one or more gene products in baseline Tregs.

113. A pharmaceutical composition comprising the isolated, cell-free population of anti-inflammatory EVs of claim 112.

114. A method of treating a disorder associated with Treg dysfunction, the method comprising administering to a subject in need of said treatment the composition of any one of claim 53-60, 108, 111, or 113.

115. A method of treating a disorder associated with Treg deficiency, the method comprising administering to a subject in need of said treatment the pharmaceutical composition of any one of claim 53-60, 108, 111, or 113.

116. A method of treating a disorder associated with overactivation of the immune system, the method comprising administering to a subject in need of said treatment the pharmaceutical composition of any one of claim 53-60, 108, 111, or 113.

117. A method of treating an inflammatory condition driven by a T cell response, the method comprising administering to a subject in need of said treatment the pharmaceutical composition of any one of claim 53-60, 108, 111, or 113.

118. A method of treating an inflammatory condition driven by a myeloid cell response, the method comprising administering to a subject in need of said treatment the pharmaceutical composition of any one of claim 53-60, 108, 111, or 113.

119. The method of claim 118, wherein the myeloid cell is a monocyte, macrophage or microglia.

120. A method of treating a neurodegenerative disorder in a subject in need thereof, the method comprising administering to a subject in need of said treatment the pharmaceutical composition of any one of claim 53-60, 108, 111, or 113.

121. The method of claim 120, wherein the neurodegenerative disease is ALS, Alzheimer's disease, Parkinson's disease, frontotemporal dementia or Huntington's disease.

122. A method of treating an autoimmune disorder in a subject in need thereof, the method comprising administering to a subject in need of said treatment the pharmaceutical composition of any one of claim 53-60, 108, 111, or 113.

123. The method of claim 122, wherein the autoimmune disorder is polymyositis, ulcerative colitis, inflammatory bowel disease, Crohn's disease, celiac disease, systemic sclerosis (scleroderma), multiple sclerosis (MS), rheumatoid arthritis (RA), Type I diabetes, psoriasis, dermatomyosititis, systemic lupus erythematosus, cutaneous lupus, myasthenia gravis, autoimmune nephropathy, autoimmune hemolytic anemia, autoimmune cytopenia, autoimmune encephalitis, autoimmune hepatitis, autoimmune uveitis, alopecia, thyroiditis or pemphigus.

124. A method of treating graft-versus-host disease in a subject in need thereof, the method comprising administering to a subject in need of said treatment the pharmaceutical composition of any one of claim 53-60, 108, 111, or 113, The method of claim 106, wherein the subject has received a bone marrow transplant, kidney transplant or liver transplant.

125. A method of improving islet graft survival in a subject in need thereof, the method comprising administering to a subject in need of said treatment the pharmaceutical composition of any one of claim 53-60, 108, 111, or 113.

126. A method of treating cardio-inflammation in a subject in need thereof, the method comprising administering to a subject in need of said treatment the pharmaceutical composition of any one of claim 53-60, 108, 111, or 113.

127. The method of claim 126, wherein the cardio-inflammation is associated with atherosclerosis, myocardial infarction, ischemic cardiomyopathy or heart failure.

128. A method of treating neuroinflammation in a subject in need thereof, the method comprising administering to a subject in need of said treatment the pharmaceutical composition of any one of claim 53-60, 108, 111, or 113.

129. The method of claim 128, wherein the neuroinflammation is associated with stroke, acute disseminated encephalomyelitis, acute optic neuritis, acute inflammatory demyelinating polyradiculoneuropathy, chronic inflammatory demyelinating polyradiculoneuropathy, Guillain-Barre syndrome, transverse myelitis, neuromyelitis optica, epilepsy, traumatic brain injury, spinal cord injury, encephalitis, central nervous system vasculitis, neurosarcoidosis, autoimmune or post-infectious encephalitis or chronic meningitis.

130. A method of treating a Tregopathy in a subject in need thereof, comprising administering to a subject in need of said treatment the pharmaceutical composition of any one of claim 53-60, 108, 111, or 113.

131. The method of claim 130, wherein the Tregopathy is caused by a FOXP3, CD25, cytotoxic T lymphocyte-associated antigen 4 (CTLA4), LPS-responsive and beige-like anchor protein (LRBA), or BTB domain and CNC homolog 2 (BACH2) gene loss-of-function mutation, or a signal transducer and activator of transcription 3 (STAT3) gain-of-function mutation.

132. The method of any one of claims 114-131, wherein the anti-inflammatory EVs are derived from Tregs that are autologous to the subject.

133. The method of any one of claims 114-131 wherein the anti-inflammatory EVs are derived from Tregs that are allogeneic to the subject.

134. The method of any one of claim 114-133, wherein the pharmaceutical composition is administered via intranasal administration.

135. The method of claim 134 wherein the intranasal administration is via aerosol inhalation or nasal drip.

136. The method of any one of claim 114-135, wherein the pharmaceutical composition is administered intravenously.

137. The method of any one of claim 114-135, wherein the pharmaceutical composition is administered by local injection.

138. The method of any one of claims 114-137, wherein the method further comprises administering to the subject a pharmaceutical composition comprising a therapeutic population of Tregs, wherein the Tregs had been ex vivo expanded and cryopreserved, and wherein the Tregs are not further expanded prior to the administering.

139. The method of claim 138, wherein the therapeutic population of Tregs is autologous to the subject.

140. The method of claim 138, wherein the therapeutic population of Tregs is allogeneic to the subject.

141. The method of any one of claims 138-140, wherein the pharmaceutical composition comprising the therapeutic population of Tregs is administered intravenously.

142. The method of any one of claims 138-141, wherein the pharmaceutical composition comprising the anti-inflammatory EVs and the pharmaceutical composition comprising the therapeutic population of Tregs are administered to the patient on the same day.

143. The method of any one of claims 114-140, wherein the isolated, cell-free population of anti-inflammatory EVs had been cryopreserved and thawed prior to being administered to the subject.

144. The method of any one of claims 114-140, wherein the isolated, cell-free population of anti-inflammatory EVs are stored overnight at 4° C. prior to being administered to the subject.

145. The method of claim 144, wherein the isolated, cell-free population of anti-inflammatory EVs had been cryopreserved then thawed and stored at 4° C. overnight prior to being administered to the subject.

146. The method of any one of claims 114-140, wherein the isolated, cell-free population of anti-inflammatory EVs had undergone at least two freeze/thaw cycles prior to being administered to the subject.

147. The method of claim 146, wherein the isolated, cell-free population of anti-inflammatory EVs had undergone about 2 to about 20 freeze/thaw cycles prior to being administered to the subject.
